(19) United States
(12) Patent Application Publication
(10) Pub. No.: US 2016/0016951 A1
(43) Pub. Date: Jan. 21, 2016

(54) NOVEL NAPHTHYRIDINES AND ISOQUINOLINES AND THEIR USE AS CDK8/19 INHIBITORS

(71) Applicants: Merck Patent GmbH, Darmstadt (DE); Cancer Research Technology, Ltd., London (GB)

(72) Inventors: Kai SCHIEMANN, Seeheim-Jugenheim (DE); Julian BLAGG, Surrey (GB); Aurelie MALLINGER, Affleville (FR); Christian RINK, Bonn (DE); Jimmy SEJBERG, London (GB); Mark HONEY, Kent (GB)

(21) Appl. No.: 14/802,042

(22) Filed: Jul. 17, 2015

Related U.S. Application Data
(60) Provisional application No. 62/181,264, filed on Jun. 18, 2015.
(60) Provisional application No. 62/025,749, filed on Jul. 17, 2014.

(51) Int. Cl. C07D 471/04; C07D 401/12; C07D 417/14; C07D 401/14; C07D 413/14; C07D 401/10

(52) U.S. Cl. CPC C07D 471/04; C07D 413/14; C07D 401/10; C07D 417/14; C07D 401/14; C07D 401/12

(57) ABSTRACT

The present invention relates to naphthyridine and isoquinoline compounds, and pharmaceutically acceptable compositions thereof, useful as inhibitors of CDK8/19, and for the treatment of CDK8/19-related disorders.

### RELATED APPLICATION

This application claims the benefit of U.S. Provisional applications 62/025,749, filed on Jul. 17, 2014 and 62/181,264, filed on Jun. 18, 2015, the content of which is incorporated by reference in its entirety.

### TECHNICAL FIELD OF THE INVENTION

The present invention relates to naphthyridine and isoquinoline compounds useful as inhibitors of CDK8/19. The invention also provides pharmaceutically acceptable compositions comprising compounds of the present invention and methods of using said compositions in the treatment of various disorders.

### BACKGROUND OF THE INVENTION

CDK8, along with its closely related isoform CDK19, is an oncogenic transcription-regulating kinase. In contrast to better-known members of the CDK family (such as CDK1, CDK2, and CDK4/6), CDK8 plays no direct role in cell cycle progression. CDK8 knockout in embryonic stem cells prevents embryonic development, due to its essential role in the pluripotent stem cell phenotype but CDK8 depletion does not inhibit the growth of normal cells.

The role of CDK8 in cancer is due to its unique function as a regulator of several transcriptional programs involved in carcinogenesis. CDK8 has been identified as an oncogene in melanoma and colon cancer, the CDK8 gene being amplified in about 50% of the latter cancers. Higher expression of CDK8 has been associated with worse prognosis in colon, breast and ovarian cancer. The known cancer-relevant activities of CDK8 include positive regulation of Wnt/β-catenin pathway, growth factor-induced transcription and TGFβ signaling. CDK8 was also shown to maintain the pluripotent phenotype of embryonic stem cells and has been associated with the cancer stem cell phenotype. DNA-damaging chemotherapeutic drugs induce TNFα, an activator of the transcription factor NFkB, in endothelial cells and in other cancer-associated stromal elements. Stroma-derived TNFα acts on tumor cells, where it induces NFkB-mediated production of related tumor-promoting cytokines CXCL1 and CXCL2. CXCL1/2 attract myeloid cells to the tumor, by binding to CXCR2 receptor on the myeloid cell surface. Myeloid cells then secrete small calcium-binding proteins 5100A8 and A9 that are associated with chronic inflammation and cancer. 5100A8/9 act on tumor cells, promoting both their metastasis and survival of chemotherapy.

CDK8 is a cyclin dependent kinase that has a conserved function in transcription as part of the Mediator complex. Taatjes, D. J., Trends Biochem Sci 35, 315-322 (2010); Conaway, R. C. and Conaway, J. W., Curr Opin Genet Dev 21, 225-230 (2011). More recently, CDK8 has been reported to as an oncogene in both colon cancer (Firestein R. et al., Nature 455:547-51 (2008); Morris E. J. et al., Nature 455:552-6 (2008); Starr T. K. et al., Science 323:1747-50 (2009)) and melanoma (Kapoor A. et al., Nature 468:1105-9 (2010)). CDK8 is upregulated and amplified in a subset of human colon tumors. CDK8 transforms immortalized cells and is required for colon cancer proliferation in vitro (Firestein, R. et al., Nature 455, 547-551 (2008)). CDK8 has also been found to be overexpressed and essential for proliferation in melanoma (Kapoor, A. et al., Nature 468, 1105-1109 (2010)). CDK8 has been shown to regulate several signaling pathways that are key regulators of both ES pluripotency and cancer. CDK8 activates the Wnt pathway by promoting expression of β-Catenin target genes (Firestein, R. et al., Nature 455, 547-551 (2008)) or by inhibiting E2F1, a potent inhibitor of β-Catenin transcriptional activity (Morris, E. J. et al., Nature 455, 552-556 (2008)). CDK8 promotes Notch target gene expression by phosphorylating the Notch intracellular domain, activating Notch enhancer complexes at target genes (Fryer C. J. et al., Mol Cell 16:509-20 (2004)). Lastly, CDK8 phosphorylation of SMAD proteins leads to activation of TGF-β/BMP target genes followed by degradation of the SMAD proteins to limit the target gene expression (Alarcon, C. et al., Cell 139, 757-769 (2009)).

### SUMMARY OF THE INVENTION

It has now been found that compounds of this invention, and pharmaceutically acceptable compositions thereof, are effective as inhibitors of CDK8/19. Such compounds have general formula I:

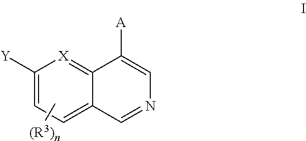

or a pharmaceutically acceptable salt thereof, wherein each of A, X, Y, and n, is as defined and described in embodiments herein.

Compounds of the present invention, and pharmaceutically acceptable compositions thereof, are useful for treating a variety of diseases, disorders or conditions, associated with CDK8/19 activity. Such diseases, disorders, or conditions include those described herein.

### DETAILED DESCRIPTION OF CERTAIN EMBODIMENTS

### 1. General Description of Compounds of the Invention

In certain embodiments, the present invention provides inhibitors of CDK8/19. In some embodiments, such compounds include those of the formulae described herein, or a pharmaceutically acceptable salt thereof, wherein each variable is as defined and described herein.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, or phosphorus (including, any oxidized form of nitrogen, sulfur, or phosphorus; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group. The term “alkynylene” refers to a bivalent alkynyl group. A substituted alkynylene chain is a group containing at least one triple bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydropyranyl, tetrahydrothiophenyl pyrrolidinyl, piperidinyl, pyrrolinyl, morpholinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds of the invention contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

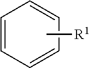

refers to at least

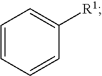

and

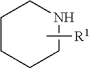

refers to at least

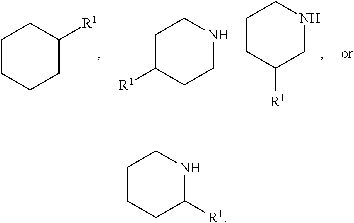

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{◯}; —(CH₂)₀₋₄R^{◯}; —O(CH₂)₀₋₄R^{◯}, —O—(CH₂)⁰⁻⁴C(O)OR^{◯}; —(CH₂)₀₋₄CH(OR^{◯)}₂; —(CH₂)₀₋₄SR^{◯}; —(CH₂)₀₋₄Ph, which are optionally substituted with R^{◯}; —(CH₂)₀₋₄O(CH₂)₀₋₁Ph which is optionally substituted with R^{◯}; —CH═CHPh, which is optionally substituted with R^{◯}; —(CH₂)₀₋₄O(CH₂)₀₋₁-pyridyl which is optionally substituted with R^{◯}; —NO₂; —CN; —N₃; —(CH₂)₀₋₄N(R^{◯)}₂; —(CH₂)₀₋₄N(R^{◯}C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯}C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯)}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯)}₂; —OP(O)R^{◯}₂; —OP(O)(OR^{◯)}₂; SiR^{◯}₃; —(C₁₋₄ straight or branched alkylene)O—N(R^{◯)}₂; or —(C₁₋₄ straight or branched)alkylene)C(O)O—N(R^{◯)}₂, wherein each R^{◯} is optionally substituted as defined below and is independently hydrogen, C₁₋₆ aliphatic, —CH₂Ph, —O(CH₂)₀₋₁Ph, —NH(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which is optionally substituted as defined below.

Suitable monovalent substituents on R^{◯} (or the ring formed by taking two independent occurrences of R^{◯} together with their intervening atoms), are independently deuterium, halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)_{0- 2}NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{\} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:
—F, —Cl, —Br, —I, deuterium,
—OH, protected hydroxy, alkoxy, oxo, thiooxo,
—NO₂, —CN, CF₃, N₃,
—NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino,
—O— alkyl, —O— alkenyl, —O— alkynyl, —O— cycloalkyl, —O-aryl, —O-heteroaryl, —O-heterocyclic,
—C(O)— alkyl, —C(O)— alkenyl, —C(O)— alkynyl, —C(O)— carbocyclyl, —C(O)-aryl, —C(O)— heteroaryl, —C(O)-heterocyclyl,
—CONH₂, —CONH— alkyl, —CONH— alkenyl, —CONH— alkynyl, —CONH-carbocyclyl, —CONH-aryl, —CONH-heteroaryl, —CONH-heterocyclyl,
—OCO₂-alkyl, —OCO₂-alkenyl, —OCO₂-alkynyl, —OCO₂-carbocyclyl, —OCO₂-aryl, —OCO₂-heteroaryl, —OCO₂-heterocyclyl, —OCONH₂, —OCONH-alkyl, —OCONH-alkenyl, —OCONH-alkynyl, —OCONH-carbocyclyl, —OCONH-aryl, —OCONH-heteroaryl, —OCONH-heterocyclyl,
—NHC(O)— alkyl, —NHC(O)— alkenyl, —NHC(O)— alkynyl, —NHC(O)— carbocyclyl, —NHC(O)-aryl, —NHC(O)-heteroaryl, —NHC(O)-heterocyclyl, —NHCO₂-alkyl, —NHCO₂-alkenyl, —NHCO₂-alkynyl, —NHCO₂-carbocyclyl, —NHCO₂-aryl, —NHCO₂-heteroaryl, —NHCO₂-heterocyclyl, —NHC(O)NH₂, —NHC(O)NH— alkyl, —NHC(O)NH— alkenyl, —NHC(O)NH— alkenyl, —NHC(O)NH— carbocyclyl, —NHC(O)NH-aryl, —NHC(O)NH-heteroaryl, —NHC(O)NH— heterocyclyl, NHC(S)NH₂, —NHC(S)NH— alkyl, —NHC(S)NH— alkenyl, —NHC(S)NH— alkynyl, —NHC(S)NH— carbocyclyl, —NHC(S)NH-aryl, —NHC(S)NH-heteroaryl, —NHC(S)NH-heterocyclyl, —NHC(NH)NH₂, —NHC(NH)NH— alkyl, —NHC(NH)NH-alkenyl, —NHC(NH)NH— alkenyl, —NHC(NH)NH— carbocyclyl, —NHC(NH)NH-aryl, —NHC(NH)NH-heteroaryl, —NHC(NH)NH— heterocyclyl, —NHC(NH)— alkyl, —NHC(NH)— alkenyl, —NHC(NH)— alkenyl, —NHC(NH)— carbocyclyl, —NHC(NH)-aryl, —NHC(NH)-heteroaryl, —NHC(NH)-heterocyclyl,
—C(NH)NH— alkyl, —C(NH)NH— alkenyl, —C(NH)NH— alkynyl, —C(NH)NH— carbocyclyl, —C(NH)NH-aryl, —C(NH)NH-heteroaryl, —C(NH)NH-heterocyclyl,
—S(O)— alkyl, —S(O)-alkenyl, —S(O)-alkynyl, —S(O)-carbocyclyl, —S(O)-aryl, —S(O)-heteroaryl, —S(O)-heterocyclyl-SO₂NH₂, —SO₂NH-alkyl, —SO₂NH-alkenyl, —SO₂NH-alkynyl, —SO₂NH-carbocyclyl, —SO₂NH-aryl, —SO₂NH-heteroaryl, —SO₂NH-heterocyclyl,
—NHSO₂-alkyl, —NHSO₂-alkenyl, —NHSO₂-alkynyl, —NHSO₂-carbocyclyl, —NHSO₂-aryl, —NHSO₂-heteroaryl, —NHSO₂-heterocyclyl,
—CH₂NH₂, —CH₂SO₂CH₃,
-mono-, di-, or tri-alkyl silyl,
-alkyl, -alkenyl, -alkynyl, -aryl, -arylalkyl, -heteroaryl, -heteroarylalkyl, -heterocycloalkyl,-cycloalkyl,-carbocyclic,-heterocyclic, polyalkoxyalkyl, polyalkoxy, -methoxymethoxy, -methoxyethoxy, —SH, —S— alkyl, —S— alkenyl, —S— alkynyl, —S— carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, tautomers, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

There is furthermore intended that a compound of the formula I includes isotope-labeled forms thereof. An isotope-labeled form of a compound of the formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the formula I by well-known methods include isotopes of hydrogen, carbon, nitrogen, oxygen, phos-phorus, fluo-rine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively. A compound of the formula I, a prodrug, thereof or a pharmaceutically acceptable salt of either which contains one or more of the above-mentioned isotopes and/or other isotopes of other atoms is intended to be part of the present invention. An isotope-labeled compound of the formula I can be used in a number of beneficial ways. For example, an isotope-labeled compound of the formula I into which, for example, a radioisotope, such as ³H or ¹⁴C, has been incorporated, is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of the formula I has therapeutic advantages owing to the higher metabolic stability of this isotope-labeled compound. Higher metabolic stability translates directly into an increased in vivo half-life or lower dosages, which under most circumstances would represent a preferred embodiment of the present invention. An isotope-labeled compound of the formula I can usually be prepared by carrying out the procedures disclosed in the synthesis schemes and the related description, in the example part and in the preparation part in the present text, replacing a non-isotope-labeled reactant by a readily available isotope-labeled reactant. Compounds of the invention may be substituted by ¹⁸F, for use as PET imaging agents.

Deuterium (²H) can also be incorporated into a compound of the formula I for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound of the formula I that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of the formula I with improved stability through resistance to such oxidative metabolism. Significant improvements in the pharmacokinetic profiles of compounds of the formula I are thereby obtained, and can be expressed quantitatively in terms of increases in the in vivo half-life (t1/2), concentration at maximum therapeutic effect (Cₘₐₓ), area under the dose response curve (AUC), and F; and in terms of reduced clearance, dose and materials costs.

The following is intended to illustrate the above: a compound of the formula I which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound of the formula I can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

As used herein, the term “modulator” is defined as a compound that binds to and/or inhibits the target with measurable affinity. In certain embodiments, a modulator has an IC₅₀ and/or binding constant of less about 50 μM. In certain embodiments, a modulator has an IC₅₀ and/or binding constant of less than about 5 μM. In certain embodiments, a modulator has an IC₅₀ and/or binding constant of between about 1 to about 5 μM. In certain embodiments, a modulator has an IC₅₀ and/or binding constant of less than about 1 μM. In certain embodiments, a modulator has an IC₅₀ and/or binding constant of between about 500 to about 1000 nM. In certain embodiments, a modulator has an IC₅₀ and/or binding constant of less than about 500 nM. In certain embodiments, a modulator has an IC₅₀ and/or binding constant of between about 100 to about 500 nM. In certain embodiments, a modulator has an IC₅₀ and/or binding constant of less than about 100 nM. In certain embodiments, a modulator has an IC₅₀ and/or binding constant of between about 10 to about 100 nM. In certain embodiments, a modulator has an IC₅₀ and/or binding constant of less than about 10 nM.

The terms “measurable affinity” and “measurably inhibit,” as used herein, means a measurable change in CDK8/19 activity between a sample comprising a compound of the present invention, or composition thereof, and CDK8/19, and an equivalent sample comprising CDK8/19, in the absence of said compound, or composition thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### 3. Description of Exemplary Compounds

According to one aspect, the present invention provides a compound of formula I,

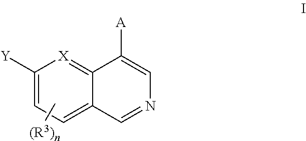

or a pharmaceutically acceptable salt thereof, wherein:
A is hydrogen, C₁₋₆ aliphatic, C₅₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted by R¹ and/or R²; or A is halogen;
X is CR or N;
Y is hydrogen, OR, SR, SO₂R, SOR, C(O)R, CO₂R, C(O)N(R)₂, SO₂N(R)₂, C(NR)N(R)₂, NRC(O)R, NRC(O)N(R)₂, NRSO₂R, N(R)₂; —CN, halogen, C₁₋₆ aliphatic, C₃₋₁₀ aryl, a 3-8 membered saturated or partially unsaturated carbocyclic ring, a 3-7 membered heterocyclic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
each R³ is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R¹ is a 5-6 membered monocyclic heteroaryl ring having 1-4 heteroatoms independently selected from N, NR, O, S, SO, or SO₂, which is optionally substituted by 1-5 of R^{A};
R² is hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or R² is halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂; or
R¹ and R², together with the atoms to which each is attached, forms an optionally substituted fused 5-6 membered heterocyclic or heteroaryl ring having 1-4 heteroatoms independently selected from N, NR, O, S, SO, or SO₂, wherein the ring is not a pyrrole, dihydro-pyrrole, or thiazole;
each R^{A} is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; and
n is 0, 1, 2, 3, or 4.

In certain embodiments, A is hydrogen, C₁₋₆ aliphatic, or C₅₋₁₀ aryl, each of which is optionally substituted by R¹ and/or R²; or A is halogen.

In certain embodiments, A is hydrogen.

In certain embodiments, A is C₁₋₆ aliphatic, optionally substituted by R¹ and/or R².

In certain embodiments, A is methyl, ethyl, propyl, i-propyl, butyl, s-butyl, t-butyl, straight chain or branched pentyl, straight chain or branched hexyl, or straight chain or branched heptyl; each of which is optionally substituted by R¹ and/or R². In certain embodiments, A is methyl.

In certain embodiments, A is halogen. In certain embodiments, A is F, Cl, Br, or I. In certain embodiments, A is Br.

In certain embodiments, A is C₅₋₁₀ aryl, optionally substituted by R¹ and/or R².

In certain embodiments, A is

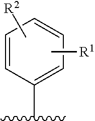

In certain embodiments, A is

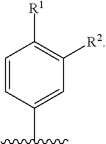

In certain embodiments, R¹ is a 5-6 membered monocyclic heteroaryl ring having 1-4 heteroatoms independently selected from N, NR, O, S, SO, or SO₂, which is optionally substituted by 1-5 of R^{A}.

In certain embodiments, R¹ is benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted by 1-5 of R^{A}.

In certain embodiments, R¹ is pyrazolyl.

In certain embodiments, R¹ is

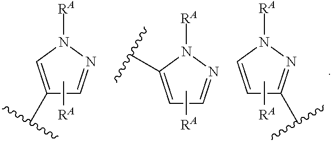

In certain embodiments, R¹ is

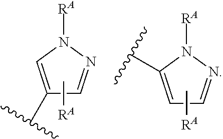

In certain embodiments, R¹ is

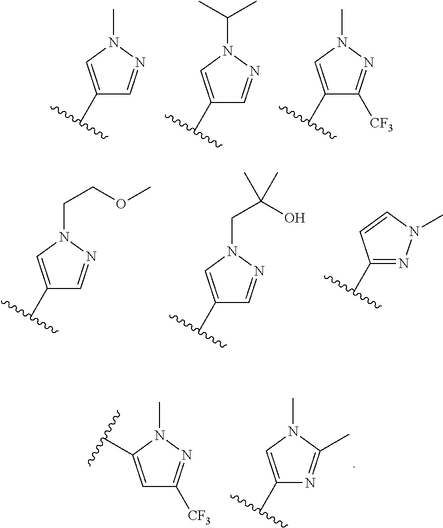

In certain embodiments, R² is hydrogen.

In certain embodiments, R² is C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, R² is halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, R² is C₁₋₆ aliphatic. In certain embodiments, R² is methyl, ethyl, propyl, i-propyl, butyl, s-butyl, t-butyl, straight chain or branched pentyl, straight chain or branched hexyl, or straight chain or branched heptyl; each of which is optionally substituted. In certain embodiments, R² is methyl.

In certain embodiments, R² is halogen. In certain embodiments, R² is F.

In certain embodiments, R¹ and R², together with the atoms to which each is attached, forms an optionally substituted 5-6 membered heterocyclic or heteroaryl ring having 1-4 heteroatoms independently selected from N, NR, O, S, SO, or SO₂, wherein the ring has at least one heteroatom selected from S, SO, and SO₂; or wherein the ring has at least one or two heteroatoms selected from N and NR.

In certain embodiments, R¹ and R², together with the atoms to which each is attached, forms an optionally substituted 5-6 membered heterocyclic or heteroaryl ring having 1-4 heteroatoms independently selected from N, NR, O, S, SO, or SO₂, wherein the ring has at least one heteroatom selected from S, SO, and SO₂.

In certain embodiments, R¹ and R², together with the atoms to which each is attached, forms an optionally substituted 5-6 membered heterocyclic or heteroaryl ring having 2-4 heteroatoms independently selected from N, NR, O, S, SO, or SO₂, wherein the ring has at least one heteroatom selected from N and NR.

In certain embodiments, R¹ and R², together with the atoms to which each is attached, forms an optionally substituted 5-6 membered heterocyclic or heteroaryl ring having 2-4 heteroatoms independently selected from N, NR, O, S, SO, or SO₂, wherein the ring has at least two heteroatoms selected from N and NR.

In certain embodiments, A

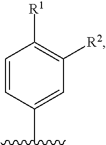

and A, R¹ and R², together with the atoms to which each is attached, is

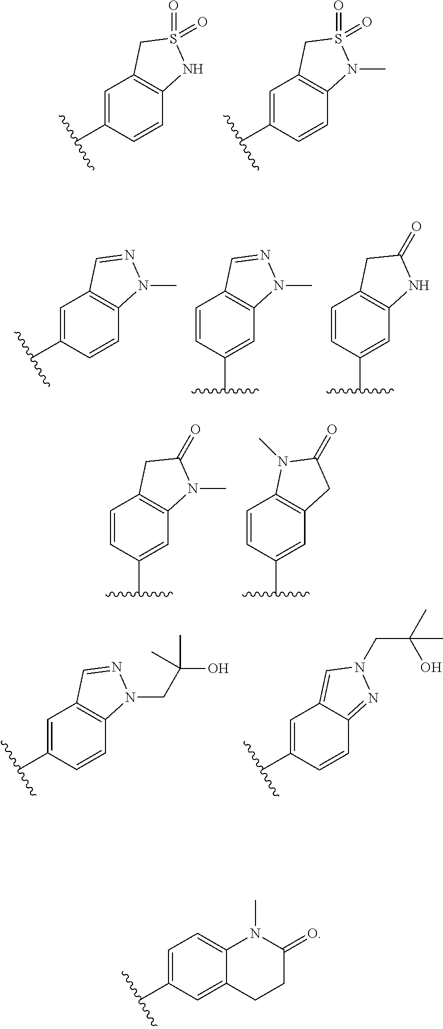

In certain embodiments, X is CR. In certain embodiments, X is CH.

In certain embodiments, X is N.

In certain embodiments, Y is hydrogen.

In certain embodiments, Y is OR, SR, SO₂R, SOR, CO₂R, C(O)N(R)₂, C(NR)N(R)₂, NRC(O)R, NRSO₂R, N(R)₂, —CN, halogen, C₁₋₆ aliphatic, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, Y is

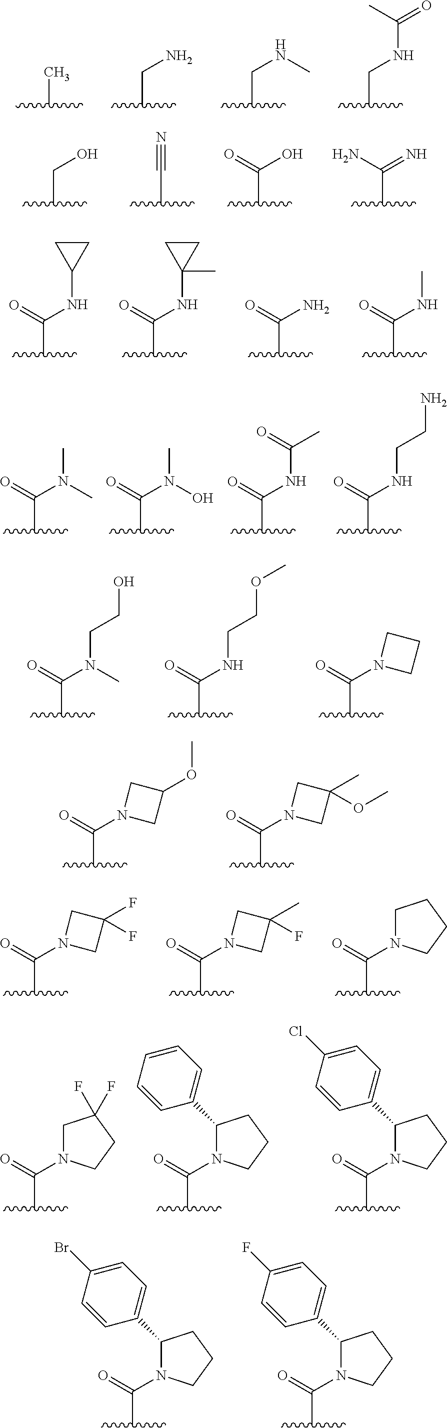

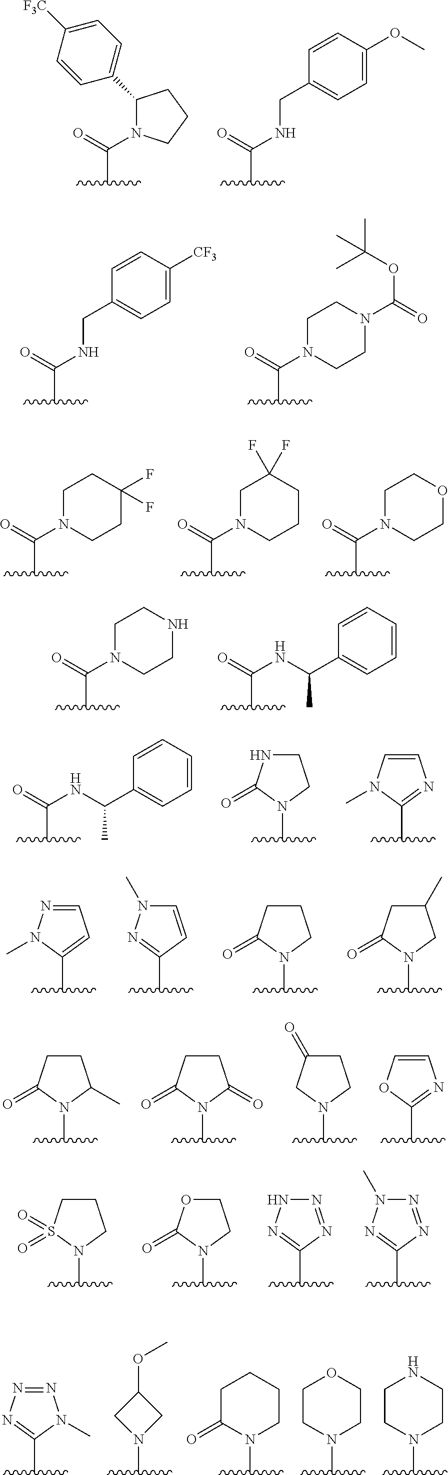

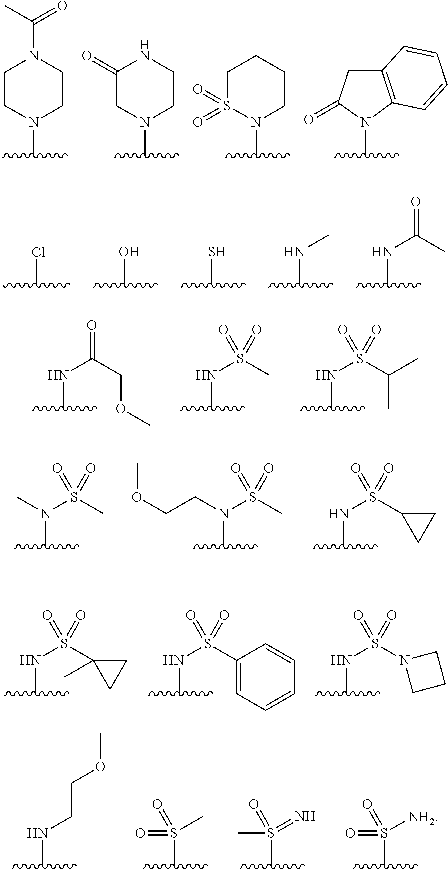

In certain embodiments, each R³ is independently hydrogen.

In certain embodiments, each R³ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R³ is independently methyl, ethyl, propyl, i-propyl, butyl, s-butyl, t-butyl, straight chain or branched pentyl, straight chain or branched hexyl, or straight chain or branched heptyl; each of which is optionally substituted.

In certain embodiments, each R³ is independently halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R³ is independently —CH₃, —NH₂, —OH, or —Cl.

In certain embodiments, each of A, X, Y, R¹, R², R³, R^{A}, and n, is as defined above and described in embodiments, classes and subclasses above and herein, singly or in combination.

In certain embodiments, the present invention provides a compound of formula II:

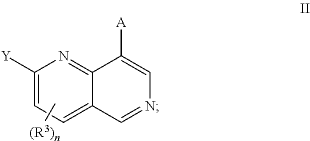

or a pharmaceutically acceptable salt thereof, wherein each of of A, Y, R¹, R², R³, R^{A}, and n, is as defined above and described in embodiments, classes and subclasses above and herein, singly or in combination.

In certain embodiments, the present invention provides a compound of formula III,

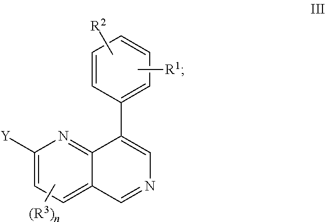

or a pharmaceutically acceptable salt thereof, wherein each of Y, R¹, R², R³, R^{A}, and n, is as defined above and described in embodiments, classes and subclasses above and herein, singly or in combination.

In certain embodiments, the present invention provides a compound of formula IV:

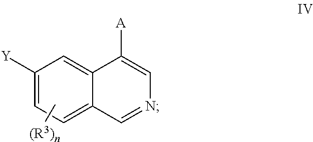

or a pharmaceutically acceptable salt thereof, wherein each of of A, Y, R¹, R², R³, R^{A}, and n, is as defined above and described in embodiments, classes and subclasses above and herein, singly or in combination.

In certain embodiments, the present invention provides a compound of formula V:

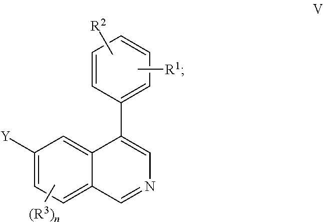

or a pharmaceutically acceptable salt thereof, wherein each of of Y, R¹, R², R³, R^{A}, and n, is as defined above and described in embodiments, classes and subclasses above and herein, singly or in combination.

In certain embodiments, the present invention provides a compound of formula VI:

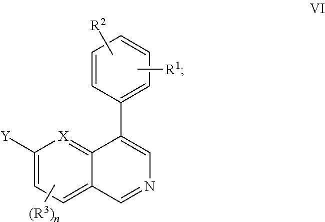

or a pharmaceutically acceptable salt thereof, wherein each of of X, Y, R¹, R², R³, R^{A}, and n, is as defined above and described in embodiments, classes and subclasses above and herein, singly or in combination.

In certain embodiments, the invention provides a compound selected from Table 1:

TABLE 1

| | |
|---|---|
| 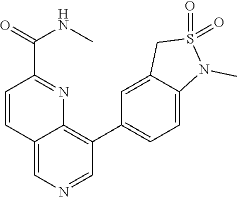 [SMILES: CNC(=O)c1ccc2cncc(-c3ccc4c(c3)CS(=O)(=O)N4C)c2n1] | 1 |
| 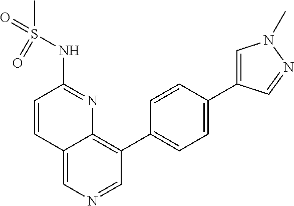 [SMILES: Cn1cc(-c2ccc(-c3cncc4ccc(NS(C)(=O)=O)nc34)cc2)cn1] | 2 |
| 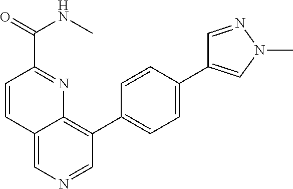 [SMILES: CNC(=O)c1ccc2cncc(-c3ccc(-c4cnn(C)c4)cc3)c2n1] | 3 |
| 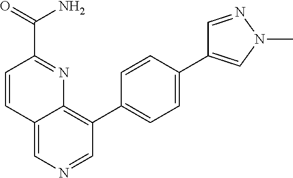 [SMILES: Cn1cc(-c2ccc(-c3cncc4ccc(C(N)=O)nc34)cc2)cn1] | 4 |
| 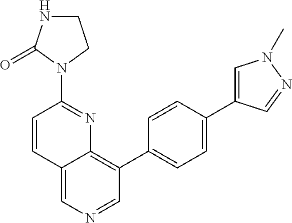 [SMILES: Cn1cc(-c2ccc(-c3cncc4ccc(N5CCNC5=O)nc34)cc2)cn1] | 5 |
| 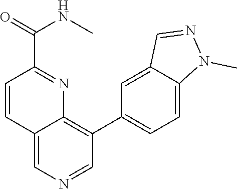 [SMILES: CNC(=O)c1ccc2cncc(-c3ccc4c(cnn4C)c3)c2n1] | 6 |
| 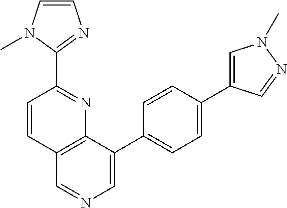 [SMILES: Cn1cc(-c2ccc(-c3cncc4ccc(-c5nccn5C)nc34)cc2)cn1] | 7 |
| 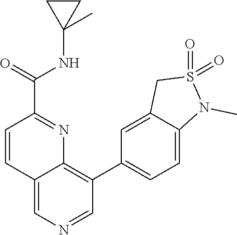 [SMILES: CN1c2ccc(-c3cncc4ccc(C(=O)NC5(C)CC5)nc34)cc2CS1(=O)=O] | 8 |
| 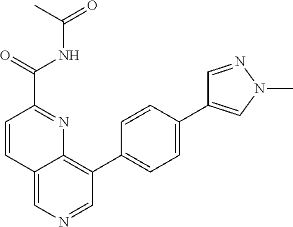 [SMILES: CC(=O)NC(=O)c1ccc2cncc(-c3ccc(-c4cnn(C)c4)cc3)c2n1] | 9 |
| 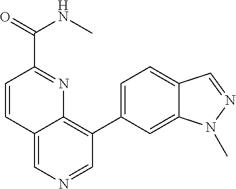 [SMILES: CNC(=O)c1ccc2cncc(-c3ccc4cnn(C)c4c3)c2n1] | 10 |
| 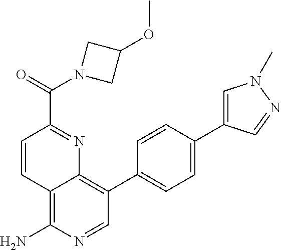 [SMILES: COC1CN(C(=O)c2ccc3c(N)ncc(-c4ccc(-c5cnn(C)c5)cc4)c3n2)C1] | 11 |
| 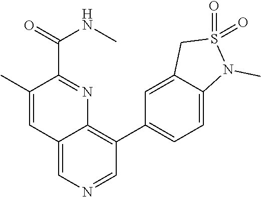 | 12 |
| 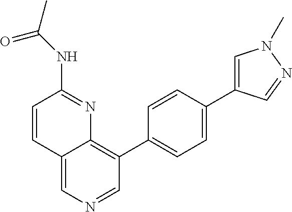 [SMILES: CC(=O)Nc1ccc2cncc(-c3ccc(-c4cnn(C)c4)cc3)c2n1] | 13 |
| 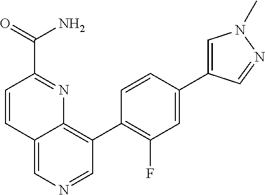 [SMILES: Cn1cc(-c2ccc(-c3cncc4ccc(C(N)=O)nc34)c(F)c2)cn1] | 14 |
| 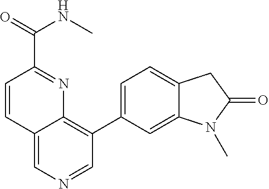 [SMILES: CNC(=O)c1ccc2cncc(-c3ccc4c(c3)N(C)C(=O)C4)c2n1] | 15 |
| 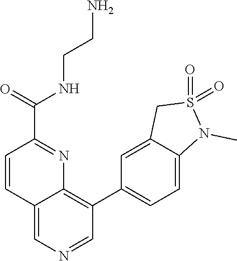 [SMILES: CN1c2ccc(-c3cncc4ccc(C(=O)NCCN)nc34)cc2CS1(=O)=O] | 16 |
| 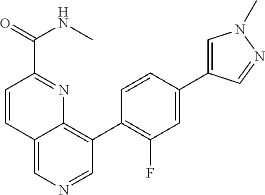 [SMILES: CNC(=O)c1ccc2cncc(-c3ccc(-c4cnn(C)c4)cc3F)c2n1] | 17 |
| 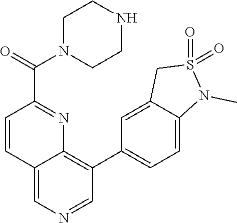 [SMILES: CN1c2ccc(-c3cncc4ccc(C(=O)N5CCNCC5)nc34)cc2CS1(=O)=O] | 18 |
| 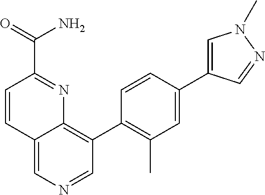 [SMILES: Cc1cc(-c2cnn(C)c2)ccc1-c1cncc2ccc(C(N)=O)nc12] | 19 |
| 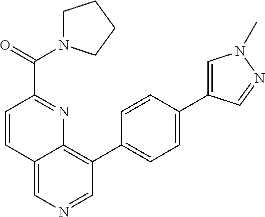 [SMILES: Cn1cc(-c2ccc(-c3cncc4ccc(C(=O)N5CCCC5)nc34)cc2)cn1] | 20 |
| 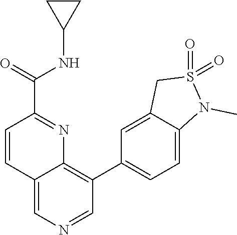 [SMILES: CN1c2ccc(-c3cncc4ccc(C(=O)NC5CC5)nc34)cc2CS1(=O)=O] | 21 |
| 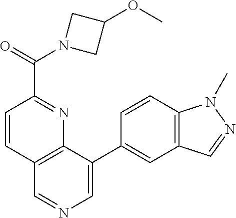 [SMILES: COC1CN(C(=O)c2ccc3cncc(-c4ccc5c(cnn5C)c4)c3n2)C1] | 22 |
| 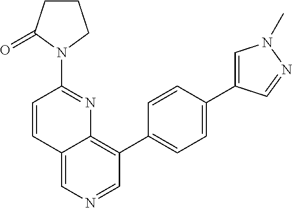 [SMILES: Cn1cc(-c2ccc(-c3cncc4ccc(N5CCCC5=O)nc34)cc2)cn1] | 23 |
| 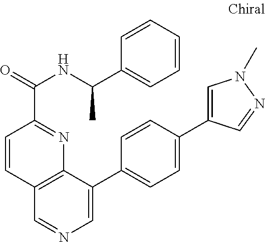 [SMILES: C[C@@H](NC(=O)c1ccc2cncc(-c3ccc(-c4cnn(C)c4)cc3)c2n1)c1ccccc1] | 24 |
| 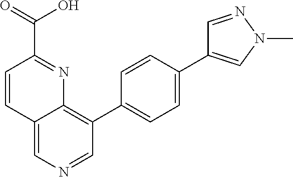 [SMILES: Cn1cc(-c2ccc(-c3cncc4ccc(C(=O)O)nc34)cc2)cn1] | 25 |
| 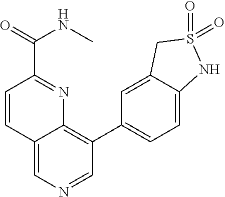 [SMILES: CNC(=O)c1ccc2cncc(-c3ccc4c(c3)CS(=O)(=O)N4)c2n1] | 26 |
| 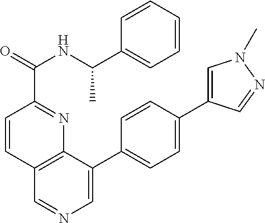 [SMILES: C[C@H](NC(=O)c1ccc2cncc(-c3ccc(-c4cnn(C)c4)cc3)c2n1)c1ccccc1] | 27 |
| 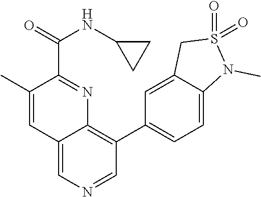 | 28 |
| 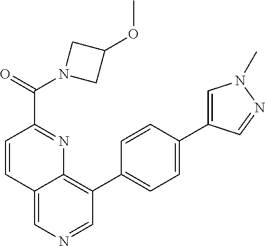 [SMILES: COC1CN(C(=O)c2ccc3cncc(-c4ccc(-c5cnn(C)c5)cc4)c3n2)C1] | 29 |
| 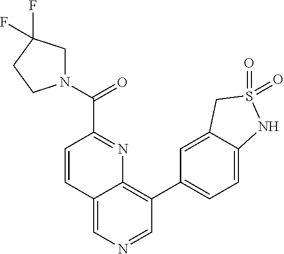 [SMILES: O=C(c1ccc2cncc(-c3ccc4c(c3)CS(=O)(=O)N4)c2n1)N1CCC(F)(F)C1] | 30 |
| 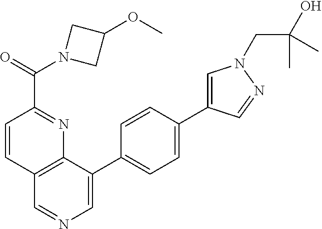 [SMILES: COC1CN(C(=O)c2ccc3cncc(-c4ccc(-c5cnn(CC(C)(C)O)c5)cc4)c3n2)C1] | 31 |
| 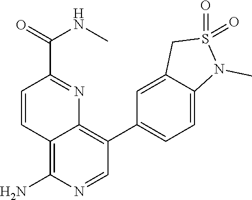 [SMILES: CNC(=O)c1ccc2c(N)ncc(-c3ccc4c(c3)CS(=O)(=O)N4C)c2n1] | 32 |
| 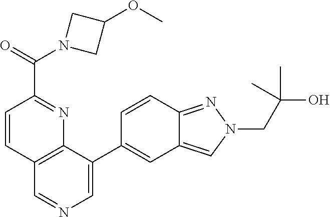 [SMILES: COC1CN(C(=O)c2ccc3cncc(-c4ccc5nn(CC(C)(C)O)cc5c4)c3n2)C1] | 33 |
| 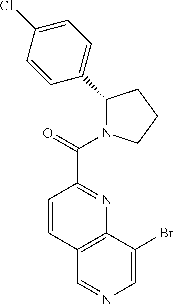 [SMILES: O=C(c1ccc2cncc(Br)c2n1)N1CCC[C@H]1c1ccc(Cl)cc1] | 34 |
| 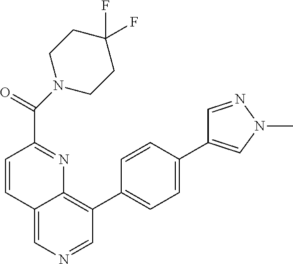 [SMILES: Cn1cc(-c2ccc(-c3cncc4ccc(C(=O)N5CCC(F)(F)CC5)nc34)cc2)cn1] | 35 |
| 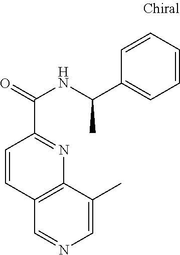 [SMILES: Cc1cncc2ccc(C(=O)N[C@H](C)c3ccccc3)nc12] | 36 |
| 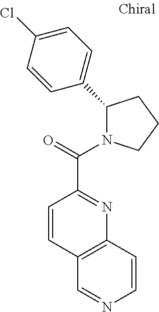 [SMILES: O=C(c1ccc2cnccc2n1)N1CCC[C@H]1c1ccc(Cl)cc1] | 37 |
| 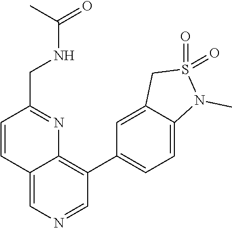 [SMILES: CC(=O)NCc1ccc2cncc(-c3ccc4c(c3)CS(=O)(=O)N4C)c2n1] | 38 |
| 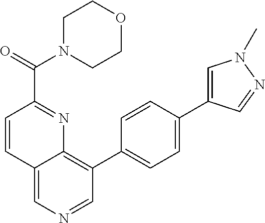 [SMILES: Cn1cc(-c2ccc(-c3cncc4ccc(C(=O)N5CCOCC5)nc34)cc2)cn1] | 39 |
| 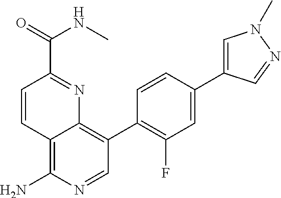 [SMILES: CNC(=O)c1ccc2c(N)ncc(-c3ccc(-c4cnn(C)c4)cc3F)c2n1] | 40 |
| 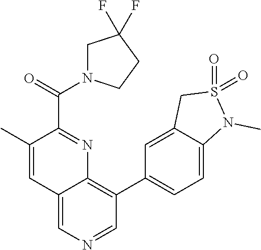 | 41 |
| 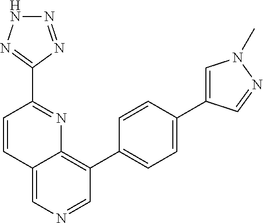 [SMILES: Cn1cc(-c2ccc(-c3cncc4ccc(-c5nn[nH]n5)nc34)cc2)cn1] | 42 |
| 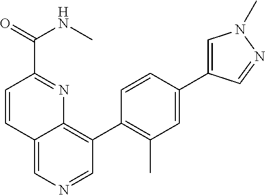 [SMILES: CNC(=O)c1ccc2cncc(-c3ccc(-c4cnn(C)c4)cc3C)c2n1] | 43 |
| 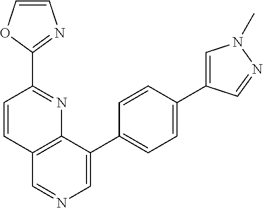 [SMILES: Cn1cc(-c2ccc(-c3cncc4ccc(-c5ncco5)nc34)cc2)cn1] | 44 |
| 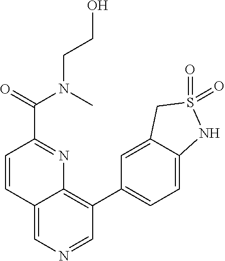 [SMILES: CN(CCO)C(=O)c1ccc2cncc(-c3ccc4c(c3)CS(=O)(=O)N4)c2n1] | 45 |
| 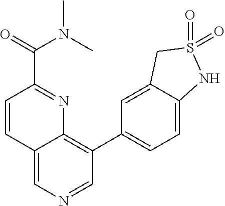 [SMILES: CN(C)C(=O)c1ccc2cncc(-c3ccc4c(c3)CS(=O)(=O)N4)c2n1] | 46 |
| 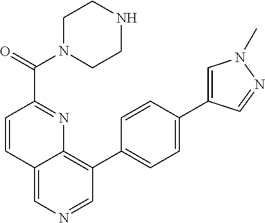 [SMILES: Cn1cc(-c2ccc(-c3cncc4ccc(C(=O)N5CCNCC5)nc34)cc2)cn1] | 47 |
| 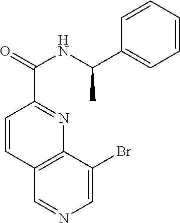 [SMILES: C[C@@H](NC(=O)c1ccc2cncc(Br)c2n1)c1ccccc1] | 48 |
| 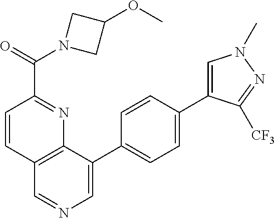 [SMILES: COC1CN(C(=O)c2ccc3cncc(-c4ccc(-c5cn(C)nc5C(F)(F)F)cc4)c3n2)C1] | 49 |
| 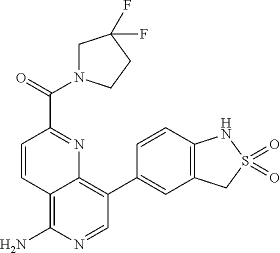 [SMILES: Nc1ncc(-c2ccc3c(c2)CS(=O)(=O)N3)c2nc(C(=O)N3CCC(F)(F)C3)ccc12] | 50 |
| 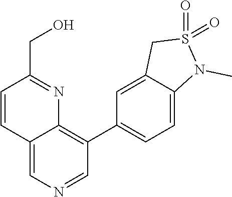 [SMILES: CN1c2ccc(-c3cncc4ccc(CO)nc34)cc2CS1(=O)=O] | 51 |
| 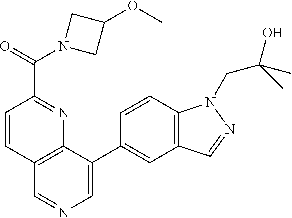 [SMILES: COC1CN(C(=O)c2ccc3cncc(-c4ccc5c(cnn5CC(C)(C)O)c4)c3n2)C1] | 52 |
| 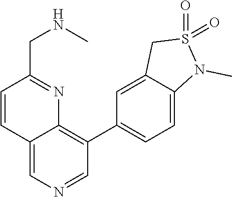 [SMILES: CNCc1ccc2cncc(-c3ccc4c(c3)CS(=O)(=O)N4C)c2n1] | 53 |
| 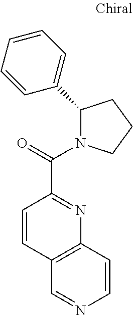 [SMILES: O=C(c1ccc2cnccc2n1)N1CCC[C@H]1c1ccccc1] | 54 |
| 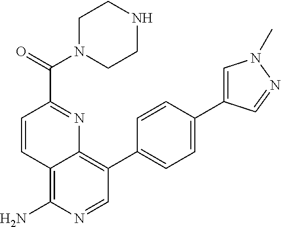 [SMILES: Cn1cc(-c2ccc(-c3cnc(N)c4ccc(C(=O)N5CCNCC5)nc34)cc2)cn1] | 55 |
| 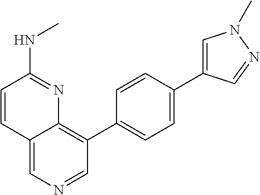 [SMILES: CNc1ccc2cncc(-c3ccc(-c4cnn(C)c4)cc3)c2n1] | 56 |
| 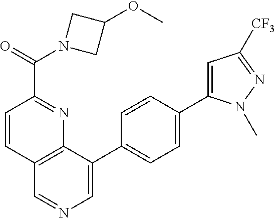 [SMILES: COC1CN(C(=O)c2ccc3cncc(-c4ccc(-c5cc(C(F)(F)F)nn5C)cc4)c3n2)C1] | 57 |
| 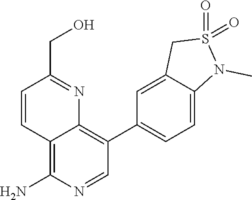 [SMILES: CN1c2ccc(-c3cnc(N)c4ccc(CO)nc34)cc2CS1(=O)=O] | 58 |
| 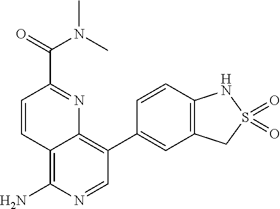 [SMILES: CN(C)C(=O)c1ccc2c(N)ncc(-c3ccc4c(c3)CS(=O)(=O)N4)c2n1] | 59 |
| 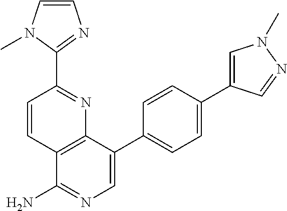 [SMILES: Cn1cc(-c2ccc(-c3cnc(N)c4ccc(-c5nccn5C)nc34)cc2)cn1] | 60 |
| 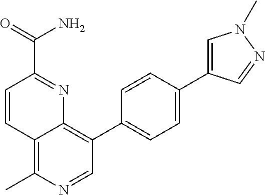 [SMILES: Cc1ncc(-c2ccc(-c3cnn(C)c3)cc2)c2nc(C(N)=O)ccc12] | 61 |
| 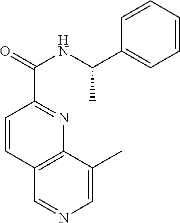 [SMILES: Cc1cncc2ccc(C(=O)N[C@@H](C)c3ccccc3)nc12] | 62 |
| 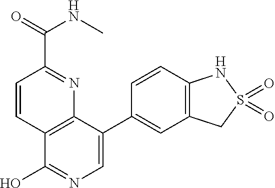 [SMILES: CNC(=O)c1ccc2c(O)ncc(-c3ccc4c(c3)CS(=O)(=O)N4)c2n1] | 63 |
| 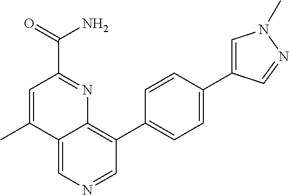 [SMILES: Cc1cc(C(N)=O)nc2c(-c3ccc(-c4cnn(C)c4)cc3)cncc12] | 64 |
| 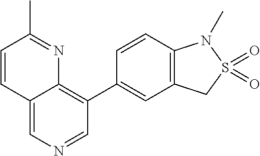 [SMILES: Cc1ccc2cncc(-c3ccc4c(c3)CS(=O)(=O)N4C)c2n1] | 65 |
| 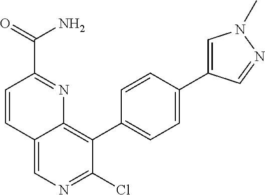 [SMILES: Cn1cc(-c2ccc(-c3c(Cl)ncc4ccc(C(N)=O)nc34)cc2)cn1] | 66 |
| 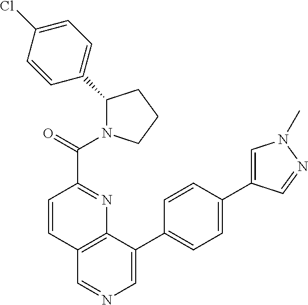 [SMILES: Cn1cc(-c2ccc(-c3cncc4ccc(C(=O)N5CCC[C@H]5c5ccc(Cl)cc5)nc34)cc2)cn1] | 67 |
| 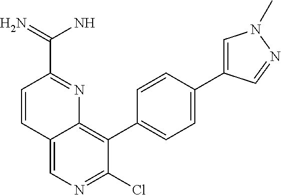 [SMILES: Cn1cc(-c2ccc(-c3c(Cl)ncc4ccc(C(=N)N)nc34)cc2)cn1] | 68 |
| 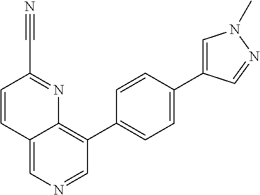 [SMILES: Cn1cc(-c2ccc(-c3cncc4ccc(C#N)nc34)cc2)cn1] | 69 |
| 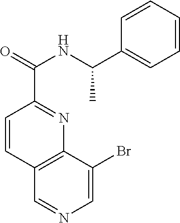 [SMILES: C[C@H](NC(=O)c1ccc2cncc(Br)c2n1)c1ccccc1] | 70 |
| 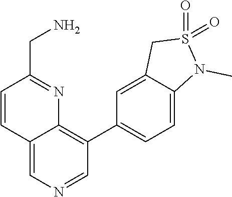 [SMILES: CN1c2ccc(-c3cncc4ccc(CN)nc34)cc2CS1(=O)=O] | 71 |
| 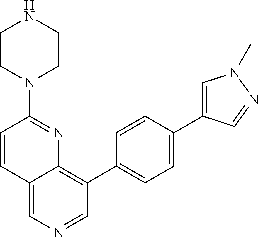 [SMILES: Cn1cc(-c2ccc(-c3cncc4ccc(N5CCNCC5)nc34)cc2)cn1] | 72 |
| 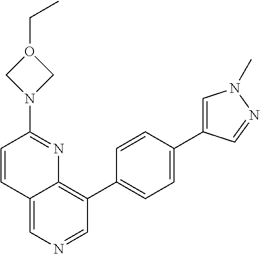 | 73 |
| 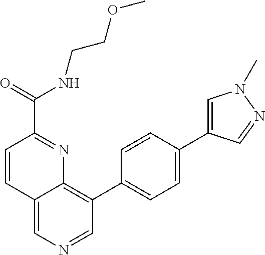 [SMILES: COCCNC(=O)c1ccc2cncc(-c3ccc(-c4cnn(C)c4)cc3)c2n1] | 74 |
| 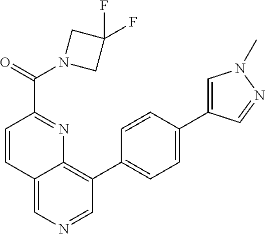 [SMILES: Cn1cc(-c2ccc(-c3cncc4ccc(C(=O)N5CC(F)(F)C5)nc34)cc2)cn1] | 75 |
| 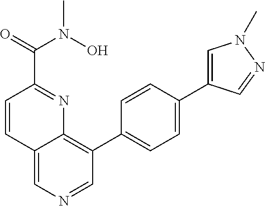 [SMILES: CN(O)C(=O)c1ccc2cncc(-c3ccc(-c4cnn(C)c4)cc3)c2n1] | 76 |
| 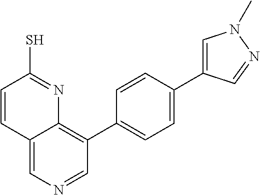 [SMILES: Cn1cc(-c2ccc(-c3cncc4ccc(S)nc34)cc2)cn1] | 77 |
| 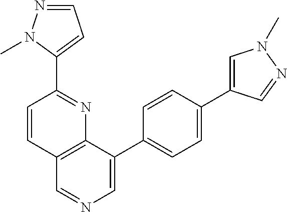 [SMILES: Cn1cc(-c2ccc(-c3cncc4ccc(-c5ccnn5C)nc34)cc2)cn1] | 78 |
| 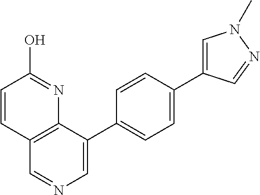 [SMILES: Cn1cc(-c2ccc(-c3cncc4ccc(O)nc34)cc2)cn1] | 79 |
| 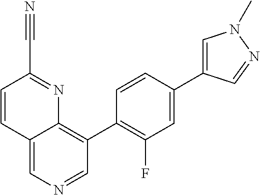 [SMILES: Cn1cc(-c2ccc(-c3cncc4ccc(C#N)nc34)c(F)c2)cn1] | 80 |
| 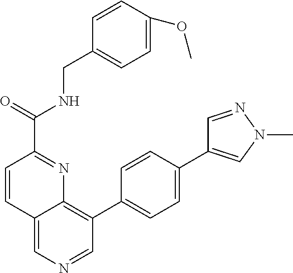 [SMILES: COc1ccc(CNC(=O)c2ccc3cncc(-c4ccc(-c5cnn(C)c5)cc4)c3n2)cc1] | 81 |
| 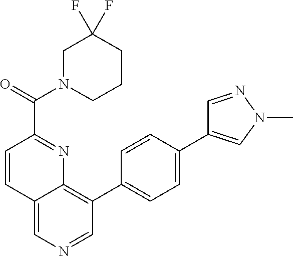 [SMILES: Cn1cc(-c2ccc(-c3cncc4ccc(C(=O)N5CCCC(F)(F)C5)nc34)cc2)cn1] | 82 |
| 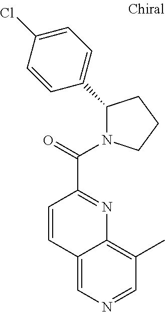 [SMILES: Cc1cncc2ccc(C(=O)N3CCC[C@H]3c3ccc(Cl)cc3)nc12] | 83 |
| 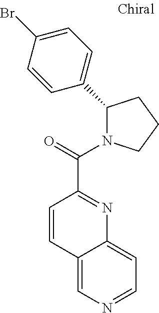 [SMILES: O=C(c1ccc2cnccc2n1)N1CCC[C@H]1c1ccc(Br)cc1] | 84 |
| 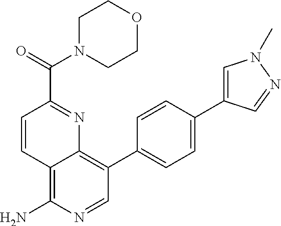 [SMILES: Cn1cc(-c2ccc(-c3cnc(N)c4ccc(C(=O)N5CCOCC5)nc34)cc2)cn1] | 85 |
| 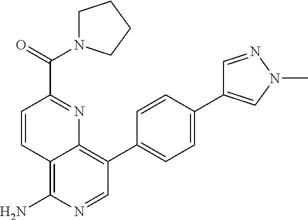 [SMILES: Cn1cc(-c2ccc(-c3cnc(N)c4ccc(C(=O)N5CCCC5)nc34)cc2)cn1] | 86 |
| 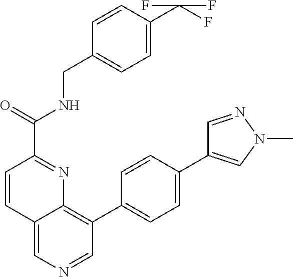 [SMILES: Cn1cc(-c2ccc(-c3cncc4ccc(C(=O)NCc5ccc(C(F)(F)F)cc5)nc34)cc2)cn1] | 87 |
| 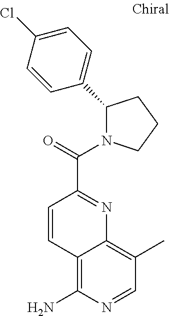 [SMILES: Cc1cnc(N)c2ccc(C(=O)N3CCC[C@H]3c3ccc(Cl)cc3)nc12] | 88 |
| 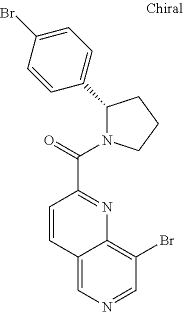 [SMILES: O=C(c1ccc2cncc(Br)c2n1)N1CCC[C@H]1c1ccc(Br)cc1] | 89 |
| 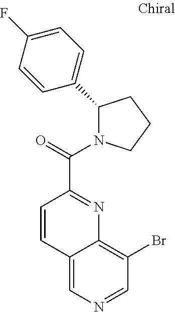 [SMILES: O=C(c1ccc2cncc(Br)c2n1)N1CCC[C@H]1c1ccc(F)cc1] | 90 |
| 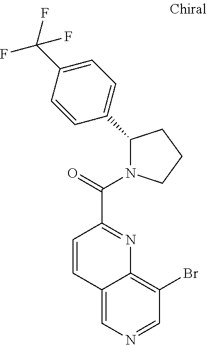 [SMILES: O=C(c1ccc2cncc(Br)c2n1)N1CCC[C@H]1c1ccc(C(F)(F)F)cc1] | 91 |
| 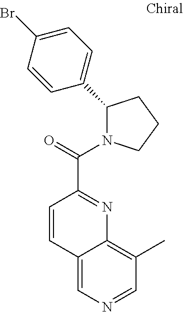 [SMILES: Cc1cncc2ccc(C(=O)N3CCC[C@H]3c3ccc(Br)cc3)nc12] | 92 |
| 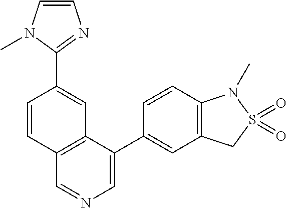 [SMILES: CN1c2ccc(-c3cncc4ccc(-c5nccn5C)cc34)cc2CS1(=O)=O] | 93 |
| 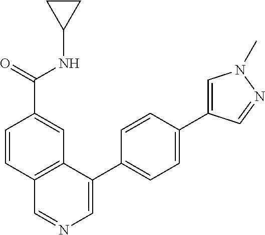 [SMILES: Cn1cc(-c2ccc(-c3cncc4ccc(C(=O)NC5CC5)cc34)cc2)cn1] | 94 |
| 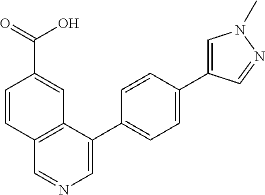 [SMILES: Cn1cc(-c2ccc(-c3cncc4ccc(C(=O)O)cc34)cc2)cn1] | 95 |
| 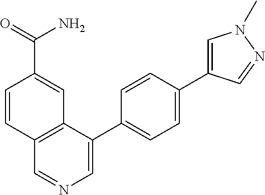 [SMILES: Cn1cc(-c2ccc(-c3cncc4ccc(C(N)=O)cc34)cc2)cn1] | 96 |
| 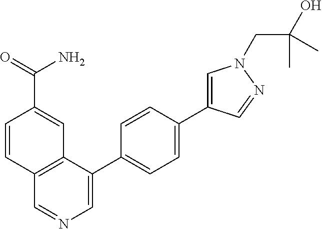 [SMILES: CC(C)(O)Cn1cc(-c2ccc(-c3cncc4ccc(C(N)=O)cc34)cc2)cn1] | 97 |
| 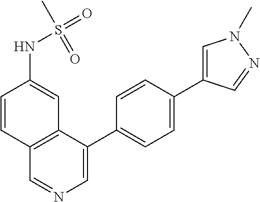 [SMILES: Cn1cc(-c2ccc(-c3cncc4ccc(NS(C)(=O)=O)cc34)cc2)cn1] | 98 |
| 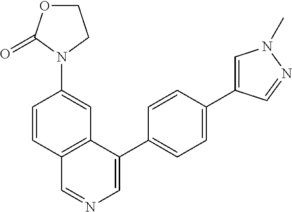 [SMILES: Cn1cc(-c2ccc(-c3cncc4ccc(N5CCOC5=O)cc34)cc2)cn1] | 99 |
| 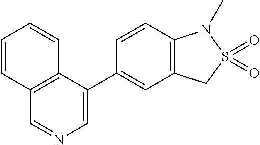 [SMILES: CN1c2ccc(-c3cncc4ccccc34)cc2CS1(=O)=O] | 100 |
| 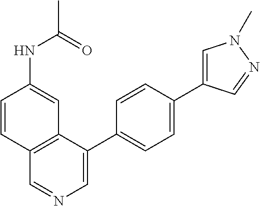 [SMILES: CC(=O)Nc1ccc2cncc(-c3ccc(-c4cnn(C)c4)cc3)c2c1] | 101 |
| 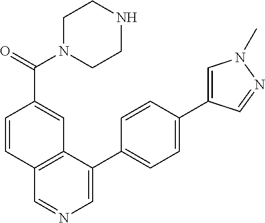 [SMILES: Cn1cc(-c2ccc(-c3cncc4ccc(C(=O)N5CCNCC5)cc34)cc2)cn1] | 102 |
| 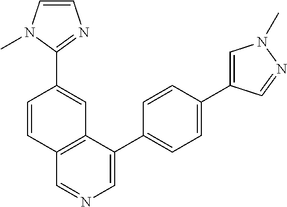 [SMILES: Cn1cc(-c2ccc(-c3cncc4ccc(-c5nccn5C)cc34)cc2)cn1] | 103 |
| 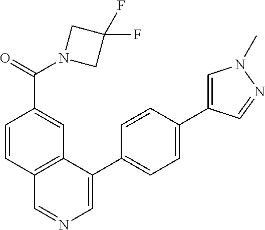 [SMILES: Cn1cc(-c2ccc(-c3cncc4ccc(C(=O)N5CC(F)(F)C5)cc34)cc2)cn1] | 104 |
| 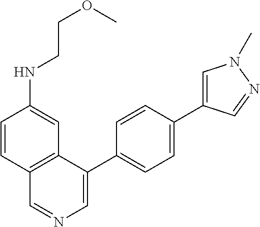 [SMILES: COCCNc1ccc2cncc(-c3ccc(-c4cnn(C)c4)cc3)c2c1] | 105 |
| 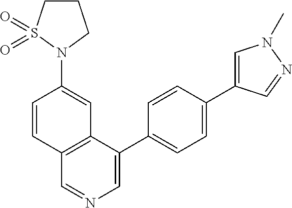 [SMILES: Cn1cc(-c2ccc(-c3cncc4ccc(N5CCCS5(=O)=O)cc34)cc2)cn1] | 106 |
| 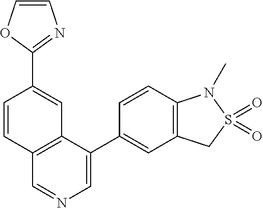 [SMILES: CN1c2ccc(-c3cncc4ccc(-c5ncco5)cc34)cc2CS1(=O)=O] | 107 |
| 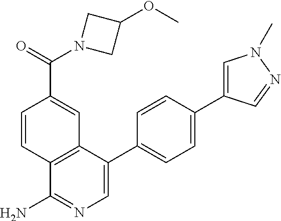 [SMILES: COC1CN(C(=O)c2ccc3c(N)ncc(-c4ccc(-c5cnn(C)c5)cc4)c3c2)C1] | 108 |
| 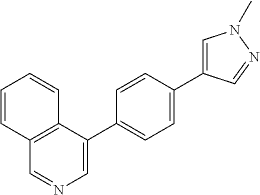 [SMILES: Cn1cc(-c2ccc(-c3cncc4ccccc34)cc2)cn1] | 109 |
| 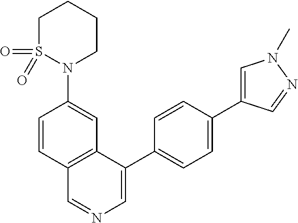 [SMILES: Cn1cc(-c2ccc(-c3cncc4ccc(N5CCCCS5(=O)=O)cc34)cc2)cn1] | 110 |
| 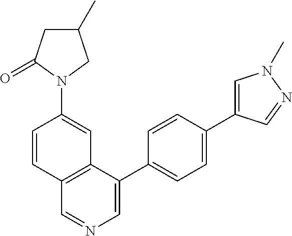 [SMILES: CC1CC(=O)N(c2ccc3cncc(-c4ccc(-c5cnn(C)c5)cc4)c3c2)C1] | 111 |
| 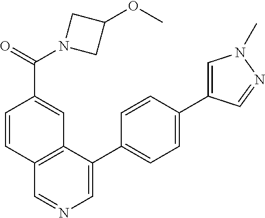 [SMILES: COC1CN(C(=O)c2ccc3cncc(-c4ccc(-c5cnn(C)c5)cc4)c3c2)C1] | 112 |
| 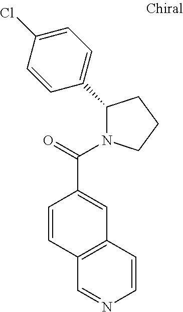 [SMILES: O=C(c1ccc2cnccc2c1)N1CCC[C@H]1c1ccc(Cl)cc1] | 113 |
| 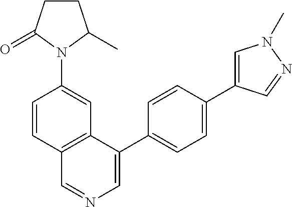 [SMILES: CC1CCC(=O)N1c1ccc2cncc(-c3ccc(-c4cnn(C)c4)cc3)c2c1] | 114 |
| 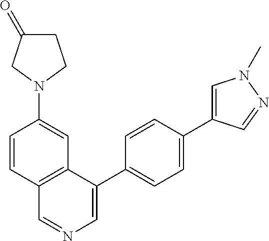 [SMILES: Cn1cc(-c2ccc(-c3cncc4ccc(N5CCC(=O)C5)cc34)cc2)cn1] | 115 |
| 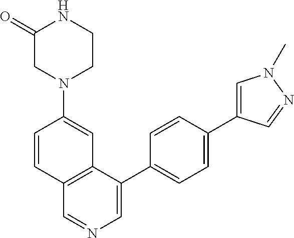 [SMILES: Cn1cc(-c2ccc(-c3cncc4ccc(N5CCNC(=O)C5)cc34)cc2)cn1] | 116 |
| 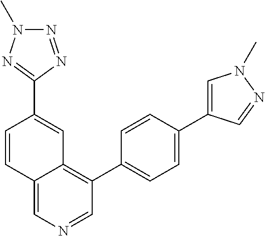 [SMILES: Cn1cc(-c2ccc(-c3cncc4ccc(-c5nnn(C)n5)cc34)cc2)cn1] | 117 |
| 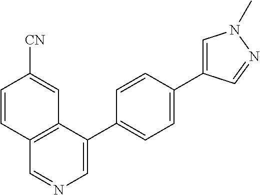 [SMILES: Cn1cc(-c2ccc(-c3cncc4ccc(C#N)cc34)cc2)cn1] | 118 |
| 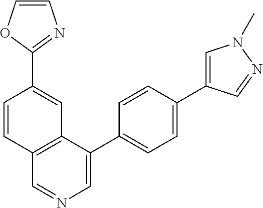 [SMILES: Cn1cc(-c2ccc(-c3cncc4ccc(-c5ncco5)cc34)cc2)cn1] | 119 |
| 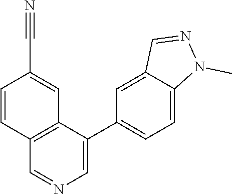 [SMILES: Cn1ncc2cc(-c3cncc4ccc(C#N)cc34)ccc21] | 120 |
| 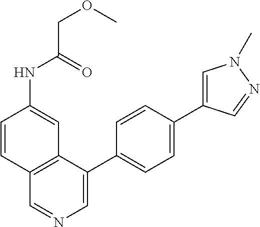 [SMILES: COCC(=O)Nc1ccc2cncc(-c3ccc(-c4cnn(C)c4)cc3)c2c1] | 121 |
| 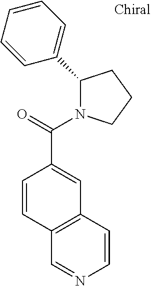 [SMILES: O=C(c1ccc2cnccc2c1)N1CCC[C@H]1c1ccccc1] | 122 |
| 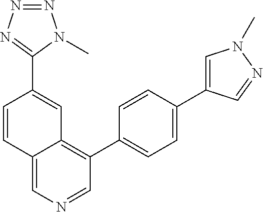 [SMILES: Cn1cc(-c2ccc(-c3cncc4ccc(-c5nnnn5C)cc34)cc2)cn1] | 123 |
| 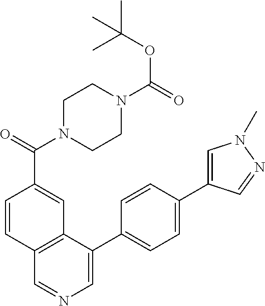 [SMILES: Cn1cc(-c2ccc(-c3cncc4ccc(C(=O)N5CCN(C(=O)OC(C)(C)C)CC5)cc34)cc2)cn1] | 124 |
| 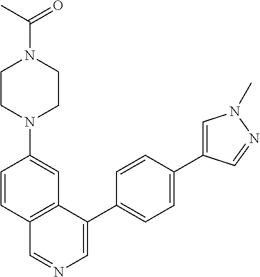 [SMILES: CC(=O)N1CCN(c2ccc3cncc(-c4ccc(-c5cnn(C)c5)cc4)c3c2)CC1] | 125 |
| 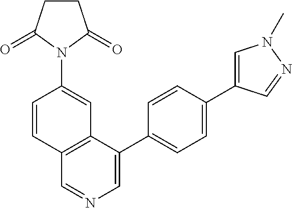 [SMILES: Cn1cc(-c2ccc(-c3cncc4ccc(N5C(=O)CCC5=O)cc34)cc2)cn1] | 126 |
| 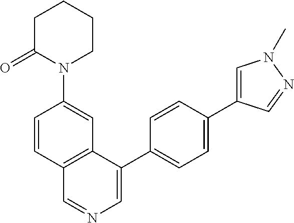 [SMILES: Cn1cc(-c2ccc(-c3cncc4ccc(N5CCCCC5=O)cc34)cc2)cn1] | 127 |
| 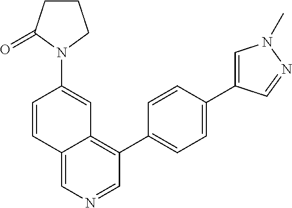 [SMILES: Cn1cc(-c2ccc(-c3cncc4ccc(N5CCCC5=O)cc34)cc2)cn1] | 128 |
| 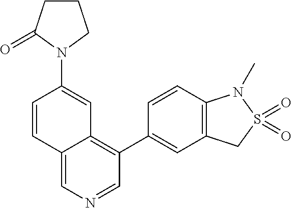 [SMILES: CN1c2ccc(-c3cncc4ccc(N5CCCC5=O)cc34)cc2CS1(=O)=O] | 129 |
| 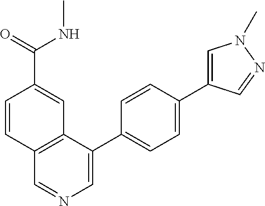 [SMILES: CNC(=O)c1ccc2cncc(-c3ccc(-c4cnn(C)c4)cc3)c2c1] | 130 |
| 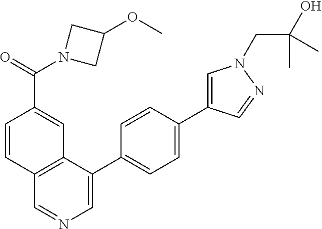 [SMILES: COC1CN(C(=O)c2ccc3cncc(-c4ccc(-c5cnn(CC(C)(C)O)c5)cc4)c3c2)C1] | 131 |
| 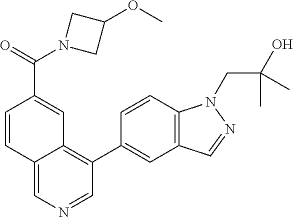 [SMILES: COC1CN(C(=O)c2ccc3cncc(-c4ccc5c(cnn5CC(C)(C)O)c4)c3c2)C1] | 132 |
| 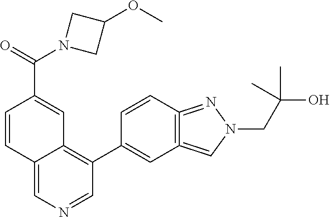 | 133 |
| 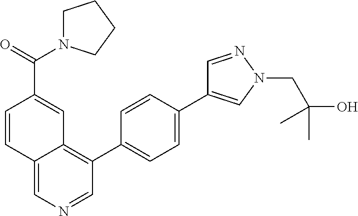 [SMILES: CC(C)(O)Cn1cc(-c2ccc(-c3cncc4ccc(C(=O)N5CCCC5)cc34)cc2)cn1] | 134 |
| 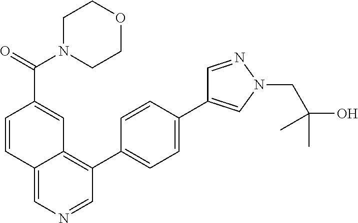 [SMILES: CC(C)(O)Cn1cc(-c2ccc(-c3cncc4ccc(C(=O)N5CCOCC5)cc34)cc2)cn1] | 135 |
| 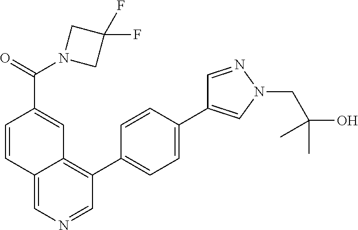 [SMILES: CC(C)(O)Cn1cc(-c2ccc(-c3cncc4ccc(C(=O)N5CC(F)(F)C5)cc34)cc2)cn1] | 136 |
| 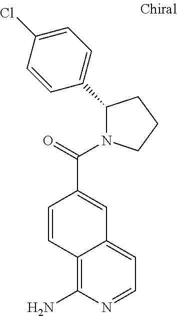 [SMILES: Nc1nccc2cc(C(=O)N3CCC[C@H]3c3ccc(Cl)cc3)ccc12] | 137 |
| 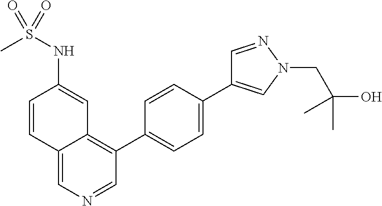 [SMILES: CC(C)(O)Cn1cc(-c2ccc(-c3cncc4ccc(NS(C)(=O)=O)cc34)cc2)cn1] | 138 |
| 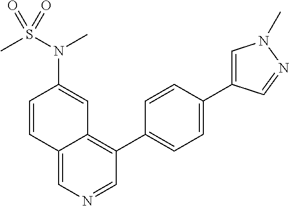 [SMILES: CN(c1ccc2cncc(-c3ccc(-c4cnn(C)c4)cc3)c2c1)S(C)(=O)=O] | 139 |
| 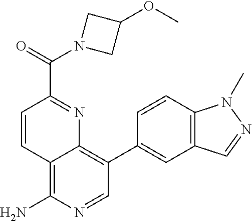 [SMILES: COC1CN(C(=O)c2ccc3c(N)ncc(-c4ccc5c(cnn5C)c4)c3n2)C1] | 140 |
| 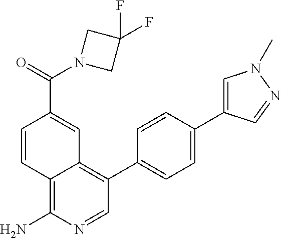 [SMILES: Cn1cc(-c2ccc(-c3cnc(N)c4ccc(C(=O)N5CC(F)(F)C5)cc34)cc2)cn1] | 141 |
| 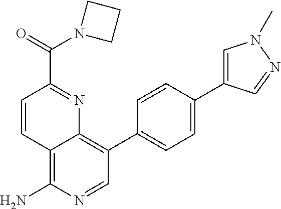 [SMILES: Cn1cc(-c2ccc(-c3cnc(N)c4ccc(C(=O)N5CCC5)nc34)cc2)cn1] | 142 |
| 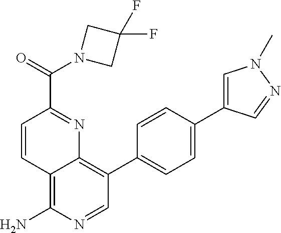 [SMILES: Cn1cc(-c2ccc(-c3cnc(N)c4ccc(C(=O)N5CC(F)(F)C5)nc34)cc2)cn1] | 143 |
| 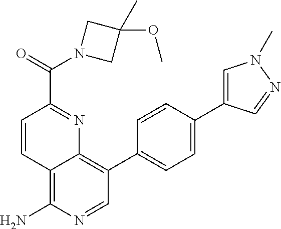 [SMILES: COC1(C)CN(C(=O)c2ccc3c(N)ncc(-c4ccc(-c5cnn(C)c5)cc4)c3n2)C1] | 144 |
| 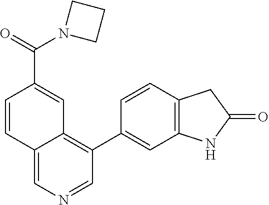 [SMILES: O=C1Cc2ccc(-c3cncc4ccc(C(=O)N5CCC5)cc34)cc2N1] | 145 |
| 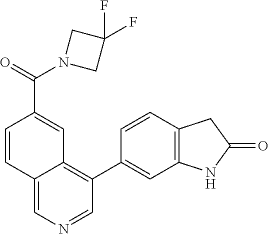 [SMILES: O=C1Cc2ccc(-c3cncc4ccc(C(=O)N5CC(F)(F)C5)cc34)cc2N1] | 146 |
| 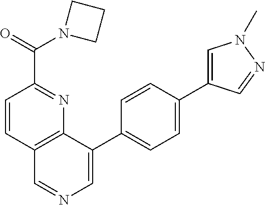 [SMILES: Cn1cc(-c2ccc(-c3cncc4ccc(C(=O)N5CCC5)nc34)cc2)cn1] | 147 |
| 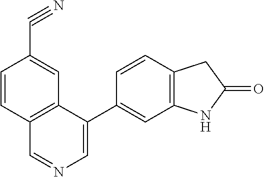 [SMILES: N#Cc1ccc2cncc(-c3ccc4c(c3)NC(=O)C4)c2c1] | 148 |
| 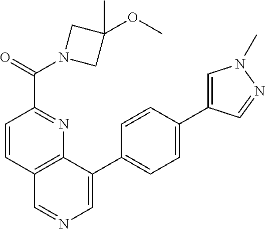 [SMILES: COC1(C)CN(C(=O)c2ccc3cncc(-c4ccc(-c5cnn(C)c5)cc4)c3n2)C1] | 149 |
| 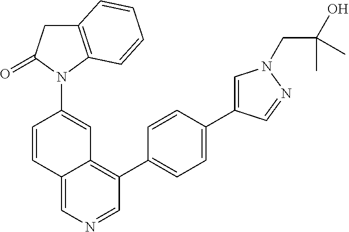 [SMILES: CC(C)(O)Cn1cc(-c2ccc(-c3cncc4ccc(N5C(=O)Cc6ccccc65)cc34)cc2)cn1] | 150 |
| 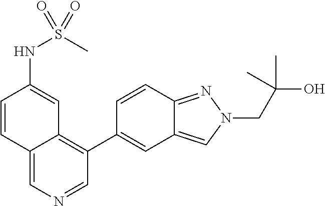 [SMILES: CC(C)(O)Cn1cc2cc(-c3cncc4ccc(NS(C)(=O)=O)cc34)ccc2n1] | 151 |
| 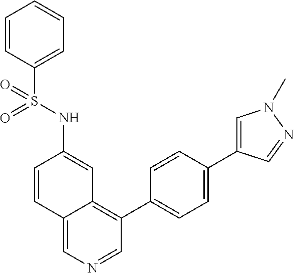 [SMILES: Cn1cc(-c2ccc(-c3cncc4ccc(NS(=O)(=O)c5ccccc5)cc34)cc2)cn1] | 152 |
| 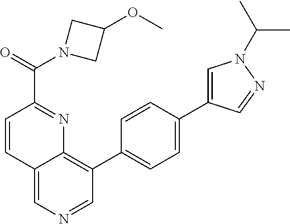 [SMILES: COC1CN(C(=O)c2ccc3cncc(-c4ccc(-c5cnn(C(C)C)c5)cc4)c3n2)C1] | 153 |
| 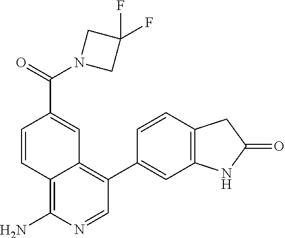 [SMILES: Nc1ncc(-c2ccc3c(c2)NC(=O)C3)c2cc(C(=O)N3CC(F)(F)C3)ccc12] | 154 |
| 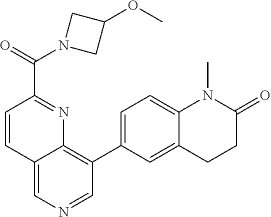 [SMILES: COC1CN(C(=O)c2ccc3cncc(-c4ccc5c(c4)CCC(=O)N5C)c3n2)C1] | 155 |
| 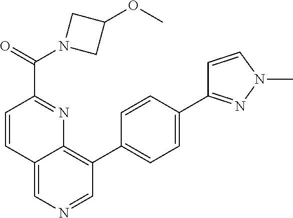 [SMILES: COC1CN(C(=O)c2ccc3cncc(-c4ccc(-c5ccn(C)n5)cc4)c3n2)C1] | 156 |
| 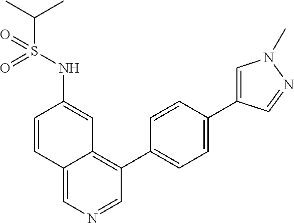 [SMILES: CC(C)S(=O)(=O)Nc1ccc2cncc(-c3ccc(-c4cnn(C)c4)cc3)c2c1] | 157 |
| 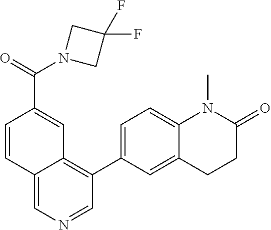 [SMILES: CN1C(=O)CCc2cc(-c3cncc4ccc(C(=O)N5CC(F)(F)C5)cc34)ccc21] | 158 |
| 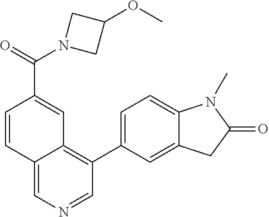 [SMILES: COC1CN(C(=O)c2ccc3cncc(-c4ccc5c(c4)CC(=O)N5C)c3c2)C1] | 159 |
| 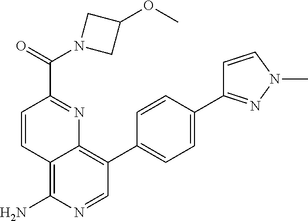 [SMILES: COC1CN(C(=O)c2ccc3c(N)ncc(-c4ccc(-c5ccn(C)n5)cc4)c3n2)C1] | 160 |
| 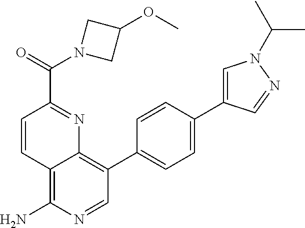 [SMILES: COC1CN(C(=O)c2ccc3c(N)ncc(-c4ccc(-c5cnn(C(C)C)c5)cc4)c3n2)C1] | 161 |
| 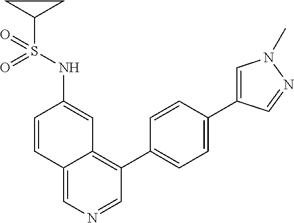 [SMILES: Cn1cc(-c2ccc(-c3cncc4ccc(NS(=O)(=O)C5CC5)cc34)cc2)cn1] | 162 |
| 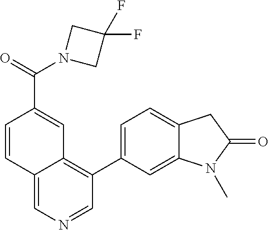 [SMILES: CN1C(=O)Cc2ccc(-c3cncc4ccc(C(=O)N5CC(F)(F)C5)cc34)cc21] | 163 |
| 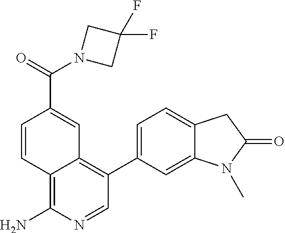 [SMILES: CN1C(=O)Cc2ccc(-c3cnc(N)c4ccc(C(=O)N5CC(F)(F)C5)cc34)cc21] | 164 |
| 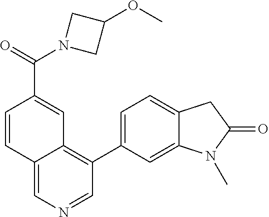 [SMILES: COC1CN(C(=O)c2ccc3cncc(-c4ccc5c(c4)N(C)C(=O)C5)c3c2)C1] | 165 |
| 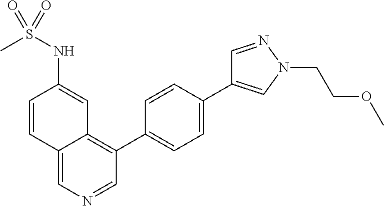 | 166 |
| 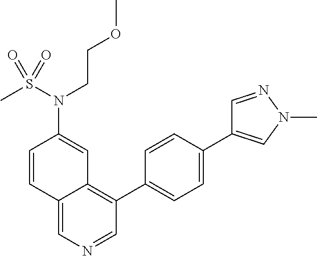 | 167 |
| 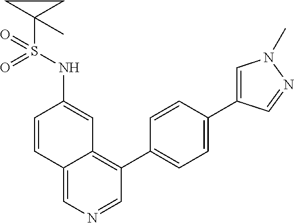 [SMILES: Cn1cc(-c2ccc(-c3cncc4ccc(NS(=O)(=O)C5(C)CC5)cc34)cc2)cn1] | 168 |
| 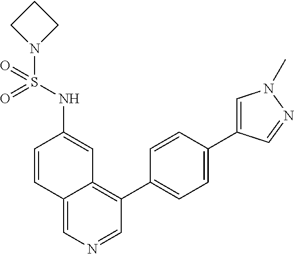 [SMILES: Cn1cc(-c2ccc(-c3cncc4ccc(NS(=O)(=O)N5CCC5)cc34)cc2)cn1] | 169 |
| 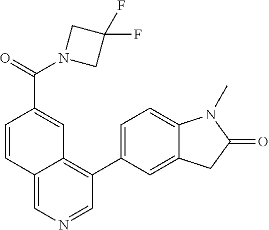 [SMILES: CN1C(=O)Cc2cc(-c3cncc4ccc(C(=O)N5CC(F)(F)C5)cc34)ccc21] | 170 |
| 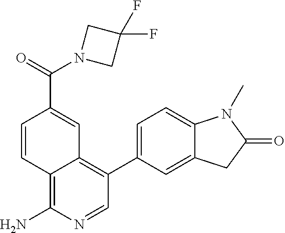 [SMILES: CN1C(=O)Cc2cc(-c3cnc(N)c4ccc(C(=O)N5CC(F)(F)C5)cc34)ccc21] | 171 |
| 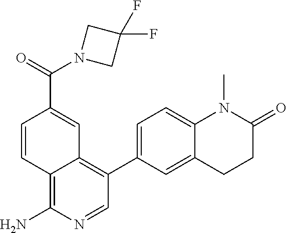 [SMILES: CN1C(=O)CCc2cc(-c3cnc(N)c4ccc(C(=O)N5CC(F)(F)C5)cc34)ccc21] | 172 |
| 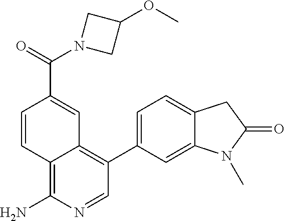 [SMILES: COC1CN(C(=O)c2ccc3c(N)ncc(-c4ccc5c(c4)N(C)C(=O)C5)c3c2)C1] | 173 |
| 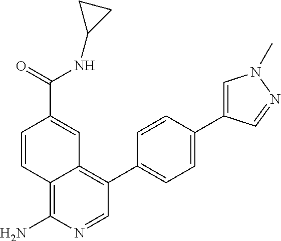 [SMILES: Cn1cc(-c2ccc(-c3cnc(N)c4ccc(C(=O)NC5CC5)cc34)cc2)cn1] | 174 |
| 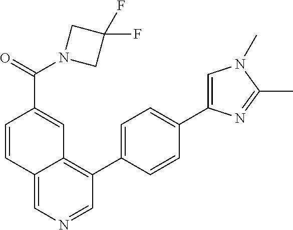 [SMILES: Cc1nc(-c2ccc(-c3cncc4ccc(C(=O)N5CC(F)(F)C5)cc34)cc2)cn1C] | 175 |
| 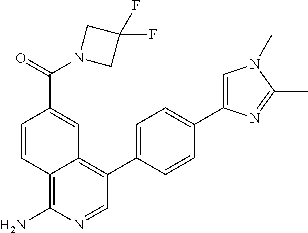 [SMILES: Cc1nc(-c2ccc(-c3cnc(N)c4ccc(C(=O)N5CC(F)(F)C5)cc34)cc2)cn1C] | 176 |
| 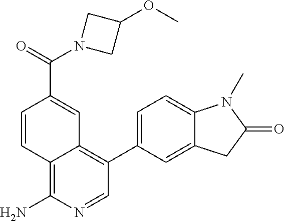 [SMILES: COC1CN(C(=O)c2ccc3c(N)ncc(-c4ccc5c(c4)CC(=O)N5C)c3c2)C1] | 177 |
| 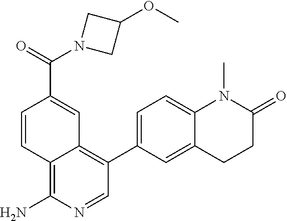 [SMILES: COC1CN(C(=O)c2ccc3c(N)ncc(-c4ccc5c(c4)CCC(=O)N5C)c3c2)C1] | 178 |
| 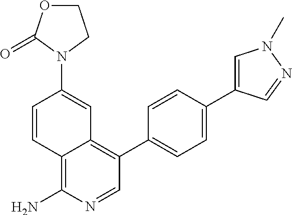 [SMILES: Cn1cc(-c2ccc(-c3cnc(N)c4ccc(N5CCOC5=O)cc34)cc2)cn1] | 179 |
| 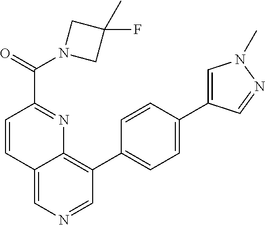 [SMILES: Cn1cc(-c2ccc(-c3cncc4ccc(C(=O)N5CC(C)(F)C5)nc34)cc2)cn1] | 180 |
| 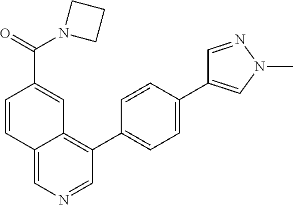 [SMILES: Cn1cc(-c2ccc(-c3cncc4ccc(C(=O)N5CCC5)cc34)cc2)cn1] | 181 |
| 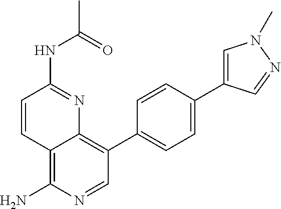 [SMILES: CC(=O)Nc1ccc2c(N)ncc(-c3ccc(-c4cnn(C)c4)cc3)c2n1] | 182 |
| 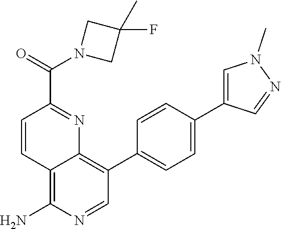 [SMILES: Cn1cc(-c2ccc(-c3cnc(N)c4ccc(C(=O)N5CC(C)(F)C5)nc34)cc2)cn1] | 184 |
| 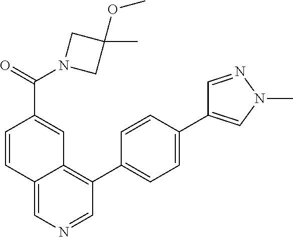 [SMILES: COC1(C)CN(C(=O)c2ccc3cncc(-c4ccc(-c5cnn(C)c5)cc4)c3c2)C1] | 185 |
| 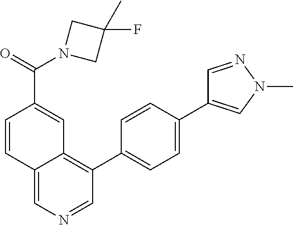 [SMILES: Cn1cc(-c2ccc(-c3cncc4ccc(C(=O)N5CC(C)(F)C5)cc34)cc2)cn1] | 186 |
| 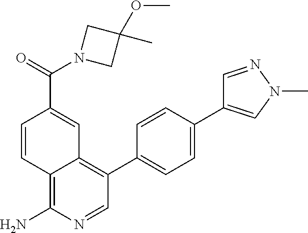 [SMILES: COC1(C)CN(C(=O)c2ccc3c(N)ncc(-c4ccc(-c5cnn(C)c5)cc4)c3c2)C1] | 187 |
| 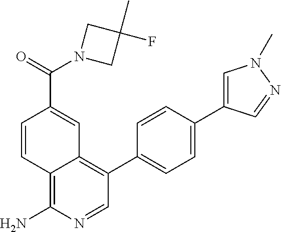 [SMILES: Cn1cc(-c2ccc(-c3cnc(N)c4ccc(C(=O)N5CC(C)(F)C5)cc34)cc2)cn1] | 188 |
| 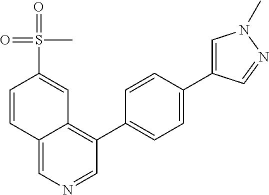 [SMILES: Cn1cc(-c2ccc(-c3cncc4ccc(S(C)(=O)=O)cc34)cc2)cn1] | 189 |
| 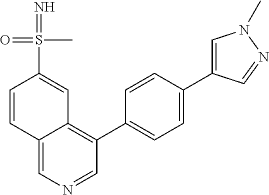 [SMILES: Cn1cc(-c2ccc(-c3cncc4ccc(S(C)(=N)=O)cc34)cc2)cn1] | 190 |
| 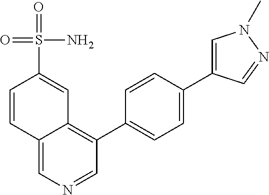 [SMILES: Cn1cc(-c2ccc(-c3cncc4ccc(S(N)(=O)=O)cc34)cc2)cn1] | 191 |
| 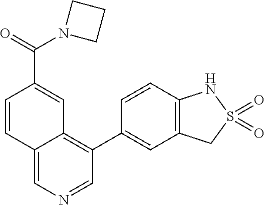 [SMILES: O=C(c1ccc2cncc(-c3ccc4c(c3)CS(=O)(=O)N4)c2c1)N1CCC1] | 192 |
| 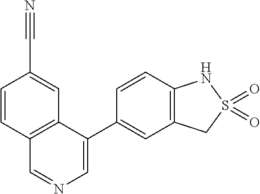 [SMILES: N#Cc1ccc2cncc(-c3ccc4c(c3)CS(=O)(=O)N4)c2c1] | 193 |
| 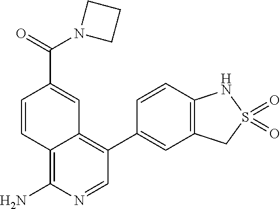 [SMILES: Nc1ncc(-c2ccc3c(c2)CS(=O)(=O)N3)c2cc(C(=O)N3CCC3)ccc12] | 194 |
| 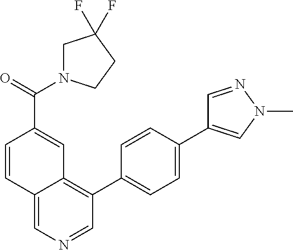 [SMILES: Cn1cc(-c2ccc(-c3cncc4ccc(C(=O)N5CCC(F)(F)C5)cc34)cc2)cn1] | 195 |
| 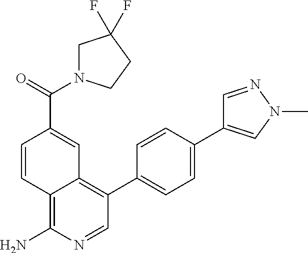 [SMILES: Cn1cc(-c2ccc(-c3cnc(N)c4ccc(C(=O)N5CCC(F)(F)C5)cc34)cc2)cn1] | 196 |
| 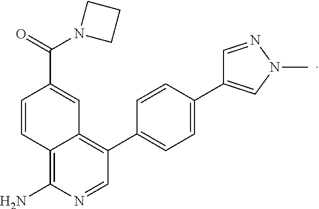 [SMILES: Cn1cc(-c2ccc(-c3cnc(N)c4ccc(C(=O)N5CCC5)cc34)cc2)cn1] | 197 |

In some embodiments, the present invention provides a compound selected from those depicted above, or a pharmaceutically acceptable salt thereof.

Various structural depictions may show a heteroatom without an attached group, radical, charge, or counterion. Those of ordinary skill in the art are aware that such depictions are meant to indicate that the heteroatom is attached to hydrogen (e.g.,

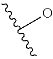 [SMILES: *O]

is understood to be

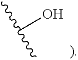 [SMILES: *O]

In certain embodiments, the compounds of the invention were synthesized in accordance with Schemes below. More specific examples of compounds made utilizing the Schemes are provided in the Examples below.

### 4. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably modulate CDK8/19 in a biological sample or in a patient. In certain embodiments, the amount of compound in compositions of this invention is such that is effective to measurably modulate CDK8/19 in a biological sample or in a patient. In certain embodiments, a composition of this invention is formulated for administration to a patient in need of such composition.

The term “patient” or “subject”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention include aqueous or oleaginous suspension. These suspensions are formulated according to techniques known in the art using suitable dispersing or wetting agents and suspending agents. The sterile injectable preparation is also be a sterile injectable solution or suspension in a non-toxic parenterally acceptable diluent or solvent, for example as a solution in 1,3-butanediol. Among the acceptable vehicles and solvents that are employed are water, Ringer's solution and isotonic sodium chloride solution. In addition, sterile, fixed oils are conventionally employed as a solvent or suspending medium.

For this purpose, any bland fixed oil employed includes synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms are also be used for the purposes of formulation.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

Alternatively, pharmaceutically acceptable compositions of this invention are administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention are also administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches are also used.

For topical applications, provided pharmaceutically acceptable compositions are formulated in a suitable ointment containing the active component suspended or dissolved in one or more carriers. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

In certain embodiments, the invention provides a method for antagonizing CDK8/19 in a patient or in a biological sample comprising the step of administering to said patient or contacting said biological sample with a compound according to the invention.

In certain embodiments, the invention is directed to the use of compounds of the invention and/or physiologically acceptable salts thereof, for antagonizing CDK8/19. The compounds are characterized by such a high affinity to CDK8/19, which ensures a reliable binding and preferably antagonization of CDK8/19. In certain embodiments, the substances are mono-specific in order to guarantee an exclusive and directed recognition with the single CDK8/19 target. In the context of the present invention, the term “recognition”—without being limited thereto—relates to any type of interaction between the specific compounds and the target, particularly covalent or non-covalent binding or association, such as a covalent bond, hydrophobic/hydrophilic interactions, van der Waals forces, ion pairs, hydrogen bonds, ligand-receptor interactions, and the like. Such association may also encompass the presence of other molecules such as peptides, proteins or nucleotide sequences. The present receptor/ligand-interaction is characterized by high affinity, high selectivity and minimal or even lacking cross-reactivity to other target molecules to exclude unhealthy and harmful impacts to the treated subject.

In certain embodiments, the present invention relates to a method for antagonizing CDK8/19 with at least one compound of formula (I) according to the invention and/or physiologically acceptable salts thereof, under conditions such that said CDK8/19 receptor is antagonized. In certain embodiments, the system is a cellular system. In other embodiments, the system is an in-vitro translation which is based on the protein synthesis without living cells. The cellular system is defined to be any subject provided that the subject comprises cells. Hence, the cellular system can be selected from the group of single cells, cell cultures, tissues, organs and animals. In certain embodiments, the method for antagonizing CDK8/19 is performed in-vitro. The prior teaching of the present specification concerning the compounds of formula (I), including any embodiments thereof, is valid and applicable without restrictions to the compounds according to formula (I) and their salts when used in the method for antagonizing CDK8/19. The prior teaching of the present specification concerning the compounds of formula (I), including any embodiments thereof, is valid and applicable without restrictions to the compounds according to formula (I) and their salts when used in the method for antagonizing CDK8/19.

In certain embodiments, the compounds according to the invention exhibit an advantageous biological activity, which is easily demonstrated in cell culture-based assays, for example assays as described herein or in prior art (cf. e.g. WO 2002/09706, which is incorporated herein by reference). In such assays, the compounds according to the invention preferably exhibit and cause an agonistic effect.

In certain embodiments, the invention provides a method for preventing, treating or ameliorating in a subject a disease, disorder, or condition that is causally related to the aberrant activity of CDK8/19 receptor, which comprises administering to the subject a therapeutically effective amount of a compound of any formulae herein, or a pharmaceutically acceptable salt thereof.

In certain embodiments, the invention provides compounds and methods for inhibiting the CDKI pathway which may have a variety of clinical applications in chemoprevention and therapy of different age-related diseases. The CDKI pathway inhibitors according to the invention show little or no cytotoxicity in normal cells. These molecules do not interfere with the cell cycle-inhibitory function of CDKIs. They also inhibit the secretion of anti-apoptotic factors by CDKI-arrested cells. These compounds selectively inhibit CDK8 and CDK19 with greater solubility and/or potency than previously described.

In other embodiments, the invention relates to the treatment of cancer using compounds of the invention. In certain embodiments, the invention relates to the prevention of the emergence of cancers (chemoprevention) and prevention of cancer recurrence or metastasis by administering these agents after tumor debulking through surgery, chemotherapy or radiation.

In other embodiments, the disorder is Alzheimer's disease, other dementias, amyloidosis, atherosclerosis, renal disease, or viral diseases. In certain embodiments the viral disease is human immunodeficiency virus (HIV) infection.

In certain embodiments, the disease or disorder is an angiogenesis disease or disorder, proliferative disease or disorder, and/or an angiogenic disease or disorder. In some embodiments, the disease or disorder is a tumor and/or cancer. Examples of cancers and cancer cells include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer.

In certain embodiments, the cancer is brain, lung, colon, epidermoid, squamous cell, bladder, gastric, pancreatic, breast, head, neck, renal, kidney, liver, ovarian, prostate, colorectal, uterine, rectal, oesophageal, testicular, gynecological, thyroid cancer, melanoma, hematologic malignancies such as acute myelogenous leukemia, multiple myeloma, chronic myelogneous leukemia, myeloid cell leukemia, glioma, Kaposi's sarcoma, or any other type of solid or liquid tumors. In some embodiments, the cancer is metastatic cancer. In some embodiments, the cancer is colorectal cancer. In some embodiments, the cancer is colon cancer.

In certain embodiments, the invention provides a method for chemoprotecting a patient at risk for developing cancer, comprising administering to the patient a small molecule compound that specifically inhibits CDK8/19. A patient at risk for cancer includes individuals who have a familial genetic profile that suggests that cancer is likely to develop. It also includes individuals who have been exposed to carcinogenic agents, such as carcinogenic chemicals or viruses or radiation.

In certain embodiments, the invention provides a method for preventing cancer metastasis or recurrence in a cancer patient who has undergone debulking treatment for a tumor, comprising administering to the patient a small molecule compound that specifically inhibits CDK8/19 following debulking of the tumor.

Debulking includes any of the procedures used to treat a primary tumor, such as surgery, chemotherapy and radiation. Despite debulking, there is always a risk of metastasis or incomplete elimination of the primary tumor, resulting in recurrence of the cancer. Administration of a small molecule compound that specifically inhibits CDK8/19 is, therefore, a useful adjuvant therapy to any type of cancer debulking.

In various embodiments, the compound of the invention may be administered alone or in combination with other treatments. A synergistic effect may be achieved by using more than one compound in the pharmaceutical composition, i.e. the compound of formula (I) is combined with at least another agent as active ingredient, which is either another compound of formula (I) or a compound of different structural scaffold. The active ingredients can be used either simultaneously or sequentially.

Included herein are methods of treatment in which at least one chemical entity provided herein is administered in combination with an anti-inflammatory agent. Anti-inflammatory agents include but are not limited to NSAIDs, non-specific and COX-2 specific cyclooxygenase enzyme inhibitors, gold compounds, corticosteroids, methotrexate, tumor necrosis factor (TNF) antagonists, immunosuppressants and methotrexate.

Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib dnd/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-monoclonal antibody (such as eculizumab or pexelizumab), a TNF antagonist, such as entanercept, or infliximab, which is an anti-TNF alpha monoclonal antibody.

Still other embodiments of the invention pertain to combinations in which at least one active agent is an immunosuppressant compound such as an immunosuppressant compound chosen from methotrexate, leflunomide, cyclosporine, tacrolimus, azathioprine, and mycophenolate mofetil.

The compounds of the invention are useful in combination with other chemotherapeutic drugs, in particular, drugs that induce apoptosis. Examples of other chemotherapeutic drugs that can be used in combination with compounds of the invention include topoisomerase I inhibitors (camptothecin or topotecan), topoisomerase II inhibitors (e.g. daunomycin and etoposide), alkylating agents (e.g. cyclophosphamide, melphalan and BCNU), tubulin directed agents (e.g. taxol and vinblastine), and biological agents (e.g. antibodies such as anti CD20 antibody, IDEC 8, immunotoxins, and cytokines).

The disclosed compounds of the formula I can be administered in combination with other known therapeutic agents, including anticancer agents. As used here, the term “anticancer agent” relates to any agent which is administered to a patient with cancer for the purposes of treating the cancer.

The anti-cancer treatment defined above may be applied as a monotherapy or may involve, in addition to the herein disclosed compounds of formula I, conventional surgery or radiotherapy or medicinal therapy. Such medicinal therapy, e.g. a chemotherapy or a targeted therapy, may include one or more, but preferably one, of the following anti-tumor agents:

Alkylating agents: such as altretamine, bendamustine, busulfan, carmustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone; apaziquone, fotemustine, glufosfamide, palifosfamide, pipobroman, trofosfamide, uramustine, TH-302⁴, VAL-083⁴;
Platinum Compounds: such as carboplatin, cisplatin, eptaplatin, miriplatine hydrate, oxaliplatin, lobaplatin, nedaplatin, picoplatin, satraplatin; lobaplatin, nedaplatin, picoplatin, satraplatin;
DNA altering agents: such as amrubicin, bisantrene, decitabine, mitoxantrone, procarbazine, trabectedin, clofarabine; amsacrine, brostallicin, pixantrone, laromustine^{1,3};
Topoisomerase Inhibitors: such as etoposide, irinotecan, razoxane, sobuzoxane, teniposide, topotecan; amonafide, belotecan, elliptinium acetate, voreloxin;
Microtubule modifiers: such as cabazitaxel, docetaxel, eribulin, ixabepilone, paclitaxel, vinblastine, vincristine, vinorelbine, vindesine, vinflunine; fosbretabulin, tesetaxel;
Antimetabolites: such as asparaginase³, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur; doxifluridine, elacytarabine, raltitrexed, sapacitabine, tegafur^{2,3}, trimetrexate;
Anticancer antibiotics: such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin; aclarubicin, peplomycin, pirarubicin;
Hormones/Antagonists: such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol; acolbifene, danazol, deslorelin, epitiostanol, orteronel, enzalutamide^{1,3};
Aromatase inhibitors: such as aminoglutethimide, anastrozole, exemestane, fadrozole, letrozole, testolactone; formestane;
Small molecule kinase inhibitors: such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib; afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib⁴, cabozantinib S-malate^{1,3}, ibrutinib^{1,3}, icotinib⁴, buparlisib², cipatinib⁴, cobimetinib^{1,3}, idelalisib^{1,3}, fedratinib¹, XL-647⁴;
Photosensitizers: such as methoxsalen³; porfimer sodium, talaporfin, temoporfin;
Antibodies: such as alemtuzumab, besilesomab, brentuximab vedotin, cetuximab, denosumab, ipilimumab, ofatumumab, panitumumab, rituximab, tositumomab, trastuzumab, bevacizumab, pertuzumab^{2,3}; catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab^{1,2,3}, onartuzumab^{1,3}, racotumomab¹, tabalumab^{1,3}, EMD-525797⁴, nivolumab^{1,3};
Cytokines: such as aldesleukin, interferon alfa², interferon alfa2a³, interferon alfa2b^{2,3}; celmoleukin, tasonermin, teceleukin, oprelvekin^{1,3}, recombinant interferon beta-1a⁴;
Drug Conjugates: such as denileukin diftitox, ibritumomab tiuxetan, iobenguane I123, prednimustine, trastuzumab emtansine, estramustine, gemtuzumab, ozogamicin, aflibercept; cintredekin besudotox, edotreotide, inotuzumab ozogamicin, naptumomab estafenatox, oportuzumab monatox, technetium (99mTc) arcitumomab^{1,3}, vintafolide^{1,3};
Vaccines: such as sipuleucel³; vitespen³, emepepimut-S³, oncoVAX⁴, rindopepimut³, troVax⁴, MGN-1601⁴, MGN-1703⁴; and
Miscellaneous: alitretinoin, bexarotene, bortezomib, everolimus, ibandronic acid, imiquimod, lenalidomide, lentinan, metirosine, mifamurtide, pamidronic acid, pegaspargase, pentostatin, sipuleucel³, sizofiran, tamibarotene, temsirolimus, thalidomide, tretinoin, vismodegib, zoledronic acid, vorinostat; celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine⁴, picibanil⁴, reolysin⁴, retaspimycin hydrochloride^{1,3}, trebananib^{2,3}, virulizin⁴, carfilzomib^{1,3}, endostatin⁴, immucothel⁴, belinostat³, MGN-1703⁴. (¹ Prop. INN (Proposed International Nonproprietary Name): ² Rec. INN (Recommended International Nonproprietary Names); ³ USAN (United States Adopted Name); ⁴ no INN).

In other embodiments, the invention provides compounds of the invention for use as a pharmaceutical especially in the treatment or prevention of the aforementioned conditions and diseases. Also provided herein is the use of the present compounds in the manufacture of a medicament for the treatment or prevention of one of the aforementioned conditions and diseases. The present invention also provides the use of a compound of the invention or a pharmaceutically acceptable salt thereof for the manufacture of a medicament for the treatment of conditions or diseases selected from CDK8/19 receptor mediated conditions or diseases.

When used to prevent the onset of a CDK8/19 related disease/disorder, the compounds of this invention will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

The method of the invention can be performed either in-vitro or in-vivo. The susceptibility of a particular cell to treatment with the compounds according to the invention can be particularly determined by in-vitro tests, whether in the course of research or clinical application. Typically, a culture of the cell is combined with a compound according to the invention at various concentrations for a period of time which is sufficient to allow the active agents to antagonize CDK8/19 activity, usually between about one hour and one week. In-vitro treatment can be carried out using cultivated cells from a biopsy sample or cell line.

The host or subject can belong to any mammalian species, for example a primate species, particularly humans; rodents, including mice, rats and hamsters; rabbits; horses, cows, dogs, cats, etc. Animal models are of interest for experimental investigations, providing a model for treatment of human disease.

For identification of a signal transduction pathway and for detection of interactions between various signal transduction pathways, suitable models or model systems have been developed, for example cell culture models and models of transgenic animals. For the determination of certain stages in the signal transduction cascade, interacting compounds can be utilized in order to modulate the signal. The compounds according to the invention can also be used as reagents for testing CDK8/19-dependent signal transduction pathways in animals and/or cell culture models or in the clinical diseases mentioned in this application.

The use according to the previous paragraphs of the specification may be either performed in-vitro or in-vivo models. The modulation can be monitored by the techniques described in the course of the present specification. In certain embodiments, the in-vitro use is preferably applied to samples of humans suffering from CDK8/19-related disorders. Testing of several specific compounds and/or derivatives thereof makes the selection of that active ingredient possible that is best suited for the treatment of the human subject. The in-vivo dose rate of the chosen derivative is advantageously pre-adjusted to the CDK8/19 susceptibility and/or severity of disease of the respective subject with regard to the in-vitro data. Therefore, the therapeutic efficacy is remarkably enhanced. Moreover, the subsequent teaching of the present specification concerning the use of the compounds according to formula (I) and its derivatives for the production of a medicament for the prophylactic or therapeutic treatment and/or monitoring is considered as valid and applicable without restrictions to the use of the compound for the antagonism of CDK8/19 activity if expedient.

The invention also relates to the use of compounds according to formula (I) and/or physiologically acceptable salts thereof for the prophylactic or therapeutic treatment and/or monitoring of diseases that are caused, mediated and/or propagated by CDK8/19 activity. Furthermore, the invention relates to the use of compounds according to formula (I) and/or physiologically acceptable salts thereof for the production of a medicament for the prophylactic or therapeutic treatment and/or monitoring of diseases that are caused, mediated and/or propagated by CDK8/19 activity. In certain embodiments, the invention provides the use of a compound according to formula I or physiologically acceptable salts thereof, for the production of a medicament for the prophylactic or therapeutic treatment of a CDK8/19-mediated disorder.

Compounds of formula (I) and/or a physiologically acceptable salt thereof can furthermore be employed as intermediate for the preparation of further medicament active ingredients. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

The compounds of formula (I) according to the invention can be administered before or following an onset of disease once or several times acting as therapy. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to booster the response and eradicate the pathogens and/or symptoms of the disease completely. Either the identical compound or different compounds can be applied. The methods of the invention can also be used to reducing the likelihood of developing a disorder or even prevent the initiation of disorders associated with CDK8/19 activity in advance or to treat the arising and continuing symptoms.

In the meaning of the invention, prophylactic treatment is advisable if the subject possesses any preconditions for the aforementioned physiological or pathological conditions, such as a familial disposition, a genetic defect, or a previously passed disease.

The invention furthermore relates to a medicament comprising at least one compound according to the invention and/or pharmaceutically usable derivatives, salts, solvates and stereoisomers thereof, including mixtures thereof in all ratios. In certain embodiments, the invention relates to a medicament comprising at least one compound according to the invention and/or physiologically acceptable salts thereof.

A “medicament” in the meaning of the invention is any agent in the field of medicine, which comprises one or more compounds of formula (I) or preparations thereof (e.g. a pharmaceutical composition or pharmaceutical formulation) and can be used in prophylaxis, therapy, follow-up or aftercare of patients who suffer from diseases, which are associated with P2X7 activity, in such a way that a pathogenic modification of their overall condition or of the condition of particular regions of the organism could establish at least temporarily.

In another aspect, the invention provides for a kit consisting of separate packs of an effective amount of a compound according to the invention and/or pharmaceutically acceptable salts, derivatives, solvates and stereoisomers thereof, including mixtures thereof in all ratios, and optionally, an effective amount of a further active ingredient. The kit comprises suitable containers, such as boxes, individual bottles, bags or ampoules. The kit may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further active ingredient in dissolved or lyophilized form.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent or delay their recurrence.

The compounds and compositions, according to the method of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention are administered orally or parenterally at dosage levels of about 0.01 mg/kg to about 100 mg/kg and preferably from about 1 mg/kg to about 50 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms optionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions are formulated according to the known art using suitable dispersing or wetting agents and suspending agents. The sterile injectable preparation are also a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This is accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also optionally comprises buffering agents.

Solid compositions of a similar type are also employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type are also employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention can also be utilized as commercial research reagents for various medical research and diagnostic uses. Such uses can include but are not limited to: use as a calibration standard for quantifying the activities of candidate CDK8/19 inhibitors in a variety of functional assays; use as blocking reagents in random compound screening, i.e. in looking for new families of CDK8/19 receptor ligands, the compounds can be used to block recovery of the presently claimed CDK8/19 compounds; use in the co-crystallization with CDK8/19 receptor, i.e. the compounds of the present invention will allow formation of crystals of the compound bound to CDK8/19, enabling the determination of receptor/compound structure by x-ray crystallography; other research and diagnostic applications, etc.; use in assays as probes for determining the expression of CDK8/19 on the surface of cells; and developing assays for detecting compounds which bind to the same site as the CDK8/19 binding ligands.

The compounds of formula (I), their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Antagonism of CDK8/19 activity in a biological sample is useful for a variety of purposes that are known to one of skill in the art. Examples of such purposes include, but are not limited to, blood transfusion, organ transplantation, biological specimen storage, and biological assays.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Compound numbers utilized in the Examples below correspond to compound numbers set forth supra.

¹H NMR was recorded on a 300, 400, or 500 MHz spectrometer, using residual signal of deuterated solvent as internal reference. Chemical shifts (δ) are reported in ppm relative to the residual solvent signal (6=2.49 ppm for 1H NMR in DMSO-d6). 1H NMR data are reported as follows: chemical shift (multiplicity, coupling constants, and number of hydrogens). Multiplicity is abbreviated as follows: s (singlet), d (doublet), t (triplet), q (quartet), m (multiplet), br (broad).

HPLC/MS-Analysis was performed under the following conditions:

### Method A

Solvent A: water+0.05% TFA
Solvent B: acetonitrile+0.05% TFA
Flow: 1 mL/min, wave length: 220 nm

### Gradient: 0.01 min 5% B

### 3.00 min 50% B

### 5.00 min 50% B

### 5.20 min 5% B

5.60 min, stop

### Column: Shim-pack VP-ODS 50-3 mm

Column temp: 40° C.

### Method B

Solvent A: water+0.05% TFA
Solvent B: acetonitrile+0.05% TFA
Flow: 1 mL/min, wave length: 220 nm

### Gradient: 0.01 min 5% B

### 2.20 min 100% B

### 3.20 min 100% B

### 3.30 min 5% B

3.60 min, stop

### Column: Shim-pack VP-ODS 50-3 mm

Column temp: 40° C.

### Method C

Solvent A: water+0.05% TFA
Solvent B: acetonitrile+0.05% TFA
Flow: 1.5 mL/min, wave length: 220 nm

### Gradient: 0.01 min 10% B

### 2.10 min 100% B

### 2.70 min 100% B

### 2.75 min 10% B

3.00 min, stop

### Column: XBridge BEH C18 2.5 μM 50-3 mm

Column temp: 45° C.

### Method D

Solvent A: water+0.05% TFA
Solvent B: acetonitrile+0.05% TFA
Flow: 1 mL/min, wave length: 220 nm

### Gradient: 0.01 min 5% B

### 2.20 min 100% B

### 3.20 min 100% B

### 3.30 min 5% B

3.60 min, stop

### Column: Shim-pack XR-ODS 2.2 μM 50-3 mm

Column temp: 40° C.

### Method E

Solvent A: water+0.1% TFA
Solvent B: acetonitrile+0.1% TFA
Flow: 1.5 mL/min, wave length: 220 nm

### Gradient: 0.01 min 10% B

### 2.00 min 100% B

### 2.60 min 100% B

### 2.70 min 10% B

3.00 min, stop

### Column: Phenomenex Kinetext 2.6 μM 50-3 mm

Column temp: 40° C.

### Method F

Solvent A: water+0.05% TFA
Solvent B: acetonitrile+0.05% TFA
Flow: 1 mL/min, wave length: 220 nm

### Gradient: 0.01 min 5% B

### 4.2 min 70% B

### 5.20 min 70% B

### 5.30 min 5% B

5.60 min, stop

### Column: Shim-pack VP-ODS 50-3 mm

Column temp: 40° C.

### Method G

Solvent A: water+0.05% TFA
Solvent B: acetonitrile+0.05% TFA
Flow: 1 mL/min, wave length: 220 nm

### Gradient: 0.01 min 5% B

### 5.00 min 50% B

### 7.90 min 50% B

### 8.10 min 5% B

8.50 min, stop

### Column: Shim-pack VP-ODS 50-3 mm

Column temp: 40° C.

### Method H

Solvent A: water+0.05% TFA
Solvent B: acetonitrile+0.05% TFA
Flow: 1 mL/min, wave length: 220 nm

### Gradient: 0.01 min 5% B

### 4.20 min 100% B

### 5.20 min 100% B

### 5.30 min 5% B

5.60 min, stop

### Column: Shim-pack VP-ODS 50-3 mm

Column temp: 40° C.

### Method I

Solvent A: water+0.05% TFA
Solvent B: acetonitrile+0.05% TFA
Flow: 1 mL/min, wave length: 220 nm

### Gradient: 0.01 min 5% B

### 1.20 min 100% B

### 2.20 min 100% B

### 2.30 min 5% B

2.60 min, stop

### Column: Shim-pack XR-ODS 2.2 μM 50-3 mm

Column temp: 40° C.

### Method J

Solvent A: water+0.05% TFA
Solvent B: acetonitrile+0.05% TFA
Flow: 1 mL/min, wave length: 220 nm

### Gradient: 0.01 min 5% B

### 3.5 min 80% B

### 5.20 min 80% B

### 5.30 min 5% B

5.60 min, stop

### Column: Shim-pack XR-ODS 50-3 mm, 2.2 μM

Column temp: 40° C.

### Method K

Solvent A: water+0.05% TFA
Solvent B: acetonitrile+0.05% TFA
Flow: 1 mL/min, wave length: 220 nm

### Gradient: 0.01 min 5% B

### 4.0 min 60% B

### 5.20 min 60% B

### 5.30 min 5% B

5.60 min, stop

### Column: Shim-pack XR-ODS 50-3 mm, 2.2 μM

Column temp: 40° C.

### Method L

Solvent A: water+0.1% TFA
Solvent B: acetonitrile+0.1% TFA
Flow: 2 mL/min, wave length: 220 nm

### Gradient: 0.2 min 1% B

### 3.8 min 100% B

4.20 min stop
Column: Chromolith Performance RP18e; 100 mm length inner diameter 3 mm

### Method M

Solvent A: methanol+0.1% formic acid
Solvent B: water+0.1% formic acid
Flow: 3 mL/min, wave length 254 nm

### Gradient: 0.0 min 90% B

### 1.25 min 10% B

### 1.75 min 10% B

### 1.90 min 90% B

### 2.0 min 90% B

Column: Merck Purospher STAR column (RP-18e, 30×4 mm)
Column temp: 40° C.

### Method N

Solvent A: methanol+0.1% formic acid
Solvent B: water+0.1% formic acid
Flow: 1.5 mL/min, wave length 254 nm

### Gradient: 0.0 min 90% B

### 2.50 min 10% B

### 3.50 min 10% B

### 3.80 min 90% B

### 4.00 min 90% B

Column: Merck Purospher STAR column (RP-18e, 30×4 mm)
Column temp: 30° C.

### Method O

Solvent A: methanol+0.1% formic acid
Solvent B: water+0.1% formic acid
Flow: 0.5 mL/min, wave length 254 nm

### Gradient: 0.0 min 90% B

### 1.25 min 10% B

### 1.75 min 10% B

### 1.90 min 90% B

### 2.0 min 90% B

Column: Phenomenex Kinetex XB-C18 column (30×2.1 mm, 1.7 u, 100 A)
Column temp: 30° C.

### Method P

Solvent A: methanol+0.1% formic acid
Solvent B: water+0.1% formic acid
Flow: 0.3 mL/min, wave length 254 nm

### Gradient: 0.0 min 90% B

### 3.00 min 10% B

### 3.50 min 10% B

### 3.80 min 90% B

### 4.00 min 90% B

Column: Merck Purospher STAR column (RP-18e, 30×4 mm)
Column temp: 30° C.

### Method Q

Solvent A: water+0.05% TFA
Solvent B: acetonitrile+0.05% TFA
Flow: 1 mL/min, wave length: 220 nm

### Gradient: 0.01 min 10% B

### 2.00 min 95% B

### 2.60 min 95% B

### 2.70 min 10% B

3.00 min, stop

### Column: ACE UlraCore 2.5 Super C18, 50 mm

Column temp: 40° C.

### Method R

Solvent A: water+0.05% FA
Solvent B: acetonitrile+0.05% FA
Flow: 1 mL/min, wave length: 220 nm

### Gradient: 0.01 min 10% B

### 1.50 min 100% B

### 2.50 min 100% B

2.60 min 10%
2.70 min stop

### Column: Phenomenex Kinetext 2.6 μM, 50-3 mm

Column temp: 40° C.

### Method S

Solvent A: water+0.1% FA
Solvent B: acetonitrile+0.1% FA
Flow: 1 mL/min, wave length: 220 nm

### Gradient: 0.01 min 10% B

### 1.10 min 100% B

### 1.60 min 100% B

1.70 mmin 10%
2.00 min stop

### Column: Phenomenex Kinetext 2.6 μM, 50-3 mm

Column temp: 40° C.

### Preparation of Boronic Ester Intermediates

### 1-methyl-4-(4-(4,4,5,5-tetramethyl-1,3,2-dioxaborolan-2-yl)phenyl)-1H-pyrazole

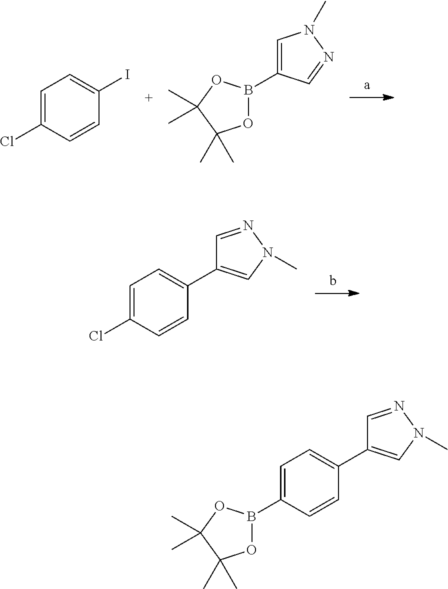

### a. 4-(4-Chlorophenyl)-1-methyl-1H-pyrazole

1-Chloro-4-iodobenzene (6.39 g, 26.8 mmol), 1-methyl-4-(4,4,5,5-tetramethyl-1,3,2-dioxaborolan-2-yl)-1H-pyrazole (5.58 g, 26.8 mmol), sodium carbonate (6.25 g, 59.0 mmol) and Pd(dppf)Cl₂.CH₂Cl₂ (2.20 g, 2.68 mmol) were loaded in a flask and then a mixture of THF/H₂O 3/1 (117 mL) was added. The reaction mixture was heated in an oil bath at 80° C. overnight. It was then concentrated under vacuum and the residue purified by column chromatography (CyHex/EtOAc) to afford the title compound as a white solid (3.80 g, 74%).

### b. 1-Methyl-4-(4-(4,4,5,5-tetramethyl-1,3,2-dioxaborolan-2-yl)phenyl)-1H-pyrazole

4-(4-Chlorophenyl)-1-methyl-1H-pyrazole (3.30 g, 17.1 mmol), bis(pinacolato)diboron (5.20 g, 20.6 mmol), potassium acetate (5.00 g, 51.4 mmol), Xphos (650 mg, 1.37 mmol) and Pd₂dba₃ (310 mg, 0.343 mmol) were loaded in a flask and then dioxane (34.3 mL) was added. The reaction mixture was stirred in an oil bath at 85° C. overnight. The solvent was evaporated and the crude product purified by column chromatography (CyHex/EtOAc) to afford the title compound as a white solid (3.9 g contaminated by 10% of 1-methyl-4-phenyl-1H-pyrazole, corrected yield 75%).

### 1-methyl-5-(4,4,5,5-tetramethyl-1,3,2-dioxaborolan-2-yl)-1,3-dihydrobenzo[c]isothiazole 2,2-dioxide and 5-(4,4,5,5-tetramethyl-1,3,2-dioxaborolan-2-yl)-1,3-dihydrobenzo[c]isothiazole 2,2-dioxide

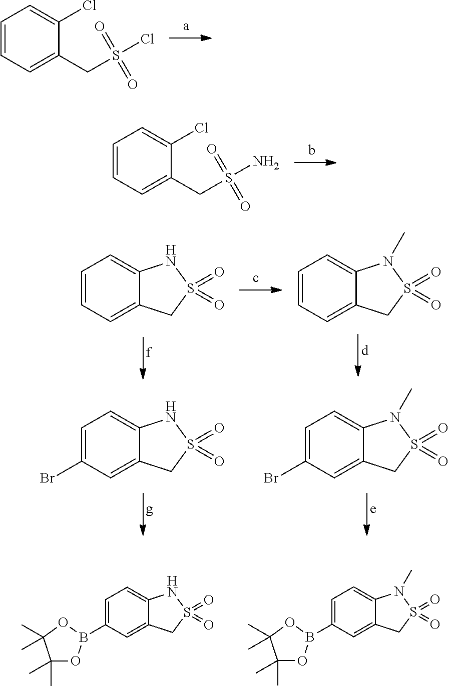

### a. (2-Chlorophenyl)methanesulfonamide

2-Chlorobenzylsulfonyl chloride (1.86 g, 8.26 mmol) was dissolved in acetone (27 mL) and then ammonium hydroxide (18.0 mL, 158 mmol) was added. The reaction was stirred for 2.5 h at rt and the solvent was evaporated. The reaction mixture was diluted with EtOAc and water was added. The two layers were separated and the aqueous layer was extracted with EtOAc. The combined organic layers were dried over magnesium sulphate and concentrated under vacuum. The crude product was purified by column chromatography (dichloromethane/EtOH) to afford the title compound as a white solid (1.50 g, 88%).

### b. 1,3-Dihydrobenzo[c]isothiazole 2,2-dioxide

(2-Chlorophenyl)methanesulfonamide (450 mg, 2.19 mmol), tris(dibenzylideneacetone)dipalladium (100 mg, 0.109 mmol), 2-di-tert-butylphosphino-2′,4′,6′-tri-isopropylbiphenyl (186 mg, 0.438 mmol) and potassium carbonate (605 mg, 4.38 mmol) were loaded in a microwave vial and THF (8.8 mL) was added. The reaction mixture was stirred at 80° C. for 13 h before being quenched with a sat. NH₄Cl solution. The solvent was then evaporated and the residue was purified by column chromatography (CyHex/acetone) to afford the title compound as a white solid (296 mg, 80%).

### c. 1-Methyl-1,3-dihydrobenzo[c]isothiazole-2,2-dioxide

To a suspension of 1,3-dihydrobenzo[c]isothiazole-2,2-dioxide (280 mg, 1.655 mmol) and potassium carbonate (229 mg, 1.66 mmol) in DMF (5 mL) was added iodomethane (414 μL, 6.62 mmol). The reaction was stirred for 6 h at rt and was then quenched with a sat. NH₄Cl solution. The reaction mixture was concentrated and purified by column chromatography (CyHex/acetone) to afford the title compound as a white solid (270 mg, 89%).

### d. 5-Bromo-1-methyl-1,3-dihydrobenzo[c]isothiazole-2,2-dioxide

1-Methyl-1,3-dihydrobenzo[c]isothiazole-2,2-dioxide (272 mg, 1.49 mmol) was dissolved in DMF (1.5 mL) and then N-bromosuccinimide (264 mg, 1.49 mmol) was added. The reaction mixture was stirred at rt for 4 h. After addition of water, the reaction mixture was concentrated. The residue was purified by column chromatography (CyHex/acetone) to afford the title compound as a white solid (330 mg, 85%).

### e. 1-Methyl-5-(4,4,5,5-tetramethyl-1,3,2-dioxaborolan-2-yl)-1,3-dihydrobenzo[c]isothiazole 2,2-dioxide

5-Bromo-1-methyl-1,3-dihydrobenzo[c]isothiazole-2,2-dioxide (267 mg, 1.02 mmol), bis(pinacolato)diboron (388 mg, 1.53 mmol), potassium acetate (300 mg, 3.06 mmol) and Pd(dppf)Cl₂.CH₂Cl₂ (42.0 mg, 0.051 mmol) were loaded in a microwave vial and DME (7.4 mL) was added. The reaction was stirred in an oil bath at 80° C. overnight. The reaction was concentrated and purified by column chromatography (CyHex/acetone) to afford the title compound as a white solid (290 mg, 92%).

### f. 5-bromo-1,3-dihydrobenzo[c]isothiazole 2,2-dioxide

1,3-Dihydro-benzo[c]isothiazole 2,2-dioxide (0.50 g, 3.14 mmol, 1.00 eq.) was solubilized in acetic acid (5 mL) at rt under nitrogen atmosphere. Bromine (0.45 g, 3.14 mmol, 1.00 eq.) in acetic acid (5 mL) was added dropwise over 5 minutes and the reaction mixture was stirred for 0.5 h. Potassium acetate (0.28 g, 3.14 mmol, 1.00 eq.) was added and the reaction mixture was concentrated to dryness. The residue was taken in 2% NaHCO₃ solution and stirred for 10 minutes. This solution was acidified to pH 2 using conc. HCl (2.5 mL) and extracted with MTBE (50 mL). The MTBE layer was washed with water (50 mL), brine solution (25 mL), dried over Na₂SO₄ and concentrated to get the crude product as brown solid. The crude product was triturated with petroleum ether (10 mL), filtered to a light brown solid (HPLC purity app. 86%) which was further purified by column chromatography using 60-120 mesh silica gel, 15% ethyl acetate in petroleum ether as eluent to get a yellow solid (HPLC purity app. 90%). The resulting product was then triturated with ethanol (5 mL), filtered and dried to get the title compounds as light yellow solid (0.35 g, 47.7%, 94% purity).

### g. 5-(4,4,5,5-tetramethyl-1,3,2-dioxaborolan-2-yl)-1,3-dihydrobenzo[c]isothiazole 2,2-dioxide

5-Bromo-1,3-dihydrobenzo[c]isothiazole 2,2-dioxide (500 mg, 2.02 mmol), bis(pinacolato)diboron (768 mg, 3.02 mmol), potassium acetate (593 mg, 6.05 mmol) and Pd(dppf)Cl₂—CH₂Cl₂ (82 mg, 0.10 mmol) were loaded in a microwave vial and DME (14.6 mL) was added. The reaction mixture was heated at 80° C. overnight. The solvent was evaporated and the crude was purified by column chromatography on silica gel (CyHex/acetone) to give the title compound (580 mg contaminated by 23% of pinacol, corrected yield 75%) as a white solid.

### 2-methyl-1-(4-(4-(4,4,5,5-tetramethyl-1,3,2-dioxaborolan-2-yl)phenyl)-1H-pyrazol-1-yl)propan-2-ol

### a. 1-(4-(4-bromophenyl)-1H-pyrazol-1-yl)-2-methylpropan-2-ol

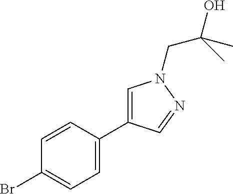 [SMILES: CC(C)(O)Cn1cc(-c2ccc(Br)cc2)cn1]

To a mixture of 4-(4-bromophenyl)pyrazole (508 mg, 2.28 mmol) and K₂CO₃ (557 mg, 4.03 mmol) in DMF (4.5 mL) was added 2,2-dimethyloxirane (0.50 mL, 5.63 mmol) and the mixture heated at 130° C. for 2 h under microwave irradiation. The reaction mixture was diluted with water (25 mL) and extracted with EtOAc (3×25 mL) and the combined org. layers dried (MgSO₄), and concentrated in vacuo to give the title compound as a white solid (644 mg, 96%).

### b. 2-methyl-1-(4-(4-(4,4,5,5-tetramethyl-1,3,2-dioxaborolan-2-yl)phenyl)-1H-pyrazol-1-yl)propan-2-ol

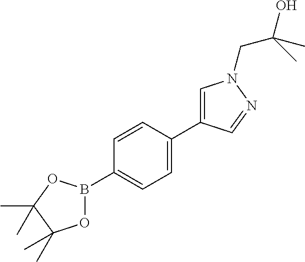 [SMILES: CC(C)(O)Cn1cc(-c2ccc(B3OC(C)(C)C(C)(C)O3)cc2)cn1]

A mixture of 1-(4-(4-bromophenyl)-1H-pyrazol-1-yl)-2-methylpropan-2-ol (642 mg, 2.18 mmol), bis(pinacolato)diboron (810 mg, 3.19 mmol), Pd₂(dba)₃ (100 mg, 0.11 mmol), XPhos (215 mg, 0.45 mmol), and potassium acetate (700 mg, 7.13 mmol) in anhydrous 1,4-dioxane (12 mL) was heated at 80° C. for 18 h. The reaction mixture was allowed to cool to rt and concentrated in vacuo. The crude material was purified by Biotage (SNAP 25 g column, cyclohexane/EtOAc 95/5->70/30) to give the title compound as a yellow oil (710 mg, 95%).

### 1-methyl-4-(4-(4,4,5,5-tetramethyl-1,3,2-dioxaborolan-2-yl)phenyl)-3-(trifluoromethyl)-1H-pyrazole

### a. 4-(4-chlorophenyl)-1-methyl-3-(trifluoromethyl)-1H-pyrazole

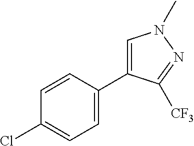 [SMILES: Cn1cc(-c2ccc(Cl)cc2)c(C(F)(F)F)n1]

A mixture of 1-methyl-3-trifluoromethyl-1H-pyrazole-4-boronic acid (50 mg, 0.26 mmol), 1-chloro-4-iodobenzene (80 mg, 0.34 mmol), K₃PO₄ (165 mg, 0.78 mmol), and Pd(dtbpf)Cl₂ (17 mg, 0.026 mmol) in a mixture of 1,4-dioxane (1.4 mL) and water (0.4 mL) was stirred at 120° C. for 30 min under microwave irradiation. The reaction mixture was concentrated in vacuo and purified by Biotage (SNAP 10 g column, cyclohexane/EtOAc 95/5->70/30) to give the title compound as a brown oil (43 mg, 64%).

### b. 1-methyl-4-(4-(4,4,5,5-tetramethyl-1,3,2-dioxaborolan-2-yl)phenyl)-3-(trifluoromethyl)-1H-pyrazole

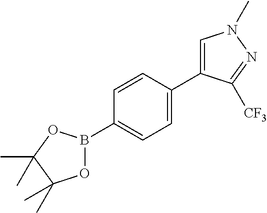 [SMILES: Cn1cc(-c2ccc(B3OC(C)(C)C(C)(C)O3)cc2)c(C(F)(F)F)n1]

A mixture of 4-(4-chlorophenyl)-1-methyl-3-(trifluoromethyl)-1H-pyrazole (275 mg, 1.06 mmol), bis(pinacolato)diboron (400 mg, 1.58 mmol), Pd₂(dba)₃ (50 mg, 0.055 mmol), XPhos (101 mg, 0.21 mmol), and potassium acetate (315 mg, 3.21 mmol) in anhydrous 1,4-dioxane (5.5 mL) was heated at 80° C. for 18 h. The reaction mixture was concentrated in vacuo and purified by Biotage (SNAP 25 g column, cyclohexane/EtOAc 90/10->50/50) to give a yellow oil (399 mg). Used without further purification.

### 1-methyl-5-(4-(4,4,5,5-tetramethyl-1,3,2-dioxaborolan-2-yl)phenyl)-3-(trifluoromethyl)-1H-pyrazole

### a. 5-(4-chlorophenyl)-1-methyl-3-(trifluoromethyl)-1H-pyrazole

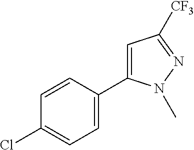 [SMILES: Cn1nc(C(F)(F)F)cc1-c1ccc(Cl)cc1]

A mixture of 1-methyl-3-trifluoromethyl-1H-pyrazole-5-boronic acid (52 mg, 0.27 mmol), 1-chloro-4-iodobenzene (79 mg, 0.33 mmol), K₃PO₄ (165 mg, 0.78 mmol), and Pd(dtbpf)Cl₂ (17 mg, 0.026 mmol) in a mixture of 1,4-dioxane (1.4 mL) and water (0.4 mL) was heated at 80° C. for 1 h under microwave irradiation. The reaction mixture was concentrated in vacuo and purified by Biotage (SNAP 10 g column, cyclohexane/EtOAc 95/5->70/30) to give the title compound as a yellow oil (44 mg, 67%).

### b. 1-methyl-5-(4-(4,4,5,5-tetramethyl-1,3,2-dioxaborolan-2-yl)phenyl)-3-(trifluoromethyl)-1H-pyrazole

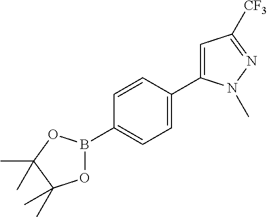 [SMILES: Cn1nc(C(F)(F)F)cc1-c1ccc(B2OC(C)(C)C(C)(C)O2)cc1]

A mixture of 5-(4-chlorophenyl)-1-methyl-3-(trifluoromethyl)-1H-pyrazole (168 mg, 0.65 mmol), bis(pinacolato)diboron (240 mg, 0.95 mmol), Pd₂(dba)₃ (30 mg, 0.033 mmol), XPhos (62 mg, 0.13 mmol), and potassium acetate (190 mg, 1.94 mmol) in anhydrous 1,4-dioxane (3.5 mL) was heated at 80° C. for 18 h. The reaction mixture was concentrated in vacuo and purified by Biotage (SNAP 25 g column, cyclohexane/EtOAc 90/10->50/50) to give the title compound as a yellow oil (189 mg, 83%).

### 2-methyl-1-(5-(4,4,5,5-tetramethyl-1,3,2-dioxaborolan-2-yl)-1H-indazol-1-yl)propan-2-ol and 2-methyl-1-(5-(4,4,5,5-tetramethyl-1,3,2-dioxaborolan-2-yl)-2H-indazol-2-yl)propan-2-ol

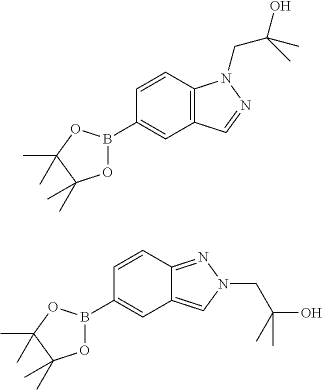

To a mixture of 1H-indazole-5-boronic acid pinacol ester (109 mg, 0.45 mmol) and K₂CO₃ (95 mg, 0.69 mmol) in DMF (1 mL) was added 2,2-dimethyloxirane (0.10 mL, 1.13 mmol) and the mixture heated at 100° C. for 2 h under microwave irradiation. The solution was concentrated in vacuo and the crude material was purified by Biotage (SNAP 25 g column, cyclohexane/EtOAc 100/0->70/30, then CH₂Cl₂/EtOH 70/30->50/50) to give 2-methyl-1-(5-(4,4,5,5-tetramethyl-1,3,2-dioxaborolan-2-yl)-1H-indazol-1-yl)propan-2-ol (76 mg, 54%) as a colourless resin and 2-methyl-1-(5-(4,4,5,5-tetramethyl-1,3,2-dioxaborolan-2-yl)-2H-indazol-2-yl)propan-2-ol (36 mg, 26%) as a colourless resin.

### 1-methyl-4-(3-methyl-4-(4,4,5,5-tetramethyl-1,3,2-dioxaborolan-2-yl)phenyl)-1H-pyrazole

### a. 4-(4-chloro-3-methylphenyl)-1-methyl-1H-pyrazole

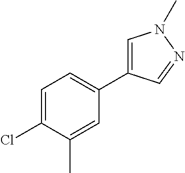

A mixture of 2-chloro-5-bromotoluene (305 mg, 1.48 mmol), 1-methylpyrazole-4-boronic acid pinacol ester (315 mg, 1.51 mmol), Pd(dppf)Cl₂ (130 mg, 0.16 mmol), and K₂CO₃ (400 mg, 2.89 mmol) in a mixture of THF (4 mL) and water (1.2 mL) was heated at 120° C. for 1 h under microwave irradiation. The reaction mixture was concentrated in vacuo and purified by Biotage (SNAP 25 g column, cyclohexane/EtOAc 90/10->50/50) to give the title compound as a brown oil (215 mg, 70%).

### b. 1-methyl-4-(3-methyl-4-(4,4,5,5-tetramethyl-1,3,2-dioxaborolan-2-yl)phenyl)-1H-pyrazole

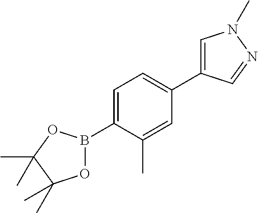 [SMILES: Cc1cc(-c2cnn(C)c2)ccc1B1OC(C)(C)C(C)(C)O1]

A mixture of 4-(4-chloro-3-methylphenyl)-1-methyl-1H-pyrazole (215 mg, 1.04 mmol), bis(pinacolato)diboron (425 mg, 1.67 mmol), Pd₂(dba)₃ (95 mg, 0.10 mmol), XPhos (198 mg, 0.42 mmol), and potassium acetate (325 mg, 3.46 mmol) in anhydrous 1,4-dioxane (4 mL) was heated at 85° C. for 18 h. The reaction mixture was allowed to cool to room temperature, water (40 mL) added, and extracted with CH₂Cl₂ (3×30 mL). The combined organic layers were filtered through a phase separator and concentrated in vacuo. The residue was purified by Biotage (SNAP 25 g column, cyclohexane/EtOAc 95/5->80/20) to give the title compound as a colourless oil (88 mg, 28%).

### 4-(3-fluoro-4-(4,4,5,5-tetramethyl-1,3,2-dioxaborolan-2-yl)phenyl)-1-methyl-1H-pyrazole

### a. 2-fluoro-4-(1-methyl-1H-pyrazol-4-yl)phenol

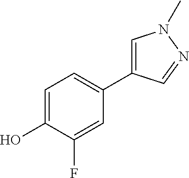

A mixture of 4-bromo-2-fluorophenol (200 mg, 1.05 mmol), 1-methylpyrazole-4-boronic acid pinacol ester (240 mg, 1.15 mmol), K₃PO₄ (670 mg, 3.16 mmol), and Pd(dtbpf)Cl₂ (70 mg, 0.11 mmol) in a mixture of 1,4-dioxane (5 mL) and water (1.4 mL) was heated at 150° C. for 1 h under microwave irradiation. The reaction mixture was then loaded directly onto a 25 g SingleStep column and purified by Biotage (CH₂Cl₂/EtOH 95/5->85/15). The residue was washed with Et₂O to give the title compound as a white solid (95 mg, 47%).

### b. 2-fluoro-4-(1-methyl-1H-pyrazol-4-yl)phenyl trifluoromethanesulfonate

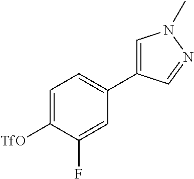 [SMILES: Cn1cc(-c2ccc(OS(=O)(=O)C(F)(F)F)c(F)c2)cn1]

To a cold (0° C.) mixture of 2-fluoro-4-(1-methyl-1H-pyrazol-4-yl)phenol (180 mg, 0.94 mmol) and DMAP (23 mg, 0.19 mmol) in anhyd. CH₂Cl₂ (6 mL) was added PhN(Tf)₂ (502 mg, 1.41 mmol) followed by Et₃N (0.26 mL, 1.85 mmol). The mixture was then allowed to warm to rt and stirred 20 min. Sat. aq. NH₄Cl (25 mL) was added and the mixture extracted with EtOAc (3×25 mL), and the combined organic layers dried (MgSO₄) and concentrated in vacuo. The crude material was purified by Biotage (SingleStep 12 g column, cyclohexane/EtOAc 75/25->50/50) to give the title compound as a colourless oil (286 mg, 94%).

### c. 4-(3-fluoro-4-(4,4,5,5-tetramethyl-1,3,2-dioxaborolan-2-yl)phenyl)-1-methyl-1H-pyrazole

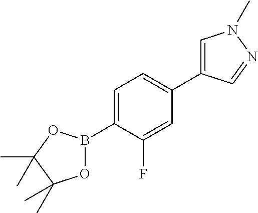 [SMILES: Cn1cc(-c2ccc(B3OC(C)(C)C(C)(C)O3)c(F)c2)cn1]

A mixture of 2-fluoro-4-(1-methyl-1H-pyrazol-4-yl)phenyl trifluoromethanesulfonate (50 mg, 0.15 mmol), bis(pinacolato)diboron (55 mg, 0.22 mmol), Pd₂(dba)₃ (9 mg, 0.0098 mmol), XPhos (19 mg, 0.040 mmol), and potassium acetate (55 mg, 0.56 mmol) in anhyd. 1,4-dioxane (1.2 mL) was heated at rt for 3 h. LCMS after 2 h shows no reaction, so the mixture was heated at 70° C. for 45 min. Concentrated in vacuo and purified by Biotage (SingleStep 12 g column, cyclohexane/EtOAc 80/20->60/40) to give the title compound as a pale yellow oil (34 mg). Used without further purification.

### 1-(2-methoxyethyl)-4-(4-(4,4,5,5-tetramethyl-1,3,2-dioxaborolan-2-yl)phenyl)-1H-pyrazole

### a. 4-(4-bromophenyl)-1-(2-methoxyethyl)-1H-pyrazole

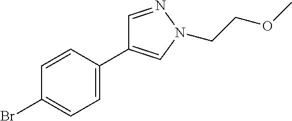 [SMILES: COCCn1cc(-c2ccc(Br)cc2)cn1]

To a mixture of 4-(4-bromophenyl)-1H-pyrazole (100 mg, 0.45 mmol) and K₂CO₃ (130 mg, 0.94 mmol) in DMF (1.4 mL) was added a solution of 1-iodo-2-methoxyethane (115 mg, 0.62 mmol) in DMF (0.3 mL), and the resulting mixture was stirred at rt for 20 h. Additional iodo-2-methoxyethane (30 mg, 0.36 mmol) in DMF (0.1 mL) was added at this point and the mixture heated at 60° C. for 4.5 h. The mixture was allowed to cool to rt and concentrated in vacuo. The residue was purified by Biotage column chromatography (SNAP 10 g column, cyclohexane/EtOAc 100/0->80/20) to give the title compound as a colourless oil (119 mg, 94%).

### b. 1-(2-methoxyethyl)-4-(4-(4,4,5,5-tetramethyl-1,3,2-dioxaborolan-2-yl)phenyl)-1H-pyrazole

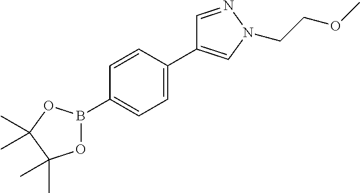

A mixture of 4-(4-bromophenyl)-1-(2-methoxyethyl)-1H-pyrazole (119 mg, 0.42 mmol), bis(pinacolato)diboron (160 mg, 0.63 mmol), Pd₂(dba)₃ (20 mg, 0.022 mmol), XPhos (41 mg, 0.086 mmol), and potassium acetate (125 mg, 1.27 mmol) in anhydrous 1,4-dioxane (2 mL) was heated at 80° C. for 18 h. The mixture was allowed to cool to rt and concentrated in vacuo. The residue was purified by Biotage column chromatography (SNAP 25 g column, CH₂Cl₂/EtOH 100/0->95/5) to give the title compound as a yellow oil (100 mg, 72%).

### 1-isopropyl-4-(4-(4,4,5,5-tetramethyl-1,3,2-dioxaborolan-2-yl)phenyl)-1H-pyrazole

### a. 4-(4-chlorophenyl)-1-isopropyl-1H-pyrazole

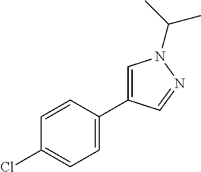

A mixture of 1-isopropyl-4-(4,4,5,5-tetramethyl-1,3,2-dioxaborolan-2-yl)-1H-pyrazole (300 mg, 1.27 mmol), 1-chloro-4-iodobenzene (458 mg, 1.92 mmol), K₃PO₄ (810 mg, 3.82 mmol) and Pd(dtbpf)Cl₂ (85 mg, 0.13 mmol) in 1,4-dioxane (5.5 mL) and water (1 mL) was stirred at 120° C. for 1 h under microwave irradiation. The mixture was then concentrated in vacuo and the residue purified by Biotage column chromatography (SNAP 25 g column, cyclohexane/EtOAc 100/0->70/30) to give the title compound as a brown oil (206 mg, 74%).

### b. 1-isopropyl-4-(4-(4,4,5,5-tetramethyl-1,3,2-dioxaborolan-2-yl)phenyl)-1H-pyrazole

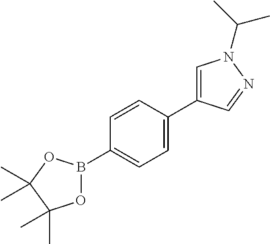 [SMILES: CC(C)n1cc(-c2ccc(B3OC(C)(C)C(C)(C)O3)cc2)cn1]

A mixture of 4-(4-chlorophenyl)-1-isopropyl-1H-pyrazole (262 mg [approx. 90% purity when pooled], 1.07 mmol), bis(pinacolato)diboron (430 mg, 1.69 mmol), Pd₂(dba)₃ (50 mg, 0.055 mmol), XPhos (105 mg, 0.22 mmol), and potassium acetate (315 mg, 3.21 mmol) in anhyd. 1,4-dioxane (5 mL) was heated at 80° C. for 24 h. The mixture was then concentrated in vacuo and the purified by Biotage column chromatography (SNAP 25 g column, cyclohexane/EtOAc 90/10->80/20) to give the title compound as a yellow oil (167 mg, 50%).

### 1-methyl-3-(4-(4,4,5,5-tetramethyl-1,3,2-dioxaborolan-2-yl)phenyl)-1H-pyrazole

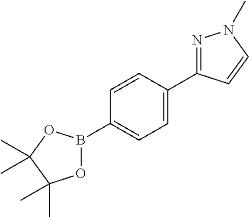 [SMILES: Cn1ccc(-c2ccc(B3OC(C)(C)C(C)(C)O3)cc2)n1]

A mixture of 3-bromo-1-methyl-1H-pyrazole (100 mg, 0.62 mmol), 1,4-bis(4,4,5,5-tetramethyl-1,3,2-dioxaborolan-2-yl)benzene (500 mg, 1.52 mmol), K₃PO₄ (400 mg, 1.88 mmol) and Pd(dtbpf)Cl₂ (41 mg, 0.063 mmol) in 1,4-dioxane (2.5 mL) and water (0.5 mL) was stirred at 120° C. for 1 h under microwave irradiation. The mixture was then concentrated in vacuo and the residue purified by Biotage column chromatography (SNAP 25 g column, CH₂Cl₂/EtOH 100/0->99/1) to give the title compound as a yellow resin (45 mg, 26%).

### 1,2-dimethyl-4-[4-(tetramethyl-1,3,2-dioxaborolan-2-yl)phenyl]-1H-imidazole

### a.1 4-(4-chlorophenyl)-1,2-dimethyl-1H-imidazole

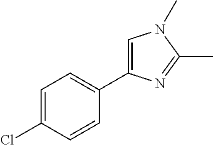

Into a 25-mL round-bottom flask purged and maintained with an inert atmosphere of nitrogen was added 4-bromo-1,2-dimethyl-1H-imidazole (100 mg, 0.57 mmol), (4-chlorophenyl)boronic acid (108 mg, 0.69 mmol), KOAc (169 mg, 1.72 mmol), Pd(dppf)Cl₂*CH₂Cl₂ (47 mg, 0.06 mmol) and dioxane (5 mL). The solution was stirred for 1 h at 100° C. The mixture was concentrated under vacuum and the residue was purified by silica gel column chromatography with ethyl acetate/petrolether (7:3). This resulted in 40 mg (34%) of 4-(4-chlorophenyl)-1,2-dimethyl-1H-imidazole as a brown solid. [M+H]⁺ 207. Rt 1.24 min (method S).

### b. 1,2-dimethyl-4-[4-(tetramethyl-1,3,2-dioxaborolan-2-yl)phenyl]-1H-imidazole

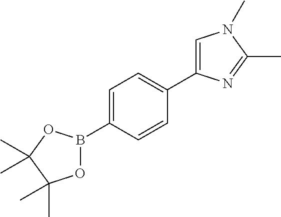

Into a 20-mL vial purged and maintained with an inert atmosphere of nitrogen was added 4-(4-chlorophenyl)-1,2-dimethyl-1H-imidazole (350 mg, 1.69 mmol), 4,4,5,5-tetramethyl-2-(tetramethyl-1,3,2-dioxaborolan-2-yl)-1,3,2-dioxaborolane (645 mg, 2.54 mmol), KOAc (499 mg, 5.08 mmol), Pd(PCy₃)₂Cl₂ (125 mg, 0.17 mmol) and dioxane (10 mL). The mixture was stirred under microwave irradiation for 1 h at 120° C. and concentrated under vacuum. The residue was purified by silica gel column chromatography with ethyl acetate/petrolether (4:1). This resulted in 360 mg (71%) of 1,2-dimethyl-4-[4-(tetramethyl-1,3,2-dioxaborolan-2-yl)phenyl]-1H-imidazole as a light brown solid. [M+H]⁺ 299.

### Example 1

### 3-{4-[4-(1-Methyl-1H-pyrazol-4-yl)-phenyl]-isoquinolin-6-yl}-oxazolidin-2-one (99)

### 1.1 6-Bromo-4-chloroisoquinoline

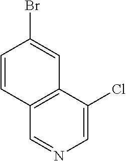 [SMILES: Clc1cncc2ccc(Br)cc12]

6-bromoisoquinoline (2 g, 9.61 mmol) in solution in sulfuryl chloride (5 mL, 61.5 mmol) was heated at 60° C. for 5 min. Another 5 mL of sulfuryl chloride were added and the reaction mixture was heated for 25 min. Sat. NaCO₃ aq. solution was added to the reaction mixture and then ethyl acetate was added. The layers were separated. The aqueous layer was extracted three times with ethyl acetate and the organic layers were combined and dried over MgSO₄. The crude was purified via biotage (dichloromethane/EtOH 99.9/0.01) to give the title compound (1.3 g, 56% yield). [M+H]⁺ 241/243/245. Rt 1.58 min (method M).

### 1.2 3-(4-Chloroisoquinolin-6-yl)-1,3-oxazolidin-2-one

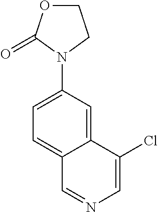 [SMILES: O=C1OCCN1c1ccc2cncc(Cl)c2c1]

Into a 25 mL round-bottom flask purged and maintained with an inert atmosphere of nitrogen, were placed 6-bromo-4-chloroisoquinoline (200 mg, 0.82 mmol), 1,3-oxazolidin-2-one (108 mg, 1.24 mmol), potassium phosphate (525 mg, 2.47 mmol), tris(dibenzylideneacetone)dipalladium(0) (85.4 mg, 0.09 mmol), 2-dicyclohexylphosphino-2′,4′,6′-triisopropylbiphenyl (95.2 mg, 0.16 mmol) and toluene (12 mL). The solution was stirred for 3 h at 80° C. The solution was extracted 3 times with 120 mL of dichloromethane and the organic layers combined and concentrated under vacuum. This resulted in 320 mg (78%) of the title compound as a light yellow solid. [M+H]⁺ 249. Rt 1.26 min (method I).

### 1.3 3-{4-[4-(1-Methyl-1H-pyrazol-4-yl)-phenyl]-isoquinolin-6-yl}-oxazolidin-2-one

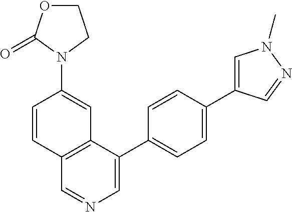 [SMILES: Cn1cc(-c2ccc(-c3cncc4ccc(N5CCOC5=O)cc34)cc2)cn1]

Into a 10 mL sealed tube purged and maintained with an inert atmosphere of nitrogen, were placed 3-(4-chloroisoquinolin-6-yl)-1,3-oxazolidin-2-one (143 mg, 0.58 mmol), 1-methyl-4-[4-(tetramethyl-1,3,2-dioxaborolan-2-yl)phenyl]-1H-pyrazole (259 mg, 0.91 mmol), potassium phosphate (267 mg, 1.26 mmol), Pd(OAc)₂ (11.7 mg, 0.05 mmol), 2-dicyclohexylphosphino-2′,6′-dimethoxybiphenyl (SPhos, 32.8 mg, 0.08 mmol) and toluene (2 mL). The solution was stirred for 1.5 h at 150° C. The mixture was concentrated under vacuum. The residue was applied to a silica gel column with dichloromethane/methanol (100:1) and further purified by prep-HPLC (water/acetonitrile). This resulted in 15.5 mg (7%) of the title compound as a white solid. 1H NMR (300 Hz, DMSO) ppm=9.26 (s, 1H), 8.43 (s, 1H), 8.28-8.26 (m, 2H), 8.11-8.08 (m, 2H), 8.01-7.99 (m, 1H), 7.77-7.75 (m, 2H), 7.58-7.56 (m, 2H), 4.47-4.43 (m, 2H), 4.16-4.12 (m, 2H), 3.91 (s, 3H). [M+H]⁺ 371. Rt 2.5 min (method H).

### Example 2

### (2-Methoxy-ethyl)-{4-[4-(1-methyl-1H-pyrazol-4-yl)-phenyl]-isoquinolin-6-yl}-amine (105)

### 2.1 4-Chloro-N-(2-methoxyethyl)isoquinolin-6-amine

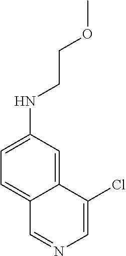 [SMILES: COCCNc1ccc2cncc(Cl)c2c1]

Into a 20 mL round-bottom flask, were placed 6-bromo-4-chloroisoquinoline (100 mg, 0.41 mmol), dioxane (10 mL), sodium tert-butoxide (47.0 mg, 0.49 mmol), (2,2′-bis(diphenylphosphino)-1,1′-binaphthyl (4.50 mg, 0.01 mmol), tris(dibenzylideneacetone)dipalladium(0) (2.00 mg, 0.002 mmol) and 2-methoxyethan-1-amine (31.0 mg, 0.41 mmol). The solution was stirred for 2 h at 100° C. in an oil bath. The mixture was concentrated under vacuum and washed with water. The aqueous phase was extracted with dichloromethane and the combined organic layers were dried over sodium sulfate and concentrated under vacuum. This resulted in 97.2 mg (99%) of the title compound as a yellow solid.

### 2.2 (2-Methoxy-ethyl)-{4-[4-(1-methyl-1H-pyrazol-4-yl)-phenyl]-isoquinolin-6-yl}-amine

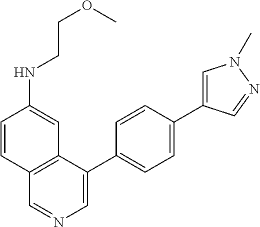 [SMILES: COCCNc1ccc2cncc(-c3ccc(-c4cnn(C)c4)cc3)c2c1]

Into a 20 mL sealed tube, were placed 4-chloro-N-(2-methoxyethyl)isoquinolin-6-amine (97.0 mg, 0.41 mmol), 1-methyl-4-[4-(tetramethyl-1,3,2-dioxaborolan-2-yl)phenyl]-1H-pyrazole (139 mg, 0.49 mmol), acetonitrile (2 mL), water (2 mL), sodium carbonate (106 mg, 1.00 mmol) and Pd(dppf)Cl₂ dichloromethane complex (17.0 mg, 0.02 mmol). The reaction mixture was irradiated with microwave radiation for 1.5 h at 150° C. The mixture was concentrated under vacuum and the crude product was purified by Flash-prep-HPLC (methanol/water). This resulted in 13.0 mg (9%) of the title compound as a yellow solid. ¹H NMR (400 MHz, DMSO-d6) ppm=8.88 (s, 1H), 8.23 (s, 1H), 8.13 (s, 1H), 7.96 (s, 1H), 7.86-7.84 (d, 1H), 7.74-7.72 (d, 2H), 7.52-7.50 (d, 2H), 7.17-7.15 (d, 1H), 6.70 (s, 1H), 6.66-6.64 (t, 1H), 3.91 (s, 3H), 3.51-3.48 (t, 2H), 3.26 (s, 3H), 3.21-3.17 (dd, 2H). [M+H]⁺ 359. Rt 1.54 min (method D).

### Example 3

### 6-(1,1-Dioxo-isothiazolidin-2-yl)-4-[4-(1-methyl-1H-pyrazol-4-yl)-phenyl]-isoquinoline (106)

### 3.1 2-(4-Chloroisoquinolin-6-yl)-2-thiazolidine-1,1-dione

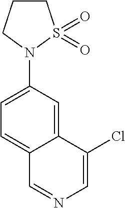 [SMILES: O=S1(=O)CCCN1c1ccc2cncc(Cl)c2c1]

Into a 25 mL round-bottom flask purged and maintained with an inert atmosphere of nitrogen, were placed 6-bromo-4-chloroisoquinoline (200 mg, 0.82 mmol), 2-thiazolidine-1,1-dione (152 mg, 1.25 mmol), tris(dibenzylideneacetone)dipalladium(0) chloroform complex (88.0 mg, 0.09 mmol), 2-dicyclohexylphosphino-2′,4′,6′-triisopropylbiphenyl (144 mg, 0.25 mmol), potassium phosphate (524 mg, 2.47 mmol) and toluene (8 mL). The solution was stirred for 3 h at 100° C. The reaction mixture was cooled to 25° C., concentrated under vacuum and diluted with 20 mL of ethyl acetate. The mixture was washed twice with 15 mL of brine. The organic layer was dried over anhydrous sodium sulfate and concentrated under vacuum. The residue was applied to a silica gel column with dichloromethane/methanol (50:1). This resulted in 170 mg (66%) of the title compound as a yellow solid.

### 3.2 6-(1,1-Dioxo-isothiazolidin-2-yl)-4-[4-(1-methyl-1H-pyrazol-4-yl)-phenyl]-isoquinoline

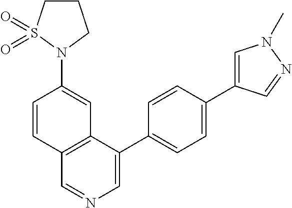 [SMILES: Cn1cc(-c2ccc(-c3cncc4ccc(N5CCCS5(=O)=O)cc34)cc2)cn1]

Into a 10 mL sealed tube, were placed 2-(4-chloroisoquinolin-6-yl)-2-thiazolidine-1,1-dione (150 mg, 0.53 mmol), 1-methyl-4-[4-(tetramethyl-1,3,2-dioxaborolan-2-yl)phenyl]-1H-pyrazole (211 mg, 0.74 mmol), Pd(dppf)Cl₂ dichloromethane complex (22.0 mg, 0.03 mmol), sodium carbonate (79.0 mg, 0.75 mmol), acetonitrile (2 mL) and water (2 mL). The reaction mixture was irradiated with microwave radiation for 1.5 h at 150° C. The reaction mixture was cooled to 20° C. The solution was extracted twice with 15 mL of ethyl acetate and the organic layers were combined and washed with 1×15 mL of brine. The organic layers was dried over anhydrous sodium sulfate and concentrated under vacuum. The residue was applied to a silica gel column with acetonitrile/water (1:1). This resulted in 90.0 mg (40%) of the title compound as a white solid. ¹H NMR (300 MHz, DMSO-d6) ppm=9.23 (s, 1H), 8.40 (s, 1H), 8.24 (d, J=10.1, 2H), 7.98 (s, 1H), 7.75 (d, J=8.1, 2H), 7.69 (dd, J=9.0, 2.0, 1H), 7.56 (d, J=8.2, 2H), 7.51-7.49 (m, 1H), 3.90 (s, 3H), 3.82 (t, J=6.4, 2H), 3.57 (t, J=7.2, 2H), 2.46-2.34 (m, 2H). [M+H]⁺ 405. Rt 2.13 min (method A).

### Example 4

### 6-(1,1-Dioxo-[1,2]thiazinan-2-yl)-4-[4-(1-methyl-1H-pyrazol-4-yl)-phenyl]-isoquinoline (110)

### 4.1 2-(4-Chloroisoquinolin-6-yl)-2-thiazinane-1,1-dione

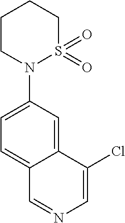 [SMILES: O=S1(=O)CCCCN1c1ccc2cncc(Cl)c2c1]

Into a 100 mL round-bottom flask purged and maintained with an inert atmosphere of nitrogen, were placed 6-bromo-4-chloroisoquinoline (150 mg, 0.62 mmol), 2-thiazinane-1,1-dione (167 mg, 1.24 mmol), tris(dibenzylideneacetone)dipalladium(0) chloroform complex (96.0 mg, 0.09 mmol), 2-dicyclohexylphosphino-2′,4′,6′-triisopropylbiphenyl (125 mg, 0.22 mmol), potassium phosphate (657 mg, 3.10 mmol) and toluene (10 mL). The solution was stirred for 3 h at 100° C. The reaction mixture was cooled to 25° C., concentrated under vacuum and diluted with 20 mL of ethyl acetate. The organic phase was washed twice with 15 mL of brine and dried over anhydrous sodium sulfate and concentrated under vacuum. The residue was applied to a silica gel column with dichloromethane/methanol (50:1). This resulted in 183 mg (80%) of the title compound as a orange solid.

### 4.2 6-(1,1-Dioxo-[1,2]thiazinan-2-yl)-4-[4-(1-methyl-1H-pyrazol-4-yl)-phenyl]-isoquinoline

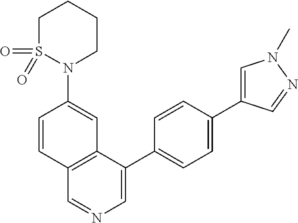 [SMILES: Cn1cc(-c2ccc(-c3cncc4ccc(N5CCCCS5(=O)=O)cc34)cc2)cn1]

Into a 10 mL sealed tube purged and maintained with an inert atmosphere of nitrogen were placed 2-(4-chloroisoquinolin-6-yl)-2-thiazinane-1,1-dione (200 mg, 0.67 mmol), 1-methyl-4-[4-(tetramethyl-1,3,2-dioxaborolan-2-yl)phenyl]-1H-pyrazole (246 mg, 0.87 mmol), Pd(dppf)Cl₂ dichloromethane complex (25.0 mg, 0.03 mmol), sodium carbonate (92.0 mg, 0.87 mmol), acetonitrile (2.5 mL) and water (2.5 mL). The reaction mixture was stirred under microwave irradiation for 1.5 h at 150° C., cooled to 25° C. and concentrated under vacuum. The aqueous solution was extracted twice with 15 mL of ethyl acetate. The combined organic layer was washed with 15 mL of brine, dried over anhydrous sodium sulfate and evaporated to dryness. The residue was applied to a silica gel column with acetonitrile/water (1:1). This resulted in 50.2 mg (17%) of the title compound as a white solid. ¹H NMR (300 MHz, DMSO-d6) ppm=9.33 (s, 1H), 8.48 (s, 1H), 8.25 (d, J=10.3, 2H), 7.99 (s, 1H), 7.79-7.76 (m, 3H), 7.70 (dd, J=8.8, 1.8, 1H), 7.56 (d, J=8.1, 2H), 3.90 (s, 3H), 3.84-3.71 (m, 2H), 3.36-3.34 (m, 2H), 2.26-2.06 (m, 2H), 1.93-1.73 (m, 2H). [M+H]⁺ 419. Rt 2.71 min (method J).

### Example 5

### 4-Methyl-1-{4-[4-(1-methyl-1H-pyrazol-4-yl)-phenyl]-isoquinolin-6-yl}-pyrrolidin-2-one (111)

### 5.1 1-(4-Chloroisoquinolin-6-yl)-4-methylpyrrolidin-2-one

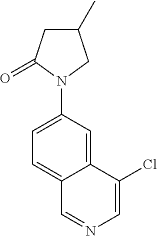 [SMILES: CC1CC(=O)N(c2ccc3cncc(Cl)c3c2)C1]

Into a 25 mL round-bottom flask purged and maintained with an inert atmosphere of nitrogen, were placed 6-bromo-4-chloroisoquinoline (50.0 mg, 0.21 mmol), 4-methylpyrrolidin-2-one (27.5 mg, 0.28 mmol), potassium phosphate (184 mg, 0.86 mmol), tris(dibenzylideneacetone)dipalladium(0) chloroform complex (19.7 mg, 0.02 mmol), 4,5-bis(diphenylphosphino)-9,9-dimethylxanthene (Xantphos, 16.7 mg, 0.03 mmol) and toluene (5 mL). The solution was stirred for 3 h at 100° C. The mixture was concentrated under vacuum. The residue was applied to a silica gel column with dichloromethane/methanol (100:1). This resulted in 23.9 mg (27%) of the title compound as a yellow solid.

### 5.2 4-Methyl-1-{4-[4-(1-methyl-1H-pyrazol-4-yl)-phenyl]-isoquinolin-6-yl}-pyrrolidin-2-one

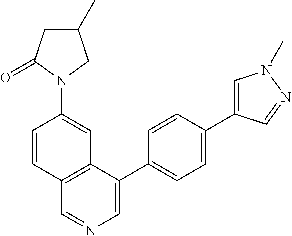 [SMILES: CC1CC(=O)N(c2ccc3cncc(-c4ccc(-c5cnn(C)c5)cc4)c3c2)C1]

Into a 10 mL vial purged and maintained with an inert atmosphere of nitrogen, were placed 1-(4-chloroisoquinolin-6-yl)-4-methylpyrrolidin-2-one (170 mg, 0.65 mmol), 1-methyl-4-[4-(tetramethyl-1,3,2-dioxaborolan-2-yl)phenyl]-1H-pyrazole (241 mg, 0.85 mmol), sodium carbonate (117 mg, 1.10 mmol), Pd(dppf)Cl₂ dichloromethane complex (43.0 mg, 0.05 mmol), acetonitrile (1 mL) and water (1 mL). The solution was stirred for 1.5 h at 150° C. The mixture was concentrated under vacuum. The residue was applied to a silica gel column with dichloromethane/methanol (100:1). This resulted in 31.8 mg (12%) of the title compound as a white solid. ¹H NMR (300 MHz, DMSO-d6) ppm=9.24 (s, 1H), 8.40-8.05 (m, 4H), 7.98 (s, 1H), 7.76 (d, 2H), 7.56 (d, 2H), 4.00 (t, 1H), 3.50 (t, 1H), 3.30 (s, 1H), 2.71-2.63 (m, 1H), 2.26-2.18 (m, 1H), 1.11 (d, 3H). [M+H]⁺ 383. Rt 1.58 min (method D).

### Example 6

### 1-{4-[4-(1-Methyl-1H-pyrazol-4-yl)-phenyl]-isoquinolin-6-yl}-pyrrolidin-3-one (115)

### 6.1 1-(4-Chloroisoquinolin-6-yl)pyrrolidin-3-ol

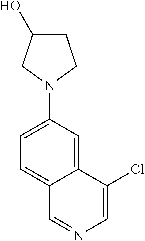 [SMILES: OC1CCN(c2ccc3cncc(Cl)c3c2)C1]

Into a 10-mL sealed tube, were placed 6-bromo-4-chloroisoquinoline (100 mg, 0.41 mmol), dioxane (5.00 mL), t-BuONa (120 mg, 1.25 mmol), BINAP (20 mg, 0.03 mmol), Pd₂(dba)₃ chloroforme complex (10.0 mg, 0.01 mmol) and pyrrolidin-3-ol hydrochloride (76.4 mg, 0.62 mmol). The mixture was stirred for 2 h at 100° C. The reaction mixture was concentrated under vacuum, water was added and extracted with dichloromethane twice. The combined organic layers were dried over sodium sulfate, filtered and concentrated to dryness. This resulted in 100 mg (98%) of the title compound as a yellow solid.

### 6.2 1-{4-[4-(1-Methyl-1H-pyrazol-4-yl)-phenyl]-isoquinolin-6-yl}-pyrrolidin-3-ol

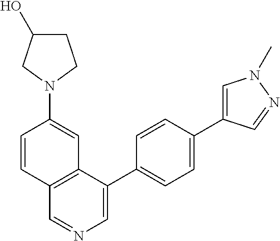 [SMILES: Cn1cc(-c2ccc(-c3cncc4ccc(N5CCC(O)C5)cc34)cc2)cn1]

Into a 30-mL sealed tube were placed 1-(4-chloroisoquinolin-6-yl)pyrrolidin-3-ol (335.00 mg, 1.35 mmol), 1-methyl-4-[4-(tetramethyl-1,3,2-dioxaborolan-2-yl)phenyl]-1H-pyrazole (536 mg, 1.89 mmol, Pd(dppf)Cl₂ dichloromethane complex (110 mg, 0.13 mmol), sodium carbonate (286 mg, 2.69 mmol), water (12 mL) and acetonitrile (12 mL). The reaction mixture was stirred under microwave irradiation for 1.5 h at 150° C. The resulting mixture was concentrated under vacuum. The residue was applied onto a silica gel column with dichloromethane/methanol (50:1). This resulted in 220 mg (44%) of the title compound as a yellow solid.

### 6.3 1-{4-[4-(1-Methyl-1H-pyrazol-4-yl)-phenyl]-isoquinolin-6-yl}-pyrrolidin-3-one

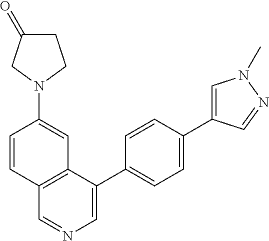 [SMILES: Cn1cc(-c2ccc(-c3cncc4ccc(N5CCC(=O)C5)cc34)cc2)cn1]

Into a 10 mL round-bottom flask, were placed 1-[4-[4-(1-methyl-1H-pyrazol-4-yl)phenyl]isoquinolin-6-yl]pyrrolidin-3-ol (50.0 mg, 0.13 mmol), DMSO (2 mL), triethylamine (150 mg, 1.48 mmol) and SO₃-pyridine (75.0 mg, 0.47 mmol). The solution was stirred for 2 h at 25° C. The mixture was concentrated under vacuum. The residue was applied to a silica gel column with dichloromethane/methanol (100:1). This resulted in 30.1 mg (63%) of the title compound as a yellow solid. ¹H NMR (300 MHz, DMSO-d6) ppm=8.99 (s, 1H), 8.21 (s, 1H), 8.12 (s, 1H), 8.02 (d, 1H), 7.87 (s, 1H), 7.72 (d, 2H), 7.52 (d, 2H), 7.253 (dd, 1H), 6.80 (d, 1H), 3.89 (s, 3H), 3.77 (s, 2H), 3.75 (t, 2H), 2.70 (t, 2H). [M+H]⁺ 369. Rt 2.39 min (method H).

### Example 7

### 4-{4-[4-(1-Methyl-1H-pyrazol-4-yl)-phenyl]-isoquinolin-6-yl}-piperazin-2-one (116)

### 7.1 4-(4-Chloro-isoquinolin-6-yl)-piperazin-2-one

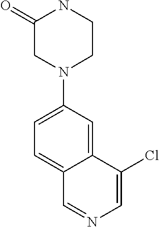 [SMILES: O=C1CN(c2ccc3cncc(Cl)c3c2)CCN1]

Into a 20 mL sealed tube, were placed 6-bromo-4-chloroisoquinoline (150 mg, 0.62 mmol), dioxane (9 mL), (2,2′-bis(diphenylphosphino)-1,1′-binaphthyl (38.5 mg, 0.06 mmol), sodium tert-butoxide (178 mg, 1.85 mmol), tris(dibenzylideneacetone)dipalladium(0) chloroform complex (32.0 mg, 0.03 mmol) and piperazin-2-one (62.0 mg, 0.62 mmol). The solution was stirred for 2 h at 100° C. in an oil bath. The mixture was concentrated under vacuum. The residue was applied to a silica gel column with dichloromethane/methanol (100:1). This resulted in 85.0 mg (53%) of the title compound as a yellow solid.

### 7.2 4-{4-[4-(1-Methyl-1H-pyrazol-4-yl)-phenyl]-isoquinolin-6-yl}-piperazin-2-one

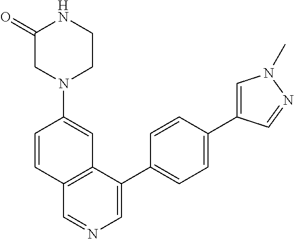 [SMILES: Cn1cc(-c2ccc(-c3cncc4ccc(N5CCNC(=O)C5)cc34)cc2)cn1]

Into a 10 mL sealed tube were placed 4-(4-chloro-isoquinolin-6-yl)-piperazin-2-one (85.0 mg, 0.32 mmol), 1-methyl-4-[4-(tetramethyl-1,3,2-dioxaborolan-2-yl)phenyl]-1H-pyrazole (129 mg, 0.45 mmol), Pd(dppf)Cl₂ dichloromethane complex (26.5 mg, 0.03 mmol), sodium carbonate (68.9 mg, 0.65 mmol), water (4.25 mL) and acetonitrile (4.25 mL). The reaction mixture was stirred under microwave irradiation for 1.5 h at 150° C. The mixture was concentrated under vacuum. The residue was applied to a silica gel column with dichloromethane/methanol (100:1). This resulted in 14.3 mg (11%) of the title compound as a yellow solid. ¹H NMR (400 MHz, CD₃OD) ppm=9.06 (s, 1H), 8.20 (s, 1H), 8.16 (d, 1H), 8.10 (s, 1H), 7.95 (s, 1H), 7.79 (d, 2H), 7.62 (dd, 1H), 7.58 (d, 2H), 7.11 (d, 1H), 3.99 (s, 5H), 3.72 (t, 2H), 3.50 (t, 2H). [M+H]⁺ 384. Rt 2.37 min (method F).

### Example 8

### 1-(4-{4-[4-(1-Methyl-1H-pyrazol-4-yl)-phenyl]-isoquinolin-6-yl}-piperazin-1-yl)-ethanone (125)

### 8.1 1-[4-(4-Chloroisoquinolin-6-yl)piperazin-1-yl]ethan-1-one

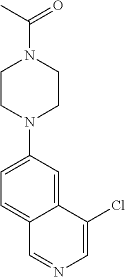 [SMILES: CC(=O)N1CCN(c2ccc3cncc(Cl)c3c2)CC1]

Into a 20 mL sealed tube, were placed 6-bromo-4-chloroisoquinoline (150 mg, 0.62 mmol), dioxane (10 mL), sodium tert-butoxide (72.0 mg, 0.75 mmol), (2,2′-bis(diphenylphosphino)-1,1′-binaphthyl (7.00 mg, 0.01 mmol), tris(dibenzylideneacetone)dipalladium(0) (3.00 mg, 0.003 mmol) and 1-(piperazin-1-yl)ethan-1-one (80.0 mg, 0.62 mmol). The solution was stirred for 2 h at 100° C. in an oil bath and concentrated under vacuum. This resulted in 150 mg (84%) of the title compound as a yellow solid.

### 8.2 1-(4-{4-[4-(1-Methyl-1H-pyrazol-4-yl)-phenyl]-isoquinolin-6-yl}-piperazin-1-yl)-ethanone

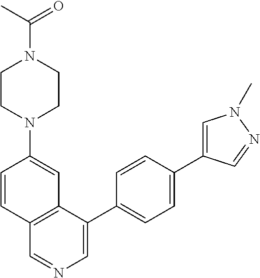 [SMILES: CC(=O)N1CCN(c2ccc3cncc(-c4ccc(-c5cnn(C)c5)cc4)c3c2)CC1]

Into a 20 mL sealed tube, were placed 1-[4-(4-chloroisoquinolin-6-yl)piperazin-1-yl]ethan-1-one (150 mg, 0.52 mmol), 1-methyl-4-[4-(tetramethyl-1,3,2-dioxaborolan-2-yl)phenyl]-1H-pyrazole (222 mg, 0.78 mmol), acetonitrile (3 mL), water (3 mL), sodium carbonate (84.0 mg, 0.79 mmol) and Pd(dppf)Cl₂ dichloromethane complex (42.0 mg, 0.05 mmol). The reaction mixture was stirred unders microwave irradiation for 1.5 h at 150° C. The mixture was concentrated under vacuum. The crude product was purified by Flash-prep-HPLC (methanol/water). This resulted in 18.0 mg (8%) of 1-(4-[4-[4-(1-methyl-1H-pyrazol-4-yl)phenyl]isoquinolin-6-yl]piperazin-1-yl)ethan-1-one as a yellow solid. ¹H NMR (400 MHz, DMSO-d6) ppm=9.06 (s, 1H), 8.26 (d, 2H), 8.05 (d, 1H), 7.97 (s, 1H), 7.75 (d, 2H), 7.60-7.54 (m, 3H), 7.05 (s, 1H), 3.91 (s, 3H), 3.62-3.55 (m, 4H), 3.28-3.27 (m, 4H), 2.03 (s, 3H). [M+H]⁺ 412. Rt 1.48 min (method B).

### Example 9

### 1-{4-[4-(1-Methyl-1H-pyrazol-4-yl)-phenyl]-isoquinolin-6-yl}-piperidin-2-one (127)

### 9.1 1-(4-Chloroisoquinolin-6-yl)piperidin-2-one

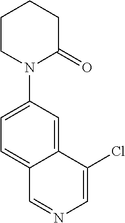 [SMILES: O=C1CCCCN1c1ccc2cncc(Cl)c2c1]

Into a 25 mL round-bottom flask purged and maintained with an inert atmosphere of nitrogen, were placed 6-bromo-4-chloroisoquinoline (100 mg, 0.41 mmol), piperidin-2-one (54.0 mg, 0.54 mmol), tris(dibenzylideneacetone)dipalladium(0) chloroform complex (22.0 mg, 0.02 mmol), 2-dicyclohexylphosphino-2′,4′,6′-triisopropylbiphenyl (24.0 mg, 0.04 mmol), potassium phosphate (262 mg, 1.23 mmol) and toluene (6 mL). The solution was stirred for 2 h at 100° C. The reaction mixture was cooled to 25° C., concentrated under vacuum and diluted with ethyl acetate. The mixture was washed with brine. The organic layer was dried over sodium sulfate and concentrated under vacuum. This resulted in 110 mg (72%) of the title compound as brown oil.

### 9.2 1-{4-[4-(1-Methyl-1H-pyrazol-4-yl)-phenyl]-isoquinolin-6-yl}-piperidin-2-one

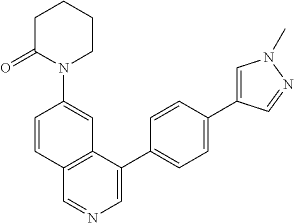 [SMILES: Cn1cc(-c2ccc(-c3cncc4ccc(N5CCCCC5=O)cc34)cc2)cn1]

Into a 10 mL sealed tube, were placed 1-(4-chloroisoquinolin-6-yl)piperidin-2-one (100 mg, 0.38 mmol), 1-methyl-4-[4-(tetramethyl-1,3,2-dioxaborolan-2-yl)phenyl]-1H-pyrazole (164 mg, 0.58 mmol), Pd(dppf)Cl₂ dichloromethane complex (16.0 mg, 0.02 mmol), sodium carbonate (58.0 mg, 0.55 mmol), acetonitrile (1.5 mL) and water (1.5 mL). The reaction mixture was irradiated with microwave radiation for 1.5 h at 150° C. The reaction mixture was cooled to 25° C. The solution was extracted twice with 10 mL of ethyl acetate. The combined organic layer was washed with 15 mL of brine. The organic layer was dried over anhydrous sodium sulfate and concentrated under vacuum. The crude product was purified by prep-HPLC (water/acetonitrile). This resulted in 15.3 mg (10%) of the title compound as a white solid. ¹H NMR (400 MHz, DMSO-d6) ppm=9.30 (s, 1H), 8.44 (s, 1H), 8.25 (s, 1H), 8.21 (d, J=8.8, 1H), 7.97 (s, 1H), 7.81-7.73 (m, 3H), 7.70 (dd, J=8.7, 1.9, 1H), 7.53 (d, J=8.2, 2H), 3.90 (s, 3H), 3.68 (t, J=5.6, 2H), 2.41 (t, J=6.4, 2H), 1.89-1.83 (m, 4H). [M+H]⁺ 383. Rt 2.93 min (method G).

### Example 10

### 1-(4-(4-(1-Methyl-1H-pyrazol-4-yl)phenyl)isoquinolin-6-yl)pyrrolidin-2-one (128)

### 10.1 1-(4-Chloroisoquinolin-6-yl)pyrrolidin-2-one

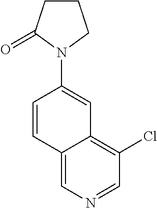 [SMILES: O=C1CCCN1c1ccc2cncc(Cl)c2c1]

A mixture of 6-bromo-4-chloroisoquinoline (50 mg, 0.206 mmol), pyrrolidinone (0.019 mL, 0.247 mmol), potassium phosphate (245 mg, 1.155 mmol), Pd₂(dba)₃ (30.2 mg, 0.033 mmol) and Xantphos (39.4 mg, 0.068 mmol) in toluene (1.8 mL) was heated to 60° C. for 1 hr. The mixture was concentrated in vacuum and the resulting brown oil was purified by chromatography on silica gel (biotage, CH₂Cl₂/EtOH, 100:0 to 96:4) and further purified by using an scx₂-cartridge (loading with CH₂Cl₂/MeOH 9/1, elution with CH₂Cl₂/1N NH₃ in MeOH 9/1) to give the title compound (38 mg, 75%) as a light brown solid.

### 10.2 1-(4-(4-(1-Methyl-1H-pyrazol-4-yl)phenyl)isoquinolin-6-yl)pyrrolidin-2-one

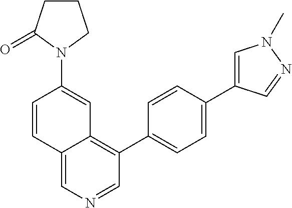 [SMILES: Cn1cc(-c2ccc(-c3cncc4ccc(N5CCCC5=O)cc34)cc2)cn1]

1-(4-Chloroisoquinolin-6-yl)pyrrolidin-2-one (35 mg, 0.142 mmol), 1-methyl-4-(4-(4,4,5,5-tetramethyl-1,3,2-dioxaborolan-2-yl)phenyl)-1H-pyrazole (40.3 mg, 0.142 mmol) and Pd(dppf)Cl₂.CH₂Cl₂ (5.19 mg, 7.09 μmol) were loaded in a microwave vial. The capped vial was evacuated using high vacuum and purged with nitrogen (each three times). Acetonitrile (1.3 mL) and aqueous sodium carbonate (0.5M, 0.397 mL, 0.199 mmol) were added and the mixture was degassed again by using the high vacuum and purged with nitrogen again (each three times). The mixture was heated in the microwave at 150° C. for 2 hr. Because the conversion was not complete additional Pd(dppf)Cl₂.CH₂Cl₂ (5.19 mg, 7.09 μmol) and 1-methyl-4-(4-(4,4,5,5-tetramethyl-1,3,2-dioxaborolan-2-yl)phenyl)-1H-pyrazole (16.13 mg, 0.057 mmol) were added and the mixture was heated in the microwave at 150° C. for another 1 h before the mixture was transferred into a flask with the help of CHCl₃ and the water was evaporated by azeotropic removal with toluene. The resulting brown solid was purified by chromatography on silica gel (biotage, CH₂Cl₂/MeOH, 100:0 to 95:5) and further purified by prep. TLC (CH₂Cl₂/MeOH, 98:2) to give the title compound (28.7 mg, 55%) as a light brown solid after evaporation with CH₂Cl₂/CyHex. ¹H NMR (500 MHz, CDCl₃/CD₃OD, 1:1) ppm=9.10 (s, 1H), 8.34 (s, 1H), 8.16 (dd, J=9.0, 2.1, 1H), 8.09 (d, J=9.0, 1H), 8.01 (s, 1H), 7.87 (s, 1H), 7.84 (s, 1H), 7.66 (d, J=8.2, 2H), 7.51 (d, J=8.2, 2H), 3.96 (s, 3H), 3.92 (t, J=7.1, 2H), 2.62 (t, J=8.1, 2H), 2.18 (tt, J=8.1, 7.1, 2H). [M+H]⁺ 369. Rt 1.2 min (method M).

### Example 11

### 1-(4-(1-Methyl-2,2-dioxido-1,3-dihydrobenzo[c]isothiazol-5-yl)isoquinolin-6-yl)pyrrolidin-2-one (129)

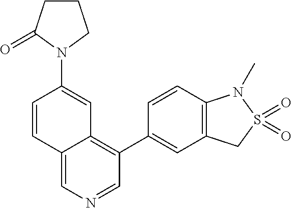 [SMILES: CN1c2ccc(-c3cncc4ccc(N5CCCC5=O)cc34)cc2CS1(=O)=O]

1-(4-(1-Methyl-2,2-dioxido-1,3-dihydrobenzo[c]isothiazol-5-yl)isoquinolin-6-yl)pyrrolidin-2-one was prepared according to the conditions provided in Example 10, using 1-methyl-5-(4,4,5,5-tetramethyl-1,3,2-dioxaborolan-2-yl)-1,3-dihydrobenzo[c]isothiazole 2,2-dioxide as the starting material. ¹H-NMR (500 MHz, CD₃OD/CDCl₃ 1/1) ppm=9.10 (s, 1H), 8.30 (s, 1H), 8.11 (d, J=9.0, 1H), 8.08 (dd, J=9.0, 2.0, 1H), 8.05-8.03 (m, 1H), 7.52 (dd, J=8.1, 1.8, 1H), 7.48 (d, J=1.8, 1H), 6.98 (d, J=8.1, 1H), 4.54 (s, 2H), 3.95 (t, J=7.1, 2H), 3.20 (s, 3H), 2.64 (t, J=8.1, 2H), 2.31-2.08 (m, 2H) [M+H]⁺ 394. Rt 1.03 min (ethod M).

### Example 12

### 5-Methyl-1-{4-[4-(1-methyl-1H-pyrazol-4-yl)-phenyl]-isoquinolin-6-yl}-pyrrolidin-2-one (114)

### 12.1 1-(4-chloroisoquinolin-6-yl)-5-methylpyrrolidin-2-one

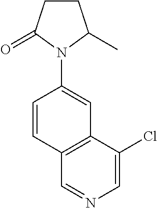 [SMILES: CC1CCC(=O)N1c1ccc2cncc(Cl)c2c1]

Into a 50-mL round-bottom flask purged and maintained with an inert atmosphere of nitrogen were placed 6-bromo-4-chloroisoquinoline (161 mg, 0.66 mmol), 5-methylpyrrolidin-2-one (81.0 mg, 0.82 mmol), potassium phosphate (394 mg, 1.86 mmol), tris(dibenzylideneacetone)dipalladium(0) chloroform complex (32.1 mg, 0.03 mmol), 4,5-bis(diphenylphosphino)-9,9-dimethylxanthene (Xantphos, 35.7 mg, 0.06 mmol) and toluene (15 mL). The mixture was stirred for 3 h at 100° C. The reaction mixture was concentrated to dryness, redissolved in 20 mL of dichloromethane and washed with water (10 mL) and brine (10 mL). The organic phase was dried over sodium sulfate, filtered and concentrated to dryness. The residue was purified by silica gel chromatography (petrol ether/ethyl acetate). This resulted in 165 mg (73%) of the title compound as a light yellow solid.

### 12.2 5-Methyl-1-{4-[4-(1-methyl-1H-pyrazol-4-yl)-phenyl]-isoquinolin-6-yl}-pyrrolidin-2-one

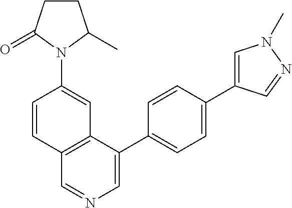 [SMILES: CC1CCC(=O)N1c1ccc2cncc(-c3ccc(-c4cnn(C)c4)cc3)c2c1]

Into a 10 mL sealed tube purged and maintained with an inert atmosphere of nitrogen were placed 1-(4-chloroisoquinolin-6-yl)-5-methylpyrrolidin-2-one (130 mg, 0.50 mmol), 1-methyl-4-[4-(tetramethyl-1,3,2-dioxaborolan-2-yl)phenyl]-1H-pyrazole (183 mg, 0.65 mmol), sodium carbonate (81.3 mg, 0.77 mmol), Pd(dppf)Cl₂ dichloromethane complex (41.9 mg, 0.05 mmol), water (2 mL) and acetonitrile (2 mL). The solution was stirred for 1.5 h at 150° C. The resulting mixture was concentrated to dryness, redissolved in 100 mL of dichloromethane and washed with water (10 mL) and brine (10 mL). The organic phase was dried over sodium sulfate, filtered and concentrated to dryness. The crude product was purified by prep-HPLC (acetonitrile/water). This resulted in 50.0 mg (26%) of the title compound as a yellow solid. 1H NMR (300 MHz, DMSO-d6) ppm=9.27 (s, 1H), 8.43 (s, 1H), 8.26-8.23 (m, 2H), 8.13 (s, 1H), 7.98 (s, 1H), 7.94-7.90 (d, 1H), 7.80-7.75 (d, 2H), 7.56-7.49 (d, 2H), 4.54-4.48 (m, 1H), 3.90 (s, 3H), 2.57-2.34 (m, 3H), 1.73-1.62 (m, 1H), 1.23 (d, 3H). [M+H]⁺ 383. Rt 1.19 min (method I).

### Example 13

### 1-(4-(4-(1-(2-hydroxy-2-methylpropyl)-1H-pyrazol-4-yl)phenyl)isoquinolin-6-yl)indolin-2-one (150)

### 1-(4-chloroisoquinolin-6-yl)indolin-2-one

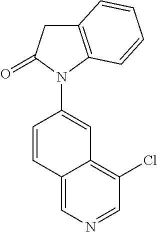 [SMILES: O=C1Cc2ccccc2N1c1ccc2cncc(Cl)c2c1]

A mixture of 6-bromo-4-chloroisoquinoline (20 mg, 0.082 mmol), 2-oxindole (20 mg, 0.15 mmol), Pd₂(dba)₃ (12 mg, 0.013 mmol), Xantphos (16 mg, 0.028 mmol) and Cs₂CO₃ (80 mg, 0.25 mmol) in 1,4-dioxane (0.5 mL) was heated at 80° C. for 1 h under microwave irradiation. The reaction mixture was concentrated in vacuo and the residue purified by Biotage (SNAP 10 g column, CH₂Cl₂/EtOH 100/0->93/7) to give a mixture of the title compound and 2-oxindole which was used in the next step without further purification.

### 1-(4-(4-(1-(2-hydroxy-2-methylpropyl)-1H-pyrazol-4-yl)phenyl)isoquinolin-6-yl)indolin-2-one (150)

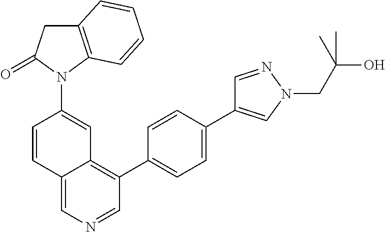 [SMILES: CC(C)(O)Cn1cc(-c2ccc(-c3cncc4ccc(N5C(=O)Cc6ccccc65)cc34)cc2)cn1]

A mixture of 1-(4-chloroisoquinolin-6-yl)indolin-2-one (55 mg, 0.093 mmol), 1-(4-(4-bromophenyl)-1H-pyrazol-1-yl)-2-methylpropan-2-ol (45 mg, 0.13 mmol), K₃PO₄ (60 mg, 0.28 mmol) and Pd(dtbpf)Cl₂ (13 mg, 0.020 mmol) in 1,4-dioxane (1 mL) and water (0.2 mL) was stirred at 120° C. for 1 h under microwave irradiation. The reaction mixture was concentrated in vacuo and purified by Biotage column chromatography (SNAP 10 g column, cyclohexane/EtOAc 100/0->0/100, then EtOAc/EtOH 100/0->85/15) to give the title compound as a pale yellow solid (7 mg, 16%). ¹H NMR (500 MHz, CDCl3) ppm=9.33 (s, 1H), 8.59 (s, 1H), 8.23 (d, J=8.6 Hz, 1H), 8.04 (d, J=1.9 Hz, 1H), 7.90 (s, 1H), 7.76 (s, 1H), 7.72 (dd, J=8.7, 2.0 Hz, 1H), 7.65 (d, J=8.2 Hz, 2H), 7.56 (d, J=8.2 Hz, 2H), 7.34 (d, J=7.3 Hz, 1H), 7.23 (t, J=7.7 Hz, 1H), 7.11 (t, J=7.5 Hz, 1H), 6.83 (d, J=7.9 Hz, 1H), 4.14 (s, 2H), 3.78 (br s, 1H), 3.75 (s, 2H), 1.23 (s, 6H). [M+H]⁺ 475, Rt 1.43 min (method O).

### Example 14

### N-(4-(4-(1-(2-hydroxy-2-methylpropyl)-1H-pyrazol-4-yl)phenyl)isoquinolin-6-yl)methanesulfonamide (138)

### N-(4-chloroisoquinolin-6-yl)methanesulfonamide

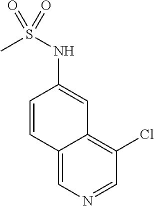 [SMILES: CS(=O)(=O)Nc1ccc2cncc(Cl)c2c1]

A mixture of 6-bromo-4-chloroisoquinoline (51 mg, 0.21 mmol), methanesulfonamide (32 mg, 0.34 mmol), Pd₂(dba)₃ (24 mg, 0.026 mmol), Xantphos (30 mg, 0.052 mmol) and Cs₂CO₃ (118 mg, 0.36 mmol) in 1,4-dioxane (1.4 mL) was heated at 150° C. for 1 h under microwave irradiation. The reaction mixture was then concentrated in vacuo and the residue purified by Biotage column chromatography (SNAP 10 g column, CH₂Cl₂/EtOH 100/0->85/15) to give the title compound as a yellow solid (52 mg, 96%).

### N-(4-(4-(1-(2-hydroxy-2-methylpropyl)-1H-pyrazol-4-yl)phenyl)isoquinolin-6-yl)methanesulfonamide (138)

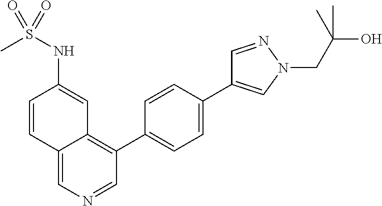 [SMILES: CC(C)(O)Cn1cc(-c2ccc(-c3cncc4ccc(NS(C)(=O)=O)cc34)cc2)cn1]

A mixture of N-(4-chloroisoquinolin-6-yl)methanesulfonamide (43 mg, 0.17 mmol), 1-(4-(4-bromophenyl)-1H-pyrazol-1-yl)-2-methylpropan-2-ol (57 mg, 0.17 mmol), K₃PO₄ (110 mg, 0.52 mmol) and Pd(dtbpf)Cl₂ (16 mg, 0.025 mmol) in a mixture of 1,4-dioxane (0.9 mL) and water (0.25 mL) was stirred at 120° C. for 1 h under microwave irradiation. The reaction mixture was concentrated in vacuo and the residue purified by Biotage column chromatography (SNAP 10 g column, cyclohexane/EtOAc 100/0->0/100, then CH₂Cl₂/EtOH 100/0->80/20) to give a pale yellow solid which was taken up in Et₂O, sonicated and filtered. The filtrate was then concentrated in vacuo to give the title compound as a cream coloured solid (9 mg, 12%). ¹H NMR (500 MHz, DMSO-d6) ppm=10.34 (s, 1H), 9.22 (s, 1H), 8.40 (s, 1H), 8.17-8.22 (m, 2H), 7.99 (s, 1H), 7.80-7.75 (m, 3H), 7.59 (dd, J=8.8, 2.2 Hz, 1H), 7.54 (d, J=8.1 Hz, 2H), 4.77 (br s, 1H), 4.07 (s, 2H), 3.08 (s, 3H), 1.11 (s, 6H). [M+H]⁺ 437, Rt 0.93 min (method O).

### Example 15

### N-(4-(2-(2-hydroxy-2-methylpropyl)-2H-indazol-5-yl)isoquinolin-6-yl)methanesulfonamide (151)

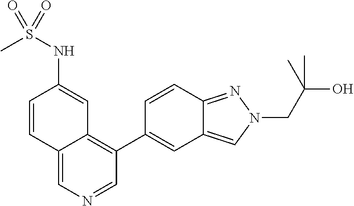 [SMILES: CC(C)(O)Cn1cc2cc(-c3cncc4ccc(NS(C)(=O)=O)cc34)ccc2n1]

A mixture of N-(4-chloroisoquinolin-6-yl)methanesulfonamide (52 mg, 0.20 mmol), 2-methyl-1-(5-(4,4,5,5-tetramethyl-1,3,2-dioxaborolan-2-yl)-2H-indazol-2-yl)propan-2-ol (67 mg, 0.21 mmol), K₃PO₄ (130 mg, 0.61 mmol) and Pd(dtbpf)Cl₂ (15 mg, 0.023 mmol) in a mixture of 1,4-dioxane (1 mL) and water (0.25 mL) was stirred at 120° C. for 1 h under microwave irradiation. The reaction mixture was then concentrated in vacuo and purified by Biotage (SNAP 10 g column, EtOAc/EtOH 100/0->65/35) to give the title compound as a white solid (4 mg, 5%). ¹H NMR (500 MHz, CD₃OD) ppm=9.18 (s, 1H), 8.40 (s, 1H), 8.38 (s, 1H), 8.20 (d, J=8.9 Hz, 1H), 7.90 (s, 1H), 7.83-7.80 (m, 2H), 7.65 (dd, J=8.9, 2.2 Hz, 1H), 7.48 (dd, J=8.9, 1.7 Hz, 1H), 4.47 (s, 2H), 3.03 (s, 3H), 1.27 (s, 6H). [M+H]⁺ 411, Rt 0.82 min (method O).

### Example 16

### N-(4-(4-(1-(2-methoxyethyl)-1H-pyrazol-4-yl)phenyl)isoquinolin-6-yl)methanesulfonamide (166)

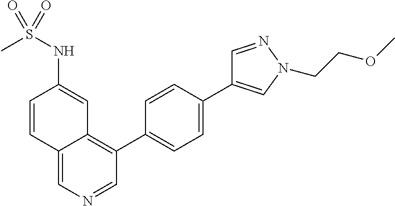

A mixture of N-(4-chloroisoquinolin-6-yl)methanesulfonamide (32 mg, 0.13 mmol), 1-(2-methoxyethyl)-4-(4-(4,4,5,5-tetramethyl-1,3,2-dioxaborolan-2-yl)phenyl)-1H-pyrazole (90 mg, 0.27 mmol), K₃PO₄ (80 mg, 0.38 mmol) and Pd(dtbpf)Cl₂ (10 mg, 0.015 mmol) in a mixture of 1,4-dioxane (0.9 mL) and water (0.2 mL) was stirred at 120° C. for 1 h under microwave irradiation. The mixture was then concentrated in vacuo and the residue purified by Biotage (SNAP 10 g column, EtOAc, then CH₂Cl₂/EtOH 97/3->92/8) to give the title compound as a cream coloured solid (2.8 mg, 5%). ¹H NMR (500 MHz, CDCl3) ppm=9.19 (s, 1H), 8.48 (s, 1H), 8.06 (d, J=9.7 Hz, 1H), 7.70-7.67 (m, 2H), 7.70-7.67 (m, 2H), 7.57 (d, J=8.2 Hz, 2H), 7.46 (d, J=8.2 Hz, 2H), 4.38 (t, J=5.1 Hz, 2H), 3.82 (t, J=5.1 Hz, 2H), 3.38 (s, 3H), 3.09 (s, 3H). [M−H]⁺ 423, Rt 0.97 min (method O).

### Example 17

### N-{4-[4-(1-Methyl-1H-pyrazol-4-yl)-phenyl]-isoquinolin-6-yl}-benzenesulfonamide (152)

### N-(4-chloroisoquinolin-6-yl)benzenesulfonamide

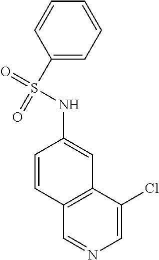 [SMILES: O=S(=O)(Nc1ccc2cncc(Cl)c2c1)c1ccccc1]

Into a 25-mL round-bottom flask purged and maintained with an inert atmosphere of nitrogen was placed 6-bromo-4-chloroisoquinoline (85 mg, 0.35 mmol), benzenesulfonamide (110 mg, 0.70 mmol), K₃PO₄ (372 mg, 1.75 mmol), xantphos (61.0 mg, 0.11 mmol), Pd₂(dba)₃*CHCl₃ (36 mg, 0.030 mmol) and toluene (4 mL). The solution was stirred for 1 h at 100° C. The mixture was concentrated under vacuum. The residue was purified on a silica gel column with ethyl acetate/petrolether (1:1). This resulted in 130 mg (99%) of N-(4-chloroisoquinolin-6-yl)benzenesulfonamide as a yellow solid. [M+H]⁺ 319. Rt 1.38 min (method D).

### N-{4-[4-(1-Methyl-1H-pyrazol-4-yl)-phenyl]-isoquinolin-6-yl}-benzenesulfonamide (152)

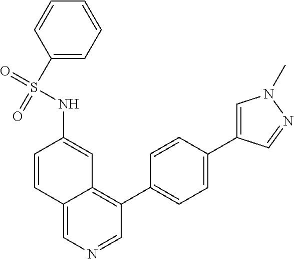 [SMILES: Cn1cc(-c2ccc(-c3cncc4ccc(NS(=O)(=O)c5ccccc5)cc34)cc2)cn1]

Into a 10-mL vial was added N-(4-chloroisoquinolin-6-yl)benzenesulfonamide (50 mg, 0.16 mmol), 1-methyl-4-[4-(tetramethyl-1,3,2-dioxaborolan-2-yl)phenyl]-1H-pyrazole (58 mg, 0.20 mmol), sodium carbonate (5.00 mg, 0.05 mmol), Pd(dppf)Cl₂*CH₂Cl₂ (13.0 mg, 0.02 mmol), acetonitrile (2 mL) and water (2 mL). The mixture was stirred for 4 h at 140° C. under microwave irradiation and concentrated under vacuum. The residue was purified by silica gel column chromatography with petrolether:ethyl acetate (1:5) and by prep-HPLC (acetonitrile/water). This resulted in 13.0 mg (19%) of N-[4-[4-(1-methyl-1H-pyrazol-4-yl)phenyl]isoquinolin-6-yl]benzenesulfonamide as a light yellow solid. ¹H NMR (400 MHz, DMSO-d6) ppm=10.98 (s, 1H), 9.12 (s, 1H), 8.31 (s, 2H), 8.09-8.03 (m, 2H), 7.78-7.73 (m, 4H), 7.63-7.43 (m, 5H), 7.34-7.32 (d, J=8 Hz, 2H), 3.93 (s, 3H). [M+H]⁺ 441. Rt 1.12 min (method Q).

### Example 18

### Propane-2-sulfonic acid {4-[4-(1-methyl-1H-pyrazol-4-yl)-phenyl]-isoquinolin-6-yl}-amide (157)

### N-(4-chloroisoquinolin-6-yl)propane-2-sulfonamide

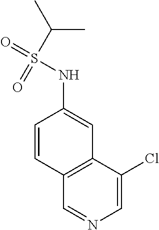 [SMILES: CC(C)S(=O)(=O)Nc1ccc2cncc(Cl)c2c1]

Into a 25-mL round-bottom flask purged and maintained with an inert atmosphere of nitrogen was added 6-bromo-4-chloroisoquinoline (100 mg, 0.41 mmol), propane-2-sulfonamide (102 mg, 0.83 mmol), K₃PO₄ (438 mg, 2.06 mmol), Pd₂(dba)₃*CHCl₃ (43.0 mg, 0.04 mmol), xantphos (72.0 mg, 0.12 mmol) and toluene (6 mL). The solution was stirred for 1 h at 100° C. and the reaction mixture was concentrated under vacuum. The residue was dissolved in 10 mL of ethyl acetate and washed with 3×10 mL of water. The crude was purified by silica gel column chromatography with methanol:dichloromethane (1:20). This resulted in 95 mg (81%) of N-(4-chloroisoquinolin-6-yl)propane-2-sulfonamide as a yellow solid. [M+H]⁺ 285. Rt 0.87 min (method Q).

### Propane-2-sulfonic acid {4-[4-(1-methyl-1H-pyrazol-4-yl)-phenyl]-isoquinolin-6-yl}-amide (157)

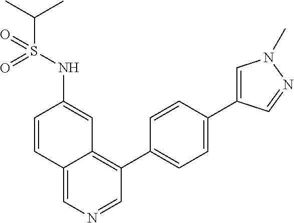 [SMILES: CC(C)S(=O)(=O)Nc1ccc2cncc(-c3ccc(-c4cnn(C)c4)cc3)c2c1]

Into a 10-mL vial purged and maintained with an inert atmosphere of nitrogen was added N-(4-chloroisoquinolin-6-yl)propane-2-sulfonamide (80 mg, 0.28 mmol), 1-methyl-4-[4-(tetramethyl-1,3,2-dioxaborolan-2-yl)phenyl]-1H-pyrazole (104 mg, 0.37 mmol), sodium carbonate (90.0 mg, 0.85 mmol), Pd(PCy₃)₂Cl₂ (21.0 mg, 0.03 mmol), water (1 mL) and dioxane (4 mL). The mixture was stirred for 1 h at 100° C. under microwave irradiation and concentrated under vacuum. The residue was purified by silica gel column chromatography with methanol:dichloromethane (1:20). The crude product was purified by prep-HPLC (acetonitrile/water). This resulted in 20 mg (18%) of N-[4-[4-(1-methyl-1H-pyrazol-4-yl)phenyl]isoquinolin-6-yl]propane-2-sulfonamide as a white solid. ¹H NMR (300 MHz, DMSO-d6) ppm=10.30 (s, 1H), 9.19 (s, 1H), 8.38 (s, 1H), 8.26 (s, 1H), 8.16 (d, J=8.7 Hz, 1H), 7.98 (s, 1H), 7.79-7.74 (m, 3H), 7.62 (d, J=9.0 Hz, 1H), 7.53 (d, J=8.1 Hz, 2H), 3.90 (s, 3H), 3.37 (m, 1H), 1.24 (d, J=6.6 Hz, 6H). [M+H]⁺ 407. Rt 1.21 min (method Q).

### Example 19

### 1-Methyl-cyclopropanesulfonic acid {4-[4-(1-methyl-1H-pyrazol-4-yl)-phenyl]-isoquinolin-6-yl}-amide (168)

### N-(4-chloroisoquinolin-6-yl)-1-methylcyclopropane-1-sulfonamide

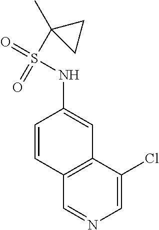 [SMILES: CC1(S(=O)(=O)Nc2ccc3cncc(Cl)c3c2)CC1]

Into a 25-mL round-bottom flask purged and maintained with an inert atmosphere of nitrogen was added 6-bromo-4-chloroisoquinoline (200 mg, 0.82 mmol), 1-methylcyclopropane-1-sulfonamide (223 mg, 1.65 mmol), K₃PO₄ (876 mg, 4.13 mmol), Pd₂(dba)₃*CHCl₃ (86.0 mg, 0.08 mmol), Xantphos (144 mg, 0.25 mmol) and toluene (5 mL). The solution was stirred for 3 h at 100° C. and the reaction mixture was concentrated under vacuum. The residue was purified by silica gel column chromatography with ethyl acetate/petroleum ether (4:1). This resulted in 240 mg (98%) of N-(4-chloroisoquinolin-6-yl)-1-methylcyclopropane-1-sulfonamide as a yellow solid. [M+H]⁺ 297. Rt 1.05 min (method S).

### 1-Methyl-cyclopropanesulfonic acid {4-[4-(1-methyl-1H-pyrazol-4-yl)-phenyl]-isoquinolin-6-yl}-amide (168)

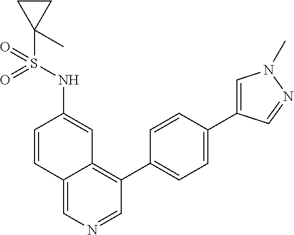 [SMILES: Cn1cc(-c2ccc(-c3cncc4ccc(NS(=O)(=O)C5(C)CC5)cc34)cc2)cn1]

Into a 10-mL vial purged and maintained with an inert atmosphere of nitrogen was added N-(4-chloroisoquinolin-6-yl)-1-methylcyclopropane-1-sulfonamide (200 mg, 0.670 mmol), 1-methyl-4-[4-(tetramethyl-1,3,2-dioxaborolan-2-yl)phenyl]-1H-pyrazole (249 mg, 0.880 mmol), sodium carbonate (215 mg, 2.03 mmol), Pd(PCy₃)₂Cl₂ (50 mg, 0.07 mmol), dioxane (4 mL) and water (1 mL). The reaction mixture was stirred under microwave irradiation for 1 h at 120° C. and concentrated under vacuum. The residue was purified by silica gel column chromatography with methanol:dichloromethane (1:20). The crude product was purified by prep-HPLC acetonitrile/water. This resulted in 20.1 mg (7%) of 1-methyl-N-[4-[4-(1-methyl-1H-pyrazol-4-yl)phenyl]isoquinolin-6-yl]cyclopropane-1-sulfonamide as a white solid. 1H NMR (300 MHz, DMSO-d6) ppm=10.35 (s, 1H), 9.20 (s, 1H), 8.38 (s, 1H), 8.26 (s, 1H), 8.17 (d, J=9.0 Hz, 1H), 7.98 (s, 1H), 7.78-7.75 (m, 3H), 7.63 (d, J=8.7 Hz, 1H), 7.50 (d, J=8.1 Hz, 2H), 3.90 (s, 3H), 1.37 (s, 3H), 1.11 (s, 2H), 0.78-0.76 (m, 2H). [M+H]⁺ 419. Rt 1.39 min (method B).

### Example 20

### Azetidine-1-sulfonic acid {4-[4-(1-methyl-1H-pyrazol-4-yl)-phenyl]-isoquinolin-6-yl}-amide (169)

### N-(4-chloroisoquinolin-6-yl)azetidine-1-sulfonamide

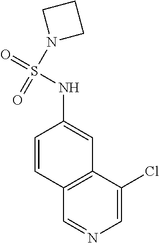 [SMILES: O=S(=O)(Nc1ccc2cncc(Cl)c2c1)N1CCC1]

Into a 25-mL round-bottom flask purged and maintained with an inert atmosphere of nitrogen was added 6-bromo-4-chloroisoquinoline (200 mg, 0.82 mmol), azetidine-1-sulfonamide (225 mg, 1.65 mmol), K₃PO₄ (876 mg, 4.13 mmol), Pd₂(dba)₃*CHCl₃ (86 mg, 0.08 mmol), xantphos (144 mg, 0.25 mmol) and toluene (5 mL). The solution was stirred for 3 h at 100° C. and the reaction mixture was concentrated under vacuum. The residue was purified by silica gel column chromatography with ethyl acetate/petroleum ether (4:1). This resulted in 240 mg (98%) of N-(4-chloroisoquinolin-6-yl)azetidine-1-sulfonamide as a yellow solid. [M+H]⁺ 298. Rt 0.70 min (method S).

### Azetidine-1-sulfonic acid {4-[4-(1-methyl-1H-pyrazol-4-yl)-phenyl]-isoquinolin-6-yl}-amide (169)

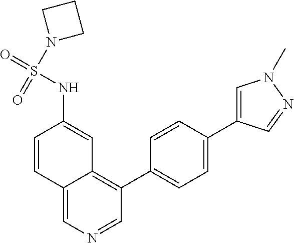 [SMILES: Cn1cc(-c2ccc(-c3cncc4ccc(NS(=O)(=O)N5CCC5)cc34)cc2)cn1]

Into a 10-mL vial purged and maintained with an inert atmosphere of nitrogen was added N-(4-chloroisoquinolin-6-yl)azetidine-1-sulfonamide (200 mg, 0.67 mmol), 1-methyl-4-[4-(tetramethyl-1,3,2-dioxaborolan-2-yl)phenyl]-1H-pyrazole (249 mg, 0.88 mmol), sodium carbonate (214 mg, 2.02 mmol), Pd(PCy₃)₂Cl₂ (50 mg, 0.07 mmol), dioxane (4 mL), water (1 mL). The reaction mixture was stirred under microwave radiation for 1 h at 120° C. and then concentrated under vacuum. The residue was purified by silica gel column chromatography with methanol:dichloromethane (1:20) and by prep-HPLC (acetonitrile/water). This resulted in 20.2 mg (7%) of N-[4-[4-(1-methyl-1H-pyrazol-4-yl)phenyl]isoquinolin-6-yl]azetidine-1-sulfonamide as a white solid. ¹H NMR (300 MHz, DMSO-d6) ppm=10.46 (s, 1H), 9.18 (s, 1H), 8.38 (s, 1H), 8.25 (s, 1H), 8.14 (d, J=9.0 Hz, 1H), 7.97 (s, 1H), 7.76 (d, J=7.8 Hz, 3H), 7.58-7.52 (m, 3H), 3.90 (s, 3H), 3.79-3.74 (m, 4H), 2.13-2.08 (m, 2H). [M+H]⁺ 420. Rt 1.12 min (method Q).

### Example 21

### N-methyl-N-(4-(4-(1-methyl-1H-pyrazol-4-yl)phenyl)isoquinolin-6-yl)methanesulfonamide (139)

### N-(4-chloroisoquinolin-6-yl)-N-methylmethanesulfonamide

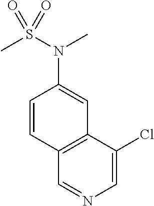 [SMILES: CN(c1ccc2cncc(Cl)c2c1)S(C)(=O)=O]

A mixture of 6-bromo-4-chloroisoquinoline (69 mg, 0.28 mmol), Xantphos (35 mg, 0.060 mmol), Pd₂(dba)₃ (27 mg, 0.029 mmol), tripotassium phosphate (261 mg, 1.02 mmol) and N-methylmethanesulfonamide (44 mg, 0.18 mmol) in toluene (1 mL) was heated in a focused microwave reactor to 125° C. for 1 h. The cooled reaction mixture was concentrated under reduced pressure and the crude material purified over a silica column using a solvent system of 0-10% EtOH in dichloromethane. [M+H]⁺ 271 Rt 1.08 min (method O)

### N-methyl-N-(4-(4-(1-methyl-1H-pyrazol-4-yl)phenyl)isoquinolin-6-yl)methanesulfonamide (139)

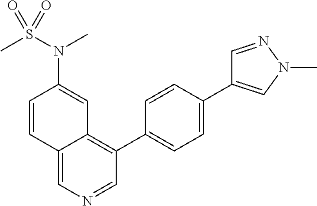

To a solution of N-(4-chloroisoquinolin-6-yl)-N-methylmethanesulfonamide (14 mg, 0.05 mmol), 1-methyl-4-(4-(4,4,5,5-tetramethyl-1,3,2-dioxaborolan-2-yl)phenyl)-1H-pyrazole (15 mg, 0.05 mmol), Pd(dppf)Cl₂ (2 mg, 2.6 μmol) in MeCN (0.8 mL) was added Na₂CO₃(0.15 mL, 0.07 mmol, 0.5 M). The reaction vessel was sealed and heated in a focused microwave reactor to 125° C. for 1.5 h. The cooled reaction mixture was concentrated under reduced pressure, and the crude product purified over a silica cartridge using a solvent system of 1-12% ethanol in dichloromethane. The title compound was isolated as a viscous oil (8 mg, 39%): ¹H NMR (400 MHz, CDCl₃) ppm=9.27 (s, 1H,), 8.56 (s, 1H), 8.09 (d, J=8.8 Hz, 1H), 7.91 (d, J=2.1 Hz, 1H), 7.87 (s, 1H), 7.75-7.72 (m, 2H), 7.66 (d, J=8.2 Hz, 2H), 7.53 (d, J=8.2 Hz, 2H), 4.00 (s, 3H), 3.39 (s, 3H), 2.88 (s, 3H). [M+H]⁺ 393. Rt 1.06 min (method O)

### Example 22

### N-(2-methoxyethyl)-N-(4-(4-(1-methyl-1H-pyrazol-4-yl)phenyl)isoquinolin-6-yl)methanesulfonamide (167)

### N-(2-methoxyethyl)methanesulfonamide

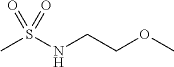

To a solution of methanesulfonyl chloride (0.14 mL, 1.75 mmol) and triethylamine (0.48 mL, 3.49 mmol) in dichloromethane (3 mL) at 0° C. was added 2-methoxyethanamine (0.15 mL, 1.75 mmol) dropwise. The reaction mixture was allowed to warm to room temperature and stirred for 16 h. The crude mixture was diluted with dichloromethane, and the organic layer washed with brine. The combined organic layers were dried over MgSO₄, filtered, and the filtrate concentrated under reduced pressure. The crude product was triturated with diethyl ether and filtered. The filtrate was concentrated under reduced pressure to give the title compound as a colourless oil (159 mg, 59%). ¹H NMR (400 MHz, CDCl₃) ppm=4.75 (1H, s, NH), 3.52 (2H, t, J=5.3 Hz, CH₂), 3.38 (3H, s, Me), 3.31 (2H, q, J=5.3 Hz, CH₂), 2.98 (3H, s, Me) N-(4-chloroisoquinolin-6-yl)-N-(2-methoxyethyl)methanesulfonamide

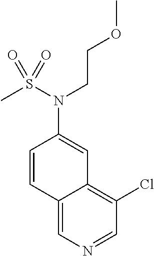 [SMILES: COCCN(c1ccc2cncc(Cl)c2c1)S(C)(=O)=O]

A suspension of 6-bromo-4-chloroisoquinoline (14 mg, 0.06 mmol), N-(2-methoxyethyl)methanesulfonamide (31 mg, 0.20 mmol), Pd₂(dba)₃ (8 mg, 9.24 umol), tripotassium phosphate (69 mg, 0.32 mmol) and Xantphos (11 mg, 0.02 mmol) in toluene (0.5 mL) was heated in a focused microwave reactor to 125° C. for 1.5 h. The volatiles were removed under reduced pressure, and the crude product was passed over a silica plug using 10% ethanol in dichloromethane. The crude product was used in the next step without further purification.

### N-(2-methoxyethyl)-N-(4-(4-(1-methyl-1H-pyrazol-4-yl)phenyl)isoquinolin-6-yl)methanesulfonamide

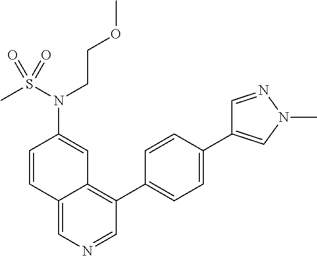

To a solution of N-(4-chloroisoquinolin-6-yl)-N-(2-methoxyethyl) methanesulfonamide (25 mg, 0.08 mmol), 1-methyl-4-(4-(4,4,5,5-tetramethyl-1,3,2-dioxaborolan-2-yl)phenyl)-1H-pyrazole (23 mg, 0.08 mmol), Pd(dppf)Cl₂ (3 mg, 14.0 μmol) in MeCN (0.8 mL) was added Na₂CO₃ (0.22 mL, 0.11 mmol, 0.5 M). The reaction vessel was sealed and heated in a focused microwave reactor to 150° C. for 1.5 h. The cooled reaction mixture was concentrated under reduced pressure, and the crude product purified over a silica cartridge using a solvent system of 0-15% ethanol in dichloromethane. ¹H NMR (400 MHz, CDCl₃) ppm=9.33 (s, 1H), 8.55 (s, 1H), 8.17 (d, J=8.8 Hz, 1H), 8.05 (d, J=2.0 Hz, 1H), 7.86 (s, 1H), 7.76 (dd, J=8.8, 2.0 Hz, 1H), 7.73 (s, 1H), 7.67 (d, J=8.2 Hz, 2H), 7.52 (d, J=8.2 Hz, 2H), 4.01 (s, 3H), 3.94 (t, J=5.5 Hz, 2H), 3.48 (t, J=5.5 Hz, 2H), 3.25 (s, 3H), 3.02 (s, 3H). [M+H]⁺ 437 Rt 1.19 min (method O)

### Example 23

### 6-(1-methyl-1H-imidazol-2-yl)-4-(4-(1-methyl-1H-pyrazol-4-yl)phenyl)isoquinoline (103)

### 4-chloro-6-(1-methyl-1H-imidazol-2-yl)isoquinoline

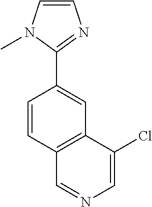 [SMILES: Cn1ccnc1-c1ccc2cncc(Cl)c2c1]

To 6-bromo-4-chloroisoquinoline (208 mg, 0.858 mmol) and Pd(PPh₃)₄ (49.6 mg, 0.043 mmol) in a microwave vial were added degassed 1,4-dioxane (3.3 mL) and 1-methyl-2-(tributylstannyl)-1H-imidazole (412 μl, 1.287 mmol). The reaction mixture was heated in an oil bath at 100° C. for 3 h. The crude was concentrated and purified via biotage (dichloromethane/EtOH 98/2 to 95/5) to give the title compound (200 mg, contaminated with some tin residue).

### 6-(1-methyl-1H-imidazol-2-yl)-4-(4-(1-methyl-1H-pyrazol-4-yl)phenyl)isoquinoline

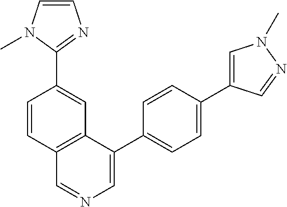 [SMILES: Cn1cc(-c2ccc(-c3cncc4ccc(-c5nccn5C)cc34)cc2)cn1]

4-chloro-6-(1-methyl-1H-imidazol-2-yl)isoquinoline (80 mg, 0.328 mmol), 1-methyl-4-(4-(4,4,5,5-tetramethyl-1,3,2-dioxaborolan-2-yl)phenyl)-1H-pyrazole (121 mg, 0.427 mmol) and Pd(dppf)Cl₂.CH₂Cl₂ (13.40 mg, 0.016 mmol) were loaded in a microwave vial and degassed acetonitrile (5.0 mL) and 0.5M sodium carbonate in water (0.9 mL, 0.460 mmol) were added. The reaction was heated under microwave irradiation at 120° C. for 60 min. The crude was purified via biotage column chromatography (dichloromethane/EtOH, 98/2 to 94/6), the fractions containing the product were then filtered on a SCX2 column and the product was released with 1N NH₃ in MeOH before being purified via preparative TLC (dichloromethane/EtOH 95/5). The product was then purified by preparative HPLC. Injections of the sample were made onto a Phenomenex Gemini column (10 μm, 250×21.2 mm, C18, Phenomenex, Torrance, USA). Chromatographic separation at room temperature was carried out using Gilson GX-281 Liquid Handler system combined with a Gilson 322 HPLC pump (Gilson, Middleton, USA) over a 15 minute gradient elution (Grad15 mins20 mls·m) from 40:60 to 100:0 methanol:water (both modified with 0.1% formic acid) at a flow rate of 20 mL/min. The fractions were concentrated and diluted in dichloromethane and sat. aq NaHCO₃ solution. The layers were separated and the aqueous layer was extracted with dichloromethane, the organics layers were dried over MgSO₄ and concentrated to give the title compound (10 mg, 8% yield). ¹H NMR (500 MHz, CDCl₃) ppm=9.30 (s, 1H), 8.57 (s, 1H), 8.17 (bs, 1H), 8.15 (d, J=8.4, 1H), 8.01 (dd, J=8.4, 1.5, 1H), 7.85 (s, 1H), 7.71 (s, 1H), 7.64 (d, J=8.5, 2H), 7.54 (d, J=8.5, 2H), 7.17 (d, J=1.1, 1H), 7.00 (d, J=1.1, 1H), 4.00 (s, 3H), 3.75 (s, 3H) [M+H]⁺ 366. Rt 1.84 min (method N).

### Example 24

### 1-methyl-5-(6-(1-methyl-1H-imidazol-2-yl)isoquinolin-4-yl)-1,3-dihydrobenzo[c]isothiazole 2,2-dioxide (93)

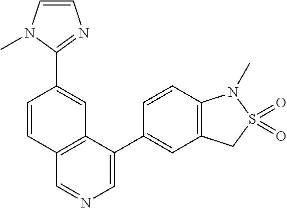 [SMILES: CN1c2ccc(-c3cncc4ccc(-c5nccn5C)cc34)cc2CS1(=O)=O]

1-methyl-5-(6-(1-methyl-1H-imidazol-2-yl)isoquinolin-4-yl)-1,3-dihydrobenzo[c]isothiazole 2,2-dioxide was prepared in the manner provided in Example 13, using 1-methyl-5-(4,4,5,5-tetramethyl-1,3,2-dioxaborolan-2-yl)-1,3-dihydrobenzo[c]isothiazole 2,2-dioxide as the starting material. ¹H NMR (500 MHz, CDCl₃) ppm=9.29 (s, 1H), 8.49 (s, 1H), 8.15 (d, J=8.4, 1H), 8.10 (s, 1H), 7.97 (dd, J=8.4, 1.5, 1H), 7.53-7.50 (m, 1H), 7.44 (s, 1H), 7.17 (d, J=1.1, 1H), 7.03 (d, J=1.1, 1H), 6.90 (d, J=8.1, 1H), 4.45 (s, 2H), 3.75 (s, 3H), 3.22 (s, 3H). [M+H]⁺ 391. Rt 1.45 min (method N).

### Example 25

### 2-(4-(4-(1-methyl-1H-pyrazol-4-yl)phenyl)isoquinolin-6-yl)oxazole (119) 2-(4-chloroisoquinolin-6-yl)oxazole

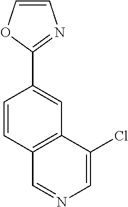 [SMILES: Clc1cncc2ccc(-c3ncco3)cc12]

To 6-bromo-4-chloroisoquinoline (109 mg, 0.449 mmol) and Pd(PPh₃)₄ (26.0 mg, 0.022 mmol) in a microwave vial were added degassed 1,4-dioxane (1.80 mL) and 2-(tributylstannyl)oxazole (206 μl, 0.674 mmol). The reaction mixture was heated in an oil bath at 100° C. for 11 h before being concentrated. The crude was purified via biotage column chromatography (dichloromethane/ethyl acetate 95/5 to 50/50) to give the title compound (65 mg, 63% yield).

### 2-(4-(4-(1-methyl-1H-pyrazol-4-yl)phenyl)isoquinolin-6-yl)oxazole

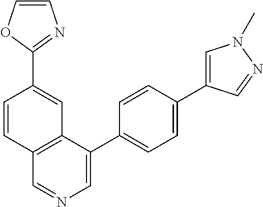 [SMILES: Cn1cc(-c2ccc(-c3cncc4ccc(-c5ncco5)cc34)cc2)cn1]

2-(4-chloroisoquinolin-6-yl)oxazole (32 mg, 0.139 mmol), 1-methyl-4-(4-(4,4,5,5-tetramethyl-1,3,2-dioxaborolan-2-yl)phenyl)-1H-pyrazole (47.3 mg, 0.166 mmol) and Pd(dppf)Cl₂.CH₂Cl₂ (5.67 mg, 6.94 μmol) were loaded in a microwave vial and then acetonitrile (1982 μl) and sodium carbonate in water (388 μl, 0.194 mmol) were added. The reaction was heated at 150° C. for 60 min. The reaction mixture was then concentrated and purified via biotage column chromatography (dichloromethane/EtOAc 100/0 to 40/60 then dichloromethane/EtOH 100/0 to 93/7 follow by dichloromethane/aq NH₃ in methanol 1:10, 100/0 to 90/10). The obtained compound was then filtered on a SCX2 column and the product was released with 1N NH₃ in methanol to give the title compound (29 mg, 59% yield). ¹H NMR (500 MHz, CDCl₃) ppm=9.31 (s, 1H), 8.66 (dd, J=1.5, 0.8, 1H), 8.59 (s, 1H), 8.34 (dd, J=8.6, 1.6, 1H), 8.16 (d, J=8.6, 0.8, 1H), 7.89 (d, J=0.8, 1H), 7.77 (d, J=0.8, 1H), 7.74 (s, 1H), 7.70-7.67 (m, 2H), 7.59-7.55 (m, 2H), 7.31 (d, J=0.8, 1H), 4.02 (s, 3H). [M+H]⁺ 353. Rt 2.91 min (method N).

### Example 26

### 1-methyl-5-(6-(oxazol-2-yl)isoquinolin-4-yl)-1,3-dihydrobenzo[c]isothiazole 2,2-dioxide (107)

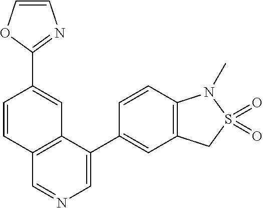 [SMILES: CN1c2ccc(-c3cncc4ccc(-c5ncco5)cc34)cc2CS1(=O)=O]

1-methyl-5-(6-(oxazol-2-yl)isoquinolin-4-yl)-1,3-dihydrobenzo[c]isothiazole 2,2-dioxide was prepared in the manner described in Example 15, using 1-methyl-5-(4,4,5,5-tetramethyl-1,3,2-dioxaborolan-2-yl)-1,3-dihydrobenzo[c]isothiazole 2,2-dioxide as the starting material. ¹H NMR (500 MHz, CDCl₃) ppm=9.31 (s, 1H), 8.55-8.49 (m, 2H), 8.35 (dd, J=8.5, 1.6, 1H), 8.17 (dd, J=8.5, 0.7, 1H), 7.80 (d, J=0.8, 1H), 7.54 (ddt, J=8.2, 1.5, 0.8, 1H), 7.45 (s, 1H), 7.32 (d, J=0.8, 1H), 6.95 (d, J=8.2, 1H), 4.49 (s, 2H), 3.27 (s, 3H) [M+H]⁺ 378. Rt 2.56 min (method N).

### Example 27

### 4-(4-(1-methyl-1H-pyrazol-4-yl)phenyl)isoquinoline-6-carbonitrile (118)

### 4-chloroisoquinoline-6-carbonitrile

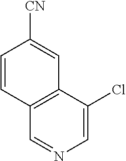 [SMILES: N#Cc1ccc2cncc(Cl)c2c1]

6-bromo-4-chloroisoquinoline (58 mg, 0.239 mmol), zinc cyanide (30.9 mg, 0.263 mmol) and Pd(PPh₃)₄ (27.6 mg, 0.024 mmol) were loaded in a microwave vial and then DMF (1.6 mL) was added. The reaction mixture was heated for 1 h at 60° C. The crude was concentrated and purified via biotage column chromatography (dichloromethane/EtOH 99.9/0.1 to 97.5/2.5) to give the title compound (29 mg, 64% yield).

### 4-(4-(1-methyl-1H-pyrazol-4-yl)phenyl)isoquinoline-6-carbonitrile

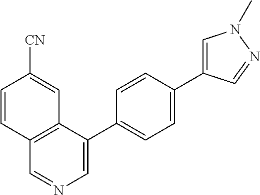 [SMILES: Cn1cc(-c2ccc(-c3cncc4ccc(C#N)cc34)cc2)cn1]

4-Chloroisoquinoline-6-carbonitrile (121 mg, 0.642 mmol), 1-methyl-4-(4-(4,4,5,5-tetramethyl-1,3,2-dioxaborolan-2-yl)phenyl)-1H-pyrazole (219 mg, 0.770 mmol) and Pd(dppf)Cl₂.CH₂Cl₂ (26.2 mg, 0.032 mmol) were loaded in a microwave vial and then acetonitrile (11.1 mL) and sodium carbonate in water (1.8 mL, 0.898 mmol) were added. The reaction was heated at 150° C. for 60 min. before being concentrated. The crude was purified via biotage column chromatography (dichloromethane/EtOH 99.9/0.1 to 98/2) to give the title compound (142 mg, 71% yield). ¹H NMR (500 MHz, CDCl₃) ppm=9.37 (s, 1H), 8.68 (s, 1H), 8.39 (d, J=1.5, 1H), 8.19 (d, J=8.4, 1H), 7.88 (d, J=0.8, 1H), 7.81 (dd, J=8.4, 1.5, 1H), 7.74 (s, 1H), 7.71-7.67 (m, 2H), 7.53-7.47 (m, 2H), 4.02 (s, 3H) [M+H]⁺ 311. Rt 2.86 min (method N).

### Example 28

### 4-(1-methyl-1H-indazol-5-yl)isoquinoline-6-carbonitrile (120)

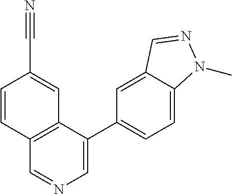 [SMILES: Cn1ncc2cc(-c3cncc4ccc(C#N)cc34)ccc21]

4-(1-methyl-1H-indazol-5-yl)isoquinoline-6-carbonitrile was prepared using the method of Example 17, using 1-methyl-1H-indazol-5-ylboronic acid as the starting material. ¹H NMR (500 MHz, CDCl₃) ppm=9.38 (s, 1H), 8.71 (s, 1H), 8.32-8.31 (m, 1H), 8.19 (d, J=8.5, 1H), 8.12 (d, J=1.0, 1H), 7.86 (s, 1H), 7.80 (dd, J=8.5, 1.5, 1H), 7.62 (dt, J=8.6, 1.0, 1H), 7.51 (dd, J=8.6, 1.5, 1H), 4.20 (s, 3H). [M+H]⁺ 285. Rt 2.77 min (method N).

### Example 29

### 4-(4-(1-methyl-1H-pyrazol-4-yl)phenyl)isoquinoline-6-carboxamide (96)

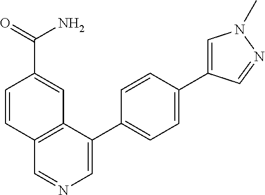 [SMILES: Cn1cc(-c2ccc(-c3cncc4ccc(C(N)=O)cc34)cc2)cn1]

A cooled solution of concentrated H₂SO₄ (1.5 ml, 0.161 mmol) containing 100 uL of water was added to 4-(4-(1-methyl-1H-pyrazol-4-yl)phenyl)isoquinoline-6-carbonitrile (Example 17) (50 mg, 0.161 mmol). The reaction mixture was heated at 50° C. for 1 h before adding 2M NaOH and some aq. NaHCO₃ solution. The aqueous layers were extracted with dichloromethane and the organic layers were concentrated. The crude was purified via biotage column chromatography (dichloromethane/EtOH 99/1 to 93/7) to give the title compound (26 mg, 49% yield). ¹H NMR (500 MHz, DMSO-d6) ppm=9.42 (s, 1H), 8.54 (s, 1H), 8.44 (dd, J=1.6, 0.9, 1H), 8.31 (d, J=8.5, 1H), 8.27 (bs, 2H), 8.14 (dd, J=8.5, 1.6, 1H), 7.99 (d, J=0.8, 1H), 7.81-7.77 (m, 2H), 7.64 (s, 1H), 7.60-7.57 (m, 2H), 3.92 (s, 3H). [M+H]⁺ 329. Rt 2.35 min. (method N).

### Example 30

### 4-(4-(1-methyl-1H-pyrazol-4-yl)phenyl)isoquinoline-6-carboxylic acid hydrochloride (95)

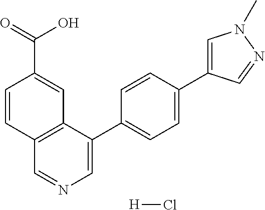 [SMILES: Cn1cc(-c2ccc(-c3cncc4ccc(C(=O)O)cc34)cc2)cn1 ; Cl]

To 4-(4-(1-methyl-1H-pyrazol-4-yl)phenyl)isoquinoline-6-carbonitrile (Example 17) (100 mg, 0.322 mmol) in ethanol (2.5 mL) was added 2 M sodium hydroxide (2.5 mL, 5.00 mmol). The reaction was heated at 100° C. for 1 h. Hydrogen chloride (5.0 mL, 5.00 mmol) was added to the reaction mixture and the solution was concentrated. Isopropanol was added to the residue, the salts were filtered and the filtrate was concentrated. 4N HCl in dioxane was added to the residue obtained. The solution was evaporated to obtained the title compound (100 mg, 85% yield). ¹H NMR (500 MHz, DMSO-d6) ppm=9.70 (s, 1H), 8.68 (s, 1H), 8.60 (s, 1H), 8.51 (d, J=8.5, 1H), 8.32-8.27 (m, 2H), 8.01 (s, 1H), 7.83 (d, J=8.0, 2H), 7.61 (d, J=8.0, 2H), 3.91 (s, 3H). [M−H]⁺ 330. Rt 2.58 min (method N).

### Example 31

### N-methyl-4-(4-(1-methyl-1H-pyrazol-4-yl)phenyl)isoquinoline-6-carboxamide (130)

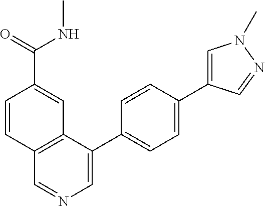 [SMILES: CNC(=O)c1ccc2cncc(-c3ccc(-c4cnn(C)c4)cc3)c2c1]

To 4-(4-(1-methyl-1H-pyrazol-4-yl)phenyl)isoquinoline-6-carboxylic acid (Example 20) in DMF (506 μL) was added HATU (27.7 mg, 0.073 mmol). The mixture was stirred for 10 min before the addition of methylamine in THF (2 M) (92 μL, 0.185 mmol). The mixture was stirred at rt for 15 min before the addition of DIPEA (46.2 μl, 0.265 mmol). The resulting solution was then stirred at rt for 2 h. The reaction mixture was concentrated and purified via biotage column chromatography (dichloromethane/EtOH 99/1 to 94/6) to give the title compound (11 mg, 53% yield). ¹H NMR (500 MHz, CDCl₃) ppm=9.31 (s, 1H), 8.58 (s, 1H), 8.32 (dt, J=1.7, 0.8, 1H), 8.15-8.10 (m, 1H), 8.02 (dd, J=8.5, 1.6, 1H), 7.85 (d, J=0.8, 1H), 7.71 (d, J=0.8, 1H), 7.67-7.63 (m, 2H), 7.54-7.49 (m, 2H), 6.34 (bs, 1H), 4.00 (s, 3H), 3.03 (d, J=4.8, 3H). [M+H]⁺ 343. Rt 2.47 min (method N).

### Example 32

### 4-[4-(1-Methyl-1H-pyrazol-4-yl)-phenyl]-isoquinoline-6-carboxylic acid cyclopropylamide (94)

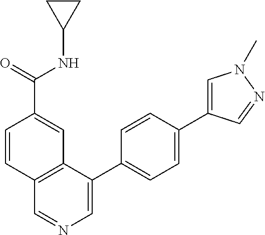 [SMILES: Cn1cc(-c2ccc(-c3cncc4ccc(C(=O)NC5CC5)cc34)cc2)cn1]

Into a 25 mL round-bottom flask, were placed 4-[4-(1-methyl-1H-pyrazol-4-yl)phenyl]isoquinoline-6-carboxylic acid (Example 20) (80.0 mg, 0.24 mmol), cyclopropanamine (28.0 mg, 0.49 mmol), 1-[bis(dimethylamino)methylene]-1H-1,2,3-triazolo[4,5-b]pyridinium 3-oxid hexafluorophosphate (HATU, 128 mg, 0.34 mmol), N,N-diisopropylethylamine (1.00 mL, 6.05 mmol) and N,N-dimethylformamide (2 mL). The solution was stirred for 2 h at 25° C. The mixture was concentrated under vacuum. The residue was applied to a silica gel column with dichloromethane/methanol (100:1). This resulted in 9.6 mg (11%) of the title compound as a white solid. ¹H NMR (400 MHz, CD₃OD): ppm=9.33 (s, 1H), 8.50 (s, 1H), 8.45 (s, 1H), 8.29 (d, 1H), 8.11 (s, 1H), 8.07 (dd, 1H), 7.95 (s, 1H), 7.80 (d, 2H), 7.59 (d, 2H), 3.99 (s, 3H), 2.91-2.86 (m, 1H), 0.85-0.80 (m, 2H), 0.67-0.66 (m, 2H). [M+H]⁺ 369. Rt 2.18 min (method H).

### Example 33

### (3,3-Difluoro-azetidin-1-yl)-{4-[4-(1-methyl-1H-pyrazol-4-yl)-phenyl]-isoquinolin-6-yl}-methanone (104)

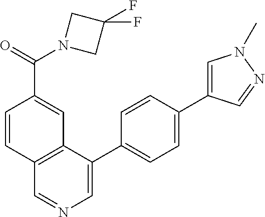 [SMILES: Cn1cc(-c2ccc(-c3cncc4ccc(C(=O)N5CC(F)(F)C5)cc34)cc2)cn1]

Into a 25 mL round-bottom flask, were placed 4-[4-(1-methyl-1H-pyrazol-4-yl)phenyl]isoquinoline-6-carboxylic acid (Example 20) (100 mg, 0.30 mmol), 3,3-difluoroazetidine hydrochloride (39.3 mg, 0.30 mmol), 1-[bis(dimethylamino)methylene]-1H-1,2,3-triazolo[4,5-b]pyridinium 3-oxid hexafluorophosphate (HATU, 127 mg, 0.33 mmol), N,N-diisopropylethylamine (0.50 mL, 3.03 mmol), and N,N-dimethylformamide (5 mL). The solution was stirred for 2 h at 25° C. The mixture was concentrated under vacuum. The residue was applied to a silica gel column with dichloromethane/methanol (100:1). This resulted in 12.7 mg (10%) of the title compound as a white solid. ¹H NMR (400 MHz, CD₃OD) ppm=9.36 (s, 1H), 8.53 (s, 1H), 8.34 (d, 1H), 8.27 (s, 1H), 8.11 (s, 1H), 8.01-7.98 (m, 1H), 7.96 (s, 1H), 7.83-7.81 (d, 2H), 7.50-7.58 (d, 2H), 4.69-4.57 (m, 4H), 3.99 (s, 3H). [M+H]⁺ 405. Rt 2.37 min (method H).

### Example 34

### (1-amino-4-(4-(1-methyl-1H-pyrazol-4-yl)phenyl)isoquinolin-6-yl)(3,3-difluoroazetidin-1-yl)methanone (141)

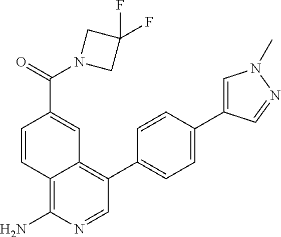 [SMILES: Cn1cc(-c2ccc(-c3cnc(N)c4ccc(C(=O)N5CC(F)(F)C5)cc34)cc2)cn1]

To (3,3-difluoroazetidin-1-yl)(4-(4-(1-methyl-1H-pyrazol-4-yl)phenyl)isoquinolin-6-yl)methanone (100 mg, 0.185 mmol) in DCM (1.9 mL) was added 3-chloroperoxybenzoic acid (83 mg, 0.371 mmol). The reaction was stirred at rt for 1 h. After addition of a solution of Na₂S₂O₅ and then NaHCO₃, the reaction mixture was diluted with DCM. The aqueous layer was extracted three times with DCM, the organic layer was dried over MgSO₄ and concentrated. To the crude material in pyridine (4.4 mL, 37.0 mmol) was added 4-toluenesulfonyl chloride (42.3 mg, 0.222 mmol) and the mixture was stirred at rt for 1 h. Ethanolamine (280 μL, 4.63 mmol) was added and the reaction mixture was stirred at rt for 45 min before being diluted with water and DCM. The aqueous layer was extracted with DCM three times and the organic layer was concentrated. The crude material was purified via Biotage column chromatography (SNAP25 g, DCM/EtOH 99/1 to 85/15) to give the title compound as a yellow solid (14 mg, 18% yield over two step). ¹H NMR (500 MHz, CDCl₃) ppm=8.07 (d, J=1.7 Hz, 1H), 8.03 (d, J=8.6 Hz, 1H), 7.98 (s, 1H), 7.85 (d, J=0.8 Hz, 1H), 7.81 (dd, J=8.6, 1.7 Hz, 1H), 7.71 (d, J=0.8 Hz, 1H), 7.66-7.61 (m, 2H), 7.46-7.40 (m, 2H), 5.67 (bs, 2H), 4.51 (s, 4H), 4.00 (s, 3H). [M+H]⁺ 420. Rt 2.03 min (method N).

### Example 35

### tert-butyl 4-(4-(4-(1-methyl-1H-pyrazol-4-yl)phenyl)isoquinoline-6-carbonyl)piperazine-1-carboxylate (124)

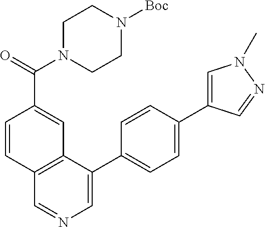 [SMILES: Cn1cc(-c2ccc(-c3cncc4ccc(C(=O)N5CCN(C(=O)OC(C)(C)C)CC5)cc34)cc2)cn1]

To a suspension of 4-(4-(1-methyl-1H-pyrazol-4-yl)phenyl)isoquinoline-6-carboxylic acid hydrochloride (Example 20) (50 mg, 0.152 mmol) in DMF (1.3 mL) was added HATU (69.3 mg, 0.182 mmol) and the mixture stirred for 20 min before the addition of tert-butyl piperazine-1-carboxylate (86 mg, 0.462 mmol). The mixture was stirred at rt for 15 min before the addition of DIPEA (116 μl, 0.662 mmol). The resulting solution was then stirred at rt overnight. The reaction mixture was concentrated (V10) and purified via biotage column chromatography (dichloromethane/EtOH, 98/2 to 90/10) to give the title compound (45 mg, 60% yield). ¹H NMR (500 MHz, CDCl₃) ppm=9.30 (s, 1H), 8.59 (s, 1H), 8.13 (d, J=8.3, 1H,), 8.01-7.98 (m, 1H), 7.86 (d, J=0.8, 1H), 7.73 (d, J=0.8, 1H), 7.68-7.62 (m, 3H), 7.53-7.48 (m, 2H,), 4.00 (s, 3H), 3.82-3.70 (m, 2H), 3.51 (d, J=18.2, 2H), 3.36 (s, 4H). [M+H]⁺ 498. Rt 2.86 min (method N).

### Example 36

### (4-(4-(1-methyl-1H-pyrazol-4-yl)phenyl)isoquinolin-6-yl)(piperazin-1-yl)methanone (102)

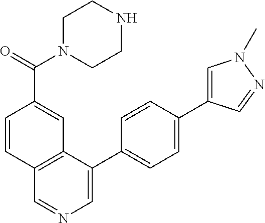 [SMILES: Cn1cc(-c2ccc(-c3cncc4ccc(C(=O)N5CCNCC5)cc34)cc2)cn1]

To a solution of tert-butyl 4-(4-(4-(1-methyl-1H-pyrazol-4-yl)phenyl)isoquinoline-6-carbonyl)piperazine-1-carboxylate (Example 24) (40 mg, 0.080 mmol) in dichloromethane (2.1 mL) was added trifluoroacetic acid (124 μl, 1.608 mmol). The reaction mixture was stirred at rt for 2 h and then concentrated. The residue was diluted with EtOAc. After addition of a sat. aq. solution of NaHCO₃, the layers were separated and the aqueous layer was extracted with EtOAc. The organic layers were dried over MgSO₄ and concentrated. The crude was purified via biotage column chromatography (dichloromethane/(MeOH/aq NH₃, 10/1), 98/2 to 92/8) to give the title product as a white solid (14 mg, 44% yield). ¹H NMR (500 MHz, CD₃OD) ppm=9.31 (s, 1H), 8.47 (s, 1H), 8.29 (d, J=8.5, 1H), 8.06 (s, 1H), 7.97 (s, 1H), 7.91 (s, 1H), 7.76-7.72 (m, 3H), 7.53 7.50 (d, J=8.4, 2H), 3.97 (s, 3H), 3.77-3.71 (bm, 2H), 3.44-3.37 (bm, 2H), 2.93-2.87 (bm, 2H), 2.79-2.73 (bm, 2H). [M+H]⁺ 398. Rt 1.8 min (method N).

### Example 37

### (3-Methoxy-azetidin-1-yl)-{4-[4-(1-methyl-1H-pyrazol-4-yl)-phenyl]-isoquinolin-6-yl}-methanone (112)

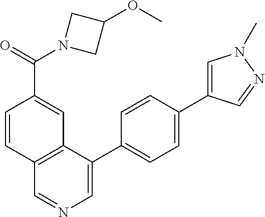 [SMILES: COC1CN(C(=O)c2ccc3cncc(-c4ccc(-c5cnn(C)c5)cc4)c3c2)C1]

Into a 25 mL round-bottom flask, were placed 4-[4-(1-methyl-1H-pyrazol-4-yl)phenyl]isoquinoline-6-carboxylic acid (100 mg, 0.30 mmol), 3-methoxyazetidine hydrochloride (37.5 mg, 0.30 mmol), 1-[bis(dimethylamino)methylene]-1H-1,2,3-triazolo[4,5-b]pyridinium 3-oxid hexafluorophosphate (HATU, 127 mg, 0.33 mmol), N,N-diisopropylethylamine (0.50 mL, 3.03 mmol) and N,N-dimethylformamide (5 mL). The solution was stirred for 2 h at 25° C. The mixture was concentrated under vacuum. The residue was applied to a silica gel column with dichloromethane/methanol (100:1). This resulted in 11.7 mg (10%) of the title compound as a white solid. ¹H NMR (400 MHz, CD₃OD): ppm=9.35 (s, 1H), 8.52 (s, 1H), 8.32 (d, 1H), 8.22 (s, 1H), 8.11 (s, 1H), 7.97 (m, 2H), 7.81 (d, 2H), 7.58 (d, 2H), 4.50-4.46 (m, 1H), 4.40-4.36 (m, 1H), 4.33-4.28 (m, 1H), 4.17-4.14 (m, 1H), 4.04-3.99 (m, 4H). [M+H]⁺ 399. Rt 2.14 min (method H).

### Example 38

### (1-amino-4-(4-(1-methyl-1H-pyrazol-4-yl)phenyl)isoquinolin-6-yl)(3-methoxyazetidin-1-yl)methanone (108)

### 6-(3-methoxyazetidine-1-carbonyl)-4-(4-(1-methyl-1H-pyrazol-4-yl)phenyl)isoquinoline 2-oxide

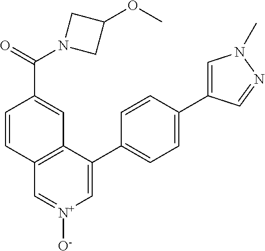 [SMILES: COC1CN(C(=O)c2ccc3c[n+]([O-])cc(-c4ccc(-c5cnn(C)c5)cc4)c3c2)C1]

To a solution of (3-methoxyazetidin-1-yl) (4-(4-(1-methyl-1H-pyrazol-4-yl)phenyl)isoquinolin-6-yl)methanone (Example 26) (30 mg, 0.075 mmol) in dichloromethane (753 μl) was added 3-chloroperoxybenzoic acid (13 mg, 0.075 mmol) and the reaction mixture was stirred at rt for 1 h. Additional 4 mg of 3-chloroperoxybenzoic acid were added. After 30 min, the conversion was still not complete and another 2 mg of 3-chloroperoxybenzoic acid were added and the reaction mixture was stirred for another 30 min at RT. 1N NaOH and dichloromethane were added to the reaction mixture and the layers were separated. The aqueous layers were extracted with dichloromethane and the organics layers were dried over MgSO₄ and concentrated. The crude product was used in the next step without any purification.

### (1-amino-4-(4-(1-methyl-1H-pyrazol-4-yl)phenyl)isoquinolin-6-yl) (3-methoxyazetidin-1-yl)methanone

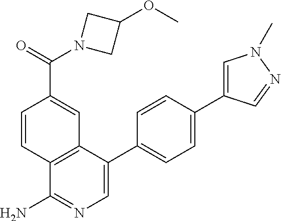 [SMILES: COC1CN(C(=O)c2ccc3c(N)ncc(-c4ccc(-c5cnn(C)c5)cc4)c3c2)C1]

To a suspension of 6-(3-methoxyazetidine-1-carbonyl)-4-(4-(1-methyl-1H-pyrazol-4-yl)phenyl)isoquinoline 2-oxide (26 mg, 0.063 mmol) in pyridine (1.5 mL, 12.55 mmol) was added 4-toluenesulfonlyl chloride (14.35 mg, 0.075 mmol) and the mixture was stirred at rt for 1 h 15 min. To the mixture was added ethanolamine (95 μL, 1.568 mmol) and the reaction mixture was stirred at rt for 1 h. The reaction mixture was diluted with water and ethyl acetate. The layers were separated and the aqueous layers were extracted three times with EtOAc. The organic layers were combined, dried over MgSO₄ and concentrated. The crude was purified via biotage column chromatography (dichloromethane/EtOH 96/4 to 82/18, single step 12 g) to give the title compound as a yellow solid (20 mg, 77% yield). ¹H NMR (500 MHz, CH₃OD) ppm=8.06 (d, J=1.4, 1H), 8.00 (s, 1H), 7.93 (d, J=8.5, 1H), 7.85 (d, J=0.8, 1H), 7.81 (d, J=8.5, 1.4, 1H), 7.71 (s, 1H), 7.61 (d, J=8.2, 2H), 7.45 (d, J=8.2, 2H), 4.40-4.30 (m, 2H), 4.26-4.21 (m, 1H), 4.11-4.05 (m, 2H), 4.00 (s, 3H), 3.30 (s, 3H). [M+H]⁺ 414. Rt 2.03 min (method N).

### Example 39

### (3-fluoro-3-methylazetidin-1-yl)(4-(4-(1-methyl-1H-pyrazol-4-yl)phenyl)isoquinolin-6-yl)methanone (186)

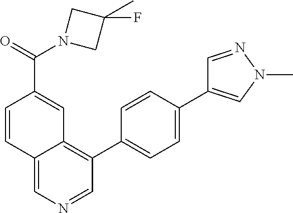 [SMILES: Cn1cc(-c2ccc(-c3cncc4ccc(C(=O)N5CC(C)(F)C5)cc34)cc2)cn1]

To a suspension of 4-(4-(1-methyl-1H-pyrazol-4-yl)phenyl)isoquinoline-6-carboxylic acid (48 mg, 0.15 mmol), HATU (62 mg, 0.16 mmol), DIPEA (0.15 mL, 0.88 mmol) in DMF (1.3 mL) was added 3-fluoro-3-methylazetidine (19 mg, 0.15 mmol), and the reaction mixture was stirred at room temperature for 2 h. The crude reaction mixture was poured onto 1M NaOH, and the organic material extracted with ethyl acetate (twice). The combined organic layers were washed with brine, dried over MgSO₄, filtered, and the filtrate concentrated under reduced pressure. The crude material was purified over a silica cartridge using a solvent system of 0-15% EtOH in dichloromethane and further purified over a silica cartridge using a solvent system of 0-30% EtOAc in dichloromethane. The title compound was isolated as a colourless solid (20 mg, 34%). ¹H NMR (400 MHz, MeOD) ppm=9.30 (s, 1H), 8.46 (s, 1H), 8.27 (d, J=8.5 Hz, 1H), 8.19 (s, 1H), 8.06 (s, 1H), 7.93 (dd, J=8.5, 1.6 Hz, 1H), 7.91 (s, 1H), 7.76 (d, J=8.2 Hz, 2H), 7.52 (d, J=8.2 Hz, 2H), 4.45-4.16 (m, 4H), 3.95 (s, 3H), 1.61 (d, J=21.6 Hz, 3H). [M+H]⁺ 401. Rt 1.33 min (method O)

### Example 40

### (1-amino-4-(4-(1-methyl-1H-pyrazol-4-yl)phenyl)isoquinolin-6-yl)(3-fluoro-3-methylazetidin-1-yl)methanone (188)

### 6-(3-fluoro-3-methylazetidine-1-carbonyl)-4-(4-(1-methyl-1H-pyrazol-4-yl)phenyl)isoquinoline 2-oxid

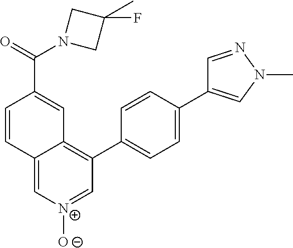 [SMILES: Cn1cc(-c2ccc(-c3c[n+]([O-])cc4ccc(C(=O)N5CC(C)(F)C5)cc34)cc2)cn1]

To a foil-covered solution of (3-fluoro-3-methylazetidin-1-yl)(4-(4-(1-methyl-1H-pyrazol-4-yl)phenyl)isoquinolin-6-yl)methanone (18 mg, 0.04 mmol) in dichloromethane (0.5 mL) at room temperature, was added mCPBA (32 mg, 0.14 mmol) in one portion. The reaction mixture was stirred at room temperature for 1 h. The crude solution was poured onto 1M NaOH, and the organic material was extracted with EtOAc. The combined organic layers were washed with brine, dried over MgSO₄, filtered, and the filtrate concentrated under reduced pressure. The crude product was used in the next step without further purification.

### (1-amino-4-(4-(1-methyl-1H-pyrazol-4-yl)phenyl)isoquinolin-6-yl)(3-fluoro-3-methylazetidin-1-yl)methanone

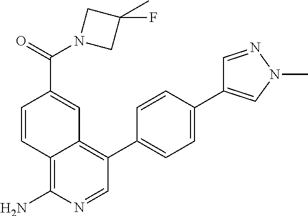 [SMILES: Cn1cc(-c2ccc(-c3cnc(N)c4ccc(C(=O)N5CC(C)(F)C5)cc34)cc2)cn1]

To 6-(3-fluoro-3-methylazetidine-1-carbonyl)-4-(4-(1-methyl-1H-pyrazol-4-yl)phenyl)isoquinoline 2-oxide (19 mg, 0.05 mmol) in pyridine (0.74 mL), was added tosyl chloride (10 mg, 0.06 mmol). The reaction mixture was stirred at room temperature for 30 min. To the reaction mixture at room temperature was added ethanolamine (69 μL, 1.14 mmol) and the resultant solution stirred at room temperature for 1 h. The crude mixture was diluted with water, and the organic material extracted with ethyl acetate. The combined organic layers were washed with brine, dried over MgSO₄, filtered, and the filtrate concentrated under reduced pressure. The crude product was purified over a silica cartridge using a solvent system of 1-16% ethanol in dichloromethane. The title compound was isolated as a yellow solid (6 mg, 32%). ¹H NMR (400 MHz, CDCl₃) ppm=8.07 (s, 1H), 8.00 (s, 1H), 7.94 (d, J=8.6 Hz, 1H), 7.84 (s, 1H), 7.79 (dd, J=8.6, 1.7 Hz, 1H), 7.70 (s, 1H), 7.61 (d, J=8.2 Hz, 2H), 7.44 (d, J=8.2 Hz, 2H), 5.35 (s, 2H), 4.41-4.33 (m, 2H), 4.22-4.08 (m, 2H) 4.00 (s, 3H), 1.63 (d, J=21.4 Hz, 3H). [M+H]⁺ 416. Rt 1.11 min (method M)

### Example 41

### (1-amino-4-(4-(1-methyl-1H-pyrazol-4-yl)phenyl)isoquinolin-6-yl)(3-methoxy-3-methylazetidin-1-yl)methanone (187)

### (3-methoxy-3-methylazetidin-1-yl) (4-(4-(1-methyl-1H-pyrazol-4-yl)phenyl)isoquinolin-6-yl)methanone

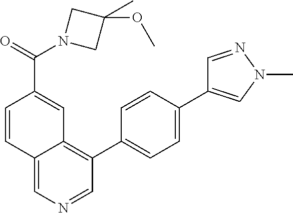 [SMILES: COC1(C)CN(C(=O)c2ccc3cncc(-c4ccc(-c5cnn(C)c5)cc4)c3c2)C1]

To a suspension of 4-(4-(1-methyl-1H-pyrazol-4-yl)phenyl)isoquinoline-6-carboxylic acid (49 mg, 0.15 mmol), HATU (62 mg, 0.16 mmol), DIPEA (0.15 mL, 0.88 mmol) in DMF (1.3) was added 3-methoxy-3-methylazetidine (22 mg, 0.16 mmol), and the reaction mixture was stirred at room temperature for 2 h. The crude reaction mixture was poured onto 1M NaOH, and the organic material extracted with ethyl acetate (twice). The combined organic layers were washed with brine, dried over MgSO₄, filtered, and the filtrate concentrated under reduced pressure. The crude material was purified over a silica cartridge using a solvent system of 0-12% ethanol in dichloromethane and further purified over a silica cartridge using a solvent system of 10-50% ethyl acetate in dichloromethane. The title compound was isolated as a colourless solid (49 mg, 81%). ¹H NMR (400 MHz, CDCl₃) ppm=9.28 (s, 1H), 8.56 (s, 1H), 8.19 (s, 1H), 8.09 (d, J=8.5 Hz, 1H), 7.86 (d, J=8.5 Hz, 1H), 7.84 (s, 1H), 7.71 (s, 1H), 7.64 (d, J=8.2 Hz, 2H), 7.50 (d, J=8.2 Hz, 2H), 4.16 (dd, J=15.1, 9.8 Hz, 2H), 3.99 (s, 3H), 3.96-3.92 (m, 2H), 3.23 (s, 3H), 1.47 (s, 3H). [M+H]⁺ 413. Rt 1.33 min (method O)

### 6-(3-methoxy-3-methylazetidine-1-carbonyl)-4-(4-(1-methyl-1H-pyrazol-4-yl)phenyl)isoquinoline 2-oxide

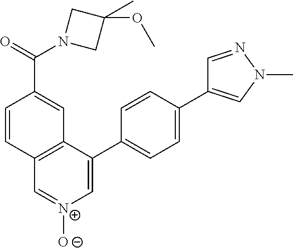 [SMILES: COC1(C)CN(C(=O)c2ccc3c[n+]([O-])cc(-c4ccc(-c5cnn(C)c5)cc4)c3c2)C1]

To a foil-covered solution of (3-methoxy-3-methylazetidin-1-yl)(4-(4-(1-methyl-1H-pyrazol-4-yl)phenyl)isoquinolin-6-yl)methanone (22 mg, 0.05 mmol) in dichloromethane (1 mL) at room temperature, was added mCPBA (49 mg, 0.21 mmol) in one portion. The reaction mixture was stirred at room temperature for 1 h. The crude solution was poured onto 1M NaOH, and the organic material was extracted with EtOAc. The combined organic layers were washed with brine, dried over MgSO₄, filtered, and the filtrate concentrated under reduced pressure. The crude product was used in the next step without further purification.

### (1-amino-4-(4-(1-methyl-1H-pyrazol-4-yl)phenyl)isoquinolin-6-yl)(3-methoxy-3-methylazetidin-1-yl)methanone

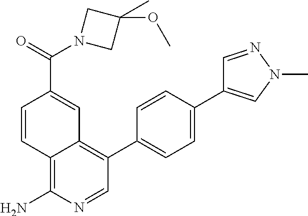 [SMILES: COC1(C)CN(C(=O)c2ccc3c(N)ncc(-c4ccc(-c5cnn(C)c5)cc4)c3c2)C1]

To 6-(3-methoxy-3-methylazetidine-1-carbonyl)-4-(4-(1-methyl-1H-pyrazol-4-yl)phenyl)isoquinoline 2-oxide (23 mg, 0.05 mmol) in pyridine (0.87 mL), was added tosyl chloride (12 mg, 0.06 mmol). The reaction mixture was stirred at room temperature for 30 min. To the reaction mixture at room temperature was added ethanolamine (81 uL, 1.34 mmol) and the resultant solution stirred at room temperature for 1 h. The crude reaction mixture was diluted with water, and the organic material extracted with ethyl acetate. The combined organic layers were washed with brine, dried over MgSO₄, filtered, and the filtrate concentrated under reduced pressure. The crude product was purified over a silica cartridge using a solvent system of 1-15% ethanol in dichloromethane. The title compound was isolated as a yellow solid (13 mg, 57%). ¹H NMR (400 MHz, CDCl₃) ppm=8.08 (d, J=1.6 Hz, 1H), 7.99 (s, 1H), 7.94 (d, J=8.5 Hz, 1H), 7.83 (s, 1H), 7.78 (dd, J=8.5 Hz, 1.7 Hz, 1H), 7.68 (s, 1H), 7.59 (d, J=8.2 Hz, 2H), 7.43 (d, J=8.2 Hz, 2H), 5.36 (s, 2H), 4.16 (dd, J=12.7, 9.7 Hz, 2H), 3.99 (s, 3H), 3.98-3.90 (m, 2H), 3.23 (s, 3H), 1.47 (s, 3H). [M+H]⁺ 428. Rt 0.91 min (method O).

### Example 42

### (3,3-difluoropyrrolidin-1-yl)(4-(4-(1-methyl-1H-pyrazol-4-yl)phenyl)isoquinolin-6-yl)methanone (195)

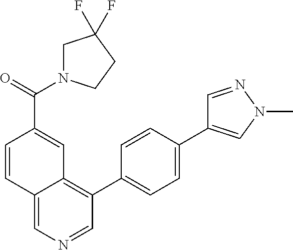 [SMILES: Cn1cc(-c2ccc(-c3cncc4ccc(C(=O)N5CCC(F)(F)C5)cc34)cc2)cn1]

To a suspension of 4-(4-(1-methyl-1H-pyrazol-4-yl)phenyl)isoquinoline-6-carboxylic acid (40 mg, 0.12 mmol), HATU (92 mg, 0.24 mmol), DIPEA (0.13 mL, 0.73 mmol) in DMF (2 mL) was added 3,3-difluoropyrrolidine (19 mg, 0.13 mmol), and the reaction mixture was stirred at room temperature for 2 h. The crude reaction mixture was poured onto 1M NaOH, and the organic material extracted with ethyl acetate (twice). The combined organic layers were washed with brine, dried over MgSO₄, filtered, and the filtrate concentrated under reduced pressure. The crude material was purified over a silica cartridge using a solvent system of 0-12% ethanol in dichloromethane to give the title compound (35 mg, 69%). ¹H NMR (400 MHz, CDCl₃) ppm=9.31 (s, 1H), 8.60 (s, 1H), 8.14 (d, J=8.4 Hz, 1H), 8.07 (d, J=22.1 Hz, 1H), 7.86 (s, 1H), 7.75-7.72 (m, 2H), 7.66 (d, J=8.2 Hz, 2H), 7.51 (d, J=8.2. Hz, 2H), 4.04-3.99 (m, 4H), 3.91 (t, J=7.5 Hz, 1H), 3.72 (t, J=12.0 Hz, 1H), 3.63 (t, J=7.5 Hz, 1H), 2.40 (ddt, J=26.7, 13.3, 7.2 Hz, 1H). [M+H]⁺ 419 Rt 1.22 min (method O).

### Example 43

### (1-amino-4-(4-(1-methyl-1H-pyrazol-4-yl)phenyl)isoquinolin-6-yl)(3,3-difluoropyrrolidin-1-yl)methanone (196)

### 6-(3,3-difluoropyrrolidine-1-carbonyl)-4-(4-(1-methyl-1H-pyrazol-4-yl)phenyl)isoquinoline 2-oxide

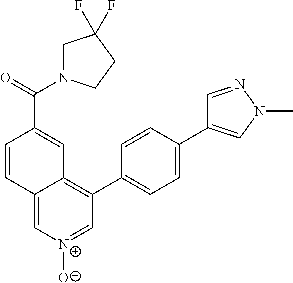 [SMILES: Cn1cc(-c2ccc(-c3c[n+]([O-])cc4ccc(C(=O)N5CCC(F)(F)C5)cc34)cc2)cn1]

To a foil-covered solution of (3,3-difluoropyrrolidin-1-yl)(4-(4-(1-methyl-1H-pyrazol-4-yl)phenyl)isoquinolin-6-yl)methanone (24 mg, 0.06 mmol) in dichloromethane (0.8 mL) at room temperature, was added mCPBA (40 mg, 0.17 mmol) in one portion. The reaction mixture was stirred at room temperature for 1 h. The crude solution was poured onto 1M NaOH, and the organic material was extracted with EtOAc. The combined organic layers were washed with brine, dried over MgSO₄, filtered, and the filtrate concentrated under reduced pressure. The crude product was used in the next step without further purification.

### (1-amino-4-(4-(1-methyl-1H-pyrazol-4-yl)phenyl)isoquinolin-6-yl)(3,3-difluoropyrrolidin-1-yl)methanone

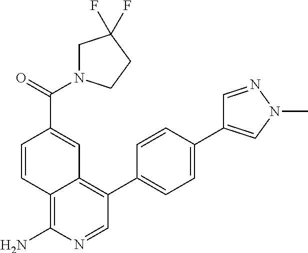 [SMILES: Cn1cc(-c2ccc(-c3cnc(N)c4ccc(C(=O)N5CCC(F)(F)C5)cc34)cc2)cn1]

To 6-(3,3-difluoropyrrolidine-1-carbonyl)-4-(4-(1-methyl-1H-pyrazol-4-yl)phenyl)isoquinoline 2-oxide (25 mg, 0.06 mmol) in pyridine (0.9 mL), was added tosyl chloride (13 mg, 0.07 mmol). The reaction mixture was stirred at room temperature for 30 min. To the reaction mixture at room temperature was added ethanolamine (0.09 mL, 1.43 mmol) and the resultant solution stirred at room temperature for 1 h. The crude reaction mixture was diluted with water, and the organic material extracted with ethyl acetate. The combined organic layers were washed with brine, dried over MgSO₄, filtered, and the filtrate concentrated under reduced pressure. The crude product was purified over a silica cartridge using a solvent system of 1-15% ethanol in dichloromethane to give the title compound (15 mg, 60%). ¹H NMR (400 MHz, CDCl₃) ppm=7.99-7.93 (m, 3H), 7.84 (s, 1H), 7.69 (s, 1H), 7.66-7.59 (m, 3H), 7.43 (d, J=8.1 Hz, 2H), 5.44 (s, 2H), 4.02-3.97 (m, 4H), 3.88 (m, 1H), 3.71 (t, J=12.0 Hz, 1H), 3.61 (t, J=7.3 Hz, 1H), 2.39 (dddt, J=33.5, 20.2, 13.6, 7.2 Hz, 2H). [M+H]⁺ 434. Rt 0.98 min (method O)

### Example 44

### 4-(4-(1-methyl-1H-pyrazol-4-yl)phenyl)-6-(2-methyl-2H-tetrazol-5-yl)isoquinoline (117); and Example 29: 4-(4-(1-methyl-1H-pyrazol-4-yl)phenyl)-6-(1-methyl-1H-tetrazol-5-yl)isoquinoline (123)

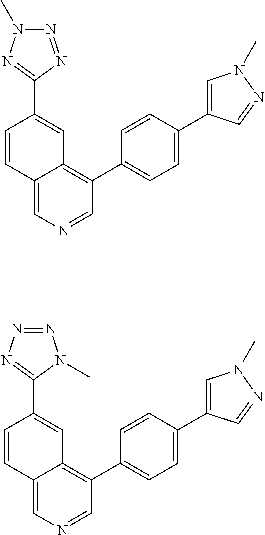 [SMILES: Cn1cc(-c2ccc(-c3cncc4ccc(-c5nnn(C)n5)cc34)cc2)cn1 ; Cn1cc(-c2ccc(-c3cncc4ccc(-c5nnnn5C)cc34)cc2)cn1]

A mixture of 4-(4-(1-methyl-1H-pyrazol-4-yl)phenyl)isoquinoline-6-carbonitrile (Example 17) (48 mg, 0.16 mmol) and sodium azide (20 mg, 0.31 mmol) in DMF (0.8 mL) was heated at 150° C. for 2.5 h under microwave irradiation. The reaction mixture was concentrated in vacuo. The residue was taken up in DMF (1 mL) and powdered KOH (15 mg, 0.27 mmol) was added. The resulting suspension was stirred for 10 min at rt before the addition of a solution of MeI (30 mg, 0.21 mmol) in DMF (0.3 mL). The mixture was then stirred at rt for 2.5 h. Water (10 mL) was then added and the mixture neutralised with 1 M HCl (˜0.3 mL) and extracted with CH₂Cl₂ (3×15 mL). The combined organic layers were washed with sat. aq. NH₄Cl (2×10 mL), filtered through a phase separator, and concentrated in vacuo. The crude material was purified by Biotage (SNAP 10 g column, CH₂Cl₂/EtOH 97/3->92/8) to give 4-(4-(1-methyl-1H-pyrazol-4-yl)phenyl)-6-(2-methyl-2H-tetrazol-5-yl)isoquinoline (12 mg, 22%) as an off-white solid and impure 4-(4-(1-methyl-1H-pyrazol-4-yl)phenyl)-6-(1-methyl-1H-tetrazol-5-yl)isoquinoline which was purified by preparative HPLC to give 4-(4-(1-methyl-1H-pyrazol-4-yl)phenyl)-6-(1-methyl-1H-tetrazol-5-yl)isoquinoline (3.3 mg, 6%) as a white solid. Example 28 ¹H NMR (500 MHz, CDCl₃) ppm=9.31 (s, 1H), 8.79 (s, 1H), 8.58 (s, 1H), 8.38 (dd, J=8.5, 1.5, 1H), 8.18 (d, J=8.6, 1H), 7.88 (s, 1H), 7.73 (s, 1H), 7.68 (d, J=8.1, 2H), 7.57 (d, J=8.2, 2H), 4.42 (s, 3H), 4.01 (s, 3H). [M+H]⁺ 368. Rt 1.47 min (method M). Example 29 ¹H NMR (500 MHz, CDCl₃) ppm=9.40 (s, 1H), 8.68 (s, 1H), 8.36 (s, 1H), 8.28 (d, J=8.5, 1H), 8.03 (dd, J=8.5, 1.7, 1H), 7.86 (s, 1H), 7.73 (s, 1H), 7.67 (d, J=8.1, 2H), 7.54 (d, J=8.1, 2H), 4.18 (s, 3H), 4.01 (s, 3H). [M+H]⁺ 368. Rt 1.29 min (method M).

### Example 45

### 4-(2,2-dioxido-1,3-dihydrobenzo[c]isothiazol-5-yl)isoquinoline-6-carbonitrile (193)

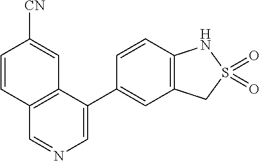 [SMILES: N#Cc1ccc2cncc(-c3ccc4c(c3)CS(=O)(=O)N4)c2c1]

4-chloroisoquinoline-6-carbonitrile (215 mg, 1.14 mmol), 5-(4,4,5,5-tetramethyl-1,3,2-dioxaborolan-2-yl)-1,3-dihydrobenzo[c]isothiazole 2,2-dioxide (370 mg, 1.25 mmol) and Pd(dppf)Cl₂.CH₂Cl₂ (47 mg, 0.057 mmol) were loaded in a microwave vial and then acetonitrile (20 mL) and sodium carbonate in water (0.5 M, 3.20 mL, 1.60 mmol) were added. The reaction mixture was heated at 150° C. for 1 h. The reaction mixture was concentrated and purified via biotage column (SNAP 25 g, DCM/EtOH 99/1 to 97/3) to give the title compound as a white solid (230 mg, 38% yield). ¹H NMR (500 MHz, DMSO) 810.82 (s, 1H), 9.49 (s, 1H), 8.60 (s, 1H), 8.43 (d, J=8.5 Hz, 1H), 8.37-3.36 (m, 1H), 8.05 (dd, J=8.5, 1.5 Hz, 1H), 7.52 (s, 1H), 7.49-7.45 (m, 1H), 7.03 (d, J=8.1 Hz, 1H), 4.66 (s, 2H). [M+H]⁺ 322. Rt 1.11 min (method M).

### Example 46

### azetidin-1-yl(4-(2,2-dioxido-1,3-dihydrobenzo[c]isothiazol-5-yl)isoquinolin-6-yl)methanone (192)

### 4-(2,2-dioxido-1,3-dihydrobenzo[c]isothiazol-5-yl)isoquinoline-6-carboxylic acid

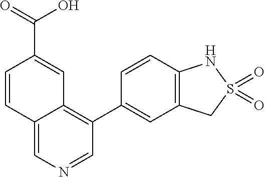 [SMILES: O=C(O)c1ccc2cncc(-c3ccc4c(c3)CS(=O)(=O)N4)c2c1]

To 4-(2,2-dioxido-1,3-dihydrobenzo[c]isothiazol-5-yl)isoquinoline-6-carbonitrile (230 mg, 0.429 mmol) in ethanol (3.33 mL) was added 2 M sodium hydroxide (3.33 mL, 6.66 mmol). The reaction was heated at 100° C. for 1 h 30. Hydrogen chloride in dioxane (6.66 mL, 6.66 mmol) was added and the reaction mixture was concentrated and the crude mixture was used in the next step.

### azetidin-1-yl(4-(2,2-dioxido-1,3-dihydrobenzo[c]isothiazol-5-yl)isoquinolin-6-yl)methanone

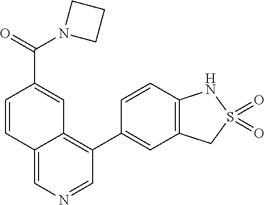 [SMILES: O=C(c1ccc2cncc(-c3ccc4c(c3)CS(=O)(=O)N4)c2c1)N1CCC1]

To 4-(2,2-dioxido-1,3-dihydrobenzo[c]isothiazol-5-yl)isoquinoline-6-carboxylic acid (200 mg, 0.590 mmol) in DMF (4.9 mL) was added HATU (269 mg, 0.708 mmol), azetidine (40 μl, 0.50 mmol) and DIPEA (227 μl, 1.298 mmol). The reaction mixture was stirred at rt overnight. The solvent was evaporated and the crude material was purified via Biotage column chromatography (single step 25 g, DCM/EtOH 99/1 to 90/10). The product obtained was solubilized in DCM and washed with water. The organic layer was dried over MgSO₄ and concentrated to give the title compound as a white solid (80 mg, 36% yield). ¹H NMR (500 MHz, CDCl₃) δ 9.30 (s, 1H), 8.49 (s, 1H), 8.18 (s, 1H), 8.12-8.08 (m, 1H), 7.84 (dd, J=8.5, 1.5 Hz, 1H), 7.40-7.36 (m, 2H), 7.03-7.00 (m, 1H), 4.48 (s, 2H), 4.27 (dt, J=15.2, 7.8 Hz, 4H), 2.42-2.32 (m, 2H). [M+H]⁺ 380. Rt 1.87 min (method N).

### Example 47

### (1-amino-4-(2,2-dioxido-1,3-dihydrobenzo[c]isothiazol-5-yl)isoquinolin-6-yl)(azetidin-1-yl)methanone (194)

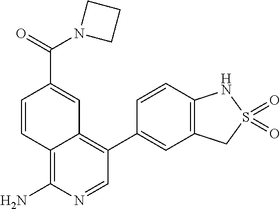 [SMILES: Nc1ncc(-c2ccc3c(c2)CS(=O)(=O)N3)c2cc(C(=O)N3CCC3)ccc12]

To a suspension of azetidin-1-yl(4-(2,2-dioxido-1,3-dihydrobenzo[c]isothiazol-5-yl)isoquinolin-6-yl)methanone (65 mg, 0.17 mmol) in DCM (6.8 mL) and MeOH (0.8 mL) was added 3-chloroperoxybenzoic acid (77 mg, 0.34 mmol). The reaction mixture was stirred at rt for 3 h. After addition of a solution of Na₂S₂O₅ and then NaHCO₃, the reaction mixture was diluted with DCM. The compound was very water soluble therefore the aqueous layer was concentrated. The residue obtained was solubilized in pyridine (8 mL). 4-toluenesulfonyl chloride (39.1 mg, 0.205 mmol) was added and the reaction mixture was stirred at rt for 2 h. An additional 1.2 eq of 4-toluenesulfonyl chloride (39.1 mg, 0.205 mmol) were added and the reaction mixture was stirred at rt for 1 h. Ethanolamine (259 μl, 4.28 mmol) was then added and the reaction mixture was stirred at rt overnight. Water and DCM were added, the aqueous layer was extracted with DCM three times and the organic layer was concentrated. The crude was purified via biotage (SNAP 25 g, DCM/EtOH 96/4 to 80/20) and by SCX column to give the title compound (5 mg, 7% yield). ¹H NMR (500 MHz, DMSO) δ 10.65 (s, 1H), 8.37 (d, J=8.7 Hz, 1H), 7.82 (d, J=1.7 Hz, 1H), 7.76 (s, 1H), 7.72 (dd, J=8.7, 1.7 Hz, 1H), 7.35 (d, J=1.7 Hz, 1H), 7.33-7.28 (m, 1H), 6.97 (d, J=8.0 Hz, 1H), 4.62 (s, 2H), 4.23 (t, J=7.7 Hz, 2H), 4.04 (t, J=7.7 Hz, 2H), 2.26 (p, J=7.7 Hz, 2H). [M+H]⁺ 395. Rt 1.51 min (method N).

### Example 48

### 4-(2-oxoindolin-6-yl)isoquinoline-6-carbonitrile (148)

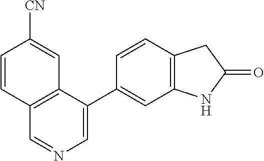 [SMILES: N#Cc1ccc2cncc(-c3ccc4c(c3)NC(=O)C4)c2c1]

4-(2-oxoindolin-6-yl)isoquinoline-6-carbonitrile was prepared in a manner similar to Example 46, using 6-(4,4,5,5-tetramethyl-1,3,2-dioxaborolan-2-yl)indolin-2-one as the starting material. ¹H NMR (500 MHz, DMSO) ppm=10.53 (s, 1H), 9.51 (s, 1H), 8.62 (s, 1H), 8.44 (d, J=8.5, 0.8 Hz, 1H), 8.33 (dt, J=1.6, 0.8 Hz, 1H), 8.05 (dd, J=8.5, 1.6 Hz, 1H), 7.42 (d, J=7.5 Hz, 1H), 7.13 (dd, J=7.5, 1.6 Hz, 1H), 6.97-6.94 (m, 1H), 3.60 (s, 2H). [M+H]⁺ 286. Rt 2.35 min (method N).

### Example 49

### 6-(6-(azetidine-1-carbonyl)isoquinolin-4-yl)indolin-2-one (145)

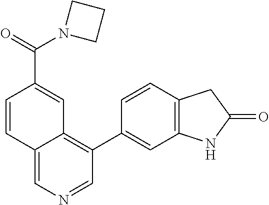 [SMILES: O=C1Cc2ccc(-c3cncc4ccc(C(=O)N5CCC5)cc34)cc2N1]

6-(6-(azetidine-1-carbonyl)isoquinolin-4-yl)indolin-2-one was prepared in a manner similar to Example 46, using 4-(2-oxoindolin-6-yl)isoquinoline-6-carbonitrile as starting material. ¹H NMR (500 MHz, CDCl₃) ppm=9.32 (s, 1H), 8.95 (s, 1H), 8.52 (s, 1H), 8.15 (bs, 1H), 8.11 (d, J=8.6, 1H), 7.88 (dd, J=8.6, 1.5 Hz, 1H), 7.39 (d, J=7.5 Hz, 1H), 7.12 (dd, J=7.5, 1.4 Hz, 1H), 7.00 (d, J=1.4 Hz, 1H), 4.24 (td, J=7.8, 5.2 Hz, 4H), 3.65 (s, 2H,), 2.41-2.30 (m, 2H). [M+H]⁺ 344. Rt 2.17 min (method N).

### Example 50

### 6-(6-(3,3-difluoroazetidine-1-carbonyl)isoquinolin-4-yl)indolin-2-one (146)

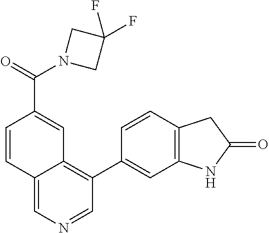 [SMILES: O=C1Cc2ccc(-c3cncc4ccc(C(=O)N5CC(F)(F)C5)cc34)cc2N1]

6-(6-(3,3-difluoroazetidine-1-carbonyl)isoquinolin-4-yl)indolin-2-one was prepared in a manner similar to Example 46, using 4-(2-oxoindolin-6-yl)isoquinoline-6-carbonitrile and 3,3-difluoroazetidine hydrochloride as starting material. ¹H NMR (500 MHz, DMSO) ppm=10.58 (s, 1H), 9.44 (s, 1H), 8.53 (s, 1H), 8.32 (d, J=8.5 Hz, 1H), 8.12 (dd, J=1.6, 0.9 Hz, 1H), 7.96 (dd, J=8.5, 1.6 Hz, 1H), 7.43 (d, J=7.5 Hz, 1H), 7.12 (dd, J=7.5, 1.5 Hz, 1H), 6.95 (d, J=1.5 Hz, 1H), 4.74 (bs, 2H), 4.50 (s, 2H), 3.61 (s, 2H). [M+H]⁺ 380. Rt 2.42 min (method N).

### Example 51

### 6-(1-amino-6-(3,3-difluoroazetidine-1-carbonyl)isoquinolin-4-yl)indolin-2-one (154)

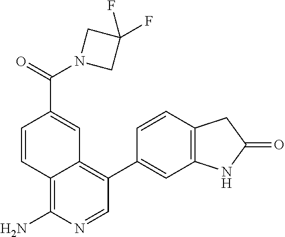 [SMILES: Nc1ncc(-c2ccc3c(c2)NC(=O)C3)c2cc(C(=O)N3CC(F)(F)C3)ccc12]

6-(1-amino-6-(3,3-difluoroazetidine-1-carbonyl)isoquinolin-4-yl)indolin-2-one was prepared in a manner similar to Example 47, using 6-(6-(3,3-difluoroazetidine-1-carbonyl)isoquinolin-4-yl)indolin-2-one as starting material. ¹H NMR (500 MHz, MeOD) ppm=8.62 (d, J=8.6 Hz, 1H), 8.10-8.03 (m, 2H), 7.62 (s, 1H), 7.47 (d, J=7.5 Hz, 1H), 7.13 (dd, J=7.5, 1.5 Hz, 1H), 7.01 (d, J=1.5 Hz, 1H), 4.74-4.48 (m, 4H, azetidine CH₂), 3.67 (s, 1H, indolinone CH₂). [M+H]⁺ 395. Rt 0.91 min (method M).

### Example 52

### 4-(4-(1-(2-hydroxy-2-methylpropyl)-1H-pyrazol-4-yl)phenyl)isoquinoline-6-carboxamide (97)

### 4-chloroisoquinoline-6-carboxamide

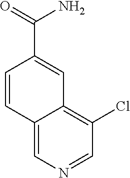 [SMILES: NC(=O)c1ccc2cncc(Cl)c2c1]

To 4-chloroisoquinoline-6-carbonitrile (101 mg, 0.54 mmol) was added an ice cold mixture of water (0.3 mL) and conc. H₂SO₄ (3.0 mL, 56.3 mmol) at 0° C. The reaction mixture was allowed to warm to rt and then heated at 50° C. for 45 min. The mixture was poured into ice cold 2 M aq. NaOH (25 mL), and the resulting mixture neutralised with sat. aq. NaHCO₃ (40 mL). Extracted with CH₂Cl₂ (4×50 mL) and the combined org. layers filtered through a phase separator and concentrated in vacuo to give the title compound as a cream coloured solid (68 mg, 62%). 4-(4-(1-(2-hydroxy-2-methylpropyl)-1H-pyrazol-4-yl)phenyl)isoquinoline-6-carboxamide

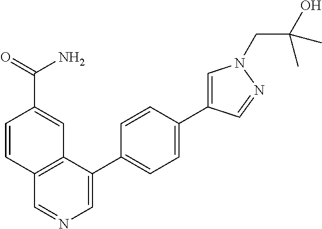 [SMILES: CC(C)(O)Cn1cc(-c2ccc(-c3cncc4ccc(C(N)=O)cc34)cc2)cn1]

A mixture of 4-chloroisoquinoline-6-carboxamide (68 mg, 0.33 mmol), 2-methyl-1-(4-(4-(4,4,5,5-tetramethyl-1,3,2-dioxaborolan-2-yl)phenyl)-1H-pyrazol-1-yl)propan-2-ol (136 mg, 0.40 mmol), K₃PO₄ (225 mg, 1.06 mmol), and Pd(dtbpf)Cl₂ (23 mg, 0.035 mmol) in a mixture of 1,4-dioxane (1.5 mL) and water (0.4 mL) was stirred at 120° C. for 1 h under microwave irradiation. The reaction mixture was concentrated in vacuo and purified by Biotage (SNAP 10 g column, cyclohexane/EtOAc 100/0->0/100, then CH₂Cl₂/EtOH 100/0->85/15) to give a brown oil which was taken up in CH₂Cl₂ and filtered. The resulting solid was washed with CH₂Cl₂ to give the title compound as an off-white solid (57 mg, 45%). ¹H NMR (500 MHz, CD₃OD) ppm=9.31 (s, 1H), 8.52 (s, 1H), 8.47 (s, 1H), 8.27 (d, J=8.6, 1H), 8.15-8.07 (m, 2H), 7.94 (s, 1H), 7.78 (d, J=8.1, 2H), 7.56 (d, J=8.2, 2H), 4.17 (s, 2H), 1.24 (s, 6H). [M+H]⁺ 387. Rt 1.08 min (method O).

### Example 53

### (4-(4-(1-(2-hydroxy-2-methylpropyl)-1H-pyrazol-4-yl)phenyl)isoquinolin-6-yl)(3-methoxyazetidin-1-yl)methanone (131)

### (4-chloroisoquinolin-6-yl)(3-methoxyazetidin-1-yl)methanone

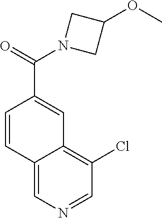 [SMILES: COC1CN(C(=O)c2ccc3cncc(Cl)c3c2)C1]

4-chloroisoquinoline-6-carbonitrile (24 mg, 0.13 mmol) was taken up in EtOH (1 mL) and 2 M NaOH (1 mL, 2.00 mmol) was added, and the resulting mixture heated at 150° C. for 1 h under microwave irradiation. The mixture was then neutralised with 2 M HCl (˜1 mL; pH˜7) and concentrated in vacuo. The residue was taken up in DMF (0.5 mL) and HATU (80 mg, 0.21 mmol) was added and the mixture stirred for 10 min before the addition of 3-methoxyazetidine hydrochloride (32 mg, 0.26 mmol) and DIPEA (0.05 mL, 0.29 mmol). The mixture was then stirred at rt for 18 h and concentrated in vacuo. The crude material was purified by Biotage (SNAP 10 g column, CH₂Cl₂/EtOH 100/0->90/10) to give the title compound as a yellow resin (30 mg, 85%) which was used without further purification.

### (4-(4-(1-(2-hydroxy-2-methylpropyl)-1H-pyrazol-4-yl)phenyl)isoquinolin-6-yl) (3-methoxyazetidin-1-yl)methanone

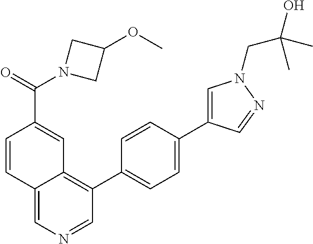 [SMILES: COC1CN(C(=O)c2ccc3cncc(-c4ccc(-c5cnn(CC(C)(C)O)c5)cc4)c3c2)C1]

A mixture of (4-chloroisoquinolin-6-yl)(3-methoxyazetidin-1-yl)methanone (30 mg, 0.11 mmol), 2-methyl-1-(4-(4-(4,4,5,5-tetramethyl-1,3,2-dioxaborolan-2-yl)phenyl)-1H-pyrazol-1-yl)propan-2-ol (42 mg, 0.12 mmol), K₃PO₄ (75 mg, 0.35 mmol), and Pd(dtbpf)Cl₂ (7 mg, 0.011 mmol) in a mixture of 1,4-dioxane (0.6 mL) and water (0.15 mL) was stirred at 120° C. for 1 h under microwave irradiation. The reaction mixture was concentrated in vacuo and purified by Biotage (SNAP g column, CH₂Cl₂/EtOH 100/0->85/15) to give the title compound as a yellow solid (23 mg, 47%). ¹H NMR (500 MHz, CDCl₃) ppm=9.93 (s, 1H), 8.57 (s, 1H), 8.16 (s, 1H), 8.11 (d, J=8.4, 1H), 7.92 (s, 1H), 7.88 (dd, J=8.5, 1.6, 1H), 7.81 (s, 1H), 7.67 (d, J=8.1, 2H), 7.51 (d, J=8.1, 2H), 4.39-4.30 (m, 2H), 4.24 (m, 1H), 4.16 (s, 2H), 4.12-4.06 (m, 2H), 3.87 (br s, 1H), 3.30 (s, 3), 1.25 (s, 6H). [M+H]⁺ 457. Rt 1.19 min (method O).

### Example 54

### (4-(1-(2-hydroxy-2-methylpropyl)-1H-indazol-5-yl)isoquinolin-6-yl)(3-methoxyazetidin-1-yl)methanone (132)

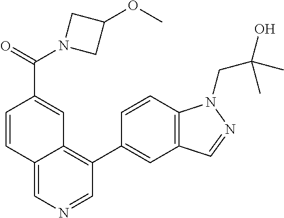 [SMILES: COC1CN(C(=O)c2ccc3cncc(-c4ccc5c(cnn5CC(C)(C)O)c4)c3c2)C1]

(4-(1-(2-hydroxy-2-methylpropyl)-1H-indazol-5-yl)isoquinolin-6-yl) (3-methoxyazetidin-1-yl)methanone was prepared according to Example 31, using 2-methyl-1-(5-(4,4,5,5-tetramethyl-1,3,2-dioxaborolan-2-yl)-1H-indazol-1-yl)propan-2-ol as the starting material. ¹H NMR (500 MHz, CDCl₃) ppm=9.32 (s, 1H), 8.60 (s, 1H), 8.20-8.10 (m, 3H), 7.91-7.85 (m, 2H), 7.64 (d, J=8.5, 1H), 7.54 (d, J=8.4, 1H), 4.44 (s, 2H), 4.40-4.28 (m, 2H), 4.22 (m, 1H), 4.10-4.03 (m, 2H), 3.63 (s, 1H), 3.29 (s, 3H), 1.32 (s, 6H). [M+H]⁺ 431. Rt 1.17 min (method M).

### Example 55

### (4-(2-(2-hydroxy-2-methylpropyl)-2H-indazol-5-yl)isoquinolin-6-yl)(3-methoxyazetidin-1-yl)methanone (133)

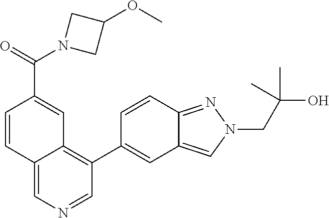 [SMILES: COC1CN(C(=O)c2ccc3cncc(-c4ccc5nn(CC(C)(C)O)cc5c4)c3c2)C1]

(4-(2-(2-hydroxy-2-methylpropyl)-2H-indazol-5-yl)isoquinolin-6-yl)(3-methoxyazetidin-1-yl)methanone was prepared according to Example 31, using 2-methyl-1-(5-(4,4,5,5-tetramethyl-1,3,2-dioxaborolan-2-yl)-2H-indazol-2-yl)propan-2-ol as the starting material. ¹H NMR (500 MHz, CDCl₃) ppm=9.31 (s, 1H), 8.61 (s, 1H), 8.18-8.06 (m, 3H), 7.88 (t, J=8.0, 2H), 7.80 (s, 1H), 7.43 (dd, J=8.8, 1.7, 1H), 4.44 (s, 2H), 4.38-4.29 (m, 2H), 4.22 (m, 1H), 4.11-4.04 (m, 2H), 3.29 (s, 3H), 1.83 (br s, 1H), 1.27 (s, 6H). [M+H]⁺ 431. Rt 1.12 min (method M).

### Example 56

### (4-(4-(1-(2-hydroxy-2-methylpropyl)-1H-pyrrol-3-yl)phenyl)isoquinolin-6-yl)(pyrrolidin-1-yl)methanone (134)

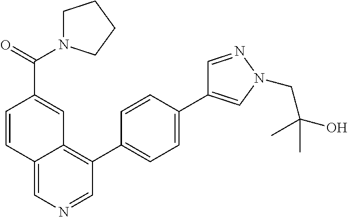 [SMILES: CC(C)(O)Cn1cc(-c2ccc(-c3cncc4ccc(C(=O)N5CCCC5)cc34)cc2)cn1]

(4-(4-(1-(2-hydroxy-2-methylpropyl)-1H-pyrrol-3-yl)phenyl)isoquinolin-6-yl)(pyrrolidin-1-yl)methanone was prepared according to Example 31, using pyrrolidine as the starting material. ¹H NMR (500 MHz, CDCl₃) ppm=9.28 (s, 1H), 8.55 (s, 1H), 8.09 (d, J=8.5, 1H), 8.06 (s, 1H), 7.91 (s, 1H), 7.80 (s, 1H), 7.75 (d, J=8.3, 1H), 7.65 (d, J=7.7, 2H), 7.51 (d, J=7.7, 2H), 4.15 (s, 2H), 3.92 (s, 1H), 3.64 (t, J=7.0, 2H), 3.34 (t, J=6.6, 2H), 1.96 (m, 2H), 1.87 (m, 2H), 1.24 (s, 6H). [M+H]⁺ 441. Rt 1.2 min Method O.

### Example 57

### (4-(4-(1-(2-hydroxy-2-methylpropyl)-1H-pyrrol-3-yl)phenyl)isoquinolin-6-yl)(morpholino)methanone (135)

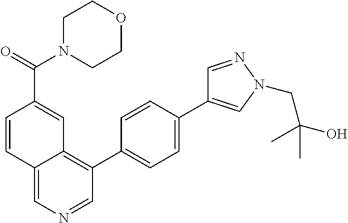 [SMILES: CC(C)(O)Cn1cc(-c2ccc(-c3cncc4ccc(C(=O)N5CCOCC5)cc34)cc2)cn1]

(4-(4-(1-(2-hydroxy-2-methylpropyl)-1H-pyrrol-3-yl)phenyl)isoquinolin-6-yl)(morpholino)methanone was prepared according to Example 31, using morpholine as the starting material. ¹H NMR (500 MHz, CDCl₃) ppm=9.30 (s, 1H), 8.57 (s, 1H), 8.11 (d, J=8.4, 1H), 7.99 (s, 1H), 7.91 (s, 1H), 7.80 (s, 1H), 7.68-7.62 (m, 3H), 7.50 (d, J=7.6, 2H), 4.15 (s, 2H), 3.89 (s, 1H), 3.83-3.72 (m, 4H), 3.63-3.55 (m, 2H), 3.43-3.35 (m, 2H), 1.24 (s, 6H). [M+H]⁺ 457. Rt 1.11 min (method O).

### Example 58

### (3,3-difluoroazetidin-1-yl)(4-(4-(1-(2-hydroxy-2-methylpropyl)-1H-pyrrol-3-yl)phenyl)isoquinolin-6-yl)methanone (136)

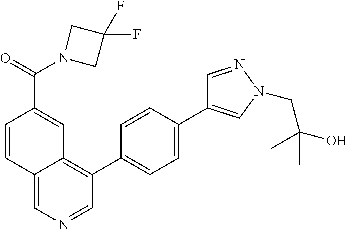 [SMILES: CC(C)(O)Cn1cc(-c2ccc(-c3cncc4ccc(C(=O)N5CC(F)(F)C5)cc34)cc2)cn1]

(3,3-difluoroazetidin-1-yl) (4-(4-(1-(2-hydroxy-2-methylpropyl)-1H-pyrrol-3-yl)phenyl)isoquinolin-6-yl)methanone was prepared according to Example 31, using 3,3-difluoroazetidine hydrochloride as the starting material. ¹H NMR (500 MHz, CDCl₃) ppm=9.32 (s, 1H), 8.60 (s, 1H), 8.19 (s, 1H), 8.14 (d, J=8.5, 1H), 7.92 (s, 1H), 7.88 (d, J=8.5, 1H), 7.81 (s, 1H), 7.68 (d, J=7.9, 2H), 7.50 (d, J=7.7, 2H), 4.51 (t, J=11.9, 4H), 4.16 (s, 2H), 1.25 (s, 6H). [M+H]⁺ 463. Rt 1.24 min (method O).

### Example 59

### 6-[6-(3-Methoxy-azetidine-1-carbonyl)-isoquinolin-4-yl]-1-methyl-3,4-dihydro-1H-quinolin-2-one (155)

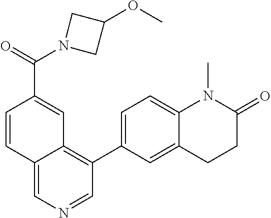 [SMILES: COC1CN(C(=O)c2ccc3cncc(-c4ccc5c(c4)CCC(=O)N5C)c3c2)C1]

Into a 10-mL vial purged and maintained with an inert atmosphere of nitrogen was added 4-chloro-6-[(3-methoxyazetidin-1-yl)carbonyl]isoquinoline (100 mg, 0.36 mmol), 1-methyl-6-(tetramethyl-1,3,2-dioxaborolan-2-yl)-1,2,3,4-tetrahydroquinolin-2-one (135 mg, 0.47 mmol), sodium carbonate (50.0 mg, 0.47 mmol), Pd(dppf)Cl₂*CH₂Cl₂ (30.0 mg, 0.04 mmol), acetonitrile (2 mL) and water (2 mL). The mixture was stirred under microwave irradiation for 1 h at 140° C. and concentrated under vacuum. The residue was purified by silica gel column chromatography with dichloromethane/methanol (20:1) and further purified by prep-HPLC (acetonitrile/water). This resulted in 30 mg (21%) of 6-[6-[(3-methoxyazetidin-1-yl)carbonyl]isoquinolin-4-yl]-1-methyl-1,2,3,4-tetrahydroquinolin-2-one as a white solid. ¹H NMR (400 MHz, DMSO-d6) ppm=9.41 (s, 1H), 8.53 (s, 1H), 8.30 (d, J=8.8 Hz, 1H), 8.07 (s, 1H), 7.93 (d, J=8.4 Hz, 1H), 7.49-7.47 (m, 2H), 7.31 (d, J=8.8 Hz, 1H), 4.47-4.43 (m, 1H), 4.28-4.25 (m, 2H), 4.13-4.11 (m, 1H), 3.89-3.86 (m, 1H), 3.35-3.32 (m, 3H), 3.22 (s, 3H), 3.00-2.96 (m, 2H), 2.66-2.62 (m, 2H). [M+H]⁺ 402. Rt 1.01 min (method Q).

### Example 60

### 5-[6-(3-Methoxy-azetidine-1-carbonyl)-isoquinolin-4-yl]-1-methyl-1,3-dihydro-indol-2-one (159)

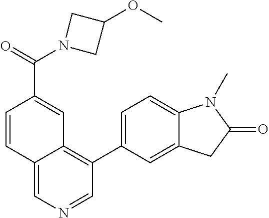 [SMILES: COC1CN(C(=O)c2ccc3cncc(-c4ccc5c(c4)CC(=O)N5C)c3c2)C1]

Into a 10-mL vial purged and maintained with an inert atmosphere of nitrogen was added 4-chloro-6-[(3-methoxyazetidin-1-yl)carbonyl]isoquinoline (60.0 mg, 0.22 mmol), 1-methyl-5-(tetramethyl-1,3,2-dioxaborolan-2-yl)-2,3-dihydro-1H-indol-2-one (77.0 mg, 0.28 mmol), KOAc (64.0 mg, 0.65 mmol), Pd(dppf)Cl₂*CH₂Cl₂ (18.0 mg, 0.02 mmol), water (3 mL) and acetonitrile (3 mL). The mixture was stirred under microwave irradiation for 1 h at 140° C. and concentrated under vacuum. The residue was purified by silica gel column chromatography with dichloromethane/methanol (20:1) and further purified by prep-HPLC (acetonitrile/water). This resulted in 20 mg (24%) of 5-[6-[(3-methoxyazetidin-1-yl)carbonyl]isoquinolin-4-yl]-1-methyl-2,3-dihydro-1H-indol-2-one as a white solid. ¹H NMR (300 MHz, DMSO-d6) ppm=9.40 (s, 1H), 8.50 (s, 1H), 8.29 (d, J=8.4 Hz, 1H), 8.05 (s, 1H), 7.91 (d, J=8.4 Hz, 1H), 7.48 (s, 1H), 7.22-7.19 (m, 2H), 4.42-4.40 (m, 1H), 4.25-4.21 (m, 2H), 4.09 (d, J=9.3 Hz, 1H), 3.89-3.85 (m, 1H), 3.66 (s, 2H), 3.21 (s, 3H), 3.16 (s, 3H). [M+H]⁺ 388. Rt 0.97 min (method Q).

### Example 61

### 6-[6-(3-Methoxy-azetidine-1-carbonyl)-isoquinolin-4-yl]-1-methyl-1,3-dihydro-indol-2-one (165)

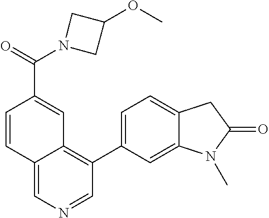 [SMILES: COC1CN(C(=O)c2ccc3cncc(-c4ccc5c(c4)N(C)C(=O)C5)c3c2)C1]

Into a 10-mL vial purged and maintained with an inert atmosphere of nitrogen was added 4-chloro-6-[(3-methoxyazetidin-1-yl)carbonyl]isoquinoline (80.0 mg, 0.29 mmol), 1-methyl-6-(tetramethyl-1,3,2-dioxaborolan-2-yl)-2,3-dihydro-1H-indol-2-one (103 mg, 0.38 mmol), KOAc (86.0 mg, 0.88 mmol), Pd(dppf)Cl₂*CH₂Cl₂ (24.0 mg, 0.03 mmol), water (3 mL) and acetonitrile (3 mL). The mixture was stirred under microwave irradiation for 1 h at 140° C. and concentrated under vacuum. The residue was purified by silica gel column chromatography with dichloromethane/methanol (20:1) and further purified by prep-HPLC (acetonitrile/water). This resulted in 20 mg (18%) of 6-[6-[(3-methoxyazetidin-1-yl)carbonyl]isoquinolin-4-yl]-1-methyl-2,3-dihydro-1H-indol-2-one as a white solid. ¹H NMR (300 MHz, DMSO-d6) ppm=9.44 (s, 1H), 8.56 (s, 1H), 8.31 (d, J=8.4 Hz, 1H), 8.06 (s, 1H), 7.93 (d, J=8.4 Hz, 1H), 7.47 (d, J=7.5 Hz, 1H), 7.22-7.18 (m, 2H), 4.43-4.41 (m, 1H), 4.26-4.24 (m, 2H), 4.10 (d, J=8.7 Hz, 1H), 3.86-3.84 (m, 1H), 3.68 (s, 2H), 3.21 (s, 3H), 3.16 (s, 3H). [M+H]⁺ 388. Rt 0.97 min (method Q).

### Example 62

### (3,3-Difluoro-azetidin-1-yl)-{4-[4-(1,2-dimethyl-1H-imidazol-4-yl)-phenyl]-isoquinolin-6-yl}-methanone (175)

### 4-chloro-6-[(3,3-difluoroazetidin-1-yl)carbonyl]isoquinoline

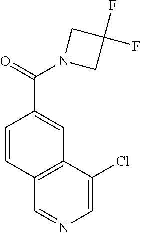 [SMILES: O=C(c1ccc2cncc(Cl)c2c1)N1CC(F)(F)C1]

Into a 25-mL round-bottom flask, was added 4-chloroisoquinoline-6-carboxylic acid (100 mg, 0.48 mmol), 3,3-difluoroazetidine hydrochloride (156 mg, 1.20 mmol), N,N-dimethylformamide (4 mL), HATU (220 mg, 0.58 mmol) and DIEA (187 mg, 1.45 mmol). The solution was stirred for 3 h at 25° C. and concentrated under vacuum. This resulted in 50 mg (37%) of 4-chloro-6-[(3,3-difluoroazetidin-1-yl)carbonyl]isoquinoline as an off-white solid. [M+H]±283. Rt 0.77 min (method S).

### (3,3-Difluoro-azetidin-1-yl)-{4-[4-(1,2-dimethyl-1H-imidazol-4-yl)-phenyl]-isoquinolin-6-yl}-methanone

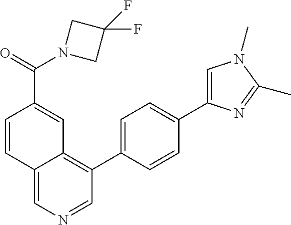 [SMILES: Cc1nc(-c2ccc(-c3cncc4ccc(C(=O)N5CC(F)(F)C5)cc34)cc2)cn1C]

Into a 10-mL vial purged and maintained with an inert atmosphere of nitrogen was added 4-chloro-6-[(3,3-difluoroazetidin-1-yl)carbonyl]isoquinoline (110 mg, 0.39 mmol), 1,2-dimethyl-4-[4-(tetramethyl-1,3,2-dioxaborolan-2-yl)phenyl]-1H-imidazole (151 mg, 0.51 mmol), KOAc (115 mg, 1.17 mmol), Pd(dppf)Cl₂*CH₂Cl₂ (32 mg, 0.04 mmol), acetonitrile (2 mL) and water (2 mL). The mixture was stirred under microwave irradiation for 1 h at 140° C. and concentrated under vacuum. The residue was purified by silica gel column chromatography with ethyl acetate/petrolether (9:1) and further purified by prep-HPLC (acetonitrile/water). This resulted in 20 mg (12%) of 6-[(3,3-difluoroazetidin-1-yl)carbonyl]-4-[4-(1,2-dimethyl-1H-imidazol-4-yl)phenyl]isoquinoline as an off-white solid. ¹H NMR (300 MHz, DMSO-d6) ppm=9.43 (s, 1H), 8.57 (s, 1H), 8.32 (d, J=8.7 Hz, 1H), 8.17 (s, 1H), 7.98-7.90 (m, 3H), 7.63 (s, 1H), 7.54 (d, J=8.1 Hz, 2H), 4.74-4.52 (m, 4H), 3.62 (s, 3H), 2.36 (s, 3H). [M+H]⁺ 419. Rt 1.38 min (method C).

### Example 63

### 6-[6-(3,3-Difluoro-azetidine-1-carbonyl)-isoquinolin-4-yl]-1-methyl-3,4-dihydro-1H-quinolin-2-one (158)

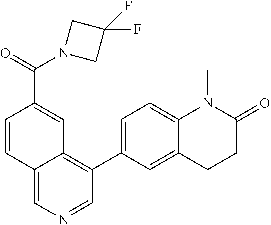 [SMILES: CN1C(=O)CCc2cc(-c3cncc4ccc(C(=O)N5CC(F)(F)C5)cc34)ccc21]

Into a 10-mL vial purged and maintained with an inert atmosphere of nitrogen was added 4-chloro-6-[(3,3-difluoroazetidin-1-yl)carbonyl]isoquinoline (100 mg, 0.35 mmol), 1-methyl-6-(tetramethyl-1,3,2-dioxaborolan-2-yl)-1,2,3,4-tetrahydroquinolin-2-one (133 mg, 0.46 mmol), KOAc (46.0 mg, 0.47 mmol), Pd(dppf)Cl₂*CH₂Cl₂ (29.0 mg, 0.04 mmol), water (3 mL) and acetonitrile (3 mL). The mixture was stirred under microwave irradiation for 1 h at 140° C. and concentrated under vacuum. The residue was dissolved in 10 mL of ethyl acetate and washed with 3×20 mL of water. The crude was purified by silica gel column chromatography with methanol:dichloromethane (1:20) and further purified by prep-HPLC (acetonitrile/water). This resulted in 20 mg (14%) of 6-[6-[(3,3-difluoroazetidin-1-yl)carbonyl]isoquinolin-4-yl]-1-methyl-1,2,3,4-tetrahydroquinolin-2-one as a white solid. ¹H NMR (300 MHz, DMSO-d6) ppm=9.43 (s, 1H), 8.54 (s, 1H), 8.32 (d, J=8.4 Hz, 1H), 8.14 (s, 1H), 7.97 (d, J=8.4 Hz, 1H), 7.48 (d, J=7.2 Hz, 2H), 7.32 (d, J=8.4 Hz, 1H), 4.77 (s, 2H), 4.52 (s, 2H), 3.32 (s, 3H), 3.01-2.96 (m, 2H), 2.67-2.62 (m, 2H). [M+H]⁺ 408. Rt 1.44 min (method Q).

### Example 64

### 6-[6-(3,3-Difluoro-azetidine-1-carbonyl)-isoquinolin-4-yl]-1-methyl-1,3-dihydro-indol-2-one (163)

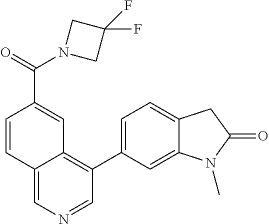 [SMILES: CN1C(=O)Cc2ccc(-c3cncc4ccc(C(=O)N5CC(F)(F)C5)cc34)cc21]

Into a 10-mL vial purged and maintained with an inert atmosphere of nitrogen was added 4-chloro-6-[(3,3-difluoroazetidin-1-yl)carbonyl]isoquinoline (60 mg, 0.21 mmol), 1-methyl-6-(tetramethyl-1,3,2-dioxaborolan-2-yl)-2,3-dihydro-1H-indol-2-one (76.0 mg, 0.28 mmol), KOAc (63.0 mg, 0.64 mmol), Pd(dppf)Cl₂*CH₂Cl₂ (18.0 mg, 0.02 mmol), water (2 mL) and acetonitrile (2 mL). The solution was stirred for 1 h at 140° C. and the mixture was concentrated under vacuum. The residue was purified by silica gel column chromatography with methanol:dichloromethane (1:20) and further purified by prep-HPLC (acetonitrile/water). This resulted in 44 mg (53%) of 6-[6-[(3,3-difluoroazetidin-1-yl)carbonyl]isoquinolin-4-yl]-1-methyl-2,3-dihydro-1H-indol-2-one as a white solid. ¹H NMR (300 MHz, DMSO-d6) ppm=9.46 (s, 1H), 8.58 (s, 1H), 8.33 (d, J=8.7 Hz, 1H), 8.13 (s, 1H), 7.97 (d, J=8.4 Hz, 1H), 7.48 (d, J=7.2 Hz, 1H), 7.22-7.18 (m, 2H), 4.75-4.52 (m, 4H), 3.68 (s, 2H), 3.20-3.16 (m, 3H). [M+H]⁺ 394. Rt 1.10 min (method Q).

### Example 65

### 5-[6-(3,3-Difluoro-azetidine-1-carbonyl)-isoquinolin-4-yl]-1-methyl-1,3-dihydro-indol-2-one (170)

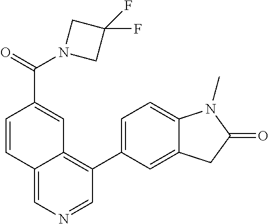 [SMILES: CN1C(=O)Cc2cc(-c3cncc4ccc(C(=O)N5CC(F)(F)C5)cc34)ccc21]

Into a 10-mL vial purged and maintained with an inert atmosphere of nitrogen was added 4-chloro-6-[(3,3-difluoroazetidin-1-yl)carbonyl]isoquinoline (80 mg, 0.28 mmol), 1-methyl-5-(tetramethyl-1,3,2-dioxaborolan-2-yl)-2,3-dihydro-1H-indol-2-one (101 mg, 0.37 mmol), KOAc (84.0 mg, 0.86 mmol), Pd(dppf)Cl₂*CH₂Cl₂ (24 mg, 0.03 mmol), water (3 mL) and acetonitrile (3 mL). The mixture was stirred under microwave irradiation for 1 h at 140° C. and concentrated under vacuum. The residue was purified by silica gel column chromatography with dichloromethane/methanol (20:1) and further purified by prep-HPLC (acetonitrile/water). This resulted in 20.4 mg (18%) of 5-[6-[(3,3-difluoroazetidin-1-yl)carbonyl]isoquinolin-4-yl]-1-methyl-2,3-dihydro-1H-indol-2-one as a white solid. ¹H NMR (300 MHz, DMSO-d6) ppm=9.42 (s, 1H), 8.51 (s, 1H), 8.32 (d, J=8.4 Hz, 1H), 8.12 (s, 1H), 7.96 (d, J=8.4 Hz, 1H), 7.49-7.48 (m, 2H), 7.22-7.19 (m, 1H), 4.75-4.51 (m, 4H), 3.66 (s, 2H), 3.21 (s, 3H). [M+H]⁺ 394. Rt 1.09 min (method Q).

### Example 66

### azetidin-1-yl(4-(4-(1-methyl-1H-pyrazol-4-yl)phenyl)isoquinolin-6-yl)methanone (181)

### azetidin-1-yl(4-chloroisoquinolin-6-yl)methanone

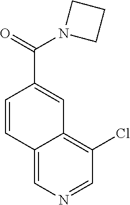 [SMILES: O=C(c1ccc2cncc(Cl)c2c1)N1CCC1]

To a suspension of 4-chloroisoquinoline-6-carboxylic acid (30 mg, 0.14 mmol), HATU (6 mg, 0.16 mmol), DIPEA (0.15 mL, 0.86 mmol) in DMF (1 mL) was added azetidine hydrochloride (13 mg, 0.14 mmol), and the reaction mixture was stirred at room temperature for 18 h. To the crude reactions mixture was added a further portion of HATU (6.0 mg, 0.16 mmol) and the mixture stirred at room temperature for 1 h. The crude reaction mixture was poured onto 1M NaOH, and the organic material extracted with ethyl acetate (twice). The combined organic layers were washed with brine, dried over MgSO₄, filtered, and the filtrate concentrated under reduced pressure. The crude material was purified over a silica cartridge using a solvent system of 0-15% EtOH in dichloromethane. [M+H]⁺ 247 Rt 1.19 min (method O) azetidin-1-yl(4-(4-(1-methyl-1H-pyrazol-4-yl)phenyl)isoquinolin-6-yl)methanone

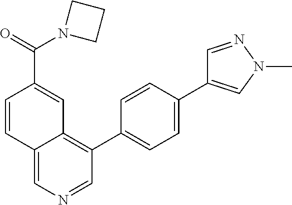 [SMILES: Cn1cc(-c2ccc(-c3cncc4ccc(C(=O)N5CCC5)cc34)cc2)cn1]

To a solution of azetidin-1-yl(4-chloroisoquinolin-6-yl)methanone (7 mg, 0.03 mmol), 1-methyl-4-(4-(4,4,5,5-tetramethyl-1,3,2-dioxaborolan-2-yl)phenyl)-1H-pyrazole (9 mg, 0.03 mmol), Pd(dppf)Cl₂ (1 mg, 1.4 umol) in MeCN (0.6 mL) was added Na₂CO₃ (0.08 mL, 0.04 mmol, 0.5 M). The reaction vessel was sealed and heated in a focused microwave reactor to 120° C. for 1.5 h. The cooled reaction mixture was concentrated under reduced pressure, and the crude product purified over a silica cartridge using a solvent system of 0-15% ethanol in dichloromethane. The title compound was isolated as a yellow solid (8 mg, 77%). ¹H NMR (400 MHz, CDCl₃) ppm=9.34 (s, 1H), 8.58 (s, 1H), 8.19 (s, 1H), 8.15 (d, J=8.3 Hz, 1H), 7.94 (d, J=8.3 Hz, 1H), 7.87 (s, 1H), 7.73 (s, 1H), 7.66 (d, J=7.6 Hz, 2H), 7.51 (d, J=7.6 Hz, 2H), 4.24 (t, J=7.7 Hz, 4H), 4.01 (s, 3H), 2.36 (p, J=7.7 Hz, 2H). [M+H]⁺ 369 Rt 1.15 min (method O)

### Example 67

### (1-amino-4-(4-(1-methyl-1H-pyrazol-4-yl)phenyl)isoquinolin-6-yl)(azetidin-1-yl)methanone (197)

### 6-(azetidine-1-carbonyl)-4-(4-(1-methyl-1H-pyrazol-4-yl)phenyl)isoquinoline 2-oxide

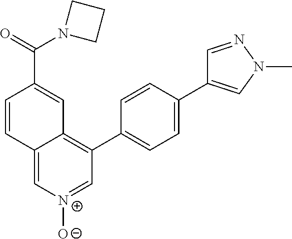 [SMILES: Cn1cc(-c2ccc(-c3c[n+]([O-])cc4ccc(C(=O)N5CCC5)cc34)cc2)cn1]

To a foil-covered solution of azetidin-1-yl(4-(4-(1-methyl-1H-pyrazol-4-yl)phenyl)isoquinolin-6-yl)methanone (17 mg, 0.046 mmol) in dichloromethane (0.8 mL) at room temperature, was added mCPBA (32 mg, 0.138 mmol) in one portion. The reaction mixture was stirred at room temperature for 1 h. The crude solution was poured onto 1M NaOH, and the organic material was extracted with EtOAc. The combined organic layers were washed with brine, dried over MgSO₄, filtered, and the filtrate concentrated under reduced pressure. The crude product was used in the next step without further purification.

### (1-amino-4-(4-(1-methyl-1H-pyrazol-4-yl)phenyl)isoquinolin-6-yl)(azetidin-1-yl)methanone

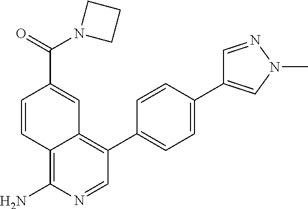 [SMILES: Cn1cc(-c2ccc(-c3cnc(N)c4ccc(C(=O)N5CCC5)cc34)cc2)cn1]

To 6-(azetidine-1-carbonyl)-4-(4-(1-methyl-1H-pyrazol-4-yl)phenyl)isoquinoline 2-oxide (18 mg, 0.05 mmol) in pyridine (0.7 mL), was added tosyl chloride (11 mg, 0.06 mmol). The reaction mixture was stirred at room temperature for 30 min. To the reaction mixture at room temperature was added ethanolamine (0.07 mL, 1.15 mmol) and the resultant solution stirred at room temperature for 1 h. The crude reaction mixture was diluted with water, and the organic material extracted with ethyl acetate. The combined organic layers were washed with brine, dried over MgSO₄, filtered, and the filtrate concentrated under reduced pressure. The crude product was purified over a silica cartridge using a solvent system of 1-15% ethanol in dichloromethane. ¹H NMR (400 MHz, CDCl₃) ppm=8.05 (d, J=1.6 Hz, 1H), 7.96 (s, 1H), 7.93 (d, J=8.6 Hz, 1H), 7.85 (s, 1H), 7.82 (dd, J=8.6, 1.6 Hz, 1H), 7.71 (s, 1H), 7.61 (d, J=8.2 Hz, 2H), 7.44 (d, J=8.2 Hz, 2H), 5.31 (s, 2H, NH₂), 4.23 (t, J=7.8 Hz, 4H, 2×CH₂), 4.01 (s, 3H), 2.34 (m, 2H, CH₂); [M+H]⁺ 384 Rt 0.95 min (method O)

### Example 68

### 6-(1-amino-6-(3-methoxyazetidine-1-carbonyl)isoquinolin-4-yl)-1-methyl-3,4-dihydroquinolin-2(1H)-one (178)

### 6-bromoisoquinolin-1-amine

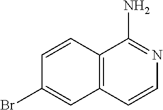 [SMILES: Nc1nccc2cc(Br)ccc12]

Into a 30-mL sealed tube, was added 6-bromo-1-chloroisoquinoline (1.50 g, 6.19 mmol), ammonia (15 mL) and dioxane (5 mL). The reaction mixture was stirred for 48 h at 120° C. in an oil bath. The solution was diluted with 20 mL of water and the aqueous layer was extracted with 2×50 mL of dichloromethane. The organic layers were combined, dried over sodium sulfate and concentrated under vacuum. The residue was purified by silica gel column chromatography with methanol:dichloromethane (2:10). This resulted in 0.50 g (36%) of 6-bromoisoquinolin-1-amine as a yellow solid. [M+H]⁺ 222/224. Rt 0.91 min (method R). 6-bromo-4-chloroisoquinolin-1-amine

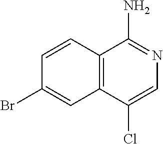 [SMILES: Nc1ncc(Cl)c2cc(Br)ccc12]

Into a 250-mL round-bottom flask, was added 6-bromoisoquinolin-1-amine (500 mg, 2.24 mmol), NCS (359 mg, 2.69 mmol) and chloroform (50 mL). The reaction mixture was stirred for 24 h at 60° C. in an oil bath. The solution was diluted with 50 mL of water and extracted with 2×50 mL of dichloromethane. The organic layers were combined, dried over sodium sulfate and concentrated under vacuum. The residue was purified by silica gel column chromatography with methanol:dichloromethane (3:10). This resulted in 400 mg (69%) of 6-bromo-4-chloroisoquinolin-1-amine as a purple solid. [M+H]⁺ 356/358. Rt 1.12 min (method R).

### Methyl 1-amino-4-chloroisoquinoline-6-carboxylate

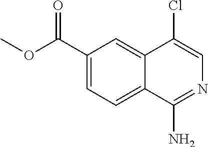 [SMILES: COC(=O)c1ccc2c(N)ncc(Cl)c2c1]

Into a 20-mL pressure tank reactor (5 atm) purged and maintained with an inert atmosphere of nitrogen was added 6-bromo-4-chloroisoquinolin-1-amine (400 mg, 1.55 mmol), Pd(dppf)Cl₂*CH₂Cl₂ (63.4 mg, 0.08 mmol), KOAc (457 mg, 4.66 mmol), N,N-dimethylformamide (5 mL) and methanol (5 mL). The solution was stirred for 2 h at 80° C. in an oil bath. The mixture was concentrated under vacuum. The residue was diluted with 50 mL of water. The solids formed were collected by filtration. The solid was suspended in 50 mL of dichloromethane. The solids formed were filtered off again. The filtrate was concentrated under vacuum. This resulted in 250 mg (68%) of methyl 1-amino-4-chloroisoquinoline-6-carboxylate as a yellow solid. [M+H]⁺ 237. Rt 0.94 min (method R).

### 1-amino-4-chloroisoquinoline-6-carboxylic acid

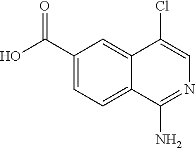 [SMILES: Nc1ncc(Cl)c2cc(C(=O)O)ccc12]

Into a 25-mL round-bottom flask, was added methyl 1-amino-4-chloroisoquinoline-6-carboxylate (250 mg, 1.06 mmol), LiOH (75.9 mg, 3.17 mmol), tetrahydrofuran (5 mL) and water (1 mL). The solution was stirred for 24 h at 60° C. in an oil bath. The mixture was concentrated under vacuum. The product was precipitated by the addition of hydrogen chloride (aq, 1 mol/L, 2 mL). The solids were collected by filtration. This resulted in 150 mg (64%) of 1-amino-4-chloroisoquinoline-6-carboxylic acid as a brown solid. [M+H]⁺ 223. Rt 1.00 min (method B). 4-chloro-6-[(3-methoxyazetidin-1-yl)carbonyl]isoquinolin-1-amine

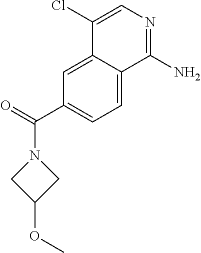 [SMILES: COC1CN(C(=O)c2ccc3c(N)ncc(Cl)c3c2)C1]

Into a 25-mL round bottom flask, was added 1-amino-4-chloroisoquinoline-6-carboxylic acid (300 mg, 1.35 mmol), 3-methoxyazetidine hydrochloride (200 mg, 1.62 mmol), HATU (769 mg, 2.02 mmol), DIEA (348 mg, 2.70 mmol) and N,N-dimethylformamide (5 mL). The solution was stirred for 3 h at rt and concentrated. The residue was purified by silica gel column chromatography with methanol:water (4:1). This resulted in 200 mg (51%) of 4-chloro-6-[(3-methoxyazetidin-1-yl)carbonyl]isoquinolin-1-amine as a yellow solid. [M+H]⁺ 292. Rt 1.16 min (method S).

### 6-[1-Amino-6-(3-methoxy-azetidine-1-carbonyl)-isoquinolin-4-yl]-1-methyl-3,4-dihydro-1H-quinolin-2-one

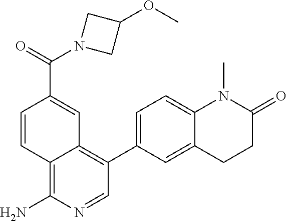 [SMILES: COC1CN(C(=O)c2ccc3c(N)ncc(-c4ccc5c(c4)CCC(=O)N5C)c3c2)C1]

Into a 10-mL sealed tube purged and maintained with an inert atmosphere of nitrogen was added 4-chloro-6-[(3-methoxyazetidin-1-yl)carbonyl]isoquinolin-1-amine (50.0 mg, 0.17 mmol), 1-methyl-6-(tetramethyl-1,3,2-dioxaborolan-2-yl)-1,2,3,4-tetrahydroquinolin-2-one (59.1 mg, 0.21 mmol), KOAc (33.6 mg, 0.34 mmol), Pd(PCy₃)₂Cl₂ (12.7 mg, 0.02 mmol), N,N-dimethylformamide (4.00 mL) and water (0.3 mL). The solution was stirred for 1.5 h at 120° C. and concentrated. The residue was purified silica gel column chromatography with methanol:dichloromethane (3:10) and further purified by prep-HPLC (acetonitrile/water). This resulted in 11.3 mg (15%) of 6-[1-amino-6-[(3-methoxyazetidin-1-yl)carbonyl]isoquinolin-4-yl]-1-methyl-1,2,3,4-tetrahydroquinolin-2-one as a yellow solid. ¹H NMR (300 Hz, DMSO-d6) ppm=8.34-8.31 (m, 1H), 7.89 (s, 1H), 7.81 (s, 1H), 7.70-7.68 (m, 1H), 7.34-7.31 (m, 2H), 7.24-7.21 (m, 1H), 7.02 (s, 2H), 4.43-4.40 (m, 1H), 4.24-4.21 (m, 2H), 4.10-4.07 (m, 1H), 3.85-3.83 (m, 1H), 3.31 (s, 3H), 3.21 (s, 3H), 2.96-2.92 (m, 2H), 2.64-2.59 (m, 2H). [M+H]⁺ 417. Rt 1.37 min (method D).

### Example 69

### 5-[1-Amino-6-(3-methoxy-azetidine-1-carbonyl)-isoquinolin-4-yl]-1-methyl-1,3-dihydro-indol-2-one (177)

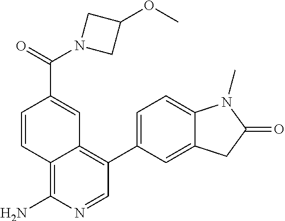 [SMILES: COC1CN(C(=O)c2ccc3c(N)ncc(-c4ccc5c(c4)CC(=O)N5C)c3c2)C1]

Into a 10-mL sealed tube purged and maintained with an inert atmosphere of nitrogen was added 4-chloro-6-[(3-methoxyazetidin-1-yl)carbonyl]isoquinolin-1-amine (50 mg, 0.17 mmol), 1-methyl-5-(tetramethyl-1,3,2-dioxaborolan-2-yl)-2,3-dihydro-1H-indol-2-one (56.2 mg, 0.21 mmol), KOAc (33.6 mg, 0.34 mmol), Pd(PCy₃)₂Cl₂ (12.7 mg, 0.02 mmol), N,N-dimethylformamide (4.00 mL) and water (0.3 mL). The solution was stirred for 1.5 h at 120° C. and concentrated. The residue was purified by silica gel column chromatography with methanol:dichloromethane (3:10) and further purified by prep-HPLC (acetonitrile/water). This resulted in 34 mg (49%) of 5-[1-amino-6-[(3-methoxyazetidin-1-yl)carbonyl]isoquinolin-4-yl]-1-methyl-2,3-dihydro-1H-indol-2-one as a yellow solid. ¹H NMR (300 Hz, DMSO-d6) ppm=8.33-8.30 (m, 1H), 7.86 (s, 1H), 7.78 (s, 1H), 7.69-7.66 (m, 1H), 7.33 (s, 1H), 7.12-7.09 (m, 1H), 6.99 (s, 2H), 4.49-4.35 (m, 1H), 4.23 (s, 2H), 4.10-4.00 (m, 1H), 3.91-3.78 (m, 1H), 3.62 (s, 2H), 3.21 (s, 3H), 3.18 (s, 3H). [M+H]⁺ 403. Rt 1.33 min (method D).

### Example 70

### 6-[1-Amino-6-(3-methoxy-azetidine-1-carbonyl)-isoquinolin-4-yl]-1-methyl-1,3-dihydro-indol-2-one (173)

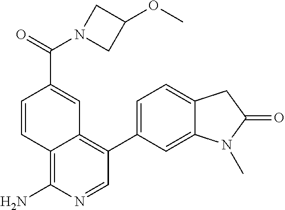 [SMILES: COC1CN(C(=O)c2ccc3c(N)ncc(-c4ccc5c(c4)N(C)C(=O)C5)c3c2)C1]

Into a 10-mL sealed tube purged and maintained with an inert atmosphere of nitrogen was added 4-chloro-6-[(3-methoxyazetidin-1-yl)carbonyl]isoquinolin-1-amine (50.0 mg, 0.17 mmol), 1-methyl-6-(tetramethyl-1,3,2-dioxaborolan-2-yl)-2,3-dihydro-1H-indol-2-one (56.2 mg, 0.21 mmol), KOAc (33.6 mg, 0.34 mmol), Pd(PCy₃)₂Cl₂ (12.7 mg, 0.02 mmol), N,N-dimethylformamide (4.00 mL) and water (0.3 mL). The solution was stirred for 1.5 h at 120° C. and concentrated. The residue was purified by silica gel column chromatography with methanol: dichloromethane (3:10) and further purified by prep-HPLC (acetonitrile/water). This resulted in 36 mg (52%) of 6-[1-amino-6-[(3-methoxyazetidin-1-yl)carbonyl]isoquinolin-4-yl]-1-methyl-2,3-dihydro-1H-indol-2-one as a yellow solid. ¹H NMR (300 Hz, DMSO-d6) ppm=8.35-8.32 (m, 1H), 7.89 (s, 1H), 7.85 (s, 1H), 7.70-7.67 (m, 1H), 7.40-7.37 (m, 1H), 7.07-7.00 (m, 4H), 4.41 (s, 1H), 4.23-4.21 (m, 2H), 4.12-4.03 (m, 1H), 3.83 (s, 1H), 3.63 (s, 2H), 3.20 (s, 3H), 3.14 (s, 3H). [M+H]⁺ 403. Rt 1.31 min (method B).

### Example 71

### {1-Amino-4-[4-(1,2-dimethyl-1H-imidazol-4-yl)-phenyl]-isoquinolin-6-yl}-(3,3-difluoro-azetidin-1-yl)-methanone (176)

### 4-chloro-6-[(3,3-difluoroazetidin-1-yl)carbonyl]isoquinolin-1-amine

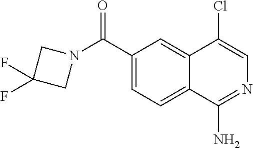 [SMILES: Nc1ncc(Cl)c2cc(C(=O)N3CC(F)(F)C3)ccc12]

Into a 10-mL sealed tube, was added 1-amino-4-chloroisoquinoline-6-carboxylic acid (300 mg, 1.35 mmol), 3,3-difluoroazetidine hydrochloride (209 mg, 1.62 mmol), HATU (769 mg, 2.02 mmol), DIEA (1.00 mL, 6.05 mmol) and N,N-dimethylformamide (3 mL). The solution was stirred for 3 h at room temperature and concentrated. The residue was purified by silica gel column chromatography with methanol:water (3:5). This resulted in 200 mg (50%) of 4-chloro-6-[(3,3-difluoroazetidin-1-yl)carbonyl]isoquinolin-1-amine as a yellow solid. [M+H]⁺ 298. Rt 0.50 min (method S).

### {1-Amino-4-[4-(1,2-dimethyl-1H-imidazol-4-yl)-phenyl]-isoquinolin-6-yl}-(3,3-difluoro-azetidin-1-yl)-methanone

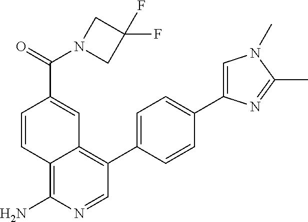 [SMILES: Cc1nc(-c2ccc(-c3cnc(N)c4ccc(C(=O)N5CC(F)(F)C5)cc34)cc2)cn1C]

Into a 10-mL sealed tube purged and maintained with an inert atmosphere of nitrogen was added 4-chloro-6-[(3,3-difluoroazetidin-1-yl)carbonyl]isoquinolin-1-amine (50 mg, 0.17 mmol), 1,2-dimethyl-4-[4-(tetramethyl-1,3,2-dioxaborolan-2-yl)phenyl]-1H-imidazole (60 mg, 0.20 mmol), KOAc (33 mg, 0.34 mmol), Pd(PCy₃)₂Cl₂ (12 mg, 0.02 mmol), N,N-dimethylformamide (4 mL) and water (0.3 mL). The solution was stirred for 1.5 h at 120° C. and concentrated. The residue was purified by silica gel column chromatography with methanol:dichloromethane (3:10). And further purified by prep-HPLC (acetonitrile/water). This resulted in 32 mg (44%) of 6-[(3,3-difluoroazetidin-1-yl)carbonyl]-4-[4-(1,2-dimethyl-1H-imidazol-4-yl)phenyl]isoquinolin-1-amine as a yellow solid. ¹H NMR (300 Hz, DMSO-d6) ppm=8.36-8.33 (m, 1H), 8.00 (s, 1H), 7.85-7.80 (m, 3H), 7.75-7.72 (m, 1H), 7.55 (s, 1H), 7.41-7.38 (m, 2H), 7.05 (s, 2H), 4.73-4.44 (m, 4H), 3.60 (s, 3H), 2.34 (s, 3H). [M+H]⁺ 434. Rt 1.85 min (method K).

### Example 72

### 6-[1-Amino-6-(3,3-difluoro-azetidine-1-carbonyl)-isoquinolin-4-yl]-1-methyl-3,4-dihydro-1H-quinolin-2-one (172)

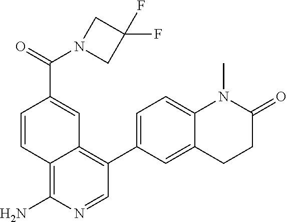 [SMILES: CN1C(=O)CCc2cc(-c3cnc(N)c4ccc(C(=O)N5CC(F)(F)C5)cc34)ccc21]

Into a 10-mL sealed tube purged and maintained with an inert atmosphere of nitrogen was added 4-chloro-6-[(3,3-difluoroazetidin-1-yl)carbonyl]isoquinolin-1-amine (50 mg, 0.17 mmol), 1-methyl-6-(tetramethyl-1,3,2-dioxaborolan-2-yl)-1,2,3,4-tetrahydroquinolin-2-one (72 mg, 0.25 mmol), KOAc (33.0 mg, 0.34 mmol), Pd(PCy₃)₂Cl₂ (12.4 mg, 0.02 mmol), N,N-dimethylformamide (5.00 mL) and water (0.5 mL). The solution was stirred for 1.5 h at 120° C. and concentrated. The residue was purified by silica gel column chromatography with methanol:dichloromethane (3:10) and further purified by prep-HPLC (acetonitrile/water). This resulted in 22 mg (31%) of 6-[1-amino-6-[(3,3-difluoroazetidin-1-yl)carbonyl]isoquinolin-4-yl]-1-methyl-1,2,3,4-tetrahydroquinolin-2-one as a yellow solid. ¹H NMR (300 Hz, DMSO-d6) ppm=8.36-8.33 (m, 1H), 7.96 (s, 1H), 7.82 (s, 1H), 7.75-7.72 (m, 1H), 7.35-7.32 (m, 2H), 7.25-7.22 (m, 1H), 7.05 (s, 2H), 4.75 (s, 2H), 4.49 (s, 2H), 3.31 (s, 3H), 2.97-2.92 (m, 2H), 2.63-2.50 (m, 2H). [M+H]⁺ 423. Rt 1.44 min (method B).

### Example 73

### 6-[1-Amino-6-(3,3-difluoro-azetidine-1-carbonyl)-isoquinolin-4-yl]-1-methyl-1,3-dihydro-indol-2-one (164)

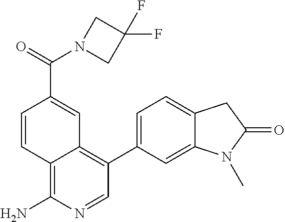 [SMILES: CN1C(=O)Cc2ccc(-c3cnc(N)c4ccc(C(=O)N5CC(F)(F)C5)cc34)cc21]

Into a 10-mL sealed tube purged and maintained with an inert atmosphere of nitrogen was added 4-chloro-6-[(3,3-difluoroazetidin-1-yl)carbonyl]isoquinolin-1-amine (60 mg, 0.20 mmol), 1-methyl-6-(tetramethyl-1,3,2-dioxaborolan-2-yl)-2,3-dihydro-1H-indol-2-one (72 mg, 0.26 mmol), KOAc (39.6 mg, 0.40 mmol), Pd(PCy₃)₂Cl₂ (14.9 mg, 0.02 mmol), N,N-dimethylformamide (3.00 mL) and water (0.5 mL). The solution was stirred for 1.5 h at 120° C. and concentrated. The residue was purified by silica gel column chromatography with methanol:dichloromethane (4:10) and further purified by prep-HPLC (acetonitrile/water). This resulted in 50 mg (61%) of 6-[1-amino-6-[(3,3-difluoroazetidin-1-yl)carbonyl]isoquinolin-4-yl]-1-methyl-2,3-dihydro-1H-indol-2-one as a yellow solid. ¹H NMR (300 Hz, DMSO-d6) ppm=8.37-8.34 (m, 1H), 7.96 (s, 1H), 7.87 (s, 1H), 7.75-7.72 (m, 1H), 7.40-7.37 (m, 1H), 7.12-7.02 (m, 4H), 4.73 (s, 2H), 4.47 (s, 2H), 3.63 (s, 2H), 3.14 (s, 3H). [M+H]⁺ 409. Rt 0.61 min (method E).

### Example 74

### 5-[1-Amino-6-(3,3-difluoro-azetidine-1-carbonyl)-isoquinolin-4-yl]-1-methyl-1,3-dihydro-indol-2-one (171)

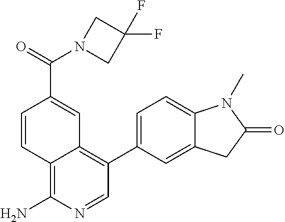 [SMILES: CN1C(=O)Cc2cc(-c3cnc(N)c4ccc(C(=O)N5CC(F)(F)C5)cc34)ccc21]

Into a 10-mL sealed tube purged and maintained with an inert atmosphere of nitrogen was added 4-chloro-6-[(3,3-difluoroazetidin-1-yl)carbonyl]isoquinolin-1-amine (50 mg, 0.17 mmol), 1-methyl-5-(tetramethyl-1,3,2-dioxaborolan-2-yl)-2,3-dihydro-1H-indol-2-one (69 mg, 0.25 mmol), KOAc (33.0 mg, 0.34 mmol), Pd(PCy₃)Cl₂ (12.4 mg, 0.02 mmol), N,N-dimethylformamide (4.00 mL) and water (0.30 mL). The solution was stirred for 1.5 h at 120° C. and concentrated. The residue was purified by silica gel column chromatography with methanol:dichloromethane (3:10) and further purified by prep-HPLC (acetonitrile/water). This resulted in 27 mg (39%) of 5-[1-amino-6-[(3,3-difluoroazetidin-1-yl)carbonyl]isoquinolin-4-yl]-1-methyl-2,3-dihydro-1H-indol-2-one as a yellow solid. ¹H NMR (300 Hz, DMSO-d6) ppm=8.35-8.33 (m, 1H), 7.93 (s, 1H), 7.79 (s, 1H), 7.74-7.71 (m, 1H), 7.33 (s, 2H), 7.13-7.10 (m, 1H), 7.03-7.00 (m, 2H), 4.80-4.60 (m, 2H), 4.60-4.47 (m, 2H), 3.62 (s, 2H), 3.19 (s, 3H). [M+H]⁺ 409. Rt 1.40 min (method B).

### Example 75

### 1-Amino-4-[4-(1-methyl-1H-pyrazol-4-yl)-phenyl]-isoquinoline-6-carboxylic acid cyclopropylamide (174)

### 1-amino-4-chloro-N-cyclopropylisoquinoline-6-carboxamide

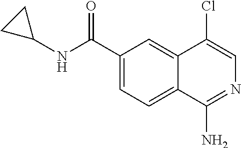 [SMILES: Nc1ncc(Cl)c2cc(C(=O)NC3CC3)ccc12]

Into a 10-mL sealed tube, was added 1-amino-4-chloroisoquinoline-6-carboxylic acid (80 mg, 0.36 mmol), cyclopropanamine (25 mg, 0.43 mmol), HATU (205 mg, 0.54 mmol), DIEA (92.9 mg, 0.72 mmol) and N,N-dimethylformamide (3.00 mL). The solution was stirred for 3 h at rt. The residue was purified by silica gel column chromatography with methanol:water (4:1). This resulted in 50 mg (53%) of 1-amino-4-chloro-N-cyclopropylisoquinoline-6-carboxamide as an orange solid. [M+H]⁺ 262.

### 1-Amino-4-[4-(1-methyl-1H-pyrazol-4-yl)-phenyl]-isoquinoline-6-carboxylic acid cyclopropylamide

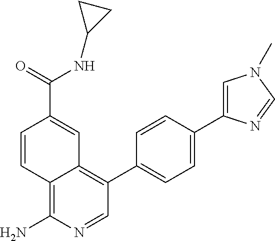 [SMILES: Cn1cnc(-c2ccc(-c3cnc(N)c4ccc(C(=O)NC5CC5)cc34)cc2)c1]

Into a 10-mL round-bottom flask purged and maintained with an inert atmosphere of nitrogen was added 1-amino-4-chloro-N-cyclopropylisoquinoline-6-carboxamide (50 mg, 0.19 mmol), 1-methyl-4-[4-(tetramethyl-1,3,2-dioxaborolan-2-yl)phenyl]-1H-pyrazole (80 mg, 0.28 mmol), Pd(PCy₃)₂Cl₂ (9.87 mg, 0.01 mmol), KOAc (39.94 mg, 0.41 mmol), N,N-dimethylformamide (4.00 mL) and water (0.3 mL). The solution was stirred for 1.5 h at 120° C. and concentrated. The residue was purified by silica gel column chromatography with methanol:dichloromethane (3:10) and further purified by prep-HPLC (acetonitrile/water). This resulted in 17 mg (23%) of 1-amino-N-cyclopropyl-4-[4-(1-methyl-1H-pyrazol-4-yl)phenyl]isoquinoline-6-carboxamide as a yellow solid. ¹H NMR (300 Hz, DMSO-d6) ppm=8.60-8.59 (m, 1H), 8.34-8.31 (m, 1H), 8.20 (s, 2H), 7.93 (s, 1H), 7.87-7.82 (m, 2H), 7.70-7.67 (m, 2H), 7.44-7.41 (m, 2H), 7.00 (s, 2H), 3.90 (s, 3H), 2.84-2.82 (m, 1H), 0.69-0.65 (m, 2H), 0.57-0.56 (m, 2H). [M+H]⁺ 384. Rt 1.38 min (method B).

### Example 76

### 3-{1-Amino-4-[4-(1-methyl-1H-pyrazol-4-yl)-phenyl]-isoquinolin-6-yl}-oxazolidin-2-one (179)

### 3-(1-amino-4-chloroisoquinolin-6-yl)-1,3-oxazolidin-2-one

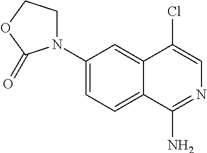 [SMILES: Nc1ncc(Cl)c2cc(N3CCOC3=O)ccc12]

Into a 10-mL sealed tube purged and maintained with an inert atmosphere of nitrogen was added 6-bromo-4-chloroisoquinolin-1-amine (150 mg, 0.58 mmol), 1,3-oxazolidin-2-one (71 mg, 0.82 mmol), K₃PO₄ (371 mg, 1.75 mmol), Pd₂(dba)₃ (27 mg, 0.03 mmol), Xantphos (34 mg, 0.06 mmol) and toluene (4 mL). The solution was stirred for 1 h at 110° C. in an oil bath and concentrated under vacuum. The residue was dissolved in 5 mL of DMF. The solids were filtered off. The residue was purified silica gel column with methanol:water (3:10). This resulted in 20 mg (13%) of 3-(1-amino-4-chloroisoquinolin-6-yl)-1,3-oxazolidin-2-one as a yellow solid. [M+H]⁺ 264.

### 3-{1-Amino-4-[4-(1-methyl-1H-pyrazol-4-yl)-phenyl]-isoquinolin-6-yl}-oxazolidin-2-one

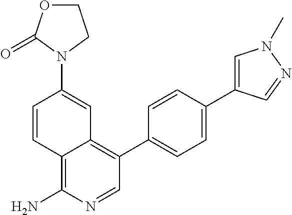 [SMILES: Cn1cc(-c2ccc(-c3cnc(N)c4ccc(N5CCOC5=O)cc34)cc2)cn1]

Into a 10-mL sealed tube purged and maintained with an inert atmosphere of nitrogen was added 3-(1-amino-4-chloroisoquinolin-6-yl)-1,3-oxazolidin-2-one (30.0 mg, 0.11 mmol), 1-methyl-4-[4-(tetramethyl-1,3,2-dioxaborolan-2-yl)phenyl]-1H-pyrazole (48.5 mg, 0.17 mmol), Pd(PCy₃)₂Cl₂ (8.40 mg, 0.01 mmol), KOAc (22.3 mg, 0.23 mmol), N,N-dimethylformamide (4.00 mL) and water (0.3 mL). The solution was stirred for 1 h at 110° C. and concentrated under vacuum. The residue was purified by silica gel column chromatography with methanol:dichloromethane (1:10) and further purified by prep-HPLC (acetonitrile/water). This resulted in 7.6 mg (17%) of 3-[1-amino-4-[4-(1-methyl-1H-pyrazol-4-yl)phenyl]isoquinolin-6-yl]-1,3-oxazolidin-2-one as a white solid. ¹H NMR (300 Hz, DMSO-d6) ppm=8.31-8.28 (m, 1H), 8.19 (m, 1H), 7.91 (s, 1H), 7.86-7.80 (m, 2H), 7.74 (s, 1H), 7.67-7.64 (m, 2H), 7.45-7.42 (m, 2H), 6.86 (s, 2H), 4.45-4.39 (m, 2H), 4.11-4.05 (m, 2H), 3.89 (s, 3H). [M+H]⁺ 385. Rt 0.66 min (method E).

### Example 77

### N-{4-[4-(1-Methyl-1H-pyrazol-4-yl)-phenyl]-isoquinolin-6-yl}-acetamide (101)

### 4-[4-(1-Methyl-1H-pyrazol-4-yl)phenyl]isoquinolin-6-amine

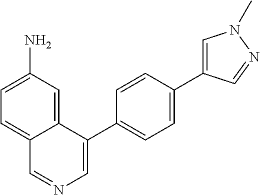 [SMILES: Cn1cc(-c2ccc(-c3cncc4ccc(N)cc34)cc2)cn1]

Into a 30 mL sealed tube, were placed 4-chloroisoquinolin-6-amine (300 mg, 1.68 mmol), 1-methyl-4-[4-(tetramethyl-1,3,2-dioxaborolan-2-yl)phenyl]-1H-pyrazole (668 mg, 2.35 mmol), Pd(dppf)Cl₂ dichloromethane complex (137 mg, 0.17 mmol), sodium carbonate (267 mg, 2.52 mmol), acetonitrile (9 mL) and water (9 mL). The reaction mixture was irradiated with microwave radiation for 1.5 h at 150° C. The reaction mixture was cooled to 25° C., concentrated under vacuum and diluted with 25 mL of ethyl acetate. The mixture was washed twice with 15 mL of brine. The organic phase was dried over anhydrous sodium sulfate and concentrated under vacuum. The residue was applied to a silica gel column with dichloromethane/methanol (10:1). This resulted in 480 mg (81%) of the title compound as a light brown solid.

### N-{4-[4-(1-Methyl-1H-pyrazol-4-yl)-phenyl]-isoquinolin-6-yl}-acetamide

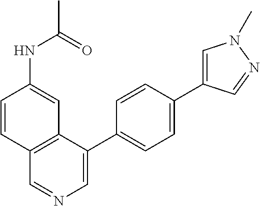 [SMILES: CC(=O)Nc1ccc2cncc(-c3ccc(-c4cnn(C)c4)cc3)c2c1]

Into a 25 mL round-bottom flask purged and maintained with an inert atmosphere of nitrogen, were placed a solution of 4-[4-(1-methyl-1H-pyrazol-4-yl)phenyl]isoquinolin-6-amine (90.0 mg, 0.30 mmol) in dichloromethane (2 mL). This was followed by the addition of a solution of acetyl acetate (60.0 mg, 0.59 mmol) in dichloromethane (0.5 mL) dropwise. To this was added N,N-diisopropylethylamine (116 mg, 0.90 mmol). The solution was stirred for 20 h at 25° C. The mixture was concentrated under vacuum. The residue was applied to a silica gel column with acetonitrile/water (1:1). This resulted in 45.0 mg (42%) of the title compound as a white solid. ¹H NMR (300 MHz, DMSO-d6) ppm=10.36 (s, 1H), 9.17 (s, 1H), 8.36 (s, 1H), 8.27 (s, 1H), 8.25 (s, 1H), 8.14 (d, J=8.8, 1H), 7.98 (s, 1H), 7.93 (d, J=8.7, 1H), 7.75 (d, J=8.0, 2H), 7.52 (d, J=8.0, 2H), 3.91 (s, 3H), 2.06 (s, 3H). [M+H]⁺ 343. Rt 0.74 min (method C).

### Example 78

### 2-Methoxy-N-{4-[4-(1-methyl-1H-pyrazol-4-yl)-phenyl]-isoquinolin-6-yl}-acetamide (121)

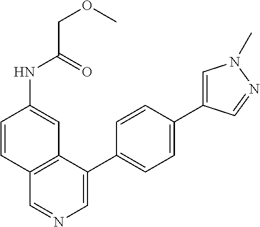 [SMILES: COCC(=O)Nc1ccc2cncc(-c3ccc(-c4cnn(C)c4)cc3)c2c1]

Into a 10 mL round-bottom flask purged and maintained with an inert atmosphere of nitrogen, were placed 4-[4-(1-methyl-1H-pyrazol-4-yl)phenyl]isoquinolin-6-amine (150 mg, 0.50 mmol), a solution of 2-methoxyacetyl chloride (82.0 mg, 0.76 mmol) in dichloromethane (1 mL), N,N-diisopropylethylamine (194 mg, 1.50 mmol) and dichloromethane (2 mL). The solution was stirred for 20 h at 20° C. The mixture was concentrated under vacuum. The residue was applied to a silica gel column with acetonitrile/water (1:1). This resulted in 55.1 mg (28%) of the title compound as a white solid. ¹H NMR (400 MHz, DMSO-d6) ppm=10.23 (s, 1H), 9.19 (s, 1H), 8.39 (s, 1H), 8.37 (s, 1H), 8.26 (s, 1H), 8.16 (d, J=8.9, 1H), 8.02 (dd, J=8.9, 1.8, 1H), 7.98 (s, 1H), 7.76 (d, J=8.2, 2H), 7.53 (d, J=8.2, 2H), 4.02 (s, 2H), 3.91 (s, 3H), 3.36 (s, 3H). [M+H]⁺ 373. Rt 1.42 min (method D).

### Example 79

### 1-{4-[4-(1-Methyl-1H-pyrazol-4-yl)-phenyl]-isoquinolin-6-yl}-pyrrolidine-2,5-dione (126)

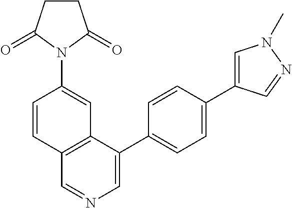 [SMILES: Cn1cc(-c2ccc(-c3cncc4ccc(N5C(=O)CCC5=O)cc34)cc2)cn1]

Into a 10 mL sealed tube, were placed 4-[4-(1-methyl-1H-pyrazol-4-yl)phenyl]isoquinolin-6-amine (90.0 mg, 0.30 mmol), oxolane-2,5-dione (90.0 mg, 0.90 mmol), triethylamine (90.0 mg, 0.89 mmol) and xylene (3 mL). The solution was stirred for 24 h at 150° C. The reaction mixture was cooled to 20° C. and was concentrated under vacuum. The residue was applied to a silica gel column with acetonitrile/water (1:1). This resulted in 80.0 mg (66%) of the title compound as a white solid. ¹H NMR (300 MHz, DMSO-d6) ppm=9.40 (s, 1H), 8.52 (s, 1H), 8.35 (d, J=8.7, 1H), 8.26 (s, 1H), 7.98 (s, 1H), 7.88 (s, 1H), 7.77 (d, J=8.2, 2H), 7.64 (dd, J=8.7, 1.7, 1H), 7.52 (d, J=8.2, 2H), 3.90 (s, 3H), 2.78 (s, 4H). [M+H]⁺ 383. Rt 1.35 min (method D).

### Example 80

### N-{4-[4-(1-Methyl-1H-pyrazol-4-yl)-phenyl]-isoquinolin-6-yl}-methanesulfonamide (98)

### N-Methanesulfonyl-N-{4-[4-(1-methyl-1H-pyrazol-4-yl)phenyl]isoquinolin-6-yl}methanesulfonamide

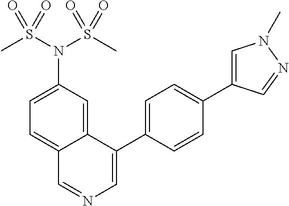 [SMILES: Cn1cc(-c2ccc(-c3cncc4ccc(N(S(C)(=O)=O)S(C)(=O)=O)cc34)cc2)cn1]

Into a 25 mL round-bottom flask purged and maintained with an inert atmosphere of nitrogen, were placed 4-[4-(1-methyl-1H-pyrazol-4-yl)phenyl]isoquinolin-6-amine (90.0 mg, 0.30 mmol), N,N-diisopropylethylamine (194 mg, 1.50 mmol) and dichloromethane (2 mL). This was followed by the addition of a solution of methanesulfonyl chloride (103 mg, 0.90 mmol) in dichloromethane (1 mL) dropwise with stifling at 0° C. The solution was stirred for 3 h at 20° C. The mixture was concentrated under vacuum. This resulted in 130 mg (81%) of the title compound as a brown solid.

### N-{4-[4-(1-Methyl-1H-pyrazol-4-yl)-phenyl]-isoquinolin-6-yl}-methanesulfonamide

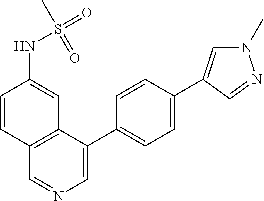 [SMILES: Cn1cc(-c2ccc(-c3cncc4ccc(NS(C)(=O)=O)cc34)cc2)cn1]

Into a 25 mL round-bottom flask, were placed N-methanesulfonyl-N-[4-[4-(1-methyl-1H-pyrazol-4-yl)phenyl]isoquinolin-6-yl]methanesulfonamide (130 mg, 0.28 mmol), sodium hydroxide (45 mg, 1.13 mmol), tetrahydrofuran (2 mL) and water (2 mL). The solution was stirred for 3 h at 80° C. The reaction mixture was cooled to 25° C., diluted with 15 mL of water and extracted twice with 15 mL of ethyl acetate. The combined organic layer was washed with 20 mL of brine. The mixture was dried over anhydrous sodium sulfate and concentrated under vacuum. The residue was applied to a silica gel column with acetonitrile/water (2:1). The crude product was re-crystallized from methanol. This resulted in 35.0 mg (30%) of the title compound as a light brown solid. ¹H NMR (300 MHz, DMSO-d6) ppm=10.34 (s, 1H), 9.21 (s, 1H), 8.40 (s, 1H), 8.26 (s, 1H), 8.19 (d, J=8.8, 1H), 7.98 (s, 1H), 7.77-7.75 (m, 3H), 7.61-7.53 (m, 3H), 3.90 (s, 3H), 3.08 (s, 3H). [M+H]⁺ 379. Rt 2.14 min (method K).

### Example 81

### Cyclopropanesulfonic acid {4-[4-(1-methyl-1H-pyrazol-4-yl)-phenyl]-isoquinolin-6-yl}-amide (162)

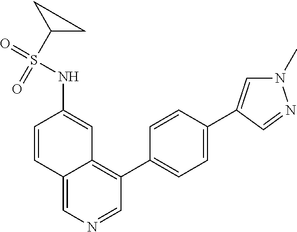 [SMILES: Cn1cc(-c2ccc(-c3cncc4ccc(NS(=O)(=O)C5CC5)cc34)cc2)cn1]

Into a 25-mL round-bottom flask purged and maintained with an inert atmosphere of nitrogen was added 4-[4-(1-methyl-1H-pyrazol-4-yl)phenyl]isoquinolin-6-amine (100 mg, 0.33 mmol), dichloromethane (4 mL), DIEA (130 mg, 1.01 mmol), cyclopropanesulfonyl chloride (71 mg, 0.51 mmol). The solution was stirred for 3 h at 25° C. The mixture was concentrated under vacuum and the crude product was purified by prep-HPLC (acetonitrile/water). This resulted in 20 mg (15%) of N-[4-[4-(1-methyl-1H-pyrazol-4-yl)phenyl]isoquinolin-6-yl]cyclopropanesulfonamide as a white solid. ¹H NMR (400 MHz, DMSO-d6) ppm=10.31 (s, 1H), 9.20 (s, 1H), 8.38 (s, 1H), 8.26 (s, 1H), 8.17 (d, J=8.8 Hz, 1H), 7.97 (s, 1H), 7.76 (d, J=8 Hz, 3H), 7.62-7.59 (m, 1H), 7.52 (d, J=8 Hz, 2H), 3.90 (s, 3H), 2.72-2.67 (m, 1H), 0.93 (d, J=6.4 Hz, 4H). [M+H]⁺ 405. Rt 1.13 min (method Q).

### Example 82

### N-{1-Amino-4-[4-(1-methyl-1H-pyrazol-4-yl)-phenyl]-isoquinolin-6-yl}-acetamide (182)

### N-(1-amino-4-chloro-6-isoquinolyl)acetamide

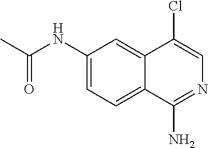 [SMILES: CC(=O)Nc1ccc2c(N)ncc(Cl)c2c1]

Into a 250-mL round bottom flask purged and maintained with an inert atmosphere of nitrogen was added 6-bromo-4-chloroisoquinolin-1-amine (150 mg, 0.58 mmol), acetamide (48 mg, 0.82 mmol), Pd₂(dba)₃ (27 mg, 0.030 mmol), xantphos (34 mg, 0.060 mmol), K₃PO₄ (371 mg, 1.75 mmol) and dioxane (100 mL). The solution was stirred for 12 h at 105° C. in an oil bath. The reaction mixture was concentrated under vacuum and the residue was dissolved in 5 mL of DMF. The solids were filtered off. The filtrate was purified by silica gel column chromatography with methanol:water (4:10). This resulted in 59.7 mg (44%) of N-(1-amino-4-chloroisoquinolin-6-yl)acetamide as a yellow solid. [M+H]⁺ 236.

### N-{1-Amino-4-[4-(1-methyl-1H-pyrazol-4-yl)-phenyl]-isoquinolin-6-yl}-acetamide

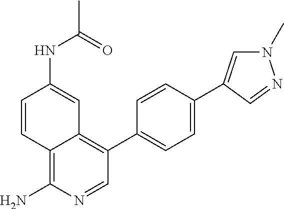 [SMILES: CC(=O)Nc1ccc2c(N)ncc(-c3ccc(-c4cnn(C)c4)cc3)c2c1]

Into a 10-mL vial purged and maintained with an inert atmosphere of nitrogen was added N-(1-amino-4-chloroisoquinolin-6-yl)acetamide (60 mg, 0.25 mmol), 1-methyl-4-[4-(tetramethyl-1,3,2-dioxaborolan-2-yl)phenyl]-1H-pyrazole (109 mg, 0.38 mmol), Pd(PCy₃)₂Cl₂ (19 mg, 0.03 mmol), KOAc (50 mg, 0.51 mmol), N,N-dimethylformamide (5 mL) and water (0.3 mL). The solution was stirred for 1 h at 110° C. and concentrated. The residue was purified by silica gel column chromatography with methanol:dichloromethane (3:10) and by prep-HPLC (acetonitrile/water). This resulted in 30 mg (33%) of N-[1-amino-4-[4-(1-methyl-1H-pyrazol-4-yl)phenyl]isoquinolin-6-yl]acetamide as a white solid. ¹H NMR (300 Hz, DMSO-d6) ppm=10.18 (s, 1H), 8.19-8.17 (m, 2H), 8.01 (s, 1H), 7.92 (m, 1H), 7.78-7.75 (m, 1H), 7.69-7.65 (m, 3H), 7.40-7.37 (m, 2H), 6.75 (s, 2H), 3.89 (s, 3H), 2.03 (s, 3H). [M+H]⁺ 358. Rt 0.64 min (method S).

### Example 83

### 6-Methanesulfonyl-4-[4-(1-methyl-1H-pyrazol-4-yl)-phenyl]-isoquinoline (189)

### 4-chloro-6-methanesulfonylisoquinoline

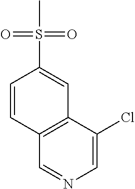

Into a 25-mL vial, was added 6-bromo-4-chloroisoquinoline (200 mg, 0.82 mmol), sodium methanesulfinate (169 mg, 1.66 mmol), CuI (315 mg, 1.65 mmol) and DMSO (5 mL). The reaction mixture was stirred for 6 h at 120° C. The solution was diluted with 50 mL of water and the aqueous layer was extracted with 3×10 mL of ethyl acetate. The organic layers were combined and concentrated under vacuum. The residue was purified by silica gel column chromatography with methanol:dichloromethane (1:25). This resulted in 170 mg (85%) of 4-chloro-6-methanesulfonylisoquinoline as an orange solid. [M+H]⁺ 242

### 6-Methanesulfonyl-4-[4-(1-methyl-1H-pyrazol-4-yl)-phenyl]-isoquinoline

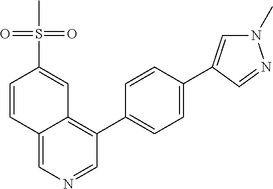 [SMILES: Cn1cc(-c2ccc(-c3cncc4ccc(S(C)(=O)=O)cc34)cc2)cn1]

Into a 10-mL vial purged and maintained with an inert atmosphere of nitrogen was added 4-chloro-6-methanesulfonylisoquinoline (150 mg, 0.62 mmol), 1-methyl-4-[4-(tetramethyl-1,3,2-dioxaborolan-2-yl)phenyl]-1H-pyrazole (230 mg, 0.81 mmol), sodium carbonate (198 mg, 1.87 mmol), Pd(PCy₃)₂Cl₂ (46 mg, 0.06 mmol), water (1 mL) and dioxane (4 mL). The reaction mixture was stirred under microwave radiation for 1 h at 120° C. and concentrated under vacuum. The residue was purified by silica gel column chromatography with ethyl acetate/petrolether (2:3). The product (40 mg) was further purified by prep-HPLC (acetonitrile/water). This resulted in 20 mg (9%) of 6-methanesulfonyl-4-[4-(1-methyl-1H-pyrazol-4-yl)phenyl]isoquinoline as a white solid. ¹H NMR (300 MHz, CDCl₃) ppm=9.41 (s, 1H), 8.70-8.68 (m, 2H), 8.29 (d, J=8.7 Hz, 1H), 8.12 (d, J=8.7 Hz, 1H), 7.86 (s, 1H), 7.72-7.67 (m, 3H), 7.52 (d, J=8.7 Hz, 2H), 4.00 (s, 3H), 3.10 (s, 3H). [M+H]⁺ 364. Rt 1.00 min (method C).

### Example 84

### 6-[imino(methane)sulfinyl]-4-[4-(1-methyl-1H-pyrazol-4-yl)phenyl]isoquinoline (190)

### 4-chloro-6-(methylsulfanyl)isoquinoline

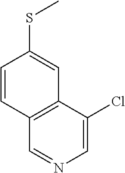

Into a 25-mL round-bottom flask purged and maintained with an inert atmosphere of nitrogen was added 6-bromo-4-chloroisoquinoline (500 mg, 2.06 mmol), (methylsulfanyl)sodium (149 mg, 2.13 mmol), Pd₂(dba)₃*CHCl₃ (106 mg, 0.10 mmol), dppf (57.0 mg, 0.10 mmol), TEA (418 mg, 4.13 mmol) and N,N-dimethylformamide (6 mL). The solution was stirred for 2.5 h at 75° C. The mixture was concentrated under vacuum and the residue was purified by silica gel column chromatography with ethyl acetate/petrolether (1:6). This resulted in 201 mg (46%) of 4-chloro-6-(methylsulfanyl)isoquinoline as a yellow solid. [M+H]⁺ 210. Rt 1.33 min (method I). 4-[4-(1-methyl-1H-pyrazol-4-yl)phenyl]-6-(methylsulfanyl)isoquinoline

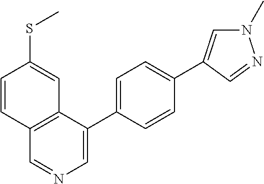 [SMILES: CSc1ccc2cncc(-c3ccc(-c4cnn(C)c4)cc3)c2c1]

Into a 20-mL vial purged and maintained with an inert atmosphere of nitrogen was added 4-chloro-6-(methylsulfanyl)isoquinoline (500 mg, 2.38 mmol), 1-methyl-4-[4-(tetramethyl-1,3,2-dioxaborolan-2-yl)phenyl]-1H-pyrazole (881 mg, 3.10 mmol), sodium carbonate (759 mg, 7.16 mmol), Pd(PCy₃)₂Cl₂ (176 mg, 0.24 mmol), dioxane (8 mL) and water (2 mL). The mixture was stirred under microwave irradiation for 1.5 h at 130° C. The mixture was concentrated under vacuum and the residue was purified by silica gel column chromatography with ethyl acetate/petrolether (7:3). This resulted in 500 mg (63%) of 4-[4-(1-methyl-1H-pyrazol-4-yl)phenyl]-6-(methylsulfanyl)isoquinoline as a yellow solid. [M+H]⁺ 332. Rt 0.72 min (method S).

### 6-methanesulfinyl-4-[4-(1-methyl-1H-pyrazol-4-yl)phenyl]isoquinoline

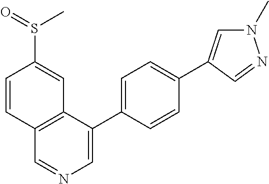 [SMILES: Cn1cc(-c2ccc(-c3cncc4ccc(S(C)=O)cc34)cc2)cn1]

Into a 25-mL round-bottom flask, was added 4-[4-(1-methyl-1H-pyrazol-4-yl)phenyl]-6-(methylsulfanyl)isoquinoline (400 mg, 1.21 mmol), methanol (5 mL), water (2 mL) and NaIO₄ (517 mg, 2.42 mmol). The solution was stirred for 4 h at 35° C. The solids were filtered off. The mixture was concentrated under vacuum and the residue was dissolved in 20 mL of dichloromethane and washed with 3×10 mL of water. The organic layer was dried and concentrated under vacuum. This resulted in 304 mg (72%) of 6-methanesulfinyl-4-[4-(1-methyl-1H-pyrazol-4-yl)phenyl]isoquinoline as a yellow solid. [M+H]⁺ 348.

### 6-[imino(methane)sulfinyl]-4-[4-(1-methyl-1H-pyrazol-4-yl)phenyl]isoquinoline

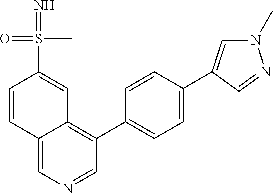 [SMILES: Cn1cc(-c2ccc(-c3cncc4ccc(S(C)(=N)=O)cc34)cc2)cn1]

Into a 25-mL round-bottom flask purged and maintained with an inert atmosphere of nitrogen was added 6-methanesulfinyl-4-[4-(1-methyl-1H-pyrazol-4-yl)phenyl]isoquinoline (200 mg, 0.58 mmol), trifluoroacetamide (196 mg, 1.73 mmol), Rh₂(OAc)₄ (26.0 mg, 0.06 mmol), PhI(OAc)₂ (371 mg, 1.15 mmol), MgO (116 mg, 2.88 mmol) and dichloromethane (5 mL). The solution was stirred for 24 h at 25° C. The reaction mixture was diluted with methanol (5 mL) and potassium carbonate (398 mg, 2.88 mmol) was added. The solution was stirred for 6 h at 25° C. The reaction mixture was concentrated under vacuum and the residue was purified by silica gel column chromatography with methanol:dichloromethane (1:25). The product was further purified by prep-HPLC (acetonitrile/water). This resulted in 15 mg (7%) of 6-[imino(methane)sulfinyl]-4-[4-(1-methyl-1H-pyrazol-4-yl)phenyl]isoquinoline as an off-white solid. ¹H NMR (300 MHz, CD₃OD) ppm=9.43 (s, 1H), 8.71 (s, 1H), 8.61 (s, 1H), 8.45 (d, J=8.4 Hz, 1H), 8.26-8.23 (m, 1H), 8.09 (s, 1H), 7.94 (s, 1H), 7.80 (d, J=8.4 Hz, 2H), 7.59 (d, J=8.4 Hz, 2H), 3.98 (s, 3H), 3.21 (s, 3H). [M+H]⁺ 363. Rt 1.10 min (method C).

### Example 85

### 4-[4-(1-Methyl-1H-pyrazol-4-yl)-phenyl]-isoquinoline-6-sulfonic acid amide (191)

### 4-chloroisoquinoline-6-sulfonyl chloride

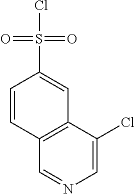 [SMILES: O=S(=O)(Cl)c1ccc2cncc(Cl)c2c1]

Into a 25-mL round-bottom flask, was added hydrogen chloride (1.50 mL, 49.4 mmol) and 4-chloroisoquinolin-6-amine (150 mg, 0.77 mmol, 92%). This was followed by the addition of NaNO₂ (50.0 mg, 0.72 mmol). The mixture was stirred for 1 h at 0° C. To the mixture was added acetic acid (4 mL) and SO₂ (50 mL). The mixture solution was stirred for 30 min and CuCl₂*.2H₂O (336 mg, 1.97 mmol) was added. The reaction mixture was stirred for 0.5 h at 25° C. The solution was diluted with 100 mL of water. The solids were collected by filtration and discarded. The filtrate was concentrated under vacuum. This resulted in 70 mg (28%) of 4-chloroisoquinoline-6-sulfonyl chloride as a yellow solid. [M+H]⁺ 263.

### 4-chloroisoquinoline-6-sulfonamide

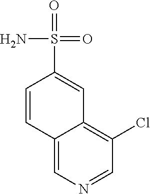 [SMILES: NS(=O)(=O)c1ccc2cncc(Cl)c2c1]

Into a 50-mL round-bottom flask was added dioxane (15 mL), 4-chloroisoquinoline-6-sulfonyl chloride (70.0 mg, 0.21 mmol, 80%) and aqueous ammonia (10 mL). The solution was stirred for 1 h at 25° C. The solution was extracted with 10 mL of dichloromethane and the organic layer concentrated under vacuum. This resulted in 30 mg (46%) of 4-chloroisoquinoline-6-sulfonamide as a yellow solid. [M+H]₊243.

### 4-[4-(1-Methyl-1H-pyrazol-4-yl)-phenyl]-isoquinoline-6-sulfonic acid amide

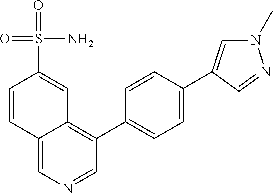 [SMILES: Cn1cc(-c2ccc(-c3cncc4ccc(S(N)(=O)=O)cc34)cc2)cn1]

Into a 30-mL vial purged and maintained with an inert atmosphere of nitrogen was added dioxane (10 mL), water (5 mL), 4-chloroisoquinoline-6-sulfonamide (50 mg, 0.16 mmol, 80%), 1-methyl-4-[4-(tetramethyl-1,3,2-dioxaborolan-2-yl)phenyl]-1H-pyrazole (61 mg, 0.21 mmol), Pd(PCy₃)₂Cl₂ (12 mg, 0.020 mmol) and sodium carbonate (52 mg, 0.49 mmol). The reaction mixture was stirred under microwave irradiation for 1.5 h at 100° C. The solids were filtered off and discarded. The filtrate was concentrated under vacuum and the residue was purified by prep-HPLC (acetonitrile/water). This resulted in 15 mg (25%) of 4-[4-(1-methyl-1H-pyrazol-4-yl)phenyl]isoquinoline-6-sulfonamide as a white solid. ¹H NMR (300 MHz, DMSO-d6) ppm=9.47 (s, 1H), 8.62 (s, 1H), 8.45-8.37 (m, 1H), 8.28 (s, 1H), 8.09 (s, 1H), 8.07-8.00 (s, 1H), 7.80-7.78 (m, 1H), 7.56-7.54 (m, 1H), 3.90 (s, 3H). [M+H]⁺ 365. Rt 1.24 min (method C).

### Example 86

### 4-(4-(1-methyl-1H-pyrazol-4-yl)phenyl)isoquinoline (109)

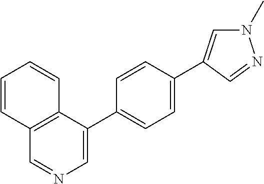 [SMILES: Cn1cc(-c2ccc(-c3cncc4ccccc34)cc2)cn1]

4-bromoisoquinoline (50 mg, 0.240 mmol), 1-methyl-4-(4-(4,4,5,5-tetramethyl-1,3,2-dioxaborolan-2-yl)phenyl)-1H-pyrazole (75 mg, 0.264 mmol) and Pd(dppf)Cl₂.CH₂Cl₂ (9.8 mg, 0.012 mmol) were loaded in a microwave vial and then degassed acetonitrile (4.1 mL) and sodium carbonate in water (673 μl, 0.336 mmol) were added. The reaction was heated at 120° C. for 60 min under microwave irradiation. The reaction mixture was concentrated and purified via biotage column chromatography (dichloromethane/EtOH 99/1 to 97/3) to give the title compound (52 mg, 76% yield). ¹H NMR (500 MHz, CDCl₃) ppm=9.27 (d, J=0.9, 1H), 8.53 (s, 1H), 8.06 (d, J=8.0, 1H), 7.99 (d, J=8.4, 1H), 7.86 (d, J=0.9, 1H), 7.71 (s, 1H), 7.72-7.68 (m, 1H), 7.67-7.63 (m, 3H), 7.55-7.51 (m, 2H), 4.00 (s, 3H) [M+H]⁺ 286. Rt 2.5 min (method N).

### Example 87

### 5-(isoquinolin-4-yl)-1-methyl-1,3-dihydrobenzo[c]isothiazole 2,2-dioxide (100)

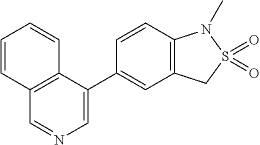 [SMILES: CN1c2ccc(-c3cncc4ccccc34)cc2CS1(=O)=O]

5-(isoquinolin-4-yl)-1-methyl-1,3-dihydrobenzo[c]isothiazole 2,2-dioxide was prepared in a manner similar to Example 41, using 1-methyl-5-(4,4,5,5-tetramethyl-1,3,2-dioxaborolan-2-yl)-1,3-dihydrobenzo[c]isothiazole 2,2-dioxide as the starting material. ¹H NMR (500 MHz, CDCl₃) ppm=9.26 (d, J=0.9, 1H), 8.45 (s, 1H), 8.08-8.04 (m, 1H), 7.89-7.85 (m, 1H), 7.71 (ddd, J=8.4, 6.8, 1.5, 1H), 7.66 (ddd, J=8.1, 6.8, 1.2, 1H,), 7.50 (ddt, J=8.1, 1.7, 0.8, 1H), 7.42 (dt, J=1.7, 1.0, 1H), 6.90 (d, J=8.1, 1H), 4.45 (d, J=1.0, 2H), 3.24 (s, 3H). [M+H]⁺ 311. Rt 1.98 min (method N).

### Example 88

### (S)-isoquinolin-6-yl(2-phenylpyrrolidin-1-yl)methanone (122)

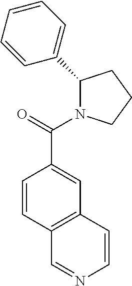 [SMILES: O=C(c1ccc2cnccc2c1)N1CCC[C@H]1c1ccccc1]

To a suspension of isoquinoline-6-carboxylic acid (50 mg, 0.289 mmol) in DMF (2.4 mL) was added HATU (132 mg, 0.346 mmol) and the mixture was stirred for 15 min before the addition of (S)-2-phenylpyrrolidine hydrochloride (58.3 mg, 0.318 mmol). The mixture was stirred at rt for 15 min before the addition of DIPEA (111 μL, 0.635 mmol). The resulting solution was then stirred at rt overnight. The reaction mixture was concentrated and purified via biotage column chromatography (dichloromethane/EtOH 99/1 to 95/5). The obtained product was solubilised in dichloromethane and washed with water three times. The organic layer was dried over MgSO₄ and concentrated to give the title compound (50 mg, 57% yield, as a mixture of rotamers). ¹H NMR (500 MHz, CDCl₃) ppm=9.30 (s, 0.43H), 9.18 (s, 0.57H), 8.60 (d, J=5.7, 0.43H), 8.47 (d, J=5.7, 0.57H), 8.06-8.02 (m, 0.86H), 7.83-7.76 (m, 1.14H), 7.72 (d, J=5.7, 0.43H), 7.52 (d, J=1.6, 0.57H), 7.43-7.32 (m, 3H), 7.29-7.19 (m, 2H), 6.98 (dd, J=7.5, 1.9, 1H), 5.40 (dd, J=7.8, 5.5, 0.43H), 4.84 (dd, J=7.7, 2.7, 0.57H), 4.04 (dt, J=12.5, 8.2, 0.57H), 3.95 (ddd, J=12.5, 7.9, 4.6, 0.57H), 3.79 (dt, J=10.6, 7.0, 0.43H), 3.62 (ddd, J=10.6, 7.3, 4.8, 0.43H), 2.53-2.43 (m, 0.43H), 2.39-2.30 (m, 0.57H), 2.13-1.85 (m, 3H). [M+H]⁺ 303. Rt 2.10 min (method N).

### Example 89

### (S)-(1,6-naphthyridin-2-yl)(2-phenylpyrrolidin-1-yl)methanone (54)

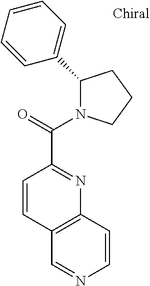 [SMILES: O=C(c1ccc2cnccc2n1)N1CCC[C@H]1c1ccccc1]

(S)-(1,6-naphthyridin-2-yl)(2-phenylpyrrolidin-1-yl)methanone was prepared in a manner similar to Example 43, using 1,6-naphthyridine-2-carboxylic acid as the starting material. ¹H NMR (500 MHz, CDCl₃) ppm=9.32 (d, J=0.9, 0.35H), 9.15 (d, J=0.9, 0.65H), 8.82 (d, J=5.9, 0.35H), 8.74 (d, J=5.9, 0.65H), 8.38 (dd, J=8.5, 0.9, 0.35H), 8.07-8.01 (m, 1H), 7.98 (dt, J=5.9, 0.9, 0.35H), 7.74 (dt, J=5.9, 0.9, 0.65H), 7.50 (d, J=8.5, 0.65H), 7.38-7.31 (m, 1.4H), 7.26-7.20 (m, 0.35H), 7.04-6.97 (m, 1.95H), 6.91-6.83 (m, 1.3H), 5.65 (dd, J=7.7, 4.8, 0.65H), 5.44 (dd, J=7.8, 4.4, 0.35H), 4.33-4.25 (m, 0.35H), 4.08-3.96 (m, 1.65H), 2.54-2.36 (m, 1H), 2.12-1.89 (m, 3H) [M+H]⁺ 304. Rt 2.46 min (method N).

### Example 90

### (S)-(2-(4-chlorophenyl)pyrrolidin-1-yl)(isoquinolin-6-yl)methanone (113)

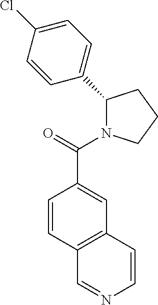 [SMILES: O=C(c1ccc2cnccc2c1)N1CCC[C@H]1c1ccc(Cl)cc1]

To a suspension of isoquinoline-6-carboxylic acid (50 mg, 0.289 mmol) in DMF (2.4 mL) was added HATU (132 mg, 0.346 mmol) and the mixture was stirred for 15 min before the addition of (S)-2-(4-chlorophenyl)pyrrolidine hydrochloride (69.3 mg, 0.318 mmol). The mixture was stirred at rt for 15 min before the addition of DIPEA (111 μL, 0.635 mmol). The resulting solution was then stirred at rt overnight. The reaction mixture was concentrated and purified via biotage column chromatography (dichloromethane/EtOH 99/1 to 95/5). The product was solubilised in dichloromethane and washed with water three times. The organic layer was dried over MgSO₄ and concentrated before being filtrated on a SCX2 column. The product was released with 1N NH₃ in MeOH to give the title compound (80 mg, 82% yield, mixture of rotamers). ¹H NMR (500 MHz, CDCl₃) ppm=9.30 (s, 0.53H), 9.19 (s, 0.47H), 8.59 (d, J=5.7, 0.53H), 8.50 (d, J=5.7, 0.47H), 8.07-7.99 (m, 1.06H), 7.81 (d, J=8.4, 0.47H), 7.78 (dd, J=8.4, 1.6, 0.53H), 7.71 (d, J=5.7, 0.53H), 7.53 (s, 0.47H), 7.41 (d, J=5.7, 0.47H), 7.37 (dd, J=8.4, 1.6, 0.47H), 7.33 (d, J=8.6, 1.06H), 7.30 (d, J=8.6, 1.06H), 7.22-7.16 (m, 0.94H), 6.90 (d, J=8.3, 0.94H), 5.33 (dd, J=7.6, 5.9, 0.53H), 4.82 (dd, J=7.6, 2.8, 0.47H), 4.02 (dt, J=12.5, 8.1, 0.47H), 3.96-3.87 (m, 0.47H), 3.78 (dt, J=11.0, 7.0, 0.53H), 3.62 (ddd, J=11.0, 7.2, 5.1, 0.53H), 2.52-2.40 (m, 0.53H), 2.40-2.29 (m, 0.47H), 2.07-1.83 (m, 3H) [M+H]⁺ 337. Rt 2.41 min (method N).

### Example 91

### (S)-(2-(4-chlorophenyl)pyrrolidin-1-yl)(1,6-naphthyridin-2-yl)methanone (37)

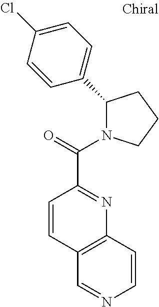 [SMILES: O=C(c1ccc2cnccc2n1)N1CCC[C@H]1c1ccc(Cl)cc1]

(S)-(2-(4-chlorophenyl)pyrrolidin-1-yl)(1,6-naphthyridin-2-yl)methanone was prepared in a manner similar to Example 45, using 1,6-naphthyridine-2-carboxylic acid as the starting material. ¹H NMR (500 MHz, CDCl₃) ppm=9.34 (d, J=0.9, 0.40H), 9.21 (d, J=0.9, 0.60H), 8.84 (d, J=5.9, 0.40H), 8.76 (d, J=5.9, 0.60H), 8.40 (dd, J=8.5, 0.9, 0.40H), 8.15 (dd, J=8.5, 0.9, 0.60H), 8.07 (d, J=8.5, 0.40H), 7.98 (dt, J=5.9, 0.9, 0.40H), 7.71 (dt, J=5.9, 0.9, 0.60H), 7.64 (d, J=8.5, 0.60H), 7.34-7.28 (m, 1.60H), 7.05-7.01 (m, 1.20H), 6.89-6.84 (m, 1.20H), 5.74 (dd, J=7.7, 4.4, 0.60H), 5.39 (dd, J=7.8, 4.8, 0.40H), 4.35-4.29 (m, 0.40H), 4.08-3.98 (m, 1.6H), 2.57-2.37 (m, 1H), 2.12-1.90 (m, 3H). [M+H]⁺ 338. Rt 1.38 min (method M).

### Example 92

### (S)-(1-aminoisoquinolin-6-yl)(2-(4-chlorophenyl)pyrrolidin-1-yl)methanone (137)

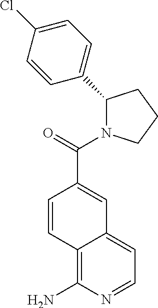 [SMILES: Nc1nccc2cc(C(=O)N3CCC[C@H]3c3ccc(Cl)cc3)ccc12]

To (S)-(2-(4-chlorophenyl)pyrrolidin-1-yl)(isoquinolin-6-yl)methanone (Example 44) (24 mg, 0.071 mmol) in dichloromethane (713 μL) was added 3-chloroperoxybenzoic acid (12.30 mg, 0.071 mmol). The reaction mixture was stirred at rt for 2 h. After addition of a solution of Na₂S₂O₅ and then NaHCO₃, the reaction mixture was diluted with dichloromethane. The aqueous layer was extracted three times with dichloromethane, the organic layer was dried over MgSO₄ and concentrated.

To the crude in solution in pyridine (1.7 mL, 14.20 mmol) was added 4-toluenesulfonyl chloride (16.24 mg, 0.085 mmol) and the mixture was stirred at rt for 45 min. To the reaction mixture was added ethanolamine (107 μl, 1.775 mmol) and the reaction mixture was stirred at rt for 45 min before being diluted with water and dichloromethane. The aqueous layer was extracted with dichloromethane three times and the organic layer was concentrated. The crude was purified via biotage column chromatography (dichloromethane/EtOH 99/1 to 85/15) and by preparative HPLC. Chromatographic separation at room temperature was carried out using Gilson GX-281 Liquid Handler system combined with a Gilson 322 HPLC pump (Gilson, Middleton, USA) over a 15 minute gradient elution (Grad15 mins20 mls·m) from 10:90 to 100:0 methanol:water (both modified with 0.1% formic acid) at a flow rate of 20 mL/min. UV-Vis spectra were acquired at 254 nm on a Gilson 156 UV-Vis detector (Gilson, Middleton, USA). Collection was triggered by UV signal, and collected using a Gilson GX-281 Liquid Handler system (Gilson, Middleton, USA). The fractions were combined and the solvent was evaporated. The residue was solubilised in dichloromethane and a solution of NaHCO₃ was added. The aqueous layer was extracted with dichloromethane and the organic layers were dried over MgSO₄ and concentrated to give the title compound (8 mg, 32% yield, mixture of rotamers). ¹H NMR (500 MHz, CDCl₃) ppm=7.99 (d, J=5.9, 0.5H), 7.92-7.87 (m, 1.5H), 7.69-7.64 (m, 1H), 7.45 (d, J=1.9, 0.5H), 7.38-7.29 (m, 2H), 7.27-7.23 (m, 1.5H), 7.10 (d, J=5.9, 0.5H), 6.97-6.92 (m, 1H), 6.81 (d, J=5.9, 0.5H), 5.45 (bs, 2H), 5.37-5.33 (m, 0.5H), 4.85 (dd, J=7.5, 2.6, 0.5H), 4.03 (dt, J=12.6, 8.2, 0.5H) 3.93 (ddd, J=12.4, 8.0, 4.3, 0.5H), 3.79 (dt, J=10.5, 6.9, 0.5H), 3.68-3.62 (m, 0.5H), 2.49 (dt, J=13.4, 6.7, 0.5H), 2.42-2.29 (m, 0.5H), 2.10-1.83 (m, 3H). [M+H]⁺ 352. Rt 2.05 min (method N).

### Example 93

### (S)-(2-(4-bromophenyl)pyrrolidin-1-yl)(1,6-naphthyridin-2-yl)methanone (84)

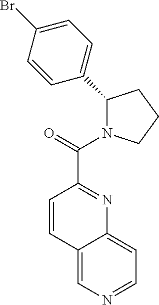 [SMILES: O=C(c1ccc2cnccc2n1)N1CCC[C@H]1c1ccc(Br)cc1]

To a suspension of 1,6-naphthyridine-2-carboxylic acid (50 mg, 0.287 mmol) in DMF (2.4 mL) was added HATU (131 mg, 0.345 mmol) and the mixture was stirred for 15 min before the addition of (S)-2-(4-bromophenyl)pyrrolidine hydrochloride (75 mg, 0.287 mmol) and DIPEA (110 μL, 0.632 mmol). The resulting solution was then stirred at rt for 2 h. The reaction mixture was concentrated and then diluted in dichloromethane and washed with water three times. The organic layer was dried over MgSO₄ and concentrated under vacuum, The crude was purified via biotage column chromatography (dichloromethane/EtOH 99/1 to 95/5). The product was filtered on a SCX2 column and released with 1N NH₃ in methanol to give title compound (96 mg, 87% yield, mixture of rotamers). ¹H NMR (500 MHz, CDCl₃) ppm=9.32 (d, J=0.9, 0.45H), 9.19 (d, J=0.9, 0.55H), 8.82 (d, J=5.9, 0.45H), 8.74 (d, J=5.9, 0.55H), 8.38 (dd, J=8.5, 0.9, 0.45H), 8.14 (dd, J=8.5, 0.9, 0.55H), 8.04 (d, J=8.5, 0.45H), 7.96 (dt, J=5.9, 0.9, 0.45H), 7.68 (d, J=5.9, 0.55H), 7.64 (d, J=8.5, 0.55H), 7.47-7.43 (m, 0.90H), 7.25-7.19 (m, 0.90H), 7.21-7.15 (m, 1.1H), 6.82-6.78 (m, 1.1H), 5.72 (dd, J=7.7, 4.3, 0.55H), 5.35 (dd, J=7.8, 4.8, 0.45H), 4.34-4.26 (m, 0.45H), 4.06-3.95 (m, 1.55H), 2.53-2.36 (m, 1H), 2.10-1.87 (m, 3H). [M+H]⁺ 382/384. Rt 2.71 min (method N).

### Example 94

### (S)-(8-bromo-1,6-naphthyridin-2-yl)(2-(4-chlorophenyl)pyrrolidin-1-yl)methanone (34)

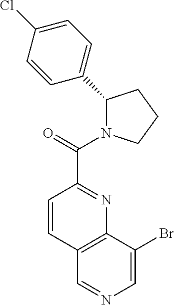 [SMILES: O=C(c1ccc2cncc(Br)c2n1)N1CCC[C@H]1c1ccc(Cl)cc1]

To a suspension of 8-bromo-1,6-naphthyridine-2-carboxylic acid (50 mg, 0.198 mmol) in DMF (1.65 mL) was added HATU (90 mg, 0.237 mmol) and the mixture was stirred for 15 min before the addition of (S)-2-(4-chlorophenyl)pyrrolidine hydrochloride (47.4 mg, 0.217 mmol). The mixture was stirred at rt for 15 min before the addition of DIPEA (76 μL, 0.435 mmol). The resulting solution was then stirred at rt for 1 h30. The reaction mixture was concentrated and purified via biotage column chromatography (dichloromethane/EtOH 100/0 to 99/1) to give the title compound (79 mg, 96% yield, mixture of rotamers). ¹H NMR (500 MHz, CDCl₃) ppm=9.24 (s, 0.45H,), 9.10 (s, 0.55H), 9.06 (s, 0.45H), 8.98 (s, 0.55H), 8.41 (d, J=8.5, 0.45H), 8.28 (d, J=8.5, 0.45H), 8.18 (d, J=8.5, 0.55H), 7.87 (d, J=8.5, 0.55H), 7.32-7.26 (m, 1.8H), 6.99-6.90 (m, 2.2H), 6.30 (dd, J=7.7, 4.3, 0.55H), 5.42 (dd, J=7.9, 4.7, 0.45H), 4.61 (ddd, J=12.1, 7.5, 5.9, 0.45H,), 4.33 (dt, J=12.1, 7.1, 0.45H), 4.04 (t, J=6.8, 1.1H), 2.61-2.52 (m, 0.55H), 2.47-2.38 (m, 0.45H), 2.16-1.88 (m, 3H). [M+H]⁺ 416/418. Rt 3.16 min (method N).

### Example 95

### (S)-(8-bromo-1,6-naphthyridin-2-yl)(2-(4-bromophenyl)pyrrolidin-1-yl)methanone (89)

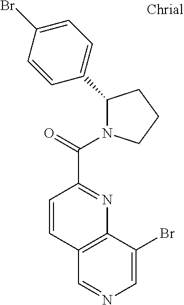 [SMILES: O=C(c1ccc2cncc(Br)c2n1)N1CCC[C@H]1c1ccc(Br)cc1]

(S)-(8-bromo-1,6-naphthyridin-2-yl)(2-(4-bromophenyl)pyrrolidin-1-yl)methanone was prepared in a manner similar to Example 49, using (S)-2-(4-bromophenyl)pyrrolidine hydrochloride as the starting material. ¹H NMR (500 MHz, CDCl₃) ppm=9.26 (s, 0.45H), 9.13 (s, 0.55H), 9.07 (s, 0.45H), 8.99 (s, 0.55H), 8.43 (d, J=8.5, 0.45H), 8.30 (d, J=8.5, 0.45H), 8.21 (d, J=8.5, 0.55H), 7.90 (d, J=8.5, 0.55H), 7.49-7.45 (m, 0.9H), 7.25-7.21 (m, 0.9H), 7.15-7.11 (m, 1.1H), 6.90-6.85 (m, 1.1H), 6.31 (dd, J=7.7, 4.3, 0.55H), 5.41 (dd, J=8.0, 4.7, 0.45H), 4.62 (ddd, J=12.3, 7.5, 5.9, 0.45H), 4.34 (dt, J=12.3, 7.1, 0.45H), 4.05 (dd, J=7.4, 6.2, 1.1H), 2.63-2.51 (m, 0.55H), 2.48-2.39 (m, 0.45H), 2.18-1.89 (m, 3H). [M+H]⁺ 459/461/463. Rt 3.08 min (method N).

### Example 96

### (S)-(8-bromo-1,6-naphthyridin-2-yl)(2-(4-fluorophenyl)pyrrolidin-1-yl)methanone (90)

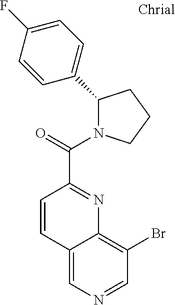 [SMILES: O=C(c1ccc2cncc(Br)c2n1)N1CCC[C@H]1c1ccc(F)cc1]

(S)-(8-bromo-1,6-naphthyridin-2-yl)(2-(4-fluorophenyl)pyrrolidin-1-yl)methanone was prepared in a manner similar to Example 49, using (S)-2-(4-fluorophenyl)pyrrolidine hydrochloride as the starting material. ¹H NMR (500 MHz, CDCl₃) ppm=9.26 (s, 0.45H), 9.11 (s, 0.55H), 9.07 (s, 0.45H), 9.00 (s, 0.55H), 8.43 (d, J=8.5, 0.45H), 8.30 (d, J=8.5, 0.45H), 8.17 (d, J=8.5, 0.55H), 7.83 (d, J=8.5, 0.55H), 7.35-7.29 (m, 0.9H), 7.08-7.01 (m, 0.9H), 6.96-6.91 (m, 1.1H), 6.70-6.64 (m, 1.1H), 6.23 (dd, J=7.6, 5.0, 0.55H), 5.44 (dd, J=7.9, 4.7, 0.45H), 4.66-4.59 (m, 0.45H), 4.32 (dt, J=12.1, 7.1, 0.45H), 4.10-4.01 (m, 1.1H), 2.63-2.51 (m, 0.55H), 2.48-2.38 (m, 0.45H), 2.20-1.88 (m, 3H). [M+H]⁺ 400/402. Rt 2.9 min (method N).

### Example 97

### (S)-(8-bromo-1,6-naphthyridin-2-yl)(2-(4-(trifluoromethyl)phenyl)pyrrolidin-1-yl)methanone (91)

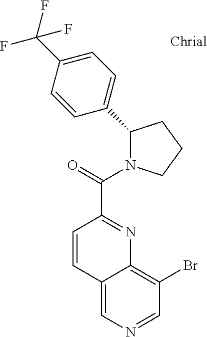 [SMILES: O=C(c1ccc2cncc(Br)c2n1)N1CCC[C@H]1c1ccc(C(F)(F)F)cc1]

(S)-(8-bromo-1,6-naphthyridin-2-yl)(2-(4-(trifluoromethyl)phenyl)pyrrolidin-1-yl)methanone was prepared in a manner similar to Example 49, using (S)-2-(4-trifluoromethylphenyl)pyrrolidine hydrochloride as the starting material. ¹H NMR (500 MHz, CDCl₃) ppm=9.27 (s, 0.5H), 9.11 (s, 0.5H), 9.08 (s, 0.5H), 8.99 (s, 0.5H), 8.44 (d, J=8.5, 0.5H), 8.29 (d, J=8.5, 0.5H), 8.20 (d, J=8.5, 0.5H), 7.94 (d, J=8.5, 0.5H), 7.64-7.59 (m, 1H), 7.49-7.44 (m, 1H), 7.32-7.28 (m, 1H), 7.17-7.13 (m, 1H), 6.43 (dd, J=7.6, 3.8, 0.5H), 5.49 (dd, J=8.0, 4.9, 0.5H), 4.70-4.62 (m, 0.5H), 4.37 (dt, J=12.1, 7.0, 0.5H), 4.08 (t, J=6.8, 1H), 2.65-2.56 (m, 0.5H), 2.53-2.43 (m, 0.5H), 2.19-1.87 (m, 3H) [M+H]⁺ 450/452. Rt 3.06 min (method N).

### Example 98

### (S)-(2-(4-chlorophenyl)pyrrolidin-1-yl)(8-methyl-1,6-naphthyridin-2-yl)methanone (83)

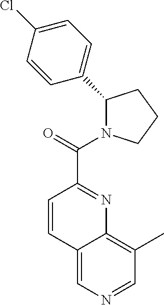 [SMILES: Cc1cncc2ccc(C(=O)N3CCC[C@H]3c3ccc(Cl)cc3)nc12]

(S)-(8-bromo-1,6-naphthyridin-2-yl)(2-(4-chlorophenyl)pyrrolidin-1-yl)methanone (Example 49) (82 mg, 0.197 mmol) and Pd(dppf)Cl₂.CH₂Cl₂ (8.04 mg, 9.84 μmol) were loaded in a microwave vial. Degassed acetonitrile (3.4 mL) and sodium carbonate in water (551 μL, 0.275 mmol) were added followed by the addition of 2,4,6-trimethyl-1,3,5,2,4,6-trioxatriborinane (14 μL, 0.098 mmol). The reaction mixture was heated in the microwave at 100° C. for 1 h. 2,4,6-trimethyl-1,3,5,2,4,6-trioxatriborinane (14 μL, 0.098 mmol) were added to the reaction mixture and the reaction mixture was heated at 120° C. for 50 min. The crude was concentrated and purified via biotage column chromatography (dichloromethane/EtOH, 99/1 to 93/7) and by prepHPLC. The fractions were concentrated and the residue was diluted in dichloromethane and washed with NaHCO₃. The organic layer was dried over MgSO₄ and concentrated to give the title compound (40 mg, 55% yield, mixture of rotamers). ¹H NMR (500 MHz, CDCl₃) ppm=9.20 (s, 0.5H), 9.07 (s, 0.5H), 8.71 (s, 0.5H), 8.59 (s, 0.5H), 8.38 (d, J=8.5, 0.5H), 8.18 (d, J=8.5, 0.5H), 8.15 (d, J=8.5, 0.5H), 7.79 (d, J=8.5, 0.5H), 7.34-7.28 (m, 2H), 7.10-7.06 (m, 1H), 6.95-6.91 (m, 1H), 5.86 (dd, J=7.6, 3.2, 0.5H), 5.42 (dd, J=7.9, 4.9, 0.5H), 4.46-4.38 (m, 0.5H), 4.19 (dt, J=11.7, 6.8, 0.5H), 4.10-3.95 (m, 1H), 2.81 (s, 1.5H), 2.52-2.38 (m, 2.5H), 2.15-1.90 (m, 3H). [M+H]⁺ 352. Rt 2.84 min (method N).

### Example 99

### (S)-(2-(4-bromophenyl)pyrrolidin-1-yl)(8-methyl-1,6-naphthyridin-2-yl)methanone (92)

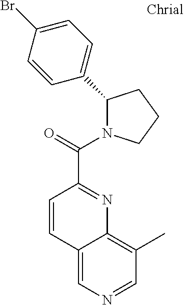 [SMILES: Cc1cncc2ccc(C(=O)N3CCC[C@H]3c3ccc(Br)cc3)nc12]

(S)-(2-(4-bromophenyl)pyrrolidin-1-yl)(8-methyl-1,6-naphthyridin-2-yl)methanone was prepared in a manner similar to Example 53, using (S)-(8-bromo-1,6-naphthyridin-2-yl)(2-(4-bromophenyl)pyrrolidin-1-yl)methanone (Example 50) as the starting material. ¹H NMR (500 MHz, CDCl₃) ppm=9.21 (s, 0.5H), 9.09 (s, 0.5H), 8.71 (s, 0.5H), 8.59 (s, 0.5H), 8.43-8.33 (m, 0.5H), 8.25-8.08 (m, 1H), 7.81 (s, 0.5H), 7.47 (d, J=7.9, 1H), 7.24 (d, J=7.9, 2H), 6.88 (d, J=7.9, 1H), 5.88-5.79 (m, 0.5H), 5.40 (dd, J=7.9, 4.8, 0.5H), 4.42 (dt, J=12.2, 6.8, 0.5H), 4.19 (dt, J=12.2, 6.6, 0.5H), 4.09-3.96 (m, 1H), 2.82 (s, 1.5H), 2.50-2.40 (m, 2.5H), 2.15-1.89 (m, 3H). [M+H]⁺ 396/398. Rt 1.43 min (method M).

### Example 100

### (S)-(2-(4-chlorophenyl)pyrrolidin-1-yl)(8-(4-(1-methyl-1H-pyrazol-4-yl)phenyl)-1,6-naphthyridin-2-yl)methanone (67)

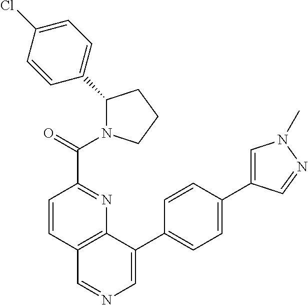 [SMILES: Cn1cc(-c2ccc(-c3cncc4ccc(C(=O)N5CCC[C@H]5c5ccc(Cl)cc5)nc34)cc2)cn1]

(S)-(8-bromo-1,6-naphthyridin-2-yl)(2-(4-chlorophenyl)pyrrolidin-1-yl)methanone (Example 49), 1-methyl-4-(4-(4,4,5,5-tetramethyl-1,3,2-dioxaborolan-2-yl)phenyl)-1H-pyrazole (30.0 mg, 0.106 mmol) and Pd(dppf)Cl₂.CH₂Cl₂ (3.92 mg, 4.80 μmol) were loaded in a microwave vial and then degassed acetonitrile (1200 μl) and sodium carbonate in water (269 μl, 0.134 mmol) were added. The reaction was heated at 120° C. for 60 min under microwave irradiation. The reaction mixture was concentrated and purified via biotage column chromatography (dichloromethane/EtOH 99/1 to 96/4). The obtained product was then filtered on a SCX-2 column, the product was released with 1N NH₃ in MeOH to give the title compound (36 mg, 76% yield, mixture of rotamers). ¹H NMR (500 MHz, CDCl₃) ppm=9.32 (s, 0.5H), 9.18 (s, 0.5H), 8.91 (s, 0.5H), 8.82 (s, 0.5H), 8.46 (d, J=8.5, 0.5H), 8.25 (d, J=8.5, 0.5H), 8.17 (d, J=8.5, 0.5H), 7.92 (d, J=8.5, 0.5H), 7.92 (d, J=0.8, 0.5H), 7.88 (d, J=0.8, 0.5H), 7.79-7.64 (m, 5H), 7.33-7.25 (m, 1H), 7.25-7.17 (m, 1H), 6.95-6.80 (m, 1H), 6.54-6.39 (m, 1H), 5.94 (dd, J=7.8, 3.9, 0.5H), 5.36 (dd, J=8.3, 4.3, 0.5H), 4.02 (s, 1.5H), 4.00 (s, 1.5H), 4.02-3.85 (m, 2H), 2.39-2.25 (m, 1H), 1.99-1.70 (m, 3H). [M+H]⁺ 494. Rt 3.04 min Method N.

### Example 101

### (S)-(5-amino-8-methyl-1,6-naphthyridin-2-yl)(2-(4-chlorophenyl)pyrrolidin-1-yl)methanone (88)

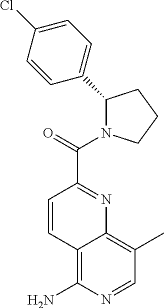 [SMILES: Cc1cnc(N)c2ccc(C(=O)N3CCC[C@H]3c3ccc(Cl)cc3)nc12]

To (S)-(2-(4-chlorophenyl)pyrrolidin-1-yl)(8-methyl-1,6-naphthyridin-2-yl)methanone (Example 53) (63 mg, 0.179 mmol) in dichloromethane (1.8 mL) was added 3-chloroperoxybenzoic acid (80 mg, 0.358 mmol) at rt for 1.5 h. After addition of a solution of Na₂S₂O₅ and then NaHCO₃, the reaction mixture was diluted with dichloromethane. The aqueous layer was extracted three times with dichloromethane, the organic layer was dried over MgSO₄ and concentrated. The residue was then solubilised in pyridine (6.5 mL) and 4-toluenesulfonyl chloride (62.5 mg, 0.328 mmol) was added. The mixture was stirred at rt for 45 min before ethanolamine (413 μL, 6.83 mmol) was added. The reaction mixture was stirred at rt for 45 min and then diluted with water and dichloromethane. The aqueous layer was extracted with dichloromethane three times and the organic layer was concentrated. The crude was purified via biotage column chromatography (dichloromethane/EtOH 99/1 to 85/15) to give the title compound (17 mg, 17% yield, mixture of rotamers). ¹H NMR (500 MHz, CDCl₃) ppm=8.27 (d, J=8.6, 0.5H), 8.06 (d, J=8.6, 0.5H), 8.04 (q, J=1.0, 0.5H), 8.00 (d, J=8.6, 0.5H), 7.93 (q, J=1.0, 0.5H), 7.66 (d, J=8.6, 0.5H), 7.33-7.25 (m, 2H), 7.10-7.05 (m, 1H), 6.94-6.89 (m, 1H), 5.94 (dd, J=7.7, 3.2, 0.5H), 5.47-5.15 (m, 2.5H), 4.41 (ddd, J=11.8, 7.6, 6.4, 0.5H), 4.19 (dt, J=11.8, 6.9, 0.5H), 4.07-3.94 (m, 1H), 2.59 (d, J=1.0, 1.5H), 2.50-2.38 (m, 1H), 2.27 (d, J=1.0, 1.5H), 2.13-1.87 (m, 3H). [M+H]⁺ 367. Rt 2.22 min (method N).

### Example 102

### (S)-8-bromo-N-(1-phenylethyl)-1,6-naphthyridine-2-carboxamide (70)

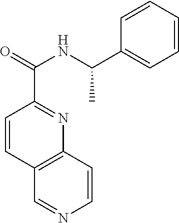 [SMILES: C[C@H](NC(=O)c1ccc2cnccc2n1)c1ccccc1]

To a suspension of 8-bromo-1,6-naphthyridine-2-carboxylic acid (100 mg, 0.395 mmol) in DMF (3.3 mL) was added HATU (180 mg, 0.474 mmol) and the mixture stirred at rt for 20 min before the addition of (S)-alpha-methylbenzylamine (151 μL, 1.186 mmol). The mixture was stirred at rt for 15 min before the addition of DIPEA (301 μL, 1.723 mmol). The resulting solution was then stirred at rt overnight and then concentrated and purified via biotage column chromatography (dichloromethane). The product obtained was filtered on a SCX-2 column and the product was released with 1N NH₃ in dichloromethane to give the title compound (70 mg, 50% yield). ¹H NMR (500 MHz, CDCl₃) ppm=9.27 (s, 1H), 9.05 (s, 1H), 8.64 (bd, J=8.3, 1H), 8.51 (d, J=8.4, 1H), 8.49 (d, J=8.4, 1H), 7.50-7.45 (m, 2H), 7.44-7.37 (m, 2H), 7.35-7.28 (m, 1H), 5.43-5.34 (m, 1H), 1.71 (d, J=6.9, 3H). [M+H]⁺ 356/358. Rt 3.16 min (method N).

### Example 103

### (R)-8-bromo-N-(1-phenylethyl)-1,6-naphthyridine-2-carboxamide (48)

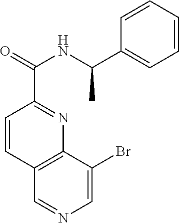 [SMILES: C[C@@H](NC(=O)c1ccc2cncc(Br)c2n1)c1ccccc1]

(R)-8-bromo-N-(1-phenylethyl)-1,6-naphthyridine-2-carboxamide was prepared in a manner similar to Example 57, using (R)-alphamethylbenzylamine as the starting material. ¹H NMR (500 MHz, CDCl₃) ppm=9.28 (s, 1H), 9.06 (s, 1H), 8.65 (bd, J=8.1, 1H), 8.52 (d, J=8.4, 1H), 8.50 (d, J=8.4, 1H), 7.50-7.45 (m, 2H), 7.43-7.38 (m, 2H), 7.34-7.29 (m, 1H), 5.43-5.35 (m, 1H), 1.72 (d, J=6.9, 3H). [M+H]⁺ 356/358. Rt 3.16 min (method N).

### Example 104

### (S)-8-methyl-N-(1-phenylethyl)-1,6-naphthyridine-2-carboxamide (62)

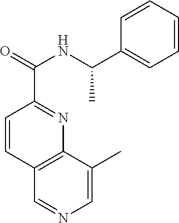 [SMILES: Cc1cncc2ccc(C(=O)N[C@@H](C)c3ccccc3)nc12]

(S)-8-bromo-N-(1-phenylethyl)-1,6-naphthyridine-2-carboxamide (Example 57) (20 mg, 0.056 mmol), trimethylboroxine (3.90 μL, 0.028 mmol) and Pd(dppf)Cl₂.CH₂Cl₂ (2.3 mg, 2.81 μmol) were loaded in a microwave vial and then degassed acetonitrile (970 μl) and sodium carbonate in water (157 μl, 0.079 mmol) were added. The reaction mixture was heated at 120° C. for 60 min and then concentrated. The crude was purified via biotage column chromatography (dichloromethane/EtOH 99/1 to 96/4) to give the title compound (12 mg, 74% yield). ¹H NMR (500 MHz, CDCl₃) ppm=9.22 (s, 1H), 8.69 (s, 1H), 8.54-8.49 (d, J=8.52, 1H), 8.45 (s, 2H), 7.48-7.45 (m, 2H), 7.40 (dd, J=8.5, 6.9, 2H), 7.33-7.29 (m, 1H), 5.45-5.36 (m, 1H), 2.78 (s, 3H), 1.71 (d, J=6.9, 3H). [M+H]⁺ 292. Rt 2.98 min (method N).

### Example 105

### (R)-8-methyl-N-(1-phenylethyl)-1,6-naphthyridine-2-carboxamide (36)

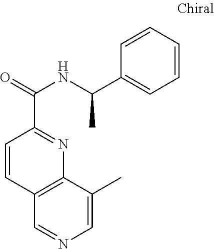 [SMILES: Cc1cncc2ccc(C(=O)N[C@H](C)c3ccccc3)nc12]

(R)-8-methyl-N-(1-phenylethyl)-1,6-naphthyridine-2-carboxamide was prepared in a manner similar to Example 59, using (R)-8-bromo-N-(1-phenylethyl)-1,6-naphthyridine-2-carboxamide (Example 58) as the starting material. ¹H NMR (500 MHz, CDCl₃) ppm=9.21 (s, 1H), 8.68 (d, J=1.1, 1H), 8.52 (d, J=9.0, 1H, NH), 8.44 (s, 2H), 7.49-7.44 (m, 2H), 7.42-7.37 (m, 2H), 7.33-7.28 (m, 1H), 5.43-5.36 (m, 1H), 2.77 (s, 3H), 1.71 (d, J=6.9, 3H). [M+H]⁺ 292. Rt 2.92 min (method P).

### Example 106

### (S)-8-(4-(1-methyl-1H-pyrazol-4-yl)phenyl)-N-(1-phenylethyl)-1,6-naphthyridine-2-carboxamide (27)

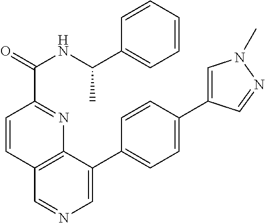 [SMILES: C[C@H](NC(=O)c1ccc2cncc(-c3ccc(-c4cnn(C)c4)cc3)c2n1)c1ccccc1]

(S)-8-bromo-N-(1-phenylethyl)-1,6-naphthyridine-2-carboxamide (Example 57) (18 mg, 0.051 mmol), 1-methyl-4-(4-(4,4,5,5-tetramethyl-1,3,2-dioxaborolan-2-yl)phenyl)-1H-pyrazole (16 mg, 0.056 mmol) and Pd(dppf)Cl₂.CH₂Cl₂ (2.1 mg, 2.53 μmol) were loaded in a microwave vial and then degassed acetonitrile (870 μL) and sodium carbonate in water (140 μL, 0.071 mmol) were added. The reaction mixture was heated at 120° C. for 60 min under microwave irradiation. The crude was concentrated and purified via biotage column chromatography (dichloromethane/EtOH 99/1 to 96/4), the product obtained was then filtered on a SCX-2 column and the title product was released with 1N NH₃ in methanol (12 mg, 55% yield). ¹H NMR (500 MHz, CDCl₃) ppm=9.35 (s, 1H), 8.93 (s, 1H), 8.55 (d, J=8.4, 1H), 8.47 (d, J=8.4, 1H), 8.43 (d, J=8.0, 1H), 7.87 (d, J=0.8, 1H), 7.77 (d, J=8.3, 2H), 7.72 (d, J=0.8, 1H), 7.58 (d, J=8.3, 2H), 7.38-7.28 (m, 5H), 5.30-5.27 (m, 1H), 4.03 (s, 3H), 1.59 (d, J=6.8, 3H). [M+H]⁺ 434. Rt 3.16 min (method N).

### Example 107

### (R)-8-(4-(1-methyl-1H-pyrazol-4-yl)phenyl)-N-(1-phenylethyl)-1,6-naphthyridine-2-carboxamide (24)

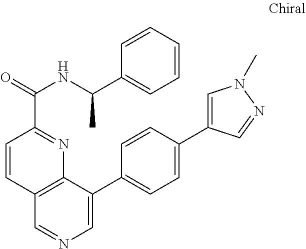 [SMILES: C[C@@H](NC(=O)c1ccc2cncc(-c3ccc(-c4cnn(C)c4)cc3)c2n1)c1ccccc1]

(R)-8-(4-(1-methyl-1H-pyrazol-4-yl)phenyl)-N-(1-phenylethyl)-1,6-naphthyridine-2-carboxamide was prepared in a manner similar to Example 61, using (R)-8-bromo-N-(1-phenylethyl)-1,6-naphthyridine-2-carboxamide (Example 58) as the starting material. ¹H NMR (500 MHz, CDCl₃) ppm=9.32 (s, 1H), 8.91 (s, 1H), 8.52 (d, J=8.5, 1H), 8.45 (d, J=8.5, 1H), 8.42 (d, J=8.2, 1H), 7.86 (d, J=0.8, 1H), 7.77-7.73 (m, 2H), 7.71 (d, J=0.8, 1H), 7.59-7.54 (m, 2H), 7.37-7.24 (m, 5H), 5.29-5.22 (m, 1H), 4.01 (s, 3H), 1.57 (d, J=6.8, 3H). [M+H]⁺ 434. Rt 3.12 min (method N).

### Example 108

### 8-(1-Methyl-2,2-dioxo-2,3-dihydro-1H-benzo[c]isothiazol-5-yl)-[1,6]naphthyridine-2-carboxylic acid methylamide (1)

### 8-Bromo-[1,6]naphthyridine-2-carboxylic acid methylamide

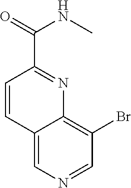

In a 12 mL screw-capped vessel 8-bromo-1,6-naphthyridine-2-carboxylic acid (1.00 g, 3.95 mmol) was treated with thionyl chloride (4.30 mL, 59.3 mmol). The yellow suspension was stirred at 70° C. for 3 h. The reaction mixture was evaporated and once more co-evaporated with toluene to dryness. To the residue, methylamine solution (2 M in tetrahydrofuran, 39.5 mL, 79.0 mmol) was added and the brown solution was stirred for 1 h at RT. The reaction mixture was allowed to stand at rt for 15 h. The mixture was evaporated to dryness. The brown residue was treated with 30 mL acetonitrile. The yellow precipitate was filtered, washed with a small amount of acetonitrile and dried on air overnight to yield in 1.20 g (86% purity, 98%) of the title compound as a yellow solid.

### 8-(1-Methyl-2,2-dioxo-2,3-dihydro-1H-benzo[c]isothiazol-5-yl)-[1,6]naphthyridine-2-carboxylic acid methylamide

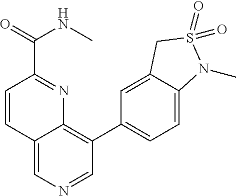 [SMILES: CNC(=O)c1ccc2cncc(-c3ccc4c(c3)CS(=O)(=O)N4C)c2n1]

To a solution of 8-bromo-[1,6]naphthyridine-2-carboxylic acid methylamide (86% purity, 80.0 mg, 0.26 mmol) in acetonitrile (10 mL) in a microwave vial was added 1-methyl-5-(4,4,5,5-tetramethyl-[1,3,2]dioxaborolan-2-yl)-1,3-dihydro-benzo[c]isothiazole 2,2-dioxide (94.8 mg, 0.31 mmol), sodium carbonate solution (0.5 M, 1.02 mL, 0.51 mmol) and Pd(dppf)Cl₂ dichloromethane complex (10.4 mg, 0.013 mmol). The closed vial was flushed with nitrogen twice and agitated under microwave irradiation at 120° C. for 1 h. The mixture was treated with ethyl acetate, filtered and evaporated to dryness. The brown residue was purified by flash chromatography (dichloromethane/methanol). The yellow solid was crystallized from diethyl ether/acetonitrile, filtered and washed with diethyl ether to yield in 39.1 mg (42%) of the title compound as a yellow solid. ¹H NMR (500 MHz, DMSO-d6) ppm=9.47 (s, 1H), 8.85 (s, 1H), 8.83 (d, J=8.5, 1H), 8.31 (d, J=8.4, 1H), 8.21-8.16 (m, 1H), 7.89-7.86 (m, 1H), 7.83 (dd, J=8.3, 1.9, 1H), 7.13 (d, J=8.3, 1H), 4.79 (s, 2H), 3.15 (s, 3H), 2.90 (d, J=4.9, 3H). [M+H]⁺ 369. Rt 2.04 min (method L).

### Example 109

### 8-(1-methyl-1H-indazol-5-yl)-[1,6]naphthyridine-2-carboxylic acid methylamide (6)

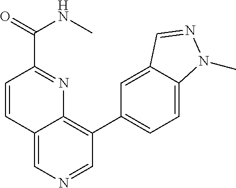 [SMILES: CNC(=O)c1ccc2cncc(-c3ccc4c(cnn4C)c3)c2n1]

8-(1-methyl-1H-indazol-5-yl)-[1,6]naphthyridine-2-carboxylic acid methylamide was prepared in a manner similar to Example 63, using 1-methylindazole-5-boronic acid as the starting material. ¹H NMR (400 MHz, DMSO-d6) ppm=9.49 (s, 1H), 8.91 (s, 1H), 8.85 (d, J=8.5, 1H), 8.30 (d, J=8.4, 1H), 8.20-8.17 (m, 1H), 8.17 (d, J=1.0, 1H), 8.13-8.07 (m, 1H), 7.89 (dd, J=8.8, 1.6, 1H), 7.81-7.77 (m, 1H), 4.13 (s, 3H), 2.86 (d, J=5.0, 3H). [M+H]⁺ 318. Rt 2.0 min (method L).

### Example 110

### 8-(1-methyl-1H-indazol-6-yl)-[1,6]naphthyridine-2-carboxylic acid methylamide (10)

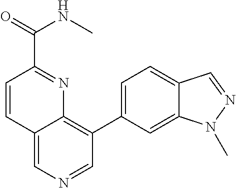 [SMILES: CNC(=O)c1ccc2cncc(-c3ccc4cnn(C)c4c3)c2n1]

8-(1-methyl-1H-indazol-6-yl)-[1,6]naphthyridine-2-carboxylic acid methylamide was prepared in a manner similar to Example 63, using 1-methylindazole-6-boronic acid as the starting material. ¹H NMR (400 MHz, DMSO-d6) ppm=9.53 (s, 1H), 8.99 (s, 1H), 8.87 (d, J=8.5, 1H), 8.32 (d, J=8.5, 1H), 8.20-8.09 (m, 3H), 7.91 (d, J=8.4, 1H), 7.61 (d, J=8.4, 1H), 4.12 (s, 3H), 2.87 (d, J=4.9, 3H). [M+H]⁺ 318. Rt 2.07 min (method L).

### Example 111

### 8-(1-methyl-2-oxo-2,3-dihydro-1H-indol-6-yl)-[1,6]naphthyridine-2-carboxylic acid methylamide (15)

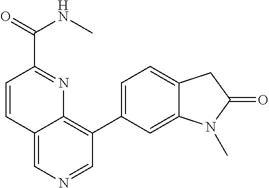 [SMILES: CNC(=O)c1ccc2cncc(-c3ccc4c(c3)N(C)C(=O)C4)c2n1]

8-(1-methyl-2-oxo-2,3-dihydro-1H-indol-6-yl)-[1,6]naphthyridine-2-carboxylic acid methylamide was prepared in a manner similar to Example 63, using 1-methyl-6-(4,4,5,5-tetramethyl-[1,3,2]dioxaborolan-2-yl)-1,3-dihydro-indol-2-one as the starting material. ¹H NMR (500 MHz, DMSO-d6) ppm=9.50 (s, 1H), 8.92 (s, 1H), 8.85 (d, J=8.5, 1H), 8.32 (d, J=8.5, 1H), 8.22-8.17 (m, 1H), 7.50 (d, J=1.5, 1H), 7.48 (dd, J=7.5, 1.5, 1H), 7.45 (d, J=7.6, 1H), 3.65 (s, 2H), 3.19 (s, 3H), 2.89 (d, J=4.9, 3H). [M+H]⁺ 333. Rt 1.93 min (method L).

### Example 112

### 8-(2,2-dioxo-2,3-dihydro-1H-benzo[c]isothiazol-5-yl)-[1,6]naphthyridine-2-carboxylic acid methylamide (26)

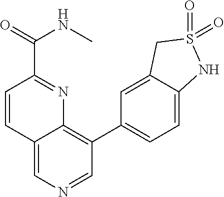 [SMILES: CNC(=O)c1ccc2cncc(-c3ccc4c(c3)CS(=O)(=O)N4)c2n1]

8-(2,2-dioxo-2,3-dihydro-1H-benzo[c]isothiazol-5-yl)-[1,6]naphthyridine-2-carboxylic acid methylamide was prepared in a manner similar to Example 63, using 5-(4,4,5,5-tetramethyl-[1,3,2]dioxaborolan-2-yl)-1,3-dihydro-benzo[c]isothiazole 2,2-dioxide as the starting material. ¹H NMR (400 MHz, DMSO-d6) ppm=10.71 (s, 1H), 9.47 (s, 1H), 8.86-8.82 (m, 2H), 8.31 (d, J=8.5, 1H), 8.22-8.15 (m, 1H), 7.81 (d, J=1.8, 1H), 7.74 (dd, J=8.2, 2.0, 1H), 7.01 (d, J=8.2, 1H), 4.66 (s, 2H), 2.90 (d, J=4.9, 3H). [M+H]⁺ 355. Rt 1.82 min (method L).

### Example 113

### 8-(1-Methyl-2,2-dioxo-2,3-dihydro-1H-benzo[c]isothiazol-5-yl)-[1,6]naphthyridine-2-carboxylic acid (1-methyl-cyclopropyl)-amide (8)

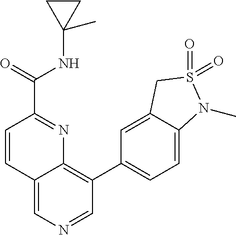 [SMILES: CN1c2ccc(-c3cncc4ccc(C(=O)NC5(C)CC5)nc34)cc2CS1(=O)=O]

8-(1-Methyl-2,2-dioxo-2,3-dihydro-1H-benzo[c]isothiazol-5-yl)-[1,6]naphthyridine-2-carboxylic acid (1-methyl-cyclopropyl)-amide was prepared in a manner similar to Example 63, using 1-methylcyclopropylamine and 1-methyl-5-(4,4,5,5-tetramethyl-[1,3,2]dioxaborolan-2-yl)-1,3-dihydro-benzo[c]isothiazole 2,2-dioxide as the starting materials. ¹H NMR (400 MHz, DMSO-d6) ppm=9.47 (s, 1H), 8.88 (s, 1H), 8.83 (d, J=8.5, 1H), 8.32 (s, 1H), 8.27 (d, J=8.4, 1H), 7.95-7.88 (m, 1H), 7.85 (dd, J=8.2, 1.9, 1H), 7.14 (d, J=8.3, 1H), 4.73 (s, 2H), 3.15 (s, 3H), 1.41 (s, 3H), 0.83-0.76 (m, 2H), 0.73-0.66 (m, 2H). [M+H]⁺ 409. Rt 2.3 min (method L).

### Example 114

### 8-(1-Methyl-2,2-dioxo-2,3-dihydro-1H-benzo[c]isothiazol-5-yl)-[1,6]naphthyridine-2-carboxylic acid cyclopropylamide (21)

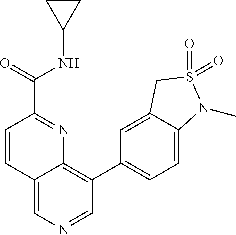 [SMILES: CN1c2ccc(-c3cncc4ccc(C(=O)NC5CC5)nc34)cc2CS1(=O)=O]

8-(1-Methyl-2,2-dioxo-2,3-dihydro-1H-benzo[c]isothiazol-5-yl)-[1,6]naphthyridine-2-carboxylic acid cyclopropylamide was prepared in a manner similar to Example 63, using cyclopropylamine and 1-methyl-5-(4,4,5,5-tetramethyl-[1,3,2]dioxaborolan-2-yl)-1,3-dihydro-benzo[c]isothiazole 2,2-dioxide as the starting materials. ¹H NMR (400 MHz, DMSO-d6) ppm=9.47 (s, 1H), 8.87 (s, 1H), 8.84 (d, J=8.4, 1H), 8.26 (d, J=8.5, 1H), 8.15 (d, J=4.5, 1H), 7.94-7.89 (m, 1H), 7.84 (dd, J=8.2, 1.9, 1H), 7.13 (d, J=8.3, 1H), 4.72 (s, 2H), 3.14 (s, 3H), 2.94-2.86 (m, 1H), 0.82-0.75 (m, 2H), 0.64-0.57 (m, 2H). [M+H]⁺ 395. Rt 2.18 min (method L).

### Example 115

### 8-(2,2-Dioxo-2,3-dihydro-1H-benzo[c]isothiazol-5-yl)-[1,6]naphthyridine-2-carboxylic acid (2-hydroxy-ethyl)-methyl-amide (45)

### 8-Bromo-[1,6]naphthyridine-2-carboxylic acid (2-hydroxy-ethyl)-methyl-amide

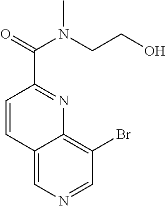 [SMILES: CN(CCO)C(=O)c1ccc2cncc(Br)c2n1]

In a 12 mL screw-capped vessel 8-bromo-1,6-naphthyridine-2-carboxylic acid (100 mg, 0.40 mmol) was dissolved in N,N-dimethylformamide (2 mL). 4-Methylmorpholine (130 μl, 1.19 mmol) was added to obtain a yellow solution. [(Benzotriazol-1-yloxy)-dimethylamino-methylene]-dimethyl-ammonium tetrafluoroborate (TBTU, 254 mg, 0.79 mmol) and 2-(methylamino)ethanol (35.6 mg, 0.47 mmol) were added and the yellow reaction solution was stirred for 2 h at 60° C. The reaction solution was treated with 20 mL of water to obtain a yellow solution. Solid sodium carbonate was added, but no precipitate was formed. Solid sodium chloride was added and the solution was extracted with dichloromethane twice. The combined organic layer was dried and evaporated to dryness. The residue was purified by flash chromatography (n-heptan/dichloromethane/methanol). The yellow solid was crystallized from diethyl ether. The solid was filtered, washed with ether and dried on air to obtain 73.0 mg (57%) of the tile compound as a white solid.

### 8-(2,2-Dioxo-2,3-dihydro-1H-benzo[c]isothiazol-5-yl)-[1,6]naphthyridine-2-carboxylic acid (2-hydroxy-ethyl)-methyl-amide

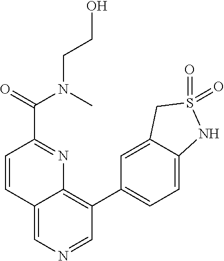 [SMILES: CN(CCO)C(=O)c1ccc2cncc(-c3ccc4c(c3)CS(=O)(=O)N4)c2n1]

To a solution of 8-bromo-[1,6]naphthyridine-2-carboxylic acid (2-hydroxy-ethyl)-methyl-amide (73.0 mg, 0.20 mmol) in acetonitrile (10 mL) in a microwave vial were added 5-(4,4,5,5-tetramethyl-[1,3,2]dioxaborolan-2-yl)-1,3-dihydro-benzo[c]isothiazole 2,2-dioxide (105 mg, 0.30 mmol), sodium carbonate solution (0.5 M, 0.81 mL, 0.41 mmol) and Pd(dppf)Cl₂ dichloromethane complex (8.27 mg, 0.01 mmol). The vial was flushed with nitrogen twice and heated at 120° C. under microwave irradiation for 1 h. The reaction mixture was treated with ethyl acetate, filtered and the filtrate was evaporated to dryness. The brown residue was purified by flash chromatography (dichloromethane/methanol). The yellow solid was dissolved in 1 mL of 1 N HCl solution, 1 mL water was added and the solution was freeze-dried overnight to obtain 15.0 mg (12%) of the HCl salt of the title compound as a white solid. ¹H NMR (500 MHz, DMSO-d6) ppm=10.75 (s, 1H), 9.52 (s, 1H), 8.90 (d, J=8.5, 1H), 8.88 (s, 1H), 8.69-8.57 (m, 2H), 8.39 (d, J=8.5, 1H), 7.84-7.78 (m, 1H), 7.73 (dd, J=8.2, 1.9, 1H), 6.99 (d, J=8.2, 1H), 4.65-4.59 (m, 4H), 3.42-3.35 (m, 2H), 2.65-2.61 (m, 3H). [M+H]⁺ 399. Rt 1.7 min (method L).

### Example 116

### 3,3-difluoro-pyrrolidin-1-yl)-[8-(2,2-dioxo-2,3-dihydro-1H-benzo[c]isothiazol-5-yl)-[1,6]naphthyridin-2-yl]-methanone (30)

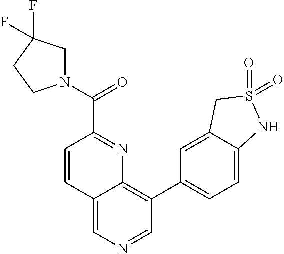 [SMILES: O=C(c1ccc2cncc(-c3ccc4c(c3)CS(=O)(=O)N4)c2n1)N1CCC(F)(F)C1]

(3,3-difluoro-pyrrolidin-1-yl)-[8-(2,2-dioxo-2,3-dihydro-1H-benzo[c]isothiazol-5-yl)-[1,6]naphthyridin-2-yl]-methanone was prepared in a manner similar to Example 63, using 3,3-difluoropyrrolidine and 5-(4,4,5,5-tetramethyl-[1,3,2]dioxaborolan-2-yl)-1,3-dihydro-benzo[c]isothiazole 2,2-dioxide as the starting material. ¹H NMR (400 MHz, DMSO-d6) ppm=10.73 (s, 1H), 9.47 (s, 1H), 8.86-8.75 (m, 2H), 8.18 (d, J=8.5, 1H), 7.68-7.65 (m, 1H), 7.63-7.57 (m, 1H), 6.95 (d, J=8.1, 1H), 4.56 (s, 2H), 4.23 (t, J=13.1, 2H), 3.78 (t, J=7.6, 2H), 2.50-2.39 (m, 2H). [M+H]⁺ 431. Rt 2.05 min (method L).

### Example 117

### 8-(2,2-Dioxo-2,3-dihydro-1H-benzo[c]isothiazol-5-yl)-[1,6]naphthyridine-2-carboxylic acid dimethylamide (46)

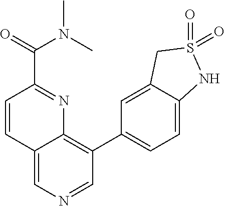 [SMILES: CN(C)C(=O)c1ccc2cncc(-c3ccc4c(c3)CS(=O)(=O)N4)c2n1]

8-(2,2-Dioxo-2,3-dihydro-1H-benzo[c]isothiazol-5-yl)-[1,6]naphthyridine-2-carboxylic acid dimethylamide was prepared in a manner similar to Example 63, using dimethylamine and 5-(4,4,5,5-tetramethyl-[1,3,2]dioxaborolan-2-yl)-1,3-dihydro-benzo[c]isothiazole 2,2-dioxide as the starting material. [M+H]⁺ 369. Rt 1.71 min (method L).

### Example 118

### 8-(1-Methyl-2,2-dioxo-2,3-dihydro-1H-benzo[c]isothiazol-5-yl)-[1,6]naphthyridine-2-carboxylic acid (2-amino-ethyl)-amide (16)

### {2-[(8-Bromo-[1,6]naphthyridine-2-carbonyl)-amino]-ethyl}-carbamic acid tert-butyl ester

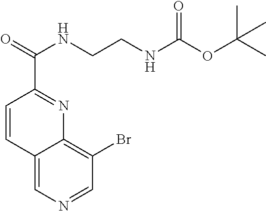 [SMILES: CC(C)(C)OC(=O)NCCNC(=O)c1ccc2cncc(Br)c2n1]

In a 12 mL screw-capped vessel 8-bromo-1,6-naphthyridine-2-carboxylic acid (300 mg, 1.19 mmol) was suspended in N,N-dimethylformamide (6 mL). 4-Methylmorpholine (587 μl, 5.34 mmol), [(Benzotriazol-1-yloxy)-dimethylamino-methylene]-dimethyl-ammonium tetrafluoroborate (TBTU, 761 mg, 2.37 mmol) and N-Boc-ethylenediamine (98% purity, 190 mg, 1.19 mmol) were added and the yellow reaction solution was stirred for 3 h at 50° C. The mixture was treated with 100 mL of water and stirred overnight. The precipitate was filtered off, washed with water and dried overnight in air to yield in 258 mg (93% purity, 51%) of the title compound as a beige solid.

### (2-{[8-(1-Methyl-2,2-dioxo-2,3-dihydro-1H-benzo[c]isothiazol-5-yl)-[1,6]naphthyridine-2-carbonyl]-amino}-ethyl)-carbamic acid tert-butyl ester

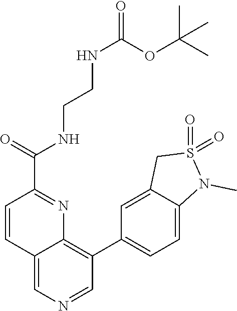 [SMILES: CN1c2ccc(-c3cncc4ccc(C(=O)NCCNC(=O)OC(C)(C)C)nc34)cc2CS1(=O)=O]

To a solution of {2-[(8-Bromo-[1,6]naphthyridine-2-carbonyl)-amino]-ethyl}-carbamic acid tert-butyl ester (93% purity, 100 mg, 0.24 mmol) in acetonitrile (10 mL) in a microwave vial were added 1-methyl-5-(4,4,5,5-tetramethyl-[1,3,2]dioxaborolan-2-yl)-1,3-dihydro-benzo[c]isothiazole 2,2-dioxide (87.3 mg, 0.28 mmol), sodium carbonate solution (0.5 M, 0.94 mL, 0.47 mmol) and Pd(dppf)Cl₂ dichloromethane complex (9.61 mg, 0.01 mmol). The vial was flushed with nitrogen twice and heated at 120° C. under microwave irradiation for 1 h. The reaction mixture was treated with ethyl acetate, filtered and the filtrate was evaporated to dryness. The brown residue was purified by flash chromatography (dichloromethane/methanol) to yield in 124 mg (86% purity, 91%) of the title compound as a yellow resin.

### 8-(1-Methyl-2,2-dioxo-2,3-dihydro-1H-benzo[c]isothiazol-5-yl)-[1,6]naphthyridine-2-carboxylic acid (2-amino-ethyl)-amide

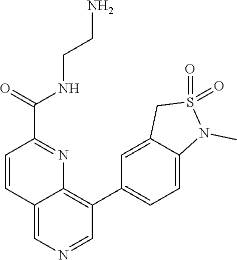 [SMILES: CN1c2ccc(-c3cncc4ccc(C(=O)NCCN)nc34)cc2CS1(=O)=O]

In a 25 ml-roundbottom flask (2-{[8-(1-Methyl-2,2-dioxo-2,3-dihydro-1H-benzo[c]isothiazol-5-yl)-[1,6]naphthyridine-2-carbonyl]-amino}-ethyl)-carbamic acid tert-butyl ester (86% purity, 124 mg, 0.21 mmol) was dissolved in methanol (3 mL) and treated with 4 N HCl solution in dioxane (1.07 mL, 4.29 mmol). The yellow solution was stirred at rt for 3 h to obtain an orange precipitate. To the mixture 2 mL of dioxane was added. The precipitate was filtered off, washed with diethyl ether and dried in air overnight to yield in 55.9 mg (60%) of the HCl salt of the title compound as an orange solid. ¹H NMR (400 MHz, DMSO-d6) ppm=9.53 (s, 1H), 8.92 (s, 1H), 8.89 (d, J=8.5, 1H), 8.46 (t, J=6.1, 1H), 8.35 (d, J=8.5, 1H), 8.08-8.02 (m, 1H), 8.02-7.92 (m, 3H), 7.89 (dd, J=8.3, 2.0, 1H), 7.15 (d, J=8.3, 1H), 4.80 (s, 2H), 3.64 (q, J=6.1, 2H), 3.15 (s, 3H), 3.10-3.00 (m, 2H). [M+H]⁺ 398. Rt 1.77 min (method L).

### Example 119

### [8-(1-Methyl-2,2-dioxo-2,3-dihydro-1H-benzo[c]isothiazol-5-yl)-[1,6]naphthyridin-2-yl]-piperazin-1-yl-methanone (18)

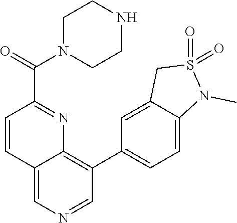 [SMILES: CN1c2ccc(-c3cncc4ccc(C(=O)N5CCNCC5)nc34)cc2CS1(=O)=O]

[8-(1-Methyl-2,2-dioxo-2,3-dihydro-1H-benzo[c]isothiazol-5-yl)-[1,6]naphthyridin-2-yl]-piperazin-1-yl-methanone was prepared in a manner similar to Example 73, using tert-butyl piperazine-1-carboxylate as the starting material. ¹H NMR (400 MHz, DMSO-d6) ppm=9.43 (s, 1H), 8.80 (s, 1H), 8.77 (d, J=8.5, 1H), 7.88 (d, J=8.4, 1H), 7.73-7.69 (m, 2H), 7.10 (d, J=8.7, 1H), 4.74 (s, 2H), 3.66-3.60 (m, 2H), 3.50-3.44 (m, 2H), 3.18-3.15 (m, 1H), 3.13 (s, 3H), 2.86-2.80 (m, 2H), 2.74-2.68 (m, 2H). [M+H]⁺ 424. Rt 1.69 min (method L).

### Example 120

### (8-(4-(1-(2-hydroxy-2-methylpropyl)-1H-pyrazol-4-yl)phenyl)-1,6-naphthyridin-2-yl)(3-methoxyazetidin-1-yl)methanone (31)

### (8-bromo-1,6-naphthyridin-2-yl)(3-methoxyazetidin-1-yl)methanone

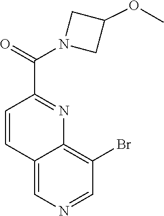 [SMILES: COC1CN(C(=O)c2ccc3cncc(Br)c3n2)C1]

To a suspension of 8-bromo-1,6-naphthyridine-2-carboxylic acid (478 mg, 1.89 mmol) in DMF (9 mL) was added HATU (815 mg, 2.14 mmol) and the mixture stirred for 5 min before the addition of 3-methoxyazetidine.HCl (500 mg, 4.05 mmol) and DIPEA (2.0 mL, 11.48 mmol). The resulting mixture was then stirred at rt for 36 h and concentrated in vacuo. The crude material was purified by Biotage (SNAP 50 g column, EtOAc, then CH₂Cl₂/EtOH 90/10) to give a pale yellow solid (214 mg, 40%).

### (8-(4-(1-(2-hydroxy-2-methylpropyl)-1H-pyrazol-4-yl)phenyl)-1,6-naphthyridin-2-yl) (3-methoxyazetidin-1-yl)methanone

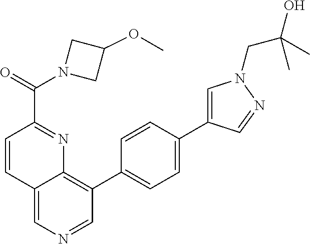 [SMILES: COC1CN(C(=O)c2ccc3cncc(-c4ccc(-c5cnn(CC(C)(C)O)c5)cc4)c3n2)C1]

A mixture of (8-bromo-1,6-naphthyridin-2-yl)(3-methoxyazetidin-1-yl)methanone (43 mg, 0.13 mmol), 2-methyl-1-(4-(4-(4,4,5,5-tetramethyl-1,3,2-dioxaborolan-2-yl)phenyl)-1H-pyrazol-1-yl)propan-2-ol (70 mg, 0.21 mmol), K₃PO₄ (125 mg, 0.59 mmol), and Pd(dtbpf)Cl₂ (10 mg, 0.015 mmol) in a mixture of 1,4-dioxane (0.6 mL) and water (0.2 mL) was stirred at 120° C. for 1 h under microwave irradiation. The reaction mixture was concentrated in vacuo and purified by Biotage (SNAP 10 g column, cyclohexane/EtOAc 90/10->50/50, then CH₂Cl₂/EtOH 97/3->80/20) to give a yellow solid which was taken up in CH₂Cl₂ and filtered. The filtrate was concentrated in vacuo to give the title compound as a pale yellow solid (22 mg, 36%). ¹H NMR (500 MHz, CDCl₃) ppm=9.33 (s, 1H), 8.83 (s, 1H), 8.48 (d, J=8.5, 1H), 8.40 (d, J=8.6, 1H), 7.90 (s, 1H), 7.79 (s, 1H), 7.66 (s, 4H), 4.51 (ddd, J=11.9, 6.1, 1.7, 1H), 4.36 (ddd, J=11.2, 6.2, 1.7, 1H), 4.30 (ddd, J=11.7, 4.0, 1.7, 1H), 4.15 (s, 2H), 4.13-4.05 (m, 2H), 3.81 (br s, 1H), 3.17 (s, 3H), 1.25 (s, 3H), 1.24 (s, 3H). [M+H]⁺ 458. Rt 1.25 min (method O).

### Example 121

### (3-methoxyazetidin-1-yl)(8-(4-(1-methyl-3-(trifluoromethyl)-1H-pyrazol-4-yl)phenyl)-1,6-naphthyridin-2-yl)methanone (49)

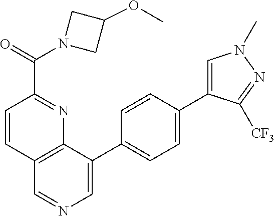 [SMILES: COC1CN(C(=O)c2ccc3cncc(-c4ccc(-c5cn(C)nc5C(F)(F)F)cc4)c3n2)C1]

(3-methoxyazetidin-1-yl) (8-(4-(1-methyl-3-(trifluoromethyl)-1H-pyrazol-4-yl)phenyl)-1,6-naphthyridin-2-yl)methanone was prepared in a manner similar to Example 75, using 1-methyl-4-(4-(4,4,5,5-tetramethyl-1,3,2-dioxaborolan-2-yl)phenyl)-3-(trifluoromethyl)-1H-pyrazole as the starting material. ¹H NMR (500 MHz, CDCl₃) ppm=9.36 (s, 1H), 8.86 (s, 1H), 8.49 (d, J=8.5, 1H), 8.42 (d, J=8.5, 1H), 7.67 (d, J=8.2, 2H), 7.60 (s, 1H), 7.56 (d, J=7.9, 2H), 4.54 (ddd, J=11.8, 6.2, 1.6, 1H), 4.36 (ddd, J=11.1, 6.2, 1.8, 1H), 4.24 (ddd, J=11.8, 3.9, 1.7, 1H), 4.12 (m, 1H), 4.08 (m, 1H), 4.05 (s, 3H), 3.21 (s, 3H). [M+H]⁺ 468. Rt 1.36 min (method O).

### Example 122

### (3-methoxyazetidin-1-yl)(8-(4-(1-methyl-3-(trifluoromethyl)-1H-pyrazol-5-yl)phenyl)-1,6-naphthyridin-2-yl)methanone (57)

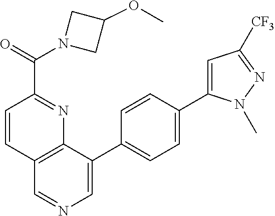 [SMILES: COC1CN(C(=O)c2ccc3cncc(-c4ccc(-c5cc(C(F)(F)F)nn5C)cc4)c3n2)C1]

(3-methoxyazetidin-1-yl)(8-(4-(1-methyl-3-(trifluoromethyl)-1H-pyrazol-5-yl)phenyl)-1,6-naphthyridin-2-yl)methanone was prepared in a manner similar to Example 75, using 1-methyl-5-(4-(4,4,5,5-tetramethyl-1,3,2-dioxaborolan-2-yl)phenyl)-3-(trifluoromethyl)-1H-pyrazole as the starting material. ¹H NMR (500 MHz, CDCl₃) ppm=9.39 (s, 1H), 8.88 (s, 1H), 8.52 (d, J=8.5, 1H), 8.42 (d, J=8.5, 1H), 7.78 (d, J=8.1, 2H), 7.60 (d, J=8.1, 2H), 6.65 (s, 1H), 4.49 (ddd, J=11.5, 6.3, 1.7, 1H), 4.36 (ddd, J=11.1, 6.1, 1.7, 1H), 4.26 (ddd, J=11.5, 4.1, 1.7, 1H), 4.13 (tt, J=6.2, 4.1, 1H), 4.08 (ddd, J=11.1, 4.0, 1.6, 1H), 4.02 (s, 3H), 3.24 (s, 3H). [M+H]⁺ 468. Rt 1.41 min (method O).

### Example 123

### (8-(1-(2-hydroxy-2-methylpropyl)-1H-indazol-5-yl)-1,6-naphthyridin-2-yl)(3-methoxyazetidin-1-yl)methanone (52)

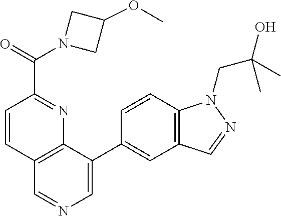 [SMILES: COC1CN(C(=O)c2ccc3cncc(-c4ccc5c(cnn5CC(C)(C)O)c4)c3n2)C1]

(8-(1-(2-hydroxy-2-methylpropyl)-1H-indazol-5-yl)-1,6-naphthyridin-2-yl) (3-methoxyazetidin-1-yl)methanone was prepared in a manner similar to Example 75, using 2-methyl-1-(5-(4,4,5,5-tetramethyl-1,3,2-dioxaborolan-2-yl)-1H-indazol-1-yl)propan-2-ol as the starting material. ¹H NMR (500 MHz, CDCl₃) ppm=9.34 (s, 1H), 8.86 (s, 1H), 8.49 (d, J=8.5, 1H), 8.40 (d, J=8.5, 1H), 8.15 (s, 1H), 7.98 (s, 1H), 7.67 (dd, J=8.8, 1.5, 1H), 7.63 (d, J=8.7, 1H), 4.42 (s, 2H), 4.38-4.28 (m, 2H), 4.15-4.10 (m, 1H), 4.06-3.98 (m, 2H), 3.67 (br s, 1H), 3.12 (s, 3H), 1.27 (d, J=8.7, 6H). [M+H]⁺ 432. Rt 1.2 min (method O).

### Example 124

### (8-(2-(2-hydroxy-2-methylpropyl)-2H-indazol-5-yl)-1,6-naphthyridin-2-yl)(3-methoxyazetidin-1-yl)methanone (33)

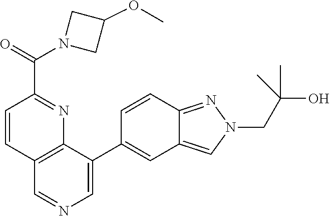 [SMILES: COC1CN(C(=O)c2ccc3cncc(-c4ccc5nn(CC(C)(C)O)cc5c4)c3n2)C1]

(8-(2-(2-hydroxy-2-methylpropyl)-2H-indazol-5-yl)-1,6-naphthyridin-2-yl) (3-methoxyazetidin-1-yl)methanone was prepared in a manner similar to Example 75, using 2-methyl-1-(5-(4,4,5,5-tetramethyl-1,3,2-dioxaborolan-2-yl)-2H-indazol-2-yl)propan-2-ol as the starting material. ¹H NMR (500 MHz, CDCl₃) ppm=9.34 (s, 1H), 8.88 (s, 1H), 8.49 (d, J=8.6, 1H), 8.41 (d, J=8.5, 1H), 8.10 (s, 1H), 7.91 (s, 1H), 7.83 (d, J=8.8, 1H), 7.61 (dd, J=8.9, 1.6, 1H), 4.47 (ddd, J=11.7, 5.8, 1.4, 1H), 4.43 (s, 2H), 4.33 (ddd, J=12.4, 7.6, 1.9, 1H), 4.23 (ddd, J=11.9, 4.0, 2.0, 1H), 4.07-4.01 (m, 2H), 3.13 (s, 3H), 2.08 (br s, 1H), 1.25 (s, 3H), 1.24 (s, 3H). [M+H]⁺ 432. Rt 1.16 min (method O).

### Example 125

### (3-methoxyazetidin-1-yl)(8-(1-methyl-1H-indazol-5-yl)-1,6-naphthyridin-2-yl)methanone (22)

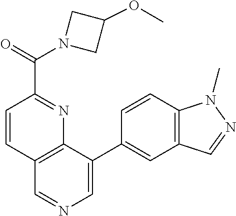 [SMILES: COC1CN(C(=O)c2ccc3cncc(-c4ccc5c(cnn5C)c4)c3n2)C1]

(3-methoxyazetidin-1-yl)(8-(1-methyl-1H-indazol-5-yl)-1,6-naphthyridin-2-yl)methanone was prepared in a manner similar to Example 75, using 1-methyl-1H-indazole-5-boronic acid as the starting material. ¹H NMR (500 MHz, CDCl₃) ppm=9.35 (s, 1H), 8.89 (s, 1H), 8.50 (d, J=8.5, 1H), 8.42 (d, J=8.5, 1H), 8.10 (d, J=1.0, 1H), 8.02 (s, 1H), 7.71 (dd, J=8.6, 1.6, 1H), 7.56 (d, J=8.7, 1H), 4.41 (m, 1H), 4.34 (m, 1H), 4.21-4.16 (m, 4H), 4.09-4.01 (m, 2H), 3.12 (s, 3H). [M+H]⁺ 374. Rt 1.21 min (method O).

### Example 126

### (5-amino-8-(1-methyl-1H-indazol-5-yl)-1,6-naphthyridin-2-yl)(3-methoxyazetidin-1-yl)methanone (140)

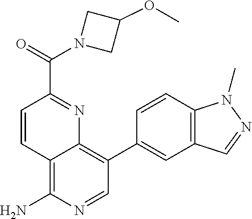 [SMILES: COC1CN(C(=O)c2ccc3c(N)ncc(-c4ccc5c(cnn5C)c4)c3n2)C1]

To a solution of (3-methoxyazetidin-1-yl) (8-(1-methyl-1H-indazol-5-yl)-1,6-naphthyridin-2-yl)methanone (19 mg, 0.051 mmol) in CH₂Cl₂ (0.4 mL) was added peracetic acid (39% in AcOH; 40 mg, 0.21 mmol) and the mixture heated at 40° C. for 2 h. The reaction mixture was then allowed to cool to rt and quenched with 1 M NaOH (5 mL), extracted with CH₂Cl₂ (3×5 mL). The combined organic layers were filtered through a phase separator and concentrated in vacuo. The resulting residue was taken up in pyridine (0.6 mL) and p-toluenesulfonyl chloride (16 mg, 0.084 mmol) was added, and the resulting mixture stirred at rt for 45 min. Ethanolamine (0.10 mL, 1.65 mmol) was then added and the mixture stirred at rt for 1 h. Water (10 mL) was then added and the mixture extracted with EtOAc (4×10 mL). The combined organic layers were washed with water (10 mL) and brine (10 mL), dried (MgSO₄), filtered and concentrated in vacuo. The resulting residue was purified by Biotage column chromatography (SNAP 10 g column, CH₂Cl₂/EtOH 100/0->85/15) to give the title compound as a yellow solid (7 mg, 35%). ¹H NMR (500 MHz, CD₂C12) ppm=8.38 (d, J=8.7 Hz, 1H), 8.26 (s, 1H), 8.23 (d, J=8.6 Hz, 1H), 8.03 (s, 1H), 7.90 (s, 1H), 7.64 (dd, J=8.6, 1.6 Hz, 1H), 7.53 (d, J=8.5 Hz, 1H), 5.49 (br s, 2H), 4.34 (ddd, J=11.4, 6.1, 1.7 Hz, 1H), 4.27 (ddd, J=11.1, 6.4, 1.7 Hz, 1H), 4.13 (s, 3H), 4.07 (ddd, J=11.3, 4.0, 1.6 Hz, 1H), 4.02 (tt, J=6.1, 4.0 Hz, 1H), 3.95 (ddd, J=11.1, 3.8, 1.7 Hz, 1H), 3.08 (s, 3H). [M+H]⁺ 389, Rt 0.86 min (method O).

### Example 127

### (8-(4-(1-isopropyl-1H-pyrazol-4-yl)phenyl)-1,6-naphthyridin-2-yl)(3-methoxyazetidin-1-yl)methanone (153)

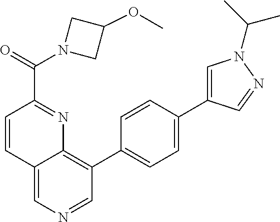 [SMILES: COC1CN(C(=O)c2ccc3cncc(-c4ccc(-c5cnn(C(C)C)c5)cc4)c3n2)C1]

A mixture of (8-bromo-1,6-naphthyridin-2-yl)(3-methoxyazetidin-1-yl)methanone (152 mg, 0.38 mmol), 1-isopropyl-4-(4-(4,4,5,5-tetramethyl-1,3,2-dioxaborolan-2-yl)phenyl)-1H-pyrazole (167 mg, 0.54 mmol), K₃PO₄ (240 mg, 1.13 mmol) and Pd(dtbpf)Cl₂ (25 mg, 0.038 mmol) in a mixture of 1,4-dioxane (1.5 mL) and water (0.4 mL) was stirred at 120° C. for 1 h under microwave irradiation. The mixture was then concentrated in vacuo and the residue purified by Biotage column chromatography (SNAP 25 g column, CH₂Cl₂/EtOH 100/0->85/15) to give the title compound as an orange oil (127 mg, 79%). ¹H NMR (500 MHz, CDCl₃) ppm=9.31 (s, 1H), 8.83 (s, 1H), 8.46 (d, J=8.6 Hz, 1H), 8.38 (d, J=8.6 Hz, 1H), 7.85 (s, 1H), 7.76 (s, 1H), 7.64 (s, 4H), 4.57 (hept, J=6.7 Hz, 1H), 4.51 (ddd, J=11.8, 6.1, 1.6 Hz, 1H), 4.35 (ddd, J=11.1, 6.2, 1.8 Hz, 1H), 4.30 (ddd, J=11.8, 4.0, 1.7 Hz, 1H), 4.13-4.03 (m, 2H), 3.17 (s, 3H), 1.58 (d, J=6.7 Hz, 6H). [M+H]⁺ 428, Rt 1.43 min (method O).

### Example 128

### (5-amino-8-(4-(1-isopropyl-1H-pyrazol-4-yl)phenyl)-1,6-naphthyridin-2-yl)(3-methoxyazetidin-1-yl)methanone (161)

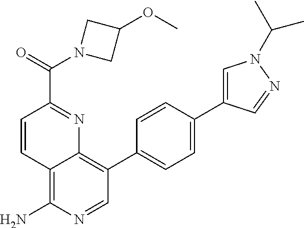 [SMILES: COC1CN(C(=O)c2ccc3c(N)ncc(-c4ccc(-c5cnn(C(C)C)c5)cc4)c3n2)C1]

To a solution of (8-(4-(1-isopropyl-1H-pyrazol-4-yl)phenyl)-1,6-naphthyridin-2-yl)(3-methoxyazetidin-1-yl)methanone (115 mg, 0.27 mmol) in CH₂Cl₂ (1.4 mL) was added peracetic acid (39% in AcOH; 0.18 mL, 1.06 mmol) and the mixture heated at 40° C. for 21 h. Additional peracetic acid (39% in AcOH; 0.05 mL, 0.30 mmol) was added at this point and the mixture heated at 40° C. for an additional 2 h. The mixture was then allowed to cool to rt and quenched with 1 M NaOH (10 mL). The aqueous layer was extracted with CH₂Cl₂ (3×15 mL) and the combined organic layers filtered through a phase separator and concentrated in vacuo. The residue was taken up in pyridine (1.5 mL) and p-toluenesulfonyl chloride (80 mg, 0.42 mmol) was added and the resulting mixture was stirred at rt for 45 min. Ethanolamine (0.45 mL, 7.44 mmol) was then added and the mixture stirred at rt for 1 h 45 min. Water (10 mL) was added and the mixture extracted with EtOAc (3×25 mL). The combined organic layers were washed with water (10 mL) and brine (10 mL), dried (MgSO₄), filtered and concentrated in vacuo. The residue was purified by Biotage column chromatography (SNAP 10 g column, cyclohexane/EtOAc 100/0->0/100, then CH₂Cl₂/EtOH 85/15->75/25) to give the title compound as a yellow solid (17 mg, 14%). ¹H NMR (500 MHz, CDCl₃) ppm=8.36 (d, J=8.7 Hz, 1H), 8.22 (d, J=8.7 Hz, 1H), 8.19 (s, 1H), 7.82 (s, 1H), 7.73 (s, 1H), 7.58 (d, J=8.2 Hz, 2H), 7.53 (d, J=8.2 Hz, 2H), 5.66 (br s, 2H), 4.56 (hept, J=6.7 Hz, 1H), 4.47-4.41 (m, 1H), 4.35-4.30 (m, 1H), 4.27-4.22 (m, 1H), 4.10-4.02 (m, 2H), 3.14 (s, 3H), 1.58 (d, J=6.7 Hz, 6H). [M+H]⁺ 443, Rt 1.01 min (method O).

### Example 129

### (3-methoxyazetidin-1-yl)(8-(4-(1-methyl-1H-pyrazol-3-yl)phenyl)-1,6-naphthyridin-2-yl)methanone (156)

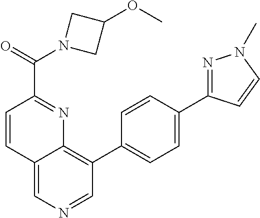 [SMILES: COC1CN(C(=O)c2ccc3cncc(-c4ccc(-c5ccn(C)n5)cc4)c3n2)C1]

A mixture of (8-bromo-1,6-naphthyridin-2-yl)(3-methoxyazetidin-1-yl)methanone (49 mg, 0.12 mmol), 1-methyl-3-(4-(4,4,5,5-tetramethyl-1,3,2-dioxaborolan-2-yl)phenyl)-1H-pyrazole (45 mg, 0.16 mmol), K₃PO₄ (98 mg, 0.46 mmol) and Pd(dtbpf)Cl₂ (11 mg, 0.017 mmol) in a mixture of 1,4-dioxane (0.7 mL) and water (0.15 mL) was stirred at 120° C. for 1 h under microwave irradiation. The mixture was then concentrated in vacuo and the residue purified by Biotage column chromatography (SNAP 10 g column, cyclohexane/EtOAc 100/0->70/30) to give the title compound as an orange resin (40 mg, 82%). ¹H NMR (500 MHz, CDCl₃) ppm=9.31 (s, 1H), 8.84 (s, 1H), 8.46 (d, J=8.6 Hz, 1H), 8.38 (d, J=8.6 Hz, 1H), 7.95 (d, J=8.3 Hz, 2H), 7.67 (d, J=8.3 Hz, 2H), 7.42 (d, J=2.3 Hz, 1H), 6.61 (d, J=2.3 Hz, 1H), 4.54-4.49 (m, 1H), 4.34 (ddd, J=10.9, 6.2, 1.8 Hz, 1H), 4.29 (ddd, J=11.7, 3.6, 1.6 Hz, 1H), 4.11-4.03 (m, 2H), 3.98 (s, 3H), 3.15 (s, 3H). [M+H]⁺ 400, Rt 1.34 min (method O).

### Example 130

### (5-amino-8-(4-(1-methyl-1H-pyrazol-3-yl)phenyl)-1,6-naphthyridin-2-yl)(3-methoxyazetidin-1-yl)methanone (160)

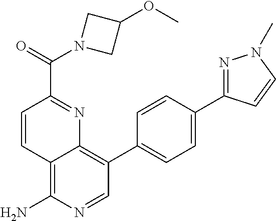 [SMILES: COC1CN(C(=O)c2ccc3c(N)ncc(-c4ccc(-c5ccn(C)n5)cc4)c3n2)C1]

To a solution of (3-methoxyazetidin-1-yl)(8-(4-(1-methyl-1H-pyrazol-3-yl)phenyl)-1,6-naphthyridin-2-yl)methanone (36 mg, 0.09 mmol) in CH₂Cl₂ (0.5 mL) was added peracetic acid (39% in AcOH; 0.06 mL, 0.35 mmol) and the mixture heated at 40° C. for 20 h. The mixture was then allowed to cool to rt and quenched with 1 M NaOH (5 mL) and extracted with CH₂Cl₂ (3×10 mL). The combined organic layers were filtered through a phase separator and concentrated in vacuo. The residue was then taken up in pyridine (0.5 mL) and p-toluenesulfonyl chloride (26 mg, 0.14 mmol) was added and the resulting mixture stirred at rt for 30 min. Ethanolamine (0.15 mL, 2.48 mmol) was then added and the mixture stirred at rt for 1 h. Water (5 mL) was added and the mixture extracted with CH₂Cl₂ (3×15 mL). The combined organic layers were washed with water (5 mL) and brine (5 mL), filtered through a phase separator and concentrated in vacuo. The residue was purified by Biotage column chromatography (SNAP 10 g column, CH₂Cl₂/EtOH 90/10->80/20) to give the title compound as a yellow solid (5 mg, 13%). ¹H NMR (500 MHz, CDCl₃) ppm=8.32 (d, J=8.7 Hz, 1H), 8.29-8.25 (m, 2H), 7.91 (d, J=8.2 Hz, 2H), 7.59 (d, J=8.2 Hz, 2H), 7.42 (d, J=2.3 Hz, 1H), 6.60 (d, J=2.3 Hz, 1H), 5.44 (br s, 2H), 4.49-4.44 (m, 1H), 4.37-4.31 (m, 1H), 4.27-4.22 (m, 1H), 4.10-4.03 (m, 2H), 3.99 (s, 3H), 3.14 (s, 3H). [M+H]⁺ 415, Rt 0.90 min (method O).

### Example 131

### (3-fluoro-3-methylazetidin-1-yl)(8-(4-(1-methyl-1H-pyrazol-4-yl)phenyl)-1,6-naphthyridin-2-yl)methanone (180)

### (8-bromo-1,6-naphthyridin-2-yl)(3-fluoro-3-methylazetidin-1-yl)methanone

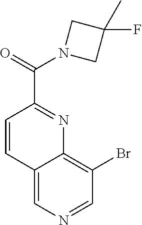 [SMILES: CC1(F)CN(C(=O)c2ccc3cncc(Br)c3n2)C1]

To 8-bromo-1,6-naphthyridine-2-carboxylic acid (260 mg, 1.03 mmol) and 3-fluoro-3-methylazetidine hydrochloride (175 mg, 1.39 mmol) in DMF (4.5 mL) was added HATU (600 mg, 1.58 mmol) and DIPEA (0.9 mL, 5.15 mmol), and the resulting mixture stirred at rt for 2 h. The mixture was then concentrated in vacuo and the residue purified by Biotage (SNAP 50 g column, CH₂Cl₂/EtOH 100/0->95/5) to give the title compound as a yellow solid (250 mg, 75%).

### (3-fluoro-3-methylazetidin-1-yl)(8-(4-(1-methyl-1H-pyrazol-4-yl)phenyl)-1,6-naphthyridin-2-yl)methanone

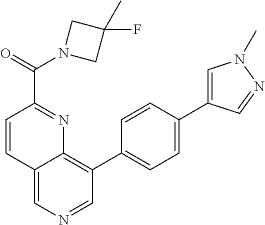 [SMILES: Cn1cc(-c2ccc(-c3cncc4ccc(C(=O)N5CC(C)(F)C5)nc34)cc2)cn1]

A mixture of (8-bromo-1,6-naphthyridin-2-yl)(3-fluoro-3-methylazetidin-1-yl)methanone (250 mg, 0.77 mmol), 1-methyl-4-(4-(4,4,5,5-tetramethyl-1,3,2-dioxaborolan-2-yl)phenyl)-1H-pyrazole (300 mg, 1.06 mmol), K₃PO₄ (500 mg, 2.36 mmol) and Pd(dtbpf)Cl₂ (56 mg, 0.086 mmol) in a mixture of 1,4-dioxane (3 mL) and water (0.5 mL) was stirred at 120° C. for 1 h under microwave irradiation. The mixture was then concentrated in vacuo and the residue purified by Biotage column chromatography (SNAP 25 g column, CH₂Cl₂/EtOH 98/2->93/7) to give the title compound as a dark yellow oil (204 mg, 66%). ¹H NMR (500 MHz, CDCl₃) ppm=9.33 (s, 1H), 8.86 (s, 1H), 8.49 (d, J=8.5 Hz, 1H), 8.39 (d, J=8.5 Hz, 1H), 7.85 (d, J=0.9 Hz, 1H), 7.71 (d, J=0.8 Hz, 1H), 7.66-7.62 (m, 4H), 4.52 (ddd, J=20.4, 12.3, 1.9 Hz, 1H), 4.39-4.30 (m, 2H), 4.16 (ddd, J=17.5, 11.7, 1.9 Hz, 1H), 4.00 (s, 3H), 1.52 (d, J=21.4 Hz, 3H). [M+H]⁺ 402, Rt 1.39 min (method O).

### Example 132

### (5-amino-8-(4-(1-methyl-1H-pyrazol-4-yl)phenyl)-1,6-naphthyridin-2-yl)(3-fluoro-3-methylazetidin-1-yl)methanone (184)

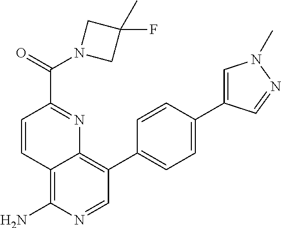 [SMILES: Cn1cc(-c2ccc(-c3cnc(N)c4ccc(C(=O)N5CC(C)(F)C5)nc34)cc2)cn1]

To a solution of (3-fluoro-3-methylazetidin-1-yl)(8-(4-(1-methyl-1H-pyrazol-4-yl)phenyl)-1,6-naphthyridin-2-yl)methanone (189 mg, 0.47 mmol) in CH₂Cl₂ (1.5 mL) was added peracetic acid (39% in AcOH; 0.35 mL, 2.06 mmol) and the mixture heated at 40° C. for 4 h. The mixture was then allowed to cool to rt and quenched with 1 M NaOH (10 mL). The aqueous layers was extracted with CH₂Cl₂ (3×20 mL) and the combined organic layers filtered through a phase separator and concentrated in vacuo. The residue was then taken up in pyridine (2 mL) and p-toluenesulfonyl chloride (135 mg, 0.71 mmol) was added and the resulting mixture stirred at rt for 30 min. Ethanolamine (0.80 mL, 13.23 mmol) was then added and the mixture stirred at rt for 1 h. Water (10 mL) was added and the mixture extracted with CH₂Cl₂ (3×20 mL). The combined organic layers were washed with water (10 mL) and brine (10 mL), filtered through a phase separator and concentrated in vacuo. The resulting residue was purified by Biotage column chromatography (SNAP 10 g column, EtOAc, then CH₂Cl₂/EtOH 95/5->85/20) to give the title compound as a yellow solid (52 mg, 27%). ¹H NMR (500 MHz, DMSO-d6) ppm=8.85 (d, J=8.7 Hz, 1H), 8.17 (s, 1H), 8.12 (s, 1H), 8.03 (d, J=8.7 Hz, 1H), 7.89 (s, 1H), 7.64 (d, J=8.1 Hz, 2H), 7.53 (d, J=8.1 Hz, 2H), 7.35 (br s, 2H), 4.35 (d, J=20.5 Hz, 2H), 4.13 (d, J=20.1 Hz, 2H), 3.89 (s, 3H), 1.47 (d, J=22.0 Hz, 3H). [M+H]⁺ 417, Rt 0.97 min (method O).

### Example 133

### azetidin-1-yl(8-(4-(1-methyl-1H-pyrazol-4-yl)phenyl)-1,6-naphthyridin-2-yl)methanone (147)

### azetidin-1-yl(8-bromo-1,6-naphthyridin-2-yl)methanone

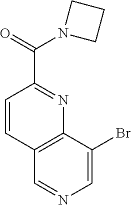 [SMILES: O=C(c1ccc2cncc(Br)c2n1)N1CCC1]

To 8-bromo-1,6-naphthyridine-2-carboxylic acid (200 mg, 0.790 mmol) in DMF (6.6 mL) was added HATU (361 mg, 0.948 mmol), azetidine (63.9 μl, 0.948 mmol) and DIPEA (304 jut, 1.74 mmol). The reaction mixture was stirred at rt. After 4 h, 361 mg of HATU were added. The reaction mixture was stirred for another hour before being concentrated. The crude material was purified via Biotage column chromatography (SNAP 25 g, 99/1 to 95/5). The product obtained were suspended in DCM and filtered. The filtrate was washed with water and the organic layers were dried and concentrated to give the title compound (200 mg, 87%). ¹H NMR (500 MHz, CDCl₃) ppm=9.22 (s, 1H), 9.00 (s, 1H), 8.43 (d, J=8.5 Hz, 1H), 8.41 (d, J=8.5 Hz, 1H), 5.08-5.01 (m, 2H), 4.34-4.28 (m, 2H), 2.49-2.40 (m, 2H). [M+H]⁺ 292/294. Rt 1.30 min (method M).

### azetidin-1-yl(8-(4-(1-methyl-1H-pyrazol-4-yl)phenyl)-1,6-naphthyridin-2-yl)methanone

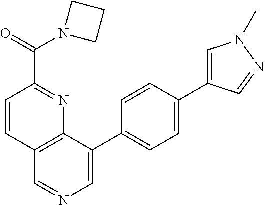 [SMILES: Cn1cc(-c2ccc(-c3cncc4ccc(C(=O)N5CCC5)nc34)cc2)cn1]

azetidin-1-yl(8-bromo-1,6-naphthyridin-2-yl)methanone (200 mg, 0.685 mmol), 1-methyl-4-(4-(4,4,5,5-tetramethyl-1,3,2-dioxaborolan-2-yl)phenyl)-1H-pyrazole (195 mg, 0.685 mmol) and Pd(dppf)Cl₂.CH₂Cl₂ (28.0 mg, 0.034 mmol) were loaded in a microwave vial and then acetonitrile (11.8 mL) and sodium carbonate in water (1.9 mL, 0.958 mmol) were added. The reaction mixture was heated at 120° C. for 1 h and concentrated. The crude was purified via biotage column chromatography (snap 25 g, DCM/EtOH 99/1 to 90/10) to give the title compound as a brown solid (167 mg, 66%). ¹H NMR (500 MHz, CDCl₃) ppm=9.32 (s, 1H), 8.83 (s, 1H), 8.48 (d, J=8.5 Hz, 1H), 8.39 (d, J=8.5 Hz, 1H), 7.86 (d, J=0.8 Hz, 1H), 7.72 (s, 1H), 7.68-7.60 (m, 4H), 4.46 (dd, J=8.8, 6.6 Hz, 2H), 4.24 (dd, J=8.4, 7.0 Hz, 2H), 4.00 (s, 3H), 2.30-2.21 (m, 2H). [M+H]⁺ 370. Rt 2.63 min (method N).

### Example 134

### (5-amino-8-(4-(1-methyl-1H-pyrazol-4-yl)phenyl)-1,6-naphthyridin-2-yl)(azetidin-1-yl)methanone (142)

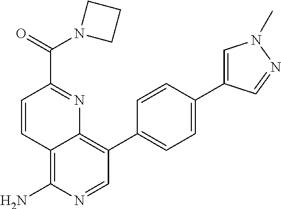 [SMILES: Cn1cc(-c2ccc(-c3cnc(N)c4ccc(C(=O)N5CCC5)nc34)cc2)cn1]

To azetidin-1-yl(8-(4-(1-methyl-1H-pyrazol-4-yl)phenyl)-1,6-naphthyridin-2-yl)methanone (136 mg, 0.368 mmol) in DCM (3.7 mL) was added 3-chloroperoxybenzoic acid (165 mg, 0.736 mmol). The reaction was stirred at rt for 1 h. An extra equivalent of mCPBA was added and the reaction mixture was stirred for another 30 min. After addition of a solution of Na₂S₂O₅ and then NaHCO₃, the reaction mixture was diluted with DCM. The aqueous layer was extracted three times with DCM, the organic layers were dried over MgSO₄ and concentrated. The residue was solubilised in pyridine (8.7 mL) and 4-toluenesulfonyl chloride (84 mg, 0.442 mmol) was added. The mixture was stirred at rt for 1 h before ethanolamine (556 μl, 9.20 mmol) was added. The reaction mixture was stirred at rt for another 2 h and diluted with water and DCM, the aqueous layer was extracted with DCM three times and the organic layers were concentrated. The crude was purified via Biotage column chromatography (SNAP25 g, DCM/EtOH 96/4 to 88/12) and the product was filtered on SCX2 column and released with 1N NH₃ in methanol to give the title compound as a orange solid (30 mg, 21% yield). ¹H NMR (500 MHz, DMSO) ppm=8.82 (d, J=8.7 Hz, 1H), 8.17 (s, 1H), 8.10 (s, 1H), 8.00 (d, J=8.6 Hz, 1H), 7.90 (d, J=0.8 Hz, 1H), 7.64-7.60 (m, 2H), 7.56-7.51 (m, 2H), 7.28 (s, 2H), 4.36-4.32 (m, 2H), 4.07-4.02 (m, 2H), 3.88 (s, 3H), 2.20-2.13 (m, 2H). [M+H]⁺ 385. Rt 2.10 min (method N).

### Example 135

### (3-methoxy-3-methylazetidin-1-yl)(8-(4-(1-methyl-1H-pyrazol-4-yl)phenyl)-1,6-naphthyridin-2-yl)methanone (149)

### (8-bromo-1,6-naphthyridin-2-yl)(3-methoxy-3-methylazetidin-1-yl)methanone

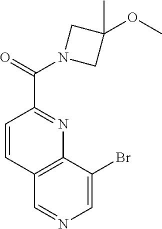 [SMILES: COC1(C)CN(C(=O)c2ccc3cncc(Br)c3n2)C1]

To 8-bromo-1,6-naphthyridine-2-carboxylic acid (199 mg, 0.787 mmol) in DMF (6.5 mL) was added HATU (359 mg, 0.945 mmol), 3-methoxy-3-methylazetidine hydrochloride (130 mg, 0.945 mmol) and DIPEA (302 μL, 1.732 mmol). The reaction mixture was stirred at rt for 19 h and concentrated. The residue was purified via Biotage column chromatography (SNAP 50 g, DCM/EtOH 99/1 to 97/3). The product obtained was then solubilised in DCM and washed twice with water. The organic layers were dried over MgSO₄ and concentrated to give the title compound (200 mg, 76%). ¹H NMR (500 MHz, CDCl₃) ppm=9.22 (s, 1H), 9.00 (s, 1H), 8.43 (s, 2H), 4.98-4.94 (m, 1H), 4.80 (dd, J=11.4, 1.7 Hz, 1H), 4.27-4.23 (m, 1H), 4.04 (dd, J=10.9, 1.7 Hz, 1H), 3.32 (s, 3H), 1.57 (s, 3H). [M+H]⁺ 336/338. Rt 1.33 min (method M).

### (3-methoxy-3-methylazetidin-1-yl)(8-(4-(1-methyl-1H-pyrazol-4-yl)phenyl)-1,6-naphthyridin-2-yl)methanone

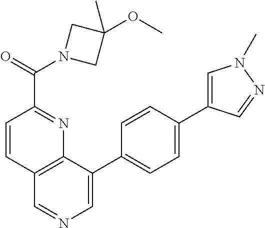 [SMILES: COC1(C)CN(C(=O)c2ccc3cncc(-c4ccc(-c5cnn(C)c5)cc4)c3n2)C1]

(8-bromo-1,6-naphthyridin-2-yl)(3-methoxy-3-methylazetidin-1-yl)methanone (200 mg, 0.595 mmol), 1-methyl-4-(4-(4,4,5,5-tetramethyl-1,3,2-dioxaborolan-2-yl)phenyl)-1H-pyrazole (169 mg, 0.595 mmol) and Pd(dppf)Cl₂.CH₂Cl₂ (24.29 mg, 0.030 mmol) were loaded in a microwave vial and then acetonitrile (10.3 mL) and sodium carbonate in water (1.6 mL, 0.833 mmol) were added. The reaction mixture was heated at 120° C. for 1 h. The reaction mixture was concentrated and purified via Biotage column chromatography (SNAP 25 g, DCM/EtOH 99/1 to 85/15). The product was filtered on a SCX-2 column and the product was released with 1N NH₃ in methanol to give the title compound as brown oil (155 mg, 63%). ¹H NMR (500 MHz, CDCl₃) ppm=9.31 (s, 1H), 8.83 (s, 1H), 8.46 (d, J=8.5 Hz, 1H), 8.36 (d, J=8.5 Hz, 1H), 7.81 (d, J=0.8 Hz, 1H), 7.68 (d, J=0.8 Hz, 1H), 7.65-7.58 (m, 4H), 4.37-4.32 (m, 1H), 4.14-4.07 (m, 2H), 3.99-3.93 (m, 4H), 3.10 (s, 3H), 1.35 (s, 3H). [M+H]⁺ 414. Rt 2.66 min (method N).

### Example 136

### (5-amino-8-(4-(1-methyl-1H-pyrazol-4-yl)phenyl)-1,6-naphthyridin-2-yl)(3-methoxy-3-methylazetidin-1-yl)methanone (144)

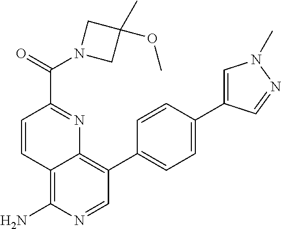 [SMILES: COC1(C)CN(C(=O)c2ccc3c(N)ncc(-c4ccc(-c5cnn(C)c5)cc4)c3n2)C1]

To (3-methoxy-3-methylazetidin-1-yl) (8-(4-(1-methyl-1H-pyrazol-4-yl)phenyl)-1,6-naphthyridin-2-yl)methanone (130 mg, 0.314 mmol) in DCM (3.1 mL) was added 3-chloroperoxybenzoic acid (141 mg, 0.629 mmol). The reaction was stirred at rt for 1 h. Additional mCPBA (141 mg) was added and the reaction mixture was stirred for another hour. After addition of a solution of Na₂S₂O₅ and then NaHCO₃, the reaction mixture was diluted with DCM. The aqueous layer was extracted three times with DCM, the organic layer was dried over MgSO₄ and concentrated. The residue was solubilised in pyridine (7.4 mL) and 4-toluenesulfonyl chloride (71.8 mg, 0.377 mmol) was added. The reaction mixture was stirred at rt for 1 h before ethanolamine (475 μl, 7.85 mmol) was added. The reaction mixture was stirred at rt for 1 h 30 and diluted with water and DCM. The aqueous layer was extracted with DCM three times and the organic layers were concentrated. The crude material was purified via Biotage column chromatography (SNAP25 g, DCM/EtOH 98/2 to 87/13). The product was filtered on a SCX-2 column and released with 1N NH₃ in methanol to give the title compound as an orange solid (50 mg, 37% yield over 2 steps). ¹H NMR (500 MHz, CDCl₃) ppm=8.39 (d, J=8.7 Hz, 1H), 8.25 (d, J=8.7 Hz, 1H), 8.22 (s, 1H), 7.80 (d, J=0.8 Hz, 1H), 7.66 (s, 1H), 7.59-7.53 (m, 4H), 5.65 (s, 2H), 4.32-4.28 (m, 1H), 4.12 (d, J=10.8 Hz, 1H), 4.05 (dd, J=11.3, 1.7 Hz, 1H), 3.99 (s, 3H), 3.95 (dd, J=10.8, 1.6 Hz, 1H), 3.10 (s, 3H), 1.35 (s, 3H). [M+H]⁺ 429. Rt 2.14 min (method N).

### Example 137

### 8-(2-fluoro-4-(1-methyl-1H-pyrazol-4-yl)phenyl)-N-methyl-1,6-naphthyridine-2-carboxamide (17)

### 8-bromo-N-methyl-1,6-naphthyridine-2-carboxamide

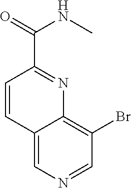

To a suspension of 8-bromo-1,6-naphthyridine-2-carboxylic acid (320 mg, 1.27 mmol) in DMF (6 mL) was added HATU (529 mg, 1.39 mmol) and the mixture stirred for 10 min before the addition of 2 M solution of MeNH₂ in THF (2.0 mL, 4.00 mmol). The mixture was stirred at rt for 15 min before the addition of DIPEA (1.0 mL, 5.74 mmol). The resulting mixture was then stirred at rt for 18 h and concentrated in vacuo. The crude was purified by Biotage (SNAP 25 g column, CH₂Cl₂/EtOH 94/6->89/11) to give the title compound as a yellow solid (326 mg, 97%).

### 8-(2-fluoro-4-(1-methyl-1H-pyrazol-4-yl)phenyl)-N-methyl-1,6-naphthyridine-2-carboxamide

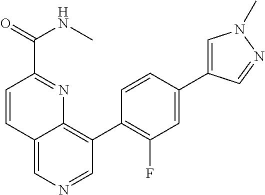 [SMILES: CNC(=O)c1ccc2cncc(-c3ccc(-c4cnn(C)c4)cc3F)c2n1]

A mixture of 8-bromo-N-methyl-1,6-naphthyridine-2-carboxamide (80 mg, 0.30 mmol), 4-(3-fluoro-4-(4,4,5,5-tetramethyl-1,3,2-dioxaborolan-2-yl)phenyl)-1-methyl-1H-pyrazole (90 mg, 0.30 mmol), K₃PO₄ (21 mg, 0.99 mmol), and Pd(dtbpf)Cl₂ (21 mg, 0.032 mmol) in a mixture of 1,4-dioxane (1.5 mL) and water (0.5 mL) was heated at 120° C. for 1 h under microwave irradiation. Concentrated in vacuo. Purified by Biotage (SingleStep 12 g column, CH₂Cl₂/EtOH 98/2->85/15) to give the title compound as a yellow solid (66 mg, 61%). ¹H NMR (500 MHz, CDCl₃) ppm=9.40 (s, 1H), 8.87 (s, 1H), 8.55 (d, J=8.5, 1H), 8.49 (d, J=8.5, 1H), 7.88 (s, 1H), 7.75 (s, 1H), 7.55 (t, J=7.7, 1H), 7.46 (dd, J=7.9, 1.7, 1H), 7.39 (dd, J=11.1, 1.7, 1H), 4.02 (s, 3H), 3.03 (d, 3H), 2.82 (s, 1H). [M+H]⁺ 362. Rt 1.31 min (method M).

### Example 138

### N-methyl-8-(2-methyl-4-(1-methyl-1H-pyrazol-4-yl)phenyl)-1,6-naphthyridine-2-carboxamide (43)

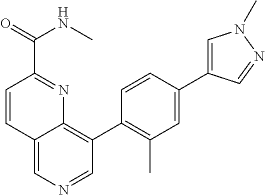 [SMILES: CNC(=O)c1ccc2cncc(-c3ccc(-c4cnn(C)c4)cc3C)c2n1]

N-methyl-8-(2-methyl-4-(1-methyl-1H-pyrazol-4-yl)phenyl)-1,6-naphthyridine-2-carboxamide was prepared in a manner similar to Example 81, using 1-methyl-4-(3-methyl-4-(4,4,5,5-tetramethyl-1,3,2-dioxaborolan-2-yl)phenyl)-1H-pyrazole as the starting material. ¹H NMR (500 MHz, CDCl₃) ppm=9.37 (s, 1H), 8.74 (s, 1H), 8.53 (d, J=8.5, 1H), 8.46 (d, J=8.4, 1H), 7.87 (s, 1H), 7.76 (br d, J=5.5, 1H), 7.72 (s, 1H), 7.50 (s, 1H), 7.45 (dd, J=7.8, 1.9, 1H), 7.31 (d, J=7.8, 1H), 3.99 (s, 3H), 2.96 (d, 3H), 2.11 (s, 3H) [M+H]⁺ 358. Rt 1.32 min (method M).

### Example 139

### 5-amino-8-(2-fluoro-4-(1-methyl-1H-pyrazol-4-yl)phenyl)-N-methyl-1,6-naphthyridine-2-carboxamide (40)

### 8-(2-fluoro-4-(1-methyl-1H-pyrazol-4-yl)phenyl)-2-(methylcarbamoyl)-1,6-naphthyridine 6-oxide

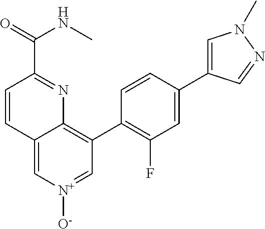 [SMILES: CNC(=O)c1ccc2c[n+]([O-])cc(-c3ccc(-c4cnn(C)c4)cc3F)c2n1]

To 8-(2-fluoro-4-(1-methyl-1H-pyrazol-4-yl)phenyl)-N-methyl-1,6-naphthyridine-2-carboxamide (Example 81) (64 mg, 0.18 mmol) in CH₂Cl₂ (1.5 mL) was added mCPBA (<77%; 53 mg, 0.24 mmol) and the resulting mixture stirred at rt for 26 h with addition of additional mCPBA (<77%; 53 mg, 0.24 mmol) after 18 h. 1 M NaOH (10 mL) was then added, the mixture extracted with CH₂Cl₂ (3×15 mL) and the combined organic layers filtered through a phase separator and concentrated in vacuo. The crude material was purified by Biotage (SNAP 10 g column, CH₂Cl₂/EtOH 100/0->92/8) to give the title compound as a pale yellow solid (15 mg, 22%).

### 5-amino-8-(2-fluoro-4-(1-methyl-1H-pyrazol-4-yl)phenyl)-N-methyl-1,6-naphthyridine-2-carboxamide

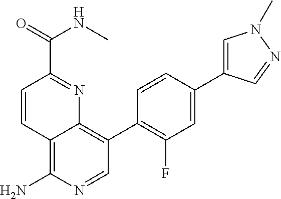 [SMILES: CNC(=O)c1ccc2c(N)ncc(-c3ccc(-c4cnn(C)c4)cc3F)c2n1]

To a solution of 8-(2-fluoro-4-(1-methyl-1H-pyrazol-4-yl)phenyl)-2-(methylcarbamoyl)-1,6-naphthyridine 6-oxide (15 mg, 0.040 mmol) in pyridine (0.70 mL) was added p-toluenesulfonyl chloride (12 mg, 0.063 mmol) and the resulting mixture stirred at rt for 45 min. Ethanolamine (0.060 mL, 0.99 mmol) was then added and the mixture stirred at rt for 1 h. Water (10 mL) was then added and the mixture extracted with EtOAc (4×10 mL). The combined organic layers were washed with water (10 mL), dried (MgSO₄), filtered and concentrated in vacuo. The crude material was purified by Biotage (SNAP 10 g column, CH₂C₁₂/EtOH 100/0->85/15) to give the title compound as a yellow solid (7 mg, 47%). ¹H NMR (500 MHz, CDCl₃) ppm=8.39 (d, J=8.7, 1H), 8.35 (d, J=8.6, 1H), 8.25 (s, 1H), 7.87-7.83 (m, 2H), 7.72 (s, 1H), 7.48 (t, J=7.7, 1H), 7.40 (dd, J=7.9, 1.8, 1H), 7.34 (dd, J=11.0, 1.7, 1H), 4.01 (s, 3H), 3.01 (d, 3H) [M+H]⁺ 377. Rt 1.05 min (method M).

### Example 140

### (8-(4-(1-methyl-1H-pyrazol-4-yl)phenyl)-1,6-naphthyridin-2-yl)(pyrrolidin-1-yl)methanone (20)

### (8-bromo-1,6-naphthyridin-2-yl)(pyrrolidin-1-yl)methanone

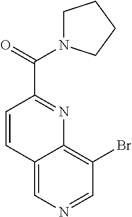 [SMILES: O=C(c1ccc2cncc(Br)c2n1)N1CCCC1]

To a stirred solution of 8-bromo-1,6-napthyridine-2-carboxylic acid (50 mg, 0.198 mmol) and HATU (98 mg, 0.257 mmol) in DMF (1 mL) was added DIPEA (0.172 mL, 0.988 mmol) and pyrrolidine (0.021 mL, 0.257 mmol). The reaction mixture was sealed and stirred at room temperature overnight. The crude reaction mixture was poured onto NaOH (10 mL, 1 M), and the organic material was extracted with EtOAc (2×10 mL). The combined organic layers were washed with brine, dried over MgSO₄, filtered, and the filtrate concentrated under reduced pressure. The crude product was purified over a silica cartridge using a solvent system of 0-12% EtOH in dichloromethane to give the title compound (16.7 mg, 28%).

### (8-(4-(1-methyl-1H-pyrazol-4-yl)phenyl)-1,6-naphthyridin-2-yl)(pyrrolidin-1-yl)methanone

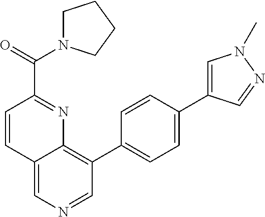 [SMILES: Cn1cc(-c2ccc(-c3cncc4ccc(C(=O)N5CCCC5)nc34)cc2)cn1]

To a stirred solution of (8-bromo-1,6-naphthyridin-2-yl)(pyrrolidin-1-yl)methanone (16.7 mg, 0.055 mmol), 1-methyl-4-(4-(4,4,5,5-tetramethyl-1,3,2-dioxaborolan-2-yl)phenyl)-1H-pyrazole (15.5 mg, 0.055 mmol) and dichloro 1,1′bis(diphenylphosphino)ferrocene palladium (II) (2.0 mg, 2.73 μmol) in acetonitrile (0.8 mL) was added Na₂CO₃ (0.153 mL, 0.076 mmol, 0.5 M). The reaction mixture was sealed and heated to 150° C. in a focused microwave reactor for 2 h. The crude mixture was cooled to room temperature, concentrated under reduced pressure and purified over a silica cartridge using a solvent system of 1-10% EtOH in dichloromethane. The title compound (8.5 mg, 41%) was isolated as a yellow oil. ¹H NMR (500 MHz, CDCl₃) ppm=9.35 (s, 1H), 8.88 (s, 1H), 8.50 (d, J=8.6, 1H), 8.26 (d, J=8.5, 1H), 7.86 (d, J=0.9, 1H), 7.71 (m, 3H), 7.62 (m, 2H), 4.00 (s, 3H), 3.73 (dt, J=6.8, 2.1, 4H), 1.88 (m, 4H). [M+H]⁺ 384. Rt 1.24 min (method O).

### Example 141

### (4,4-difluoropiperidin-1-yl)(8-(4-(1-methyl-1H-pyrazol-4-yl)phenyl)-1,6-naphthyridin-2-yl)methanone (35)

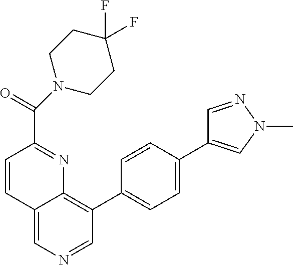 [SMILES: Cn1cc(-c2ccc(-c3cncc4ccc(C(=O)N5CCC(F)(F)CC5)nc34)cc2)cn1]

(4,4-difluoropiperidin-1-yl)(8-(4-(1-methyl-1H-pyrazol-4-yl)phenyl)-1,6-naphthyridin-2-yl)methanone was prepared in a manner similar to Example 84, using 4,4-difluoropiperidine hydrochloride and 1-methyl-4-(4-(4,4,5,5-tetramethyl-1,3,2-dioxaborolan-2-yl)phenyl)-1H-pyrazole as the starting materials. ¹H NMR (500 MHz, CDCl₃) ppm=9.34 (s, 1H), 8.89 (s, 1H), 8.51 (d, J=8.4, 1H), 8.01 (d, J=8.4, 1H), 7.84 (d, J=0.8, 1H), 7.69 (m, 1H), 7.65 (m, 2H), 7.60 (m, 2H), 3.99 (s, 3H), 3.91 (m, 2H), 3.69 (m, 2H), 2.09 (tt, J=13.2, 6.7, 2H), 1.81 (tt, J=13.2, 5.8, 2H). [M+H]⁺ 434. Rt 1.25 min (method O).

### Example 142

### (8-(4-(1-methyl-1H-pyrazol-4-yl)phenyl)-1,6-naphthyridin-2-yl)(morpholino)methanone (39)

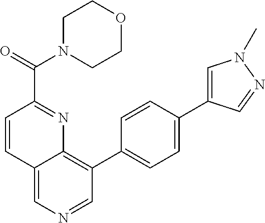 [SMILES: Cn1cc(-c2ccc(-c3cncc4ccc(C(=O)N5CCOCC5)nc34)cc2)cn1]

(8-(4-(1-methyl-1H-pyrazol-4-yl)phenyl)-1,6-naphthyridin-2-yl)(morpholino)methanone was prepared in a manner similar to Example 84, using morpholine and 1-methyl-4-(4-(4,4,5,5-tetramethyl-1,3,2-dioxaborolan-2-yl)phenyl)-1H-pyrazole as the starting materials. ¹H NMR (500 MHz, CDCl₃) ppm=9.33 (s, 1H), 8.89 (s, 1H), 8.49 (d, J=8.5, 1H), 8.04 (d, J=8.5, 1H), 7.86 (s, 1H), 7.66 (m, 5H), 4.01 (s, 3H), 3.81 (m, 5H), 3.58 (m, 3H). [M+H]⁺ 400. Rt 2.46 min (method N).

### Example 143

### (5-amino-8-(4-(1-methyl-1H-pyrazol-4-yl)phenyl)-1,6-naphthyridin-2-yl)(pyrrolidin-1-yl)methanone (86)

### 8-(4-(1-methyl-1H-pyrazol-4-yl)phenyl)-2-(pyrrolidine-1-carbonyl)-1,6-naphthyridine 6-oxide

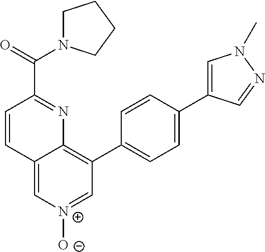 [SMILES: Cn1cc(-c2ccc(-c3c[n+]([O-])cc4ccc(C(=O)N5CCCC5)nc34)cc2)cn1]

To a solution of 8-(4-(1-methyl-1H-pyrazol-4-yl)phenyl)-1,6-naphthyridin-2-yl)(pyrrolidin-1-yl)methanone (Example 84) (20 mg, 0.052 mmol) in dichloromethane (1 mL) was added mCPBA (36 mg, 0.156 mmol) in one portion, and the reaction mixture was stirred at room temperature for 1.5 h. The crude reaction mixture was poured onto NaOH (5 mL, 1 M), and the organic material extracted twice with dichloromethane. The combined organic layers were washed with brine, dried over MgSO₄, filtered, and the filtrate concentrated under reduced pressure. The crude product (15 mg, 72%) was isolated as a yellow solid.

### (5-amino-8-(4-(1-methyl-1H-pyrazol-4-yl)phenyl)-1,6-naphthyridin-2-yl)(pyrrolidin-1-yl)methanone

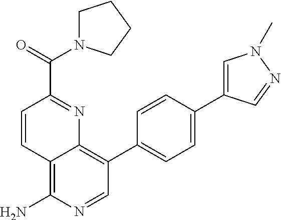 [SMILES: Cn1cc(-c2ccc(-c3cnc(N)c4ccc(C(=O)N5CCCC5)nc34)cc2)cn1]

To (8-(4-(1-methyl-1H-pyrazol-4-yl)phenyl)-1,6-naphthyridin-2-yl)(pyrrolidin-1-yl)methanone (14 mg, 0.035 mmol) in pyridine (567 μL, 7.01 mmol) was added 4-toluenesulfonyl chloride (8.0 mg, 0.042 mmol), and the reaction mixture was stirred at room temperature for 30 min. To the stirred reaction mixture was added ethanolamine (53 μL, 0.876 mmol) in two portions, and the resulting solution was stirred at room temperature for 1 h. The crude reaction mixture was diluted with water and the organic material was extracted with ethyl acetate. The organic layer was washed with brine, dried over MgSO₄, filtered and the filtrate concentrated under reduced pressure. The crude material was purified over a silica cartridge using a solvent system of 0-15% EtOH in dichloromethane to give the title compound (8 mg, 57%) as a yellow solid. ¹H NMR (500 MHz, CDCl₃) ppm=8.63 (d, J=8.7, 1H), 8.17 (d, J=8.6, 1H), 8.09 (s, 1H), 7.83 (d, J=0.8, 1H), 7.69 (d, J=0.8, 1H), 7.57 (s, 4H), 6.71 (s, 2H), 4.00 (s, 3H), 3.69 (td, J=6.8, 3.6, 4H), 1.86 (m, 4H) [M+H]⁺ 399. Rt 0.94 min (method O).

### Example 144

### (5-amino-8-(4-(1-methyl-1H-pyrazol-4-yl)phenyl)-1,6-naphthyridin-2-yl)(morpholino)methanone (85)

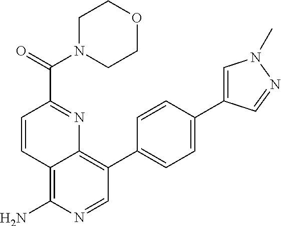 [SMILES: Cn1cc(-c2ccc(-c3cnc(N)c4ccc(C(=O)N5CCOCC5)nc34)cc2)cn1]

(5-amino-8-(4-(1-methyl-1H-pyrazol-4-yl)phenyl)-1,6-naphthyridin-2-yl)(morpholino) methanone was prepared in a manner similar to Example 87, using (8-(4-(1-methyl-1H-pyrazol-4-yl)phenyl)-1,6-naphthyridin-2-yl)(morpholino)methanone (Example 86) as the starting material. ¹H NMR (500 MHz, CDCl₃) ppm=8.49 (d, J=8.6, 1H), 8.19 (s, 1H), 7.95 (d, J=8.6, 1H), 7.83 (d, J=0.9, 1H), 7.69 (s, 1H), 7.56 (s, 4H), 6.05 (s, 2H), 3.99 (s, 3H), 3.80 (m, 6H), 3.53 (t, J=4.7, 2H) [M+H]⁺ 415. Rt 0.84 min (method O).

### Example 145

### (3,3-difluoropiperidin-1-yl)(8-(4-(1-methyl-1H-pyrazol-4-yl)phenyl)-1,6-naphthyridin-2-yl)methanone (82)

### (8-bromo-1,6-naphthyridin-2-yl)(3,3-difluoropiperidin-1-yl)methanone

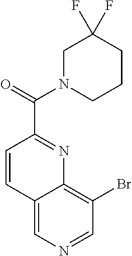 [SMILES: O=C(c1ccc2cncc(Br)c2n1)N1CCCC(F)(F)C1]

To a stirred solution of 8-bromo-1,6-napthyridine-2-carboxylic acid (50 mg, 0.198 mmol) and HATU (98 mg, 0.257 mmol) in DMF (1 mL) was added DIPEA (0.206 mL, 1.186 mmol) and 3,3-difluoropiperidine hydrochloride (31.1 mg, 0.198 mmol). The reaction mixture was sealed and stirred at room temperature overnight. The crude reaction mixture was poured onto NaOH (10 mL, 1M), and the organic material was extracted with EtOAc (2×10 mL). The combined organic layers were washed with brine, dried over MgSO₄, filtered, and the filtrate concentrated under reduced pressure. The crude product was purified over a silica cartridge using a solvent system of 0-12% EtOH in dichloromethane to give the title compound (68 mg, 97%).

### (3,3-difluoropiperidin-1-yl) (8-(4-(1-methyl-1H-pyrazol-4-yl)phenyl)-1,6-naphthyridin-2-yl)methanone

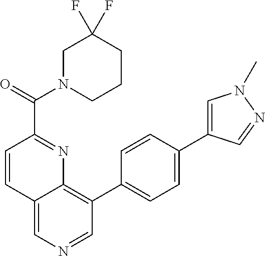 [SMILES: Cn1cc(-c2ccc(-c3cncc4ccc(C(=O)N5CCCC(F)(F)C5)nc34)cc2)cn1]

To a stirred solution of (8-bromo-1,6-naphthyridin-2-yl)(3,3-difluoropiperidin-1-yl)methanone (20.0 mg, 0.056 mmol), 1-methyl-4-(4-(4,4,5,5-tetramethyl-1,3,2-dioxaborolan-2-yl)phenyl)-1H-pyrazole (16.0 mg, 0.056 mmol) and dichloro 1,1′bis(diphenylphosphino)ferrocene palladium (II) (2.3 mg, 0.056 mmol) in acetonitrile (0.8 mL) was added Na₂CO₃ (0.157 mL, 0.079 mmol, 0.5 M). The reaction mixture was sealed and heated to 125° C. in a focused microwave reactor for 90 min. The crude mixture was cooled to room temperature, concentrated under reduced pressure and purified over a silica cartridge using a solvent system of 1-12% EtOH in dichloromethane to give the title compound (17 mg, 70%) as a yellow oil. ¹H NMR (500 MHz, CDCl₃) ppm=9.35 (s, 1H), 8.90 (s, 1H), 8.52 (t, J=6.9, 1H), 8.04 (t, J=9.0, 1H), 7.87 (d, J=4.5, 1H), 7.71 (m, 3H), 7.63 (m, 2H), 4.01 (s, 3H), 3.80 (m, 1H), 3.62 (s, 1H), 2.60 (m, 4H), 1.92 (m, 1H), 1.59 (m, 1H). [M+H]⁺ 434. Rt 1.35 min (method M).

### Example 146

### N-(4-methoxybenzyl)-8-(4-(1-methyl-1H-pyrazol-4-yl)phenyl)-1,6-naphthyridine-2-carboxamide (81)

### 8-bromo-N-(4-methoxybenzyl)-1,6-naphthyridine-2-carboxamide

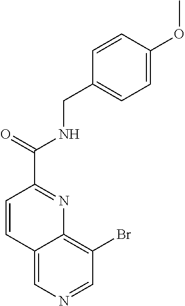 [SMILES: COc1ccc(CNC(=O)c2ccc3cncc(Br)c3n2)cc1]

To a stirred solution of thionyl chloride (25.6 μL, 0.351 mmol) in DMF (1 mL) was added 8-bromo-1,6-naphthyridine-2-carboxylic acid (44.4 mg, 0.175 mmol). The reaction mixture was sealed and heated to 75° C. for 5 h. The mixture was cooled to room temperature and the volatiles were removed under reduced pressure. The crude material was dissolved in dichloromethane (1.0 mL) and 4-methoxybenzylamine (0.027 mL, 0.210 mmol) and triethylamine (0.025 mL, 0.175 mmol) were added. The reaction mixture was sealed and stirred at room temperature overnight. The solution was poured onto saturated NaHCO₃ and extracted twice with ethyl acetate. The combined organic layers were washed with brine, dried over MgSO₄, filtered and the filtrate concentrated under reduced pressure. The crude product was purified over a silica cartridge using a solvent system of 0-10% EtOH in dichloromethane. The appropriate fractions were re-purified by four runs over a preparative TLC plate using a solvent system of 1% MeOH in dichloromethane to give the title compound (18 mg, 27%).

### N-(4-methoxybenzyl)-8-(4-(1-methyl-1H-pyrazol-4-yl)phenyl)-1,6-naphthyridine-2-carboxamide

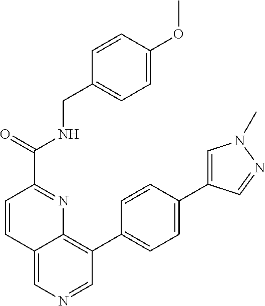 [SMILES: COc1ccc(CNC(=O)c2ccc3cncc(-c4ccc(-c5cnn(C)c5)cc4)c3n2)cc1]

To a stirred solution of 8-bromo-N-(4-methoxybenzyl)-1,6-naphthyridine-2-carboxamide (18.0 mg, 0.048 mmol), 1-methyl-4-(4-(4,4,5,5-tetramethyl-1,3,2-dioxaborolan-2-yl)phenyl)-1H-pyrazole (13.7 mg, 0.056 mmol) and dichloro 1,1′bis(diphenylphosphino)ferrocene palladium (II) (1.77 mg, 2.42 μmol) in acetonitrile (0.8 mL) was added Na₂CO₃ (0.135 mL, 0.068 mmol, 0.5 M). The reaction mixture was sealed and heated to 125° C. in a focused microwave reactor for 90 min. The crude mixture was cooled to room temperature, concentrated under reduced pressure and the residue purified over a silica cartridge using a solvent system of 0-10% EtOH in dichloromethane. Further purification via preparative TLC using 4 runs in 1% MeOH in dichloromethane gave the title compound (6 mg, 28%) as a brown solid. ¹H NMR (500 MHz, CDCl₃) ppm=9.36 (s, 1H), 8.92 (s, 1H), 8.56 (d, J=8.5, 1H), 8.50 (d, J=8.5, 1H), 8.26 (d, J=5.8, 1H), 7.82 (d, J=0.8, 1H), 7.71 (m, 2H), 7.67 (d, J=0.8, 1H), 7.49 (m, 2H), 7.29 (s, 1H), 6.90 (m, 2H), 4.59 (d, J=5.5, 2H), 4.02 (m, 4H), 3.80 (s, 3H) [M+H]⁺ 450. Rt 3.07 min (method N).

### Example 147

### 8-(4-(1-methyl-1H-pyrazol-4-yl)phenyl)-N-(4-(trifluoromethyl)benzyl)-1,6-naphthyridine-2-carboxamide (87)

### 8-bromo-N-(4-(trifluoromethyl)benzyl)-1,6-naphthyridine-2-carboxamide

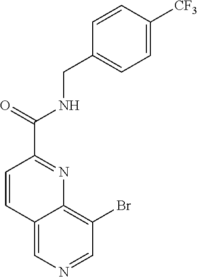 [SMILES: O=C(NCc1ccc(C(F)(F)F)cc1)c1ccc2cncc(Br)c2n1]

To a stirred solution of oxalyl chloride (17.3 μL, 0.198 mmol) in toluene (1 mL) was added 8-bromo-1,6-naphthyridine-2-carboxylic acid (50.0 mg, 0.198 mmol) followed by two drops of DMF. After stirring the reaction mixture at room temperature for 4 h, the volatiles were removed under reduced pressure. The crude material was dissolved in dichloromethane (1.0 mL) and (4-(trifluoromethyl)phenyl)methanamine (0.031 mL, 0.217 mmol) and triethylamine (0.055 mL, 0.395 mmol) were added. The reaction mixture was sealed and stirred at room temperature overnight. The solution was poured onto saturated NaHCO₃ and the organic material was extracted twice with ethylacetate. The combined organic layers were washed with brine, dried over MgSO₄, filtered and the filtrate concentrated under reduced pressure. The crude product was purified over a silica cartridge using a solvent system of 0-10% EtOH in dichloromethane to give the title compound (40 mg, 49%) as a colourless crystalline solid.

### 8-(4-(1-methyl-1H-pyrazol-4-yl)phenyl)-N-(4-(trifluoromethyl)benzyl)-1,6-naphthyridine-2-carboxamide

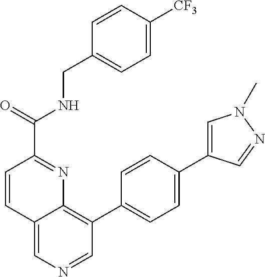 [SMILES: Cn1cc(-c2ccc(-c3cncc4ccc(C(=O)NCc5ccc(C(F)(F)F)cc5)nc34)cc2)cn1]

To a stirred solution of 8-bromo-N-(4-(trifluoromethyl)benzyl)-1,6-naphthyridine-2-carboxamide (20 mg, 0.049 mmol), 1-methyl-4-(4-(4,4,5,5-tetramethyl-1,3,2-dioxaborolan-2-yl)phenyl)-1H-pyrazole (13.9 mg, 0.049 mmol) and dichloro 1,1′bis(diphenylphosphino)ferrocene palladium (II) (1.99 mg, 2.44 μmol) in acetonitrile (0.8 mL) was added Na₂CO₃ (0.137 mL, 0.068 mmol, 0.5 M). The reaction mixture was sealed and heated to 125° C. in a focused microwave reactor for 90 min. The crude mixture was cooled to room temperature, concentrated under reduced pressure and the residue purified over a silica cartridge using a solvent system of 0-10% EtOH in dichloromethane to give the title compound (9 mg, 38%) as a brown solid. ¹H NMR (500 MHz, CDCl₃) ppm=9.38 (s, 1H), 8.92 (s, 1H), 8.59 (d, J=8.4, 1H), 8.50 (d, J=8.4, 1H), 8.39 (t, J=6.1, 1 H), 7.82 (s, 1H), 7.72 (m, 2H), 7.64 (s, 1H), 7.61 (d, J=8.0, 2H), 7.54 (m, 2H), 7.46 (d, J=8.0, 2H), 4.73 (d, J=5.9, 2H), 4.00 (s, 3H) [M+H]⁺ 488. Rt 1.47 min (method O).

### Example 148

### 5-Amino-8-(1-methyl-2,2-dioxo-2,3-dihydro-1H-benzo[c]isothiazol-5-yl)-[1,6]naphthyridine-2-carboxylic acid methylamide (32)

### 8-Bromo-6-oxy-[1,6]naphthyridine-2-carboxylic acid methylamide

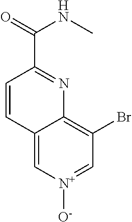 [SMILES: CNC(=O)c1ccc2c[n+]([O-])cc(Br)c2n1]

In a 12 mL screw-capped vessel 8-bromo-[1,6]naphthyridine-2-carboxylic acid methylamide (200 mg, 0.65 mmol) was dissolved in dichloromethane (4 mL) and treated with 3-chloroperoxybenzoic acid (191 mg, 0.78 mmol) at rt. The orange solution was stirred overnight at room temperature. Additional 3-chloroperoxybenzoic acid (191 mg, 0.78 mmol) was added and the reaction mixture was stirred for an additional 3 h at rt. The mixture was treated with 1 N NaOH solution and dichloromethane and the layers separated. The organic layer was washed with water, dried over sodium sulfate, filtered and evaporated to dryness to yield 57 mg (28%) of the title compound as pale beige solid.

### 5-Amino-8-bromo-[1,6]naphthyridine-2-carboxylic acid methylamide

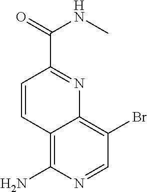 [SMILES: CNC(=O)c1ccc2c(N)ncc(Br)c2n1]

In a 12 mL screw-capped vessel 8-Bromo-6-oxy-[1,6]naphthyridine-2-carboxylic acid methylamide (59.0 mg, 0.19 mmol) was suspended in pyridine (3 mL). Toluene-4-sulfonyl chloride (43.1 mg, 0.23 mmol) was added and the reaction mixture was stirred for 30 minutes at rt. Ethanolamine (282 μl, 4.72 mmol) was added. The reaction mixture turned suddenly dark red and was stirred for additional 30 minutes. The red reaction mixture was diluted with water and extracted with ethyl acetate. The organic layer was washed with water, dried over sodium sulfate, filtered and evaporated to dryness to yield 49 mg (79%) of the title compound as a brown solid. 5-Amino-8-(1-methyl-2,2-dioxo-2,3-dihydro-1H-benzo[c]isothiazol-5-yl)-[1,6]naphthyridine-2-carboxylic acid methylamide

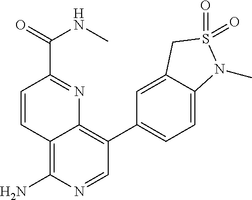 [SMILES: CNC(=O)c1ccc2c(N)ncc(-c3ccc4c(c3)CS(=O)(=O)N4C)c2n1]

In a microwave vial, to a solution of 5-amino-8-bromo-[1,6]naphthyridine-2-carboxylic acid methylamide (49.0 mg, 0.15 mmol) in N,N-dimethylformamide (2.5 mL) were added 1-methyl-5-(4,4,5,5-tetramethyl-[1,3,2]dioxaborolan-2-yl)-1,3-dihydro-benzo[c]isothiazole 2,2-dioxide (55.0 mg, 0.18 mmol), sodium carbonate solution (0.5 M, 0.59 mL, 0.30 mmol) and Pd(dppf)Cl₂ dichloromethane complex (6.05 mg, 0.01 mmol). The vial was flushed with nitrogen twice and stirred at 120° C. for 1 h under microwave irradiation. The reaction mixture was treated with water and solid sodium chloride. The resulting brown precipitate was filtered off, washed with water and purified by flash chromatography (dichloromethane/methanol) to give impure material. This was treated with acetonitrile, filtered, washed with acetonitrile and dried to yield 16.6 mg (29%) of the title compound as a yellow solid. ¹H NMR (400 MHz, DMSO-d6) ppm=8.87 (d, J=8.6, 1H), 8.14 (s, 1H), 8.09 (d, J=8.5, 1H), 8.03 (q, J=4.8, 1H), 7.72-7.68 (m, 1H), 7.68-7.63 (m, 1H), 7.29 (s, 2H), 7.04 (d, J=8.3, 1H), 4.73 (s, 2H), 3.11 (s, 3H), 2.88 (d, J=4.9, 3H). [M+H]⁺ 384. Rt 1.95 min (method L).

### Example 149

### (5-Amino-8-(2,2-dioxido-1,3-dihydrobenzo[c]isothiazol-5-yl)-1,6-naphthyridin-2-yl)(3,3-difluoropyrrolidin-1-yl)methanone (50)

### (8-Bromo-1,6-naphthyridin-2-yl)(3,3-difluoropyrrolidin-1-yl)methanone

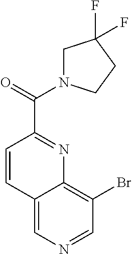 [SMILES: O=C(c1ccc2cncc(Br)c2n1)N1CCC(F)(F)C1]

To a mixture of 8-bromo-1,6-naphthyridine-2-carboxylic acid (300 mg, 1.186 mmol) in DMF (15 mL) was added 3,3-difluoropyrrolidine hydrochloride (494 mg, 3.44 mmol), triethylamine (1.972 mL, 14.23 mmol) and HATU (1488 mg, 3.91 mmol) and the mixture was stirred at rt for 1 h. The mixture was diluted with water and EtOAc and the layers were separated. The organic layer was washed with water. The combined organic layers were dried over MgSO₄ and concentrated in vacuo. The resulting brown oil was purified by chromatography on silica gel (Biotage, CH₂Cl₂/EtOH, 100:0 to 97:3) to give the title compound (395 mg, 97%) as a light brown solid.

### 8-Bromo-2-(3,3-difluoropyrrolidine-1-carbonyl)-1,6-naphthyridine 6-oxide

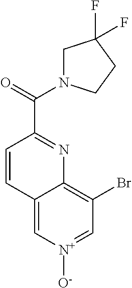 [SMILES: O=C(c1ccc2c[n+]([O-])cc(Br)c2n1)N1CCC(F)(F)C1]

To a mixture of (8-bromo-1,6-naphthyridin-2-yl)(3,3-difluoropyrrolidin-1-yl)methanone (360 mg, 1.052 mmol) in CH₂Cl₂ (3.6 mL) was added 3-chloroperoxybenzoic acid (286 mg, 1.157 mmol) and the mixture was stirred at rt overnight. The mixture was diluted with 1N NaOH and CH₂Cl₂ and the layers were separated. The aqueous layer was extracted with CH₂Cl₂ twice. The combined organic layers were washed with water, dried over MgSO₄ and concentrated in vacuo. The residue was purified by chromatography on silica gel (Biotage, CH₂Cl₂/EtOH, 100:0 to 94:6) to give the title compound (290 mg, 77%) as a white solid.

### (5-Amino-8-bromo-1,6-naphthyridin-2-yl)(3,3-difluoropyrrolidin-1-yl)methanone

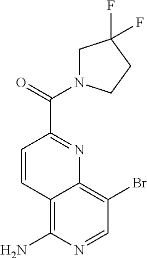 [SMILES: Nc1ncc(Br)c2nc(C(=O)N3CCC(F)(F)C3)ccc12]

To a suspension of 8-bromo-2-(3,3-difluoropyrrolidine-1-carbonyl)-1,6-naphthyridine 6-oxide (0.285 g, 0.796 mmol) in pyridine (15.45 ml, 119 mmol) was added 4-toluenesulfonyl chloride (0.197 g, 1.035 mmol) and the mixture was stirred at rt for 30 min. Ethanolamine (1.20 ml, 19.89 mmol) was then added and the mixture stirred at rt for 10 min. Diluted with water and EtOAc. The layers were separated and the aqueous layer was extracted with EtOAc three times. The combined organic layers were washed with water, dried over MgSO₄ and concentrated in vacuo. The resulting residue was purified by chromatography on silica gel (Biotage, CH₂Cl₂/EtOH, 100:0 to 95:5) to give the title compound (270 mg, 95%) as a yellow solid.

### (5-Amino-8-(2,2-dioxido-1,3-dihydrobenzo[c]isothiazol-5-yl)-1,6-naphthyridin-2-yl) (3,3-difluoropyrrolidin-1-yl)methanone

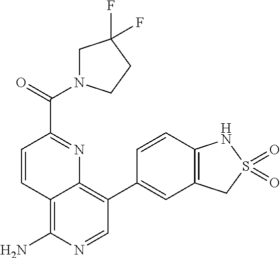 [SMILES: Nc1ncc(-c2ccc3c(c2)CS(=O)(=O)N3)c2nc(C(=O)N3CCC(F)(F)C3)ccc12]

(5-Amino-8-bromo-1,6-naphthyridin-2-yl) (3,3-difluoropyrrolidin-1-yl)methanone (40 mg, 0.112 mmol), 5-(4,4,5,5-tetramethyl-1,3,2-dioxaborolan-2-yl)-1,3-dihydrobenzo[c]isothiazole 2,2-dioxide (52.9 mg, 0.134 mmol) and Pd(PPh₃)₄ (4.10 mg, 5.60 μmol) were loaded in a microwave vial. The vial was capped and evacuated using high vacuum and purged with nitrogen (each three times). Acetonitrile (2 mL) and aqueous sodium carbonate (0.5M, 0.448 mL, 0.224 mmol) were added and the vial evacuated using high vacuum and purged with nitrogen (each three times). The mixture was heated at 120° C. for 1 h under microwave irradiation. After this time additional Pd(PPh₃)₄ (8.20 mg, 0.011 mmol) was added and the mixture was heated at 120° C. for 1 h under microwave irradiation. The mixture was then concentrated in vacuo and the resulting brown solid was purified by chromatography on silica gel (Biotage, CH₂Cl₂/EtOH, 100:0 to 96:4) and further purified by prep. HPLC (Gilson, methanol/water+0.1% formic acid gradient) to give the title compound (5.7 mg, 11%, mixture of rotamers) as a sticky yellow solid. ¹H NMR (500 MHz, DMSO-d6) ppm=10.55 (s, 1H), 8.83 (d, J=8.6, 1H), 8.08 (s, 0.35H), 8.07 (s, 0.65H), 7.97 (d, J=8.6, 0.65H), 7.90 (d, J=8.6, 0.35H), 7.48 (s, 1H), 7.46-7.40 (m, 1H), 7.30 (s, 2H), 6.90 (d, J=8.1, 0.35H), 6.86 (d, J=8.1, 0.65H), 4.56-4.46 (m, 2H), 4.18 (t, J=13.0, 1H), 3.95-3.89 (m, 1H), 3.75 (t, J=7.5, 2H), 2.48-2.38 (m, 2H). [M+H]⁺ 446. Rt 0.94 min (method M).

### Example 150

### 5-amino-8-(2,2-dioxido-1,3-dihydrobenzo[c]isothiazol-5-yl)-N,N-dimethyl-1,6-naphthyridine-2-carboxamide (59)

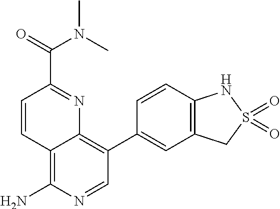 [SMILES: CN(C)C(=O)c1ccc2c(N)ncc(-c3ccc4c(c3)CS(=O)(=O)N4)c2n1]

5-Amino-8-(2,2-dioxido-1,3-dihydrobenzo[c]isothiazol-5-yl)-N,N-dimethyl-1,6-naphthyridine-2-carboxamide was prepared in a manner similar to Example 93, using dimethylamine as the starting material. ¹H NMR (500 MHz, CD₃OD/CDCl₃, 1:1) ppm=8.64 (d, J=8.6, 1H), 8.01 (s, 1H), 7.71 (d, J=8.6, 1H), 7.55 (s, 1H), 7.47-7.44 (m, 2H), 6.92 (d, J=8.7, 1H), 4.43 (s, 2H), 3.13 (s, 3H), 3.07 (s, 3H) [M+H]⁺ 384. Rt 0.73 min (method M).

### Example 151

### 8-[4-(1-Methyl-1H-pyrazol-4-yl)-phenyl]-[1,6]naphthyridine-2-carboxylic acid (25)

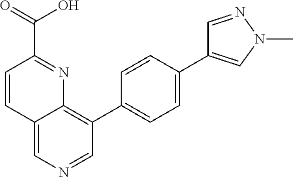 [SMILES: Cn1cc(-c2ccc(-c3cncc4ccc(C(=O)O)nc34)cc2)cn1]

In a 100 mL screw-capped vessel 8-bromo-1,6-naphthyridine-2-carboxylic acid (900 mg, 3.56 mmol) was suspended in acetonitrile (30 mL). 1-Methyl-4-[4-(4,4,5,5-tetramethyl-[1,3,2]dioxaborolan-2-yl)-phenyl]-1H-pyrazole (1.38 g, 4.27 mmol), sodium carbonate solution (0.5 M, 21.3 mL, 10.7 mmol) and Pd(dppf)Cl₂ dichloromethane complex (145 mg, 0.18 mmol) were added. The reaction mixture was flushed with nitrogen and stirred at 70° C. for 16 h. The reaction mixture was treated with ethyl acetate and water. The precipitate was filtered off. The layers were separated and the aqueous layer was adjusted to pH 6 with conc. HCl. The solution was lyophilized and the light brown residue was treated with methanol and filtered. The filtrate was evaporated to dryness to afford 1.40 g (86% purity, 100%) of the title compound as a light brown solid. ¹H NMR (500 MHz, DMSO-d6) ppm=13.64 (s, 1H), 9.49 (s, 1H), 8.91 (s, 1H), 8.82 (d, J=8.5, 1H), 8.27 (d, J=8.4, 1H), 8.25 (s, 1H), 7.99-7.95 (m, 1H), 7.87-7.83 (m, 2H), 7.76-7.70 (m, 2H), 3.90 (s, 3H). [M+H]⁺ 331. Rt 2.1 min (method L).

### Example 152

### 8-[4-(1-Methyl-1H-pyrazol-4-yl)-phenyl]-[1,6]naphthyridine-2-carboxylic acid methylamide (3)

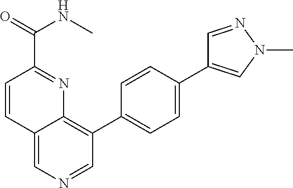 [SMILES: CNC(=O)c1ccc2cncc(-c3ccc(-c4cnn(C)c4)cc3)c2n1]

In a 12 mL screw-capped vessel 8-[4-(1-methyl-1H-pyrazol-4-yl)-phenyl]-[1,6]naphthyridine-2-carboxylic acid (Example 95) (100 mg, 0.26 mmol) was treated with thionyl chloride (450 μl, 6.20 mmol). The yellow suspension was stirred at 70° C. for 3 h. The reaction mixture was evaporated to dryness. To the residue in a screw-capped vessel was added a 2 M solution of methylamine in tetrahydrofuran (2.60 mL, 5.20 mmol) and the solution stirred for 1 h at rt. The reaction mixture was evaporated to dryness and the brown residue purified by preparative HPLC (acetonitrile/water) to give after lyophilising the trifluoroacetate salt of the (17.3 mg, 14%) as a yellow solid. ¹H NMR (500 MHz, DMSO-d6) ppm=9.50 (s, 1H), 8.90 (s, 1H), 8.85 (d, J=8.5, 1H), 8.31 (d, J=8.4, 1H), 8.24 (s, 1H), 8.17 (q, J=5.0, 4.4, 1H), 7.96 (s, 1H), 7.88-7.84 (m, 2H), 7.77-7.73 (m, 2H), 3.91 (s, 3H), 2.90 (d, J=4.9, 3H). [M+H]⁺ 344. Rt 2.3 min (method L).

### Example 153

### 8-[4-(1-Methyl-1H-pyrazol-4-yl)-phenyl]-[1,6]naphthyridine-2-carboxylic acid amide (4)

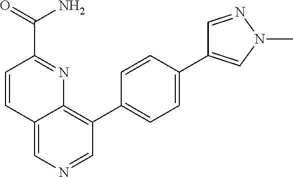 [SMILES: Cn1cc(-c2ccc(-c3cncc4ccc(C(N)=O)nc34)cc2)cn1]

In a 12 mL screw-capped vessel 8-[4-(1-methyl-1H-pyrazol-4-yl)-phenyl]-[1,6]naphthyridine-2-carboxylic acid (Example 95) (86% purity, 100 mg, 0.26 mmol) was treated with thionyl chloride (0.50 mL, 6.89 mmol). The yellow suspension was stirred at 70° C. for 5 h. The reaction mixture was evaporated dryness. To the residue in the screw-capped vessel was added a 0.5 M solution of ammonia in 1,4-dioxane (5.21 mL, 2.61 mmol) and the resulting solution was stirred at rt overnight. The reaction mixture was evaporated to dryness and the residue purified by preparative HPLC (acetonitrile/water) to yield the trifluoroacetate salt of the title compound (11.9 mg, 10%) as a yellow solid. ¹H NMR (500 MHz, DMSO-d6) ppm=9.51 (s, 1H), 8.91 (s, 1H), 8.86 (d, J=8.5, 1H), 8.32 (d, J=8.5, 1H), 8.24 (s, 1H), 8.03-7.98 (m, 1H), 7.97-7.94 (m, 1H), 7.87-7.83 (m, 2H), 7.78-7.74 (m, 2H), 7.65-7.59 (m, 1H), 3.90 (s, 3H). [M+H]⁺ 330. Rt 2.24 min (method L).

### Example 154

### 8-[4-(1-Methyl-1H-pyrazol-4-yl)-phenyl]-[1,6]naphthyridine-2-carboxylic acid acetyl-amide (9)

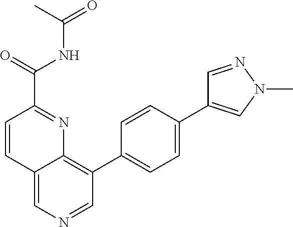 [SMILES: CC(=O)NC(=O)c1ccc2cncc(-c3ccc(-c4cnn(C)c4)cc3)c2n1]

In a 12 mL screw-capped vessel 8-[4-(1-methyl-1H-pyrazol-4-yl)-phenyl]-[1,6]naphthyridine-2-carboxylic acid amide (35.7 mg, 0.10 mmol) was suspended in dry tetrahydrofuran (0.50 mL, 6.17 mmol). To this solution at −78° C., lithium bis(trimethylsilyl)amide (1.06 M) solution in tetrahydrofuran/ethylbenzene (0.20 mL, 0.20 mmol) was added dropwise. The reaction mixture was stirred for 1 h at this temperature and warmed up to 0° C. Acetic anhydride (57.2 μl, 0.61 mmol) was added dropwise and stifling was continued for 4 h. The reaction mixture was treated with water and dichloromethane and then filtered through a phase separator to isolate the organic phase, which was then evaporated to dryness. The residue was purified by preparative HPLC (acetonitrile/water) to give the trifluoracetic acid salt of the title compound (3.30 mg, 7%) as a yellow solid. ¹H NMR (500 MHz, DMSO-d6) ppm=10.37 (s, 1H), 9.56 (s, 1H), 8.97 (s, 1H), 8.96-8.94 (m, 1H), 8.40-8.36 (m, 1H), 8.25 (s, 1H), 7.98-7.94 (m, 1H), 7.90-7.84 (m, 2H), 7.79-7.73 (m, 2H), 3.91 (s, 3H), 2.44 (s, 3H). [M+H]⁺ 372. Rt 2.51 min (method L).

### Example 155

### (3-Methoxyazetidin-1-yl)(8-(4-(1-methyl-1H-pyrazol-4-yl)phenyl)-1,6-naphthyridin-2-yl)methanone (29)

### 8-(4-(1-Methyl-1H-pyrazol-4-yl)phenyl)-1,6-naphthyridine-2-carboxylic acid

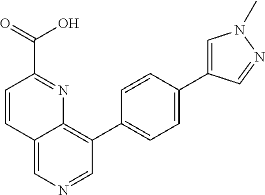 [SMILES: Cn1cc(-c2ccc(-c3cncc4ccc(C(=O)O)nc34)cc2)cn1]

8-Bromo-1,6-naphthyridine-2-carboxylic acid (300 mg, 1.186 mmol), 1-methyl-4-(4-(4,4,5,5-tetramethyl-1,3,2-dioxaborolan-2-yl)phenyl)-1H-pyrazole (337 mg, 1.186 mmol) and Pd(dppf)Cl₂—CH₂Cl₂ (43.4 mg, 0.059 mmol) were loaded in a microwave vial. The capped vial was evacuated using high vacuum and purged with nitrogen (each three times). Acetonitrile (10 mL) and aqueous sodium carbonate (0.5M, 3.32 mL, 1.660 mmol) were added and the mixture was evacuated using high vacuum and purged with nitrogen (each three times). The mixture was heated at 120° C. for 1 h under microwave irradiation. The mixture was then evaporated to dryness and the resulting brown solid was purified by chromatography on silica gel (Biotage, CH₂Cl₂/MeOH+0.3% CH₃CO₂H, 90:10 to 50:50) to give the title compound (600 mg, 92%, purity 60%) as a grey solid.

### (3-Methoxyazetidin-1-yl)(8-(4-(1-methyl-1H-pyrazol-4-yl)phenyl)-1,6-naphthyridin-2-yl)methanone

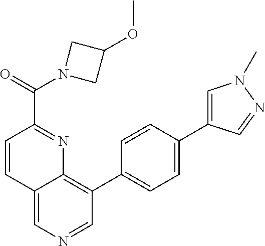 [SMILES: COC1CN(C(=O)c2ccc3cncc(-c4ccc(-c5cnn(C)c5)cc4)c3n2)C1]

To a mixture of 8-(4-(1-Methyl-1H-pyrazol-4-yl)phenyl)-1,6-naphthyridine-2-carboxylic acid (70 mg, 0.127 mmol) in DMF (3 mL) was added 3-methoxy-azetidine hydrochloride (55.0 mg, 0.445 mmol), triethylamine (0.211 mL, 1.526 mmol) and HATU (169 mg, 0.445 mmol) and the mixture was stirred at rt for 1 h. The mixture was diluted with water and EtOAc and the layers were separated. The organic layer was washed with water and the combined organic layers were dried over MgSO₄ and concentrated in vacuo. The resulting brown oil was purified by chromatography on silica gel (Biotage, CH₂Cl₂/EtOH, 100:0 to 95:5) and further purified by ion exchange (SCX2 cartridge, loading with CH₂Cl₂/MeOH 9/1, elution with CH₂Cl₂/1N NH₃ in MeOH 9/1) to give the title compound (30 mg, 59%) as light yellow solid. ¹H NMR (500 MHz, CDCl₃) ppm=9.31 (s, 1H), 8.83 (s, 1H), 8.46 (d, J=8.5, 1H), 8.38 (d, J=8.5, 1H), 7.82 (d, J=0.8, 1H), 7.70 (d, J=0.8, 1H), 7.66-7.58 (m, 4H), 4.50 (ddd, J=11.9, 6.0, 1.6, 1H), 4.39-4.25 (m, 2H), 4.14-4.02 (m, 2H), 3.98 (s, 3H), 3.17 (s, 3H). [M+H]⁺ 400. Rt 2.62 min (method N).

### Example 156

### (3,3-Difluoroazetidin-1-yl)(8-(4-(1-methyl-1H-pyrazol-4-yl)phenyl)-1,6-naphthyridin-2-yl)methanone (75)

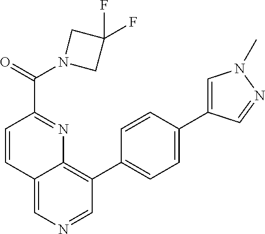 [SMILES: Cn1cc(-c2ccc(-c3cncc4ccc(C(=O)N5CC(F)(F)C5)nc34)cc2)cn1]

(3,3-Difluoroazetidin-1-yl) (8-(4-(1-methyl-1H-pyrazol-4-yl)phenyl)-1,6-naphthyridin-2-yl)methanone was prepared in a manner similar to Example 99, using 3,3-difluoroazetidine hydrochloride as the starting material. ¹H NMR (500 MHz, CDCl₃) ppm=9.35 (s, 1H), 8.87 (s, 1H), 8.51 (d, J=8.5, 1H), 8.40 (d, J=8.5, 1H), 7.85 (d, J=0.9, 1H), 7.71 (s, 1H), 7.66-7.55 (m, 4H), 4.74-4.59 (m, 2H), 4.57-4.41 (m, 2H), 3.99 (s, 3H). [M+H]⁺ 406. Rt 2.79 min (method N).

### Example 157

### (5-amino-8-(4-(1-methyl-1H-pyrazol-4-yl)phenyl)-1,6-naphthyridin-2-yl)(3,3-difluoroazetidin-1-yl)methanone (143)

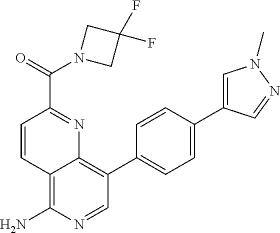 [SMILES: Cn1cc(-c2ccc(-c3cnc(N)c4ccc(C(=O)N5CC(F)(F)C5)nc34)cc2)cn1]

To (3,3-difluoroazetidin-1-yl) (8-(4-(1-methyl-1H-pyrazol-4-yl)phenyl)-1,6-naphthyridin-2-yl)methanone (61 mg, 0.150 mmol) in DCM (1.5 mL) was added 3-chloroperoxybenzoic acid (67.4 mg, 0.301 mmol). The reaction was stirred at rt for 1 h. Additional mCPBA (67 mg) was added and the reaction mixture was stirred for another 30 min. After addition of a solution of Na₂S₂O₅ and then NaHCO₃, the reaction mixture was diluted with DCM. The aqueous layer was extracted three times with DCM, the organic layers were dried over MgSO₄ and concentrated. The residue was solubilised in pyridine (3.5 mL) and 4-toluenesulfonyl chloride (34.3 mg, 0.180 mmol) was added. The mixture was stirred at rt for 1 h before ethanolamine (227 μL, 3.75 mmol) was added. The reaction mixture was stirred at rt for 1 h 30 and diluted with water and DCM, the aqueous layer was extracted with DCM three times and the organic layer were concentrated. The crude material was purified via Biotage column chromatography (SNAP10 g, DCM/EtOH 97/3 to 90/10) to give the title compound as a yellow solid (25 mg, 39% yield). ¹H NMR (500 MHz, DMSO) ppm=8.87 (d, J=8.6 Hz, 1H), 8.18 (s, 1H), 8.11 (s, 1H), 8.07 (d, J=8.6 Hz, 1H), 7.89 (d, J=0.8 Hz, 1H), 7.64 (d, J=8.2 Hz, 2H), 7.56-7.50 (m, 2H), 7.36 (s, 2H), 4.70-4.61 (m, 2H), 4.52-4.44 (m, 2H), 3.89 (s, 3H). [M+H]⁺ 421. Rt 2.21 min (method N).

### Example 158

### N-(2-Methoxyethyl)-8-(4-(1-methyl-1H-pyrazol-4-yl)phenyl)-1,6-naphthyridine-2-carboxamide (74)

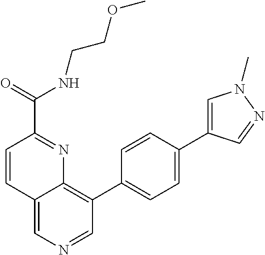 [SMILES: COCCNC(=O)c1ccc2cncc(-c3ccc(-c4cnn(C)c4)cc3)c2n1]

N-(2-Methoxyethyl)-8-(4-(1-methyl-1H-pyrazol-4-yl)phenyl)-1,6-naphthyridine-2-carboxamide was prepared in a manner similar to Example 99, using 2-methoxy ethylamine as the starting material. ¹H NMR (500 MHz, CDCl₃) ppm=9.35 (br s, 1H), 8.92 (br s, 1H), 8.54 (d, J=8.4, 1H), 8.47 (d, J=8.4, 1H), 8.36 (t, J=5.7, 1H), 7.87 (s, 1H), 7.79 (d, J=8.3, 1H), 7.72 (s, 1H), 7.67 (d, J=8.3, 2H), 4.00 (s, 3H), 3.67 (td, J=5.7, 4.7, 2H), 3.56 (dd, J=5.7, 4.7, 2H), 3.33 (s, 3H). [M+H]⁺ 388. Rt 2.68 min (method N).

### Example 159

### N-Hydroxy-N-methyl-8-(4-(1-methyl-1H-pyrazol-4-yl)phenyl)-1,6-naphthyridine-2-carboxamide (76)

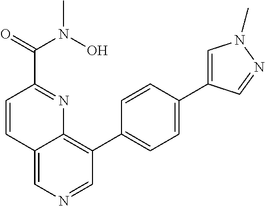 [SMILES: CN(O)C(=O)c1ccc2cncc(-c3ccc(-c4cnn(C)c4)cc3)c2n1]

N-Hydroxy-N-methyl-8-(4-(1-methyl-1H-pyrazol-4-yl)phenyl)-1,6-naphthyridine-2-carboxamide was prepared in a manner similar to Example 99, using N-methyl hydroxylamine hydrochloride as the starting material. ¹H NMR (500 MHz, CDCl₃) ppm=13.26 (br s, 1H), 9.43 (br s, 1H), 8.95 (br s, 1H), 8.66 (d, J=8.3, 1H), 8.57 (d, J=8.3, 1H), 7.88 (s, 1H), 7.74 (s, 1H), 7.69 (d, J=7.8, 2H), 7.63 (d, J=7.8, 2H), 4.00 (s, 3H), 3.48 (s, 3H). [M+H]⁺ 360. Rt 2.38 min (method N).

### Example 160

### (5-Amino-8-(4-(1-methyl-1H-pyrazol-4-yl)phenyl)-1,6-naphthyridin-2-yl)(3-methoxyazetidin-1-yl)methanone (11)

### 2-(3-Methoxyazetidine-1-carbonyl)-8-(4-(1-methyl-1H-pyrazol-4-yl)phenyl)-1,6-naphthyridine 6-oxide

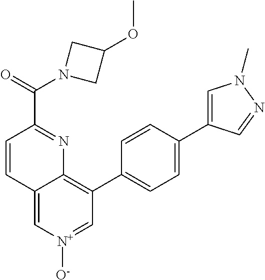 [SMILES: COC1CN(C(=O)c2ccc3c[n+]([O-])cc(-c4ccc(-c5cnn(C)c5)cc4)c3n2)C1]

To a mixture of (3-methoxyazetidin-1-yl)(8-(4-(1-methyl-1H-pyrazol-4-yl)phenyl)-1,6-naphthyridin-2-yl)methanone (Example 99) (20 mg, 0.050 mmol) in CH₂Cl₂ (0.5 mL) was added 3-chloroperoxybenzoic acid (14.83 mg, 0.060 mmol) and the mixture was stirred at rt for 1.5 h. The mixture was diluted with 1 N NaOH and CH₂Cl₂ and the layers were separated. The aqueous layer was extracted with CH₂Cl₂ twice. The combined organic layers were dried over MgSO₄ and concentrated in vacuum. The residue was purified by chromatography on silica gel (Biotage, CH₂Cl₂/EtOH, 100:0 to 91:9) to give the title compound (14 mg, 67%) as a yellow solid.

### (5-Amino-8-(4-(1-methyl-1H-pyrazol-4-yl)phenyl)-1,6-naphthyridin-2-yl) (3-methoxyazetidin-1-yl)methanone

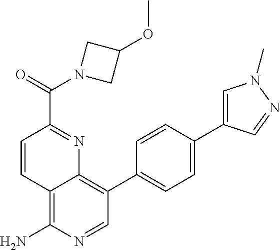 [SMILES: COC1CN(C(=O)c2ccc3c(N)ncc(-c4ccc(-c5cnn(C)c5)cc4)c3n2)C1]

To a suspension of 2-(3-methoxyazetidine-1-carbonyl)-8-(4-(1-methyl-1H-pyrazol-4-yl)phenyl)-1,6-naphthyridine 6-oxide (14 mg, 0.034 mmol) in pyridine (792 μl, 6.74 mmol) was added 4-toluenesulfonyl chloride (7.71 mg, 0.040 mmol) and the mixture was stirred at rt for 30 min. Ethanolamine (50.9 μl, 0.842 mmol) was then added and the mixture was stirred at rt for 45 min. The mixture was then diluted with water and EtOAc. The layers were separated and the aqueous layer was extracted with EtOAc three times. The combined organic layers were washed with water, dried over MgSO₄ and concentrated in vacuo. The resulting residue was purified by chromatography on silica gel (Biotage, CH₂Cl₂/EtOH, 100:0 to 80:20) and further purified by ion exchange (SCX2 cartridge, loading with CH₂Cl₂/MeOH 9/1, elution with CH₂Cl₂/1N NH₃ in MeOH 9/1) followed by further purification by prep. HPLC (Gilson, acetonitrile/water gradient) to give the title compound (6.5 mg, 47%) as a yellow solid. ¹H NMR (500 MHz, CDCl₃) ppm=8.33 (d, J=8.6, 1H), 8.27 (d, J=8.6, 1H), 8.24 (s, 1H), 7.82 (d, J=0.8, 1H), 7.68 (d, J=0.8, 1H), 7.60-7.55 (m, 4H), 5.40 (s, 2H), 4.51-4.42 (m, 1H), 4.37-4.31 (m, 1H), 4.26 (ddd, J=11.8, 3.9, 1.8, 1H), 4.11-4.02 (m, 2H), 3.99 (s, 3H), 3.16 (s, 3H). [M+H]⁺ 415. Rt 0.95 min (method M).

### Example 161

### (8-(4-(1-Methyl-1H-pyrazol-4-yl)phenyl)-1,6-naphthyridin-2-yl)(piperazin-1-yl)methanone (47)

### tert-Butyl 4-(8-(4-(1-methyl-1H-pyrazol-4-yl)phenyl)-1,6-naphthyridine-2-carbonyl)piperazine-1-carboxylate

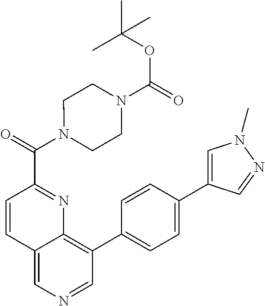 [SMILES: Cn1cc(-c2ccc(-c3cncc4ccc(C(=O)N5CCN(C(=O)OC(C)(C)C)CC5)nc34)cc2)cn1]

To a mixture of 8-[4-(1-methyl-1H-pyrazol-4-yl)-phenyl]-[1,6]naphthyridine-2-carboxylic acid (Example 95) (100 mg, 0.182 mmol) in DMF (5 mL) was added tert-butyl piperazine-1-carboxylate (118 mg, 0.636 mmol), triethylamine (0.302 mL, 2.180 mmol) and HATU (242 mg, 0.636 mmol) and the mixture was stirred at rt for 1 h. The mixture was diluted with water and EtOAc and the layers were separated. The organic layer was washed with water. The combined organic layers were dried over MgSO₄ and concentrated in vacuo. The residue was purified by chromatography on silica gel (Biotage, CH₂Cl₂/EtOH, 100:0 to 94:4) to give the title compound (66 mg, 73%) as a pale yellow oil.

### (8-(4-(1-Methyl-1H-pyrazol-4-yl)phenyl)-1,6-naphthyridin-2-yl) (piperazin-1-yl)methanone

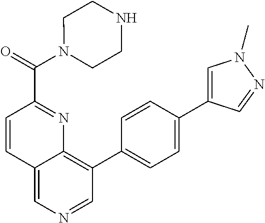 [SMILES: Cn1cc(-c2ccc(-c3cncc4ccc(C(=O)N5CCNCC5)nc34)cc2)cn1]

To a solution of tert-butyl 4-(8-(4-(1-methyl-1H-pyrazol-4-yl)phenyl)-1,6-naphthyridine-2-carbonyl)piperazine-1-carboxylate (66 mg, 0.132 mmol) in CH₂Cl₂ (3.5 mL) was added trifluoroacetic acid (0.483 mL, 2.65 mmol) at 0° C. and the mixture was stirred at rt for 2 h. After this time the trifluoroacetic acid was removed by azeotropic distillation with toluene and the residue was purified by chromatography on silica gel (Biotage, CH₂Cl₂/EtOH, 100:0 to 85:15), further purified by ion exchange (SCX2-cartridge, loading with CH₂Cl₂/MeOH 9/1, elution with CH₂Cl₂/1N NH₃ in MeOH 9/1) and finally purified by prep. HPLC (Gilson, acetonitrile/water gradient) to give the title compound (21.5 mg, 76%) as a pale yellow solid. ¹H NMR (500 MHz, CDCl₃) ppm=9.29 (s, 1H), 8.87 (s, 1H), 8.46 (d, J=8.5, 1H), 7.96 (d, J=8.5, 1H), 7.84 (s, 1H), 7.71 (s, 1H), 7.69 (d, J=8.3, 2H), 7.60 (d, J=8.3, 2H), 3.98 (s, 3H), 3.82-3.77 (m, 2H), 3.72-3.67 (m, 2H), 3.01-2.95 (m, 2H), 2.80-2.74 (m, 2H). [M+H]⁺ 399. Rt 1.87 min (method N).

### Example 162

### (5-Amino-8-(4-(1-methyl-1H-pyrazol-4-yl)phenyl)-1,6-naphthyridin-2-yl)(piperazin-1-yl)methanone (55)

### 2-(4-(tert-Butoxycarbonyl)piperazine-1-carbonyl)-8-(4-(1-methyl-1H-pyrazol-4-yl)phenyl)-1,6-naphthyridine 6-oxide

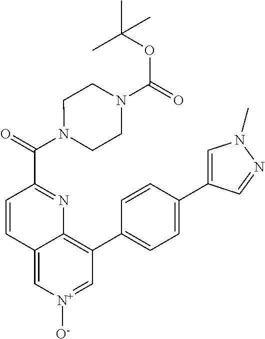 [SMILES: Cn1cc(-c2ccc(-c3c[n+]([O-])cc4ccc(C(=O)N5CCN(C(=O)OC(C)(C)C)CC5)nc34)cc2)cn1]

To a mixture of tert-butyl 4-(8-(4-(1-methyl-1H-pyrazol-4-yl)phenyl)-1,6-naphthyridine-2-carbonyl)piperazine-1-carboxylate (171 mg, 0.343 mmol) in CH₂Cl₂ (3 mL) was added 3-chloroperoxybenzoic acid (93 mg, 0.377 mmol) and the mixture was stirred at rt for 1 h. The mixture was diluted with 1N NaOH and CH₂Cl₂ and the layers were separated. The aqueous layer was extracted with CH₂Cl₂ twice. The combined organic layers were dried over MgSO₄ and concentrated in vacuo. The residue was purified by chromatography on silica gel (Biotage, CH₂Cl₂/EtOH, 100:0 to 80:20) to give the title compound (125 mg, 71%) as a yellow solid.

### 105.2 tert-Butyl 4-(5-amino-8-(4-(1-methyl-1H-pyrazol-4-yl)phenyl)-1,6-naphthyridine-2-carbonyl)piperazine-1-carboxylate

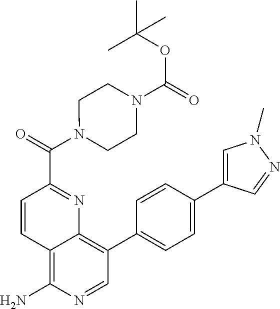 [SMILES: Cn1cc(-c2ccc(-c3cnc(N)c4ccc(C(=O)N5CCN(C(=O)OC(C)(C)C)CC5)nc34)cc2)cn1]

To a suspension of 2-(4-(tert-butoxycarbonyl)piperazine-1-carbonyl)-8-(4-(1-methyl-1H-pyrazol-4-yl)phenyl)-1,6-naphthyridine 6-oxide (120 mg, 0.233 mmol) in pyridine (5483 μL, 46.6 mmol) was added 4-toluenesulfonyl chloride (53.4 mg, 0.280 mmol) and the mixture was stirred at rt for 40 min. Ethanolamine (353 μl, 5.83 mmol) was then added and the mixture was stirred at rt for 45 min. The mixture was then diluted with water and EtOAc. The layers were separated and the aqueous layer was extracted with EtOAc three times. The combined organic layers were washed with water, dried over MgSO₄ and concentrated in vacuo. The resulting residue was purified by chromatography on silica gel (Biotage, CH₂Cl₂/EtOH, 100:0 to 90:10) to give a yellow solid (32 mg) which was combined with the precipitate formed in the aqueous layer (53 mg, isolated by filtration) to give the title compound (85 mg, 71%) as a yellow solid.

### (5-Amino-8-(4-(1-methyl-1H-pyrazol-4-yl)phenyl)-1,6-naphthyridin-2-yl)(piperazin-1-yl)methanone

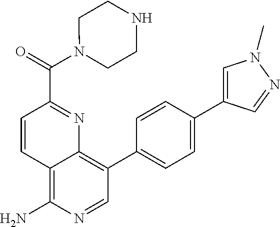 [SMILES: Cn1cc(-c2ccc(-c3cnc(N)c4ccc(C(=O)N5CCNCC5)nc34)cc2)cn1]

To a solution of tert-butyl 4-(5-amino-8-(4-(1-methyl-1H-pyrazol-4-yl)phenyl)-1,6-naphthyridine-2-carbonyl)piperazine-1-carboxylate (85 mg, 0.166 mmol) in CH₂Cl₂ (4.5 mL) was added trifluoroacetic acid (0.483 mL, 3.31 mmol) at 0° C. and the mixture was stirred at rt for 1 h. After this time the trifluoroacetic acid was removed by azeotropic distillation with toluene and the residue was purified by chromatography on silica gel (Biotage, CH₂Cl_{2/25}% aq. NH₄OH in MeOH (9/1), 100:0 to 50:50) and further purified by ion exchange (SCX2 cartridge, loading with CH₂Cl₂/MeOH 9/1, elution with CH₂Cl₂/1N NH₃ in MeOH 9/1) to give a yellow residue. This residue was triturated with hot EtOAc and the residue filtered off to give 40 mg of material, which was further purified by prep. HPLC (Gilson, acetonitrile/water+0.1% formic acid gradient) to give the title compound (38 mg, 56%) as a yellow solid. ¹H NMR (500 MHz, DMSO-d6) ppm=8.79 (d, J=8.6, 1H), 8.17 (s, 1H), 8.13 (s, 1H), 7.90 (s, 1H), 7.65 (d, J=8.5, 1H), 7.60 (d, J=8.4, 2H), 7.57 (d, J=8.4, 2H), 7.26 (s, 2H), 3.88 (s, 3H), 3.61-3.53 (m, 2H), 3.44-3.37 (m, 2H), 2.80-2.71 (m, 2H), 2.70-2.59 (m, 2H) [M+H]⁺ 414. Rt 0.69 min (method M).

### Example 163

### [8-(1-Methyl-2,2-dioxo-2,3-dihydro-1H-benzo[c]isothiazol-5-yl)-[1,6]naphthyridin-2-yl]-methanol (51)

### 8-Bromo-[1,6]naphthyridine-2-carboxylic acid methyl ester

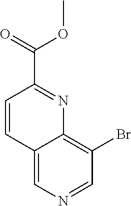

In a 100 mL flask 8-bromo-1,6-naphthyridine-2-carboxylic acid (1.00 g, 3.95 mmol) was suspended in methanol (20 mL). Thionyl chloride (0.89 mL, 11.9 mmol) was added dropwise at rt. The reaction mixture was stirred at rt overnight and then evaporated to dryness. The yellow residue was treated with water and solid sodium carbonate. The yellow precipitate was filtered off, washed with water and dried at 60° C. for 4 h to yield 985 mg (92% purity, 86%) of the title compound as a yellow solid.

### (8-Bromo-[1,6]naphthyridin-2-yl)-methanol

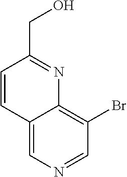 [SMILES: OCc1ccc2cncc(Br)c2n1]

In a 50 mL screw-capped vessel 8-bromo-[1,6]naphthyridine-2-carboxylic acid methyl ester (500 mg, 1.72 mmol) was dissolved in methanol (20 mL). Sodium borohydride (130 mg, 3.45 mmol) was added and the reaction mixture was stirred overnight at rt. The reaction mixture was evaporated to dryness. The yellow residue was treated with 30 mL water and was adjusted to pH 7 with 1 N HCl. Dichloromethane was added and the mixture was filtered through a phase separator. The organic layer was evaporated to dryness. The yellow residue was purified by flash chromatography (dichloromethane/methanol) to yield 236 mg (57%) of the title compound as a white solid.

### [8-(1-Methyl-2,2-dioxo-2,3-dihydro-1H-benzo[c]isothiazol-5-yl)-[1,6]naphthyridin-2-yl]-methanol

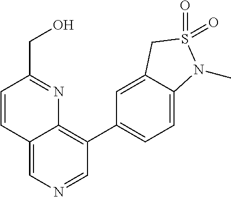 [SMILES: CN1c2ccc(-c3cncc4ccc(CO)nc34)cc2CS1(=O)=O]

In a microwave vial (8-bromo-[1,6]naphthyridin-2-yl)-methanol (50.0 mg, 0.21 mmol) was suspended in acetonitrile (2 mL). 1-Methyl-5-(4,4,5,5-tetramethyl-[1,3,2]dioxaborolan-2-yl)-1,3-dihydro-benzo[c]isothiazole 2,2-dioxide (77.6 mg, 0.25 mmol), 0.5 M aqueous sodium carbonate solution (837 μl, 0.42 mmol) and Pd(dppf)Cl₂ dichloromethane complex (8.54 mg, 0.01 mmol) were added. The vial was flushed with nitrogen and stirred at 120° C. for 1 h under microwave irradiation. The reaction mixture was treated with ethyl acetate, filtered and the filtrate was evaporated to dryness. The residue was purified by flash chromatography (dichloromethane/methanol). The solid residue was treated with diethyl ether, filtered off, washed with diethyl ether and air-dried to yield 27.2 mg (37%) of the title compound as a cream coloured solid. [M+H]⁺ 342. Rt 1.74 min (method L).

### Example 164

### 5-Amino-8-(1-methyl-2,2-dioxo-2,3-dihydro-1H-benzo[c]isothiazol-5-yl)-[1,6]naphthyridin-2-yl]-methanol (58)

### [8-(1-Methyl-2,2-dioxo-2,3-dihydro-1H-benzo[c]isothiazol-5-yl)-6-oxy-[1,6]naphthyridin-2-yl]-methanol

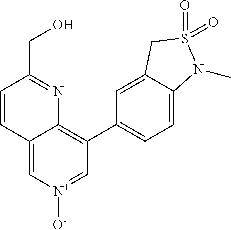 [SMILES: CN1c2ccc(-c3c[n+]([O-])cc4ccc(CO)nc34)cc2CS1(=O)=O]

In a 12 mL screw-capped vessel [8-(1-methyl-2,2-dioxo-2,3-dihydro-1H-benzo[c]isothiazol-5-yl)-[1,6]naphthyridin-2-yl]methanol (Example 106) (75.0 mg, 0.22 mmol) was dissolved in dichloromethane (6 mL) and treated with 3-chloroperoxybenzoic acid (108 mg, 0.44 mmol). The orange solution was stirred overnight at room temperature. The mixture was treated with 1 N NaOH solution, dichloromethane added and the organic phase was separated. The organic layer was washed with water, dried over sodium sulfate, filtered and evaporated to dryness to yield 17.0 mg (22%) of the title compound as a yellow solid.

### [5-Amino-8-(1-methyl-2,2-dioxo-2,3-dihydro-1H-benzo[c]isothiazol-5-yl)-[1,6]naphthyridin-2-yl]-methanol

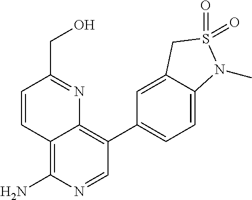 [SMILES: CN1c2ccc(-c3cnc(N)c4ccc(CO)nc34)cc2CS1(=O)=O]

In a 50 mL flask [8-(1-methyl-2,2-dioxo-2,3-dihydro-1H-benzo[c]isothiazol-5-yl)-6-oxy-[1,6]naphthyridin-2-yl]-methanol (17.0 mg, 0.05 mmol) was dissolved in pyridine (3 mL). Toluene-4-sulfonyl chloride (14.1 mg, 0.07 mmol) was added and the reaction mixture was stirred for 30 minutes at rt. Ethanolamine (92.3 μl, 1.54 mmol) was added at rt. The reaction mixture turned suddenly to dark red and was stirred for additional 30 minutes at rt. The reaction mixture was evaporated to dryness and the residue was purified by preparative HPLC (acetonitrile/water) to yield the trifluoroacetate acid salt of the title compound (5.40 mg, 19%) as a yellow solid. ¹H NMR (500 MHz, DMSO-d6) ppm=14.31-12.57 (m, 1H), 8.98 (d, J=8.7, 1H), 8.95-8.70 (m, 2H), 7.92 (s, 1H), 7.88 (d, J=8.7, 1H), 7.64-7.58 (m, 2H), 7.05 (d, J=8.5, 1H), 4.73 (s, 2H), 4.70 (s, 2H), 3.10 (s, 3H). [M+H]⁺ 357. Rt 1.78 min (method L).

### Example 165

### Methyl-[8-(1-methyl-2,2-dioxo-2,3-dihydro-1H-benzo[c]isothiazol-5-yl)-[1,6]naphthyridin-2-ylmethyl]-amine (53)

### 2-Chloromethyl-8-(1-methyl-2,2-dioxo-2,3-dihydro-1H-benzo[c]isothiazol-5-yl)-[1,6]naphthyridine

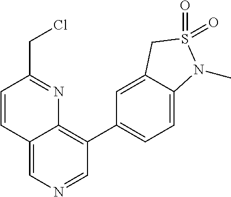 [SMILES: CN1c2ccc(-c3cncc4ccc(CCl)nc34)cc2CS1(=O)=O]

In a 100 mL flask [8-(1-methyl-2,2-dioxo-2,3-dihydro-1H-benzo[c]isothiazol-5-yl)-[1,6]naphthyridin-2-yl]methanol (Example 106) (89.0 mg, 0.26 mmol) was dissolved in dichloromethane (3 mL). Triethylamine (130 μl, 1.04 mmol) and methanesulfonyl chloride (30.3 μl, 0.39 mmol) were added at rt. The reaction solution was stirred overnight at rt. The mixture was evaporated to dryness to give 46 mg (50% purity, 25%) of the title compound as a brown solid.

### Methyl-[8-(1-methyl-2,2-dioxo-2,3-dihydro-1H-benzo[c]isothiazol-5-yl)-[1,6]naphthyridin-2-ylmethyl]-amine

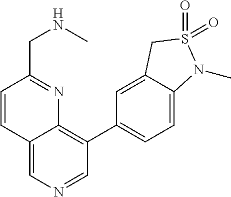 [SMILES: CNCc1ccc2cncc(-c3ccc4c(c3)CS(=O)(=O)N4C)c2n1]

In a 12 mL screw-capped vessel 2-chloromethyl-8-(1-methyl-2,2-dioxo-2,3-dihydro-1H-benzo[c]isothiazol-5-yl)-[1,6]naphthyridine (50% purity, 46.0 mg, 0.09 mmol) was dissolved in a 2 M solution of methylamine in tetrahydrofuran (2.00 mL, 4.00 mmol) at rt and the mixture was stirred for 20 h at rt. The mixture was evaporated to dryness and purified by HPLC (acetonitrile/water) to yield the trifluoroacetate acid salt of the title compound (3.60 mg, 9%) as a pale yellow solid. [M+H]⁺ 355. Rt 1.56 min (method L).

### Example 166

### C-[8-(1-Methyl-2,2-dioxo-2,3-dihydro-1H-benzo[c]isothiazol-5-yl)-[1,6]naphthyridin-2-yl]-methylamine (71)

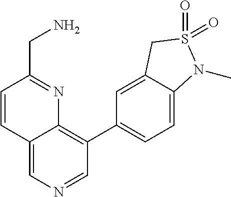 [SMILES: CN1c2ccc(-c3cncc4ccc(CN)nc34)cc2CS1(=O)=O]

In a 12 mL screw-capped vessel [8-(1-methyl-2,2-dioxo-2,3-dihydro-1H-benzo[c]isothiazol-5-yl)-[1,6]naphthyridin-2-yl]-methanol (Example 106) (200 mg, 0.59 mmol) was dissolved in dichloromethane (5 mL). Triethylamine (293 μL, 2.34 mmol) and methanesulfonyl chloride (68.1 μL, 0.88 mmol) were added. The reaction mixture was stirred for 2 h at rt. A 7 N solution of ammonia in methanol (2.00 mL, 14.0 mmol) was added and the mixture heated at 50° C. for 15 h. The reaction mixture was evaporated to dryness. The residue was purified by flash chromatography (dichloromethane/methanol) to give 50.0 mg (23%) of the title compound as a brown solid. [M+H]⁺ 341. Rt 1.5 min (method L).

### Example 167

### N-[8-(1-Methyl-2,2-dioxo-2,3-dihydro-1H-benzo[c]isothiazol-5-yl)-[1,6]naphthyridin-2-ylmethyl]-acetamide (38)

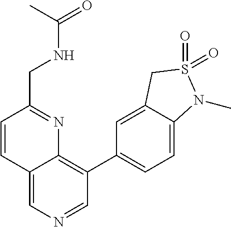 [SMILES: CC(=O)NCc1ccc2cncc(-c3ccc4c(c3)CS(=O)(=O)N4C)c2n1]

In a 12 mL screw-capped vessel C-[8-(1-Methyl-2,2-dioxo-2,3-dihydro-1H-benzo[c]isothiazol-5-yl)-[1,6]naphthyridin-2-yl]-methylamine (Example 109) (50.0 mg, 0.14 mmol) was dissolved in pyridine (1 mL). Acetyl chloride (19.9 μl, 0.28 mmol) was added and the reaction mixture was stirred at rt overnight. The reaction mixture was evaporated to dryness. The oily residue was purified by preparative HPLC (acetonitrile/water) to yield the trifluoroacetate salt of the title compound (3.80 mg, 5%) as a yellow solid. [M+H]⁺ 383. Rt 1.69 min (method L).

### Example 168

### 3-Methyl-8-(1-methyl-2,2-dioxo-2,3-dihydro-1H-benzo[c]isothiazol-5-yl)-[1,6]naphthyridine-2-carboxylic acid methylamide (12)

### 8-Bromo-3-methyl-[1,6]naphthyridine-2-carboxylic acid methylamide

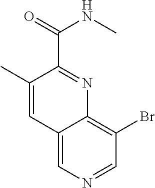 [SMILES: CNC(=O)c1nc2c(Br)cncc2cc1C]

In a screw-capped vessel 8-bromo-3-methyl-[1,6]naphthyridine-2-carboxylic acid (40.0 mg, 0.15 mmol) was dissolved in N,N-dimethylformamide (2 mL). 4-Methylmorpholine (49.4 μL, 0.45 mmol) and [2-(1H-benzotriazol-1-yl)-1,1,3,3-tetramethyluronium tetrafluoroborate (TBTU) (96.2 mg, 0.30 mmol) and a 2 M solution of methylamine in tetrahydrofuran (0.75 mL, 1.50 mmol) were added at rt and the pale solution was stirred for 3 days at 70° C. The reaction solution was evaporated to dryness. Water and dichloromethane were added and the organic phase separated. The organic layer was dried over sodium sulfate, filtered and evaporated to dryness. The residue was purified by flash chromatography (dichloromethane/methanol) to yield 10.6 mg (88% purity, 23%) of the title compound as a pale yellow solid.

### 3-Methyl-8-(1-methyl-2,2-dioxo-2,3-dihydro-1H-benzo[c]isothiazol-5-yl)-[1,6]naphthyridine-2-carboxylic acid methylamide

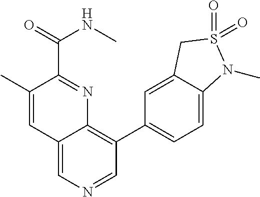

In a microwave vial 8-bromo-3-methyl-[1,6]naphthyridine-2-carboxylic acid methylamide (88% purity, 10.6 mg, 0.03 mmol) was dissolved in acetonitrile (2 mL). 1-Methyl-5-(4,4,5,5-tetramethyl-[1,3,2]dioxaborolan-2-yl)-1,3-dihydro-benzo[c]isothiazole 2,2-dioxide (12.6 mg, 0.04 mmol), 0.5 M aqueous sodium carbonate solution (0.14 mL, 0.07 mmol) and Pd(dppf)Cl2 dichloromethane complex (1.39 mg, 0.002 mmol) were added at rt. The vial was flushed with nitrogen and stirred at 120° C. for 1 h under microwave irradiation. The reaction mixture was treated with ethyl acetate, filtered and the filtrate was evaporated to dryness. The dark brown residue was purified by preparative HPLC (acetonitrile/water) to yield the trifluoracetate salt of the title compound (1.90 mg, 11%) of as a yellow solid. [M+H]⁺ 383. Rt 1.91 min (method L).

### Example 169

### 3-Methyl-8-(1-methyl-2,2-dioxo-2,3-dihydro-1H-benzo[c]isothiazol-5-yl)-[1,6]naphthyridine-2-carboxylic acid cyclopropylamide (28)

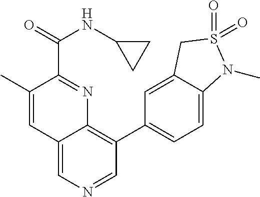

3-Methyl-8-(1-methyl-2,2-dioxo-2,3-dihydro-1H-benzo[c]isothiazol-5-yl)-[1,6]naphthyridine-2-carboxylic acid cyclopropylamide was prepared in a manner similar to Example 111, using cyclopropylamine as the starting material. ¹H NMR (400 MHz, DMSO-d6) ppm=9.36 (s, 1H), 8.77 (s, 1H), 8.53 (s, 1H), 8.49 (d, J=4.7, 1H), 7.89-7.81 (m, 2H), 7.15-7.08 (m, 1H), 4.71 (s, 2H), 3.13 (s, 3H), 2.94-2.84 (m, 1H), 2.58 (s, 3H), 0.78-0.72 (m, 2H), 0.58-0.53 (m, 2H). [M+H]⁺ 409. Rt 2.08 min (method L).

### Example 170

### (3,3-Difluoro-pyrrolidin-1-yl)-[3-methyl-8-(1-methyl-2,2-dioxo-2,3-dihydro-1H-benzo[c]isothiazol-5-yl)-[1,6]naphthyridin-2-yl]-methanone (41)

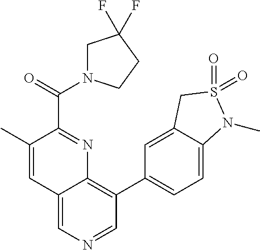

(3,3-Difluoro-pyrrolidin-1-yl)-[3-methyl-8-(1-methyl-2,2-dioxo-2,3-dihydro-1H-benzo[c]isothiazol-5-yl)-[1,6]naphthyridin-2-yl]methanone was prepared in a manner similar to Example 111, using 3,3,-difluoropyrrolidine as the starting material. ¹H NMR (400 MHz, DMSO-d6) ppm=9.40-9.35 (m, 1H), 8.74 (d, J=3.8, 1H), 8.59 (dd, J=4.8, 1.1, 1H), 7.72-7.67 (m, 2H), 7.09 (dd, J=9.9, 8.1, 1H), 4.71 (d, J=8.9, 2H), 3.98 (t, J=13.0, 1H), 3.86-3.74 (m, 2H), 3.52 (t, J=7.4, 1H), 3.12 (s, 3H), 2.54-2.44 (m, 5H). [M+H]⁺ 459. Rt 2.19 min (method L).

### Example 171

### 8-(4-(1-methyl-1H-pyrazol-4-yl)phenyl)-1,6-naphthyridin-2-ol (79)

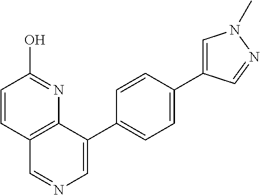 [SMILES: Cn1cc(-c2ccc(-c3cncc4ccc(O)nc34)cc2)cn1]

8-Bromo-1,6-naphthyridin-2-ol (150 mg, 0.667 mmol), 1-methyl-4-(4-(4,4,5,5-tetramethyl-1,3,2-dioxaborolan-2-yl)phenyl)-1H-pyrazole (227 mg, 0.800 mmol) and Pd(dppf)Cl₂ dichloromethane complex (27.2 mg, 0.033 mmol) were loaded in a microwave vial. A degassed 0.5 M solution of sodium carbonate in water (1.9 mL, 0.933 mmol) and degassed acetonitrile (11.5 mL) were then added. The reaction mixture was heated at 120° C. for 1 h under microwave irradiation. The reaction mixture was concentrated in vacuo and the residue purified by column chromatography (Biotage, dichloromethaneCH₂Cl₂/EtOH 98/2 to 94/6) to give the title compound (190 mg, 94% yield). ¹H NMR (500 MHz, CDCl₃) ppm=8.83 (s, 1H), 8.54 (s, 1H), 7.90 (d, J=9.6, 1H), 7.85 (d, J=0.7, 1H), 7.72 (s, 1H), 7.68 (d, J=8.0, 2H), 7.44 (d, J=8.0, 2H), 6.75 (d, J=9.5, 1H), 4.01 (s, 3H). [M+H]⁺ 303. Rt 1.95 min (method N).

### Example 172

### 2-(1-methyl-1H-imidazol-2-yl)-8-(4-(1-methyl-1H-pyrazol-4-yl)phenyl)-1,6-naphthyridine (78)

### 2-chloro-8-(4-(1-methyl-1H-pyrazol-4-yl)phenyl)-1,6-naphthyridine

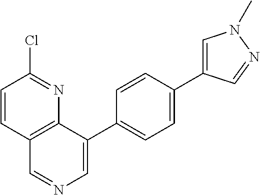 [SMILES: Cn1cc(-c2ccc(-c3cncc4ccc(Cl)nc34)cc2)cn1]

A mixture of 8-(4-(1-methyl-1H-pyrazol-4-yl)phenyl)-1,6-naphthyridin-2-ol (Example 114) (50 mg, 0.165 mmol) and few drops of DMF in phosphorus oxychloride (1.1 mL) was heated at 70° C. for 1.5 h and at 80° C. for 3.5 h. The reaction mixture was transferred into a flask and excess POCl₃ was evaporated by azeotropic removal with toluene. dichloromethane Saturated aqueous NaHCO₃ was added and the mixture was subsequently concentrated. The crude was purified via biotage column chromatography (dichloromethane/EtOH 99/1 to 95/5) to give the title compound (28 mg, 53%).

### 2-(1-methyl-1H-imidazol-2-yl)-8-(4-(1-methyl-1H-pyrazol-4-yl)phenyl)-1,6-naphthyridine

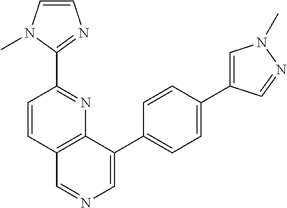 [SMILES: Cn1cc(-c2ccc(-c3cncc4ccc(-c5nccn5C)nc34)cc2)cn1]

To 2-chloro-8-(4-(1-methyl-1H-pyrazol-4-yl)phenyl)-1,6-naphthyridine (30.0 mg, 0.094 mmol) and Pd(PPh₃)₄ (5.4 mg, 4.68 μmol) in a microwave vial were added degassed 1,4-dioxane (360 μL) and 1-methyl-2-(tributylstannyl)-1H-imidazole (44.5 μL, 0.140 mmol). The reaction mixture was heated with an oil bath to 100° C. for 7 h. The reaction mixture was cooled to room temperature and concentrated under reduced pressure. The crude product was purified via biotage column chromatography (dichloromethane/EtOH 99/1 to 90/10). The product obtained was filtered twice on a SCX column and the product was released with 1N NH₃ in methanol to give the title compound (10 mg, 29%). ¹H NMR (500 MHz, CDCl₃) ppm=9.23 (s, 1H), 8.80 (s, 1H), 8.56 (d, J=8.7, 1H), 8.37 (d, J=8.7, 1H), 7.86 (d, J=0.8, 1H), 7.75-7.69 (m, 3H), 7.64-7.59 (m, 2H), 7.21 (d, J=1.1, 1H), 7.00 (d, J=1.1, 1H), 4.01 (s, 3H), 3.97 (s, 3H). [M+H]⁺ 367. Rt 2.49 min (method N).

### Example 173

### 2-(8-(4-(1-methyl-1H-pyrazol-4-yl)phenyl)-1,6-naphthyridin-2-yl)oxazole (44)

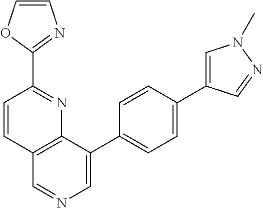 [SMILES: Cn1cc(-c2ccc(-c3cncc4ccc(-c5ncco5)nc34)cc2)cn1]

2-(8-(4-(1-methyl-1H-pyrazol-4-yl)phenyl)-1,6-naphthyridin-2-yl)oxazole was prepared in a manner similar to Example 115, using 2-(tributylstannyl)oxazole as the starting material. ¹H NMR (500 MHz, CDCl₃) ppm=9.36 (s, 1H), 8.93 (s, 1H), 8.61-8.52 (m, 2H), 7.98-7.93 (m, 3H), 7.89 (d, J=0.8, 1H), 7.75 (s, 1H), 7.73-7.69 (m, 2H), 7.44 (d, J=0.8, 1H), 4.01 (s, 3H). [M+H]⁺ 354. Rt 2.72 min (method N).

### Example 174

### 2-(1-methyl-1H-imidazol-2-yl)-8-(4-(1-methyl-1H-pyrazol-4-yl)phenyl)-1,6-naphthyridin-5-amine (60)

### 2-(1-methyl-1H-imidazol-2-yl)-8-(4-(1-methyl-1H-pyrazol-4-yl)phenyl)-1,6-naphthyridine 6-oxide

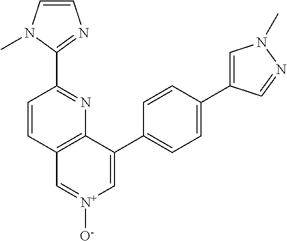 [SMILES: Cn1cc(-c2ccc(-c3c[n+]([O-])cc4ccc(-c5nccn5C)nc34)cc2)cn1]

To a solution of 2-(1-methyl-1H-imidazol-2-yl)-8-(4-(1-methyl-1H-pyrazol-4-yl)phenyl)-1,6-naphthyridine (Example 115) (0.034 g, 0.094 mmol) in dichloromethane (0.940 mL) was added 3-chloroperoxybenzoic acid (0.016 g, 0.094 mmol) and the reaction mixture was stirred at rt for 1 h. Additional 3-chloroperoxybenzoic acid (0.016 g) was added and the reaction mixture was stirred for another 1 h. 1N NaOH and dichloromethane were added to the reaction mixture and the layers were separated. The aqueous layer was extracted with dichloromethane and the organics layers were dried over MgSO₄, filtered, and the filtrate concentrated under reduced pressure. The crude material was purified via biotage column chromatography (dichloromethane/EtOH, 98/2 to 85/15, single step 12 g) to give the title compound (5 mg, 14% yield).

### 2-(1-methyl-1H-imidazol-2-yl)-8-(4-(1-methyl-1H-pyrazol-4-yl)phenyl)-1,6-naphthyridin-5-amine

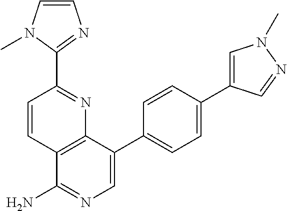 [SMILES: Cn1cc(-c2ccc(-c3cnc(N)c4ccc(-c5nccn5C)nc34)cc2)cn1]

To a suspension of 2-(1-methyl-1H-imidazol-2-yl)-8-(4-(1-methyl-1H-pyrazol-4-yl)phenyl)-1,6-naphthyridine 6-oxide (5 mg, 0.013 mmol) in pyridine (300 μL, 2.61 mmol) was added 4-toluenesulfonlyl chloride (3 mg, 0.016 mmol) and the mixture was stirred at rt for 25 min. To the mixture was added ethanolamine (19.77 μl, 0.327 mmol) and the reaction mixture was stirred at rt for 1 h before being diluted with water and ethyl acetate. The layers were separated and the aqueous layers were extracted three times with EtOAc. The organic layers were combined, dried over MgSO₄, filtered and the filtrate concentrated under reduced pressure. The crude was purified via preparative TLC (dichloromethane/EtOH 90/10). The product was solubilised with dichloromethane and a drop of methanol and then washed with water twice to remove trace of ethanolamine. The organic layer was dried over MgSO₄, filtered and the filtrate concentrated under reduced pressure to give the title compound (1.7 mg, 34%). ¹H NMR (500 MHz, CH₃OD) ppm=8.68 (d, J=8.8, 1H), 8.23 (d, J=8.8, 1H), 8.02 (2s, 1H), 7.88 (s, 1H), 7.66 (d, J=8.3, 2H), 7.61 (d, J=8.3, 2H), 7.22 (s, 1H,), 7.14 (s, 1H), 3.97 (s, 3H), 3.91 (s, 3H). [M−H]⁺ 382. Rt 1.92 min (method N).

### Example 175

### 8-(4-(1-methyl-1H-pyrazol-4-yl)phenyl)-2-(1-methyl-1H-pyrazol-5-yl)-1,6-naphthyridine (7)

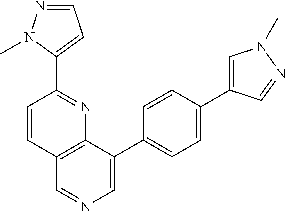 [SMILES: Cn1cc(-c2ccc(-c3cncc4ccc(-c5ccnn5C)nc34)cc2)cn1]

2-chloro-8-(4-(1-methyl-1H-pyrazol-4-yl)phenyl)-1,6-naphthyridine (20 mg, 0.062 mmol), 1-methyl-3-(4,4,5,5-tetramethyl-1,3,2-dioxaborolan-2-yl)-1H-pyrazole (15.57 mg, 0.075 mmol) and Pd(dppf)Cl₂.CH₂Cl₂ (2.55 mg, 3.12 μmol) were loaded in a microwave vial then degassed sodium carbonate in water (175 μl, 0.087 mmol) and degassed acetonitrile (1075 μl) were added. The reaction mixture was heated at 120° C. for 60 min under microwave irradiation. The crude was purified via biotage column chromatography (Dichloromethane/EtOH 99.9/0.1 to 95/5) to give the title compound (15 mg, 66% yield). ¹H NMR (500 MHz, CDCl₃) ppm=9.26 (bs, 1H), 8.85 (bs, 1H), 8.39 (d, J=8.6, 1H), 7.90 (d, J=8.6, 1H), 7.87 (d, J=0.8, 1H), 7.76-7.73 (m, 2H), 7.72 (d, J=0.8, 1H), 7.65-7.61 (m, 2H), 7.56 (d, J=2.0, 1H), 6.87 (d, J=2.0, 1H), 4.15 (s, 3H), 4.01 (s, 3H). [M+H]⁺ 367. Rt 3.19 min (method N).

### Example 176

### N-(8-(4-(1-Methyl-1H-pyrazol-4-yl)phenyl)-1,6-naphthyridin-2-yl)acetamide (13)

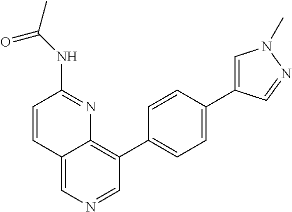 [SMILES: CC(=O)Nc1ccc2cncc(-c3ccc(-c4cnn(C)c4)cc3)c2n1]

A mixture of 2-chloro-8-(4-(1-methyl-1H-pyrazol-4-yl)phenyl)-1,6-naphthyridine (30.0 mg, 0.094 mmol), acetamide (8.3 mg, 0.140 mmol), xantphos (4.3 mg, 7.48 μmol), palladium diacetate (1.1 mg, 4.68 μmol) and caesium carbonate (25.8 mg, 0.122 mmol) in dioxane (1 mL) was stirred at 80° C. for 2 h. The mixture was concentrated in vacuum and the resulting residue was purified by chromatography on silica gel (biotage, CH₂Cl₂/EtOH, 100:0 to 96:4) to give the title compound (23 mg, 72%) as a colourless solid. ¹H-NMR (500 MHz, CDCl₃) ppm=9.28 (bs, 1H), 9.13 (s, 1H), 8.73 (s, 1H), 8.47 (d, J=8.9, 1H), 8.27 (d, J=8.9, 1H), 7.71 (s, 1H), 7.67 (d, J=8.2, 2H), 7.57 (s, 1H), 7.45 (d, J=8.2, 1H), 3.92 (s, 3H), 2.10 (s, 3H). [M+H]⁺ 344. Rt 1.31 min (method M).

### Example 177

### 1-(8-(4-(1-Methyl-1H-pyrazol-4-yl)phenyl)-1,6-naphthyridin-2-yl)pyrrolidin-2-one (23)

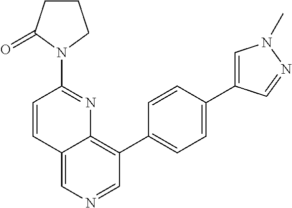 [SMILES: Cn1cc(-c2ccc(-c3cncc4ccc(N5CCCC5=O)nc34)cc2)cn1]

A mixture of 2-chloro-8-(4-(1-methyl-1H-pyrazol-4-yl)phenyl)-1,6-naphthyridine (30.0 mg, 0.094 mmol), pyrrolidinone (27.9 mg, 0.327 mmol), potassium phosphate (111.0 mg, 0.524 mmol), Pd₂(dba)₃ (13.7 mg, 0.015 mmol) and xantphos (17.86 mg, 0.031 mmol) in toluene (0.8 mL) was heated to 80° C. for 2 h. The reaction mixture was cooled to room temperature and concentrated under reduced pressure. The residue was purified by chromatography on silica gel (biotage, CH₂Cl₂/EtOH, 100:0 to 95/5) and further purified by using a scx2-cartridge (loading with CH₂Cl₂/MeOH 9/1, elution with CH₂Cl₂/1N NH₃ in MeOH 9/1) to give the product (28 mg, 81%) as a colourless solid. ¹H-NMR (500 MHz, MeOD/CDCl₃, 1:1) ppm=9.07 (s, 1H), 8.71 (d, J=9.2, 1H), 8.65 (s, 1H), 8.36 (d, J=9.2, 1H), 7.86 (s, 1H), 7.84 (s, 1H), 7.77 (d, J=8.2, 2H), 7.62 (d, J=8.2, 2H), 4.13 (t, J=7.2, 2H), 3.95 (s, 3H), 2.71 (t, J=8.1, 2H), 2.21-2.07 (m, 2H). [M+H]⁺ 370. Rt 1.36 min (method M).

### Example 178

### N-(8-(4-(1-Methyl-1H-pyrazol-4-yl)phenyl)-1,6-naphthyridin-2-yl)methanesulfonamide (2)

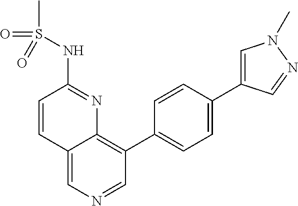 [SMILES: Cn1cc(-c2ccc(-c3cncc4ccc(NS(C)(=O)=O)nc34)cc2)cn1]

N-(8-(4-(1-Methyl-1H-pyrazol-4-yl)phenyl)-1,6-naphthyridin-2-yl)methanesulfonamide was prepared in a manner similar to Example 120, using methanesulfonamide as the starting material. ¹H-NMR (500 MHz, DMSO-d6) ppm=11.42 (bs, 1H), 9.17 (s, 1H), 8.76 (s, 1H), 8.50 (d, J=8.8, 1H,), 8.24 (d, J=0.8, 1H), 7.95 (d, J=0.8, 1H), 7.88 (d, J=8.3, 2H), 7.69 (d, J=8.3, 2H), 7.19 (d, J=8.8, 1H), 3.89 (s, 3H), 3.28 (s, 3H). [M+H]⁺ 380. Rt 1.21 min (method M).

### Example 179

### 1-(8-(4-(1-methyl-1H-pyrazol-4-yl)phenyl)-1,6-naphthyridin-2-yl)imidazolidin-2-one (5)

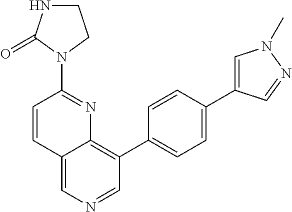 [SMILES: Cn1cc(-c2ccc(-c3cncc4ccc(N5CCNC5=O)nc34)cc2)cn1]

A mixture of 2-chloro-8-(4-(1-methyl-1H-pyrazol-4-yl)phenyl)-1,6-naphthyridine (27 mg, 0.084 mmol), 2-imidazolidone (26 mg, 0.30 mmol), K₃PO₄ (100 mg, 0.47 mmol), Pd₂(dba)₃ (12 mg, 0.013 mmol), and Xantphos (16 mg, 0.028 mmol) in toluene (0.7 mL) was evacuated and purged with Ar repeatedly (×3) and then heated at 80° C. for 17 h. The crude reaction mixture was concentrated in vacuo and the residue purified by Biotage (SingleStep 12 g column, CH₂Cl₂/EtOH 98/2->94/6) to give the title compound as a yellow solid (4.3 mg, 14%). ¹H NMR (500 MHz, CD₃OD) ppm=9.43 (s, 1H), 8.97 (d, J=9.4, 1H), 8.72 (s, 1H), 8.62 (d, J=9.4, 1H), 8.09 (s, 1H), 7.93 (s, 1H), 7.90 (d, J=8.4, 2H), 7.77 (d, J=8.4, 2H), 4.24 (m, 2H), 3.98 (s, 3H), 3.59 (m, 2H). [M+1]⁺ 371. Rt 1.23 min (method M).

### Example 180

### 2-(3-methoxyazetidin-1-yl)-8-(4-(1-methyl-1H-pyrazol-4-yl)phenyl)-1,6-naphthyridine (73)

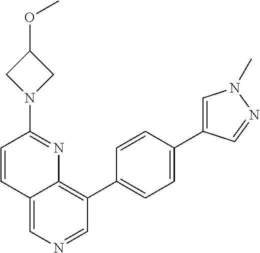 [SMILES: COC1CN(c2ccc3cncc(-c4ccc(-c5cnn(C)c5)cc4)c3n2)C1]

2-(3-methoxyazetidin-1-yl)-8-(4-(1-methyl-1H-pyrazol-4-yl)phenyl)-1,6-naphthyridine was prepared in a manner similar to Example 122, using 3-methoxyazetidine hydrochloride as the starting material. ¹H NMR (500 MHz, CDCl₃) ppm=8.86 (s, 1H), 8.63 (s, 1H), 7.95 (d, J=8.9, 1H), 7.86 (d, J=8.3, 2H), 7.84 (s, 1H), 7.68 (s, 1H), 7.57 (d, J=8.3, 2H), 6.62 (d, J=8.9, 1H), 4.40-4.30 (m, 3H), 4.07-4.02 (m, 2H), 3.97 (s, 3H), 3.35 (s, 3H). [M+H]⁺ 372. Rt 1.22 min (method M).

### Example 181

### N-methyl-8-(4-(1-methyl-1H-pyrazol-4-yl)phenyl)-1,6-naphthyridin-2-amine (56)

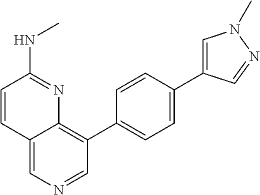 [SMILES: CNc1ccc2cncc(-c3ccc(-c4cnn(C)c4)cc3)c2n1]

A solution of 2-chloro-8-(4-(1-methyl-1H-pyrazol-4-yl)phenyl)-1,6-naphthyridine (25 mg, 0.078 mmol), methylamine hydrochloride (29 mg, 0.43 mmol), and Et₃N (0.10 mL, 0.71 mmol) in NMP (0.5 mL) was heated at 220° C. for 3 h under microwave irradiation. The reaction mixture was concentrated in vacuo and the residue purified by Biotage SingleStep 12 g column, Dichloromethane/EtOH 97/3->92/8) to give the title compound as a pale yellow solid (12 mg, 45%). ¹H NMR (500 MHz, CDCl₃) ppm=8.83 (s, 1H), 8.60 (s, 1H), 7.92-7.82 (m, 4H), 7.68 (s, 1H), 7.57 (d, J=8.3, 2H), 6.71 (d, J=9.0, 1H), 5.36 (br s, 1H), 3.98 (s, 3H), 3.05 (d, J=4.7, 3H). [M+H]⁺ 316. Rt 1.07 min (method M).

### Example 182

### 8-(4-(1-methyl-1H-pyrazol-4-yl)phenyl)-2-(piperazin-1-yl)-1,6-naphthyridine (72)

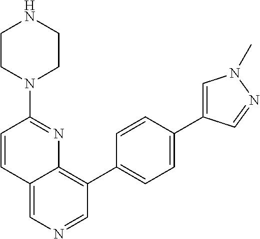 [SMILES: Cn1cc(-c2ccc(-c3cncc4ccc(N5CCNCC5)nc34)cc2)cn1]

8-(4-(1-methyl-1H-pyrazol-4-yl)phenyl)-2-(piperazin-1-yl)-1,6-naphthyridine was prepared in a manner similar to Example 124, using piperazine as the starting material. ¹H NMR (500 MHz, CDCl₃) ppm=8.89 (s, 1H), 8.63 (s, 1H), 8.03 (d, J=9.2, 1H), 7.85 (s, 1H), 7.81 (d, J=8.2, 2H), 7.70 (s, 1H), 7.59 (d, J=8.3, 2H), 7.04 (d, 1H), 3.98 (s, 3H), 3.83 (m, 4H), 3.05 (m, 4H). [M+H]⁺ 371. Rt 0.75 min (method M).

### Example 183

### 8-(4-(1-methyl-1H-pyrazol-4-yl)phenyl)-1,6-naphthyridine-2-carbonitrile (69)

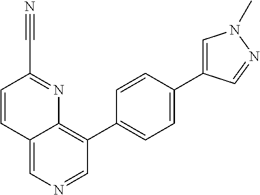 [SMILES: Cn1cc(-c2ccc(-c3cncc4ccc(C#N)nc34)cc2)cn1]

A mixture of 2-chloro-8-(4-(1-methyl-1H-pyrazol-4-yl)phenyl)-1,6-naphthyridine (25.0 mg, 0.078 mmol), zinc cyanide (10.1 mg, 0.086 mmol) and Pd(PPh₃)₄ (9.0 mg, 7.79 μmol) was heated in a focused microwave reactor at 60° C. for 1 h. The mixture was diluted with a small amount of CHCl₃/MeOH (1/1, 1 mL), EtOAc and water and the layers were separated. The aqueous layer was extracted with EtOAc twice. The combined organic layers were dried over MgSO₄, filtered, and the filtrate concentrated in vacuum. The resulting brown oil was purified by chromatography on silica gel (biotage, CH₂Cl₂/EtOH, 100:0 to 97:3) and further purified by using a scx2-cartridge (loading with CH₂Cl₂/MeOH 9/1, elution with CH₂Cl₂/1N NH₃ in MeOH 9/1) to give the product (16.8 mg, 69%) as a yellow solid. ¹H-NMR (500 MHz, DMSO-d6) ppm=9.56 (s, 1H), 8.96 (d, J=8.4, 1H), 8.96 (s, 1H), 8.27 (d, J=8.4, 1H), 8.25 (s, 1H), 7.98 (d, J=0.8, 1H), 7.78-7.72 (m, 4H), 3.90 (s, 3H). [M+H]⁺ 312. Rt 1.37 min (method M).

### Example 184

### 8-(4-(1-methyl-1H-pyrazol-4-yl)phenyl)-2-(2H-tetrazol-5-yl)-1,6-naphthyridine (42)

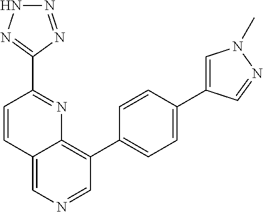 [SMILES: Cn1cc(-c2ccc(-c3cncc4ccc(-c5nn[nH]n5)nc34)cc2)cn1]

A mixture of 8-(4-(1-methyl-1H-pyrazol-4-yl)phenyl)-1,6-naphthyridine-2-carbonitrile (26 mg, 0.084 mmol) and sodium azide (10 mg, 0.15 mmol) in DMF (0.5 mL) was heated at 180° C. for 2 h under microwave irradiation. Water (10 mL) added and washed with CH₂Cl₂ (4×10 mL). The aqueous layer was concentrated in vacuo, MeOH (20 mL) added, and filtered. The filtrate was concentrated and the residue purified by ion exchange (SCX-2) to give the title compound as a pale yellow solid (7 mg, 24%). ¹H NMR (500 MHz, CD₃OD) ppm=9.34 (s, 1H), 8.79 (s, 1H), 8.73 (d, J=8.5, 1H), 8.46 (d, J=8.6, 1H), 8.09 (s, 1H), 7.94 (s, 1H), 7.91 (d, J=8.0, 2H), 7.77 (d, J=8.0, 2H), 3.99 (s, 3H). [M+H]⁺ 355; Rt 1.24 min (method M).

### Example 185

### 8-(2-methyl-4-(1-methyl-1H-pyrazol-4-yl)phenyl)-1,6-naphthyridine-2-carboxamide (19)

### 8-(2-methyl-4-(1-methyl-1H-pyrazol-4-yl)phenyl)-1,6-naphthyridin-2-ol

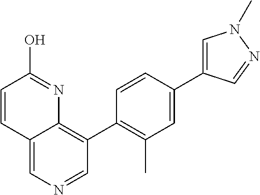 [SMILES: Cc1cc(-c2cnn(C)c2)ccc1-c1cncc2ccc(O)nc12]

A mixture of 8-bromo-1,6-naphthyridin-2(1H)-one (100 mg, 0.44 mmol), 1-methyl-4-(3-methyl-4-(4,4,5,5-tetramethyl-1,3,2-dioxaborolan-2-yl)phenyl)-1H-pyrazole (243 mg, 0.82 mmol), and Pd(dppf)Cl₂—CH₂Cl₂ (20 mg, 0.024 mmol) in a mixture of MeCN (3 mL) and 0.5 M aq. Na₂CO₃ (1.4 mL, 0.70 mmol) was heated at 120° C. for 1 h under microwave irradiation. The crude reaction mixture was concentrated in vacuo and the residue was purified by Biotage (SingleStep 25 g column, CH₂Cl₂/EtOH 98/2->93/7) to give the title compound as a cream coloured solid (113 mg, 80%).

### 2-chloro-8-(2-methyl-4-(1-methyl-1H-pyrazol-4-yl)phenyl)-1,6-naphthyridine

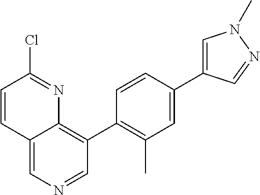 [SMILES: Cc1cc(-c2cnn(C)c2)ccc1-c1cncc2ccc(Cl)nc12]

To a suspension of 8-(2-methyl-4-(1-methyl-1H-pyrazol-4-yl)phenyl)-1,6-naphthyridin-2-ol (113 mg, 0.36 mmol) in anhyd. MeCN (2.5 mL) was added POCl₃ (0.15 mL, 1.61 mmol) followed by DMF (0.1 mL, 1.29 mmol), and the mixture heated at reflux for 1.5 h. The reaction was allowed to cool to rt and quenched by the careful addition of water (5 mL) and sat. aq. NaHCO₃ (20 mL) was added carefully. The mixture was extracted with CH₂Cl₂ (4×15 mL) and the combined org. layers washed with sat. aq. NaHCO₃ (10 mL) and sat. aq. NH₄Cl (20 mL), dried (NaSO₄), filtered, and the filtrate concentrated in vacuo. The crude product was purified by Biotage (SingleStep 12 g column, CH₂Cl₂/EtOH 98/2->92/8) to give the title compound as a dark red oil (108 mg, 90%).

### 8-(2-methyl-4-(1-methyl-1H-pyrazol-4-yl)phenyl)-1,6-naphthyridine-2-carbonitrile

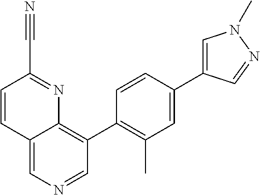 [SMILES: Cc1cc(-c2cnn(C)c2)ccc1-c1cncc2ccc(C#N)nc12]

A mixture of 2-chloro-8-(2-methyl-4-(1-methyl-1H-pyrazol-4-yl)phenyl)-1,6-naphthyridine (108 mg, 0.32 mmol), Zn(CN)₂ (42 mg, 0.36 mmol), and Pd(PPh₃)₄ (38 mg, 0.033 mmol) in anhyd. DMF (1.2 mL) was heated at 100° C. for 2.5 h under microwave irradiation. Water (10 mL) and EtOAc (30 mL) added, the layers separated, and the aqueous layer extracted with EtOAc (2×15 mL). The combined organic layers were washed with sat. aq. NH₄Cl (2×15 mL), dried (Na₂SO₄), filtered, and the filtrate concentrated in vacuo. The crude product was purified by Biotage (SingleStep 25 g column, CH₂Cl₂/EtOH 100/0->97/3) to give the title compound as a yellow resin (51 mg, 49%). ¹H NMR (500 MHz, CDCl₃) ppm=9.41 (s, 1H), 8.83 (s, 1H), 8.54 (d, J=8.4, 1H), 7.87-7.82 (m, 2H), 7.69 (s, 1H), 7.48 (s, 1H), 7.43 (dd, J=7.9, 1.8, 1H), 7.28 (d, J=7.8, 1H), 3.98 (s, 3H), 2.11 (s, 3H). [M+H]⁺ 326. Rt 1.34 min (method M).

### 8-(2-methyl-4-(1-methyl-1H-pyrazol-4-yl)phenyl)-1,6-naphthyridine-2-carboxamide

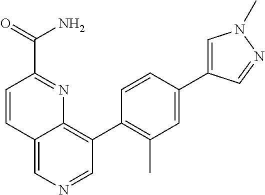 [SMILES: Cc1cc(-c2cnn(C)c2)ccc1-c1cncc2ccc(C(N)=O)nc12]

To 8-(2-methyl-4-(1-methyl-1H-pyrazol-4-yl)phenyl)-1,6-naphthyridine-2-carbonitrile (46 mg, 0.14 mmol) was added an ice cold mixture of water (0.1 mL) and conc. H₂SO₄ (1.5 mL, 28.10 mmol) at 0° C. The reaction mixture was then heated at 50° C. for 1 h. The mixture was then dropped into ice cold 2 M aq. NaOH (15 mL), and the resulting mixture neutralised with sat. aq. NaHCO₃ (˜30 mL). The solution was extracted with CH₂Cl₂ (3×20 mL), and the combined organic layers washed with water (15 mL), dried (MgSO₄), and concentrated in vacuo. The crude product was purified by Biotage (SingleStep 12 g column, CH₂Cl₂/EtOH 100/0->50/50) to give the title compound as a pale yellow solid (38 mg, 78%). ¹H NMR (500 MHz, CDCl₃) ppm=9.40 (s, 1H), 8.77 (s, 1H), 8.55 (d, J=8.4, 1H), 8.44 (d, J=8.4, 1H), 7.85 (s, 1H), 7.71 (s, 1H), 7.66 (d, J=4.5, 1H), 7.48 (s, 1H), 7.43 (dd, J=7.8, 1.8, 1H), 7.30 (d, J=7.8, 1H), 6.19 (d, J=4.4, 1H), 3.98 (s, 3H), 2.11 (s, 3H). [M+H]⁺ 344. Rt 1.26 min (method M).

### Example 186

### 8-(2-fluoro-4-(1-methyl-1H-pyrazol-4-yl)phenyl)-1,6-naphthyridine-2-carbonitrile (80)

### 8-(2-fluoro-4-(1-methyl-1H-pyrazol-4-yl)phenyl)-1,6-naphthyridin-2-ol

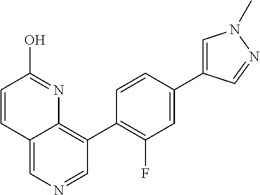 [SMILES: Cn1cc(-c2ccc(-c3cncc4ccc(O)nc34)c(F)c2)cn1]

A mixture of 8-bromo-1,6-naphthyridin-2(1H)-one (50 mg, 0.22 mmol), 4-(3-fluoro-4-(4,4,5,5-tetramethyl-1,3,2-dioxaborolan-2-yl)phenyl)-1-methyl-1H-pyrazole (52 mg, 0.17 mmol), K₃PO₄ (123 mg, 0.58 mmol), and Pd(dtbpf)Cl₂ (11 mg, 0.017 mmol) in a mixture of 1,4-dioxane (1 mL) and water (0.3 mL) was heated at 150° C. for 1 h under microwave irradiation. The cooled reaction mixture was concentrated in vacuo and purified by Biotage (SNAP 10 g column, CH₂Cl₂/EtOH 98/2->92/8) to give the title compound as a pale yellow oil (38 mg, 69%). 2-chloro-8-(2-fluoro-4-(1-methyl-1H-pyrazol-4-yl)phenyl)-1,6-naphthyridine

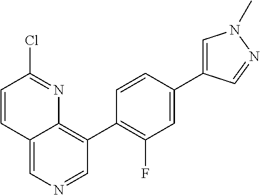 [SMILES: Cn1cc(-c2ccc(-c3cncc4ccc(Cl)nc34)c(F)c2)cn1]

To a suspension of 8-(2-fluoro-4-(1-methyl-1H-pyrazol-4-yl)phenyl)-1,6-naphthyridin-2-ol (38 mg, 0.12 mmol) in anhyd. MeCN (2 mL) was added POCl₃ (0.050 mL, 0.53 mmol) followed by DMF (0.033 mL, 0.43 mmol), and the mixture heated at reflux for 1 h. The reaction was allowed to cool to rt and quenched by the careful addition of water (2 mL), and sat. aq. NaHCO₃ (10 mL) was then added carefully, followed by extraction with CH₂Cl₂ (4×10 mL), and the combined org. layers washed with sat. aq. NaHCO₃ (10 mL) and sat. aq. NH₄Cl (2×10 mL). The organics were dried (NaSO₄), and concentrated in vacuo to give the title compound as a brown solid (39 mg, 97%).

### 8-(2-fluoro-4-(1-methyl-1H-pyrazol-4-yl)phenyl)-1,6-naphthyridine-2-carbonitrile

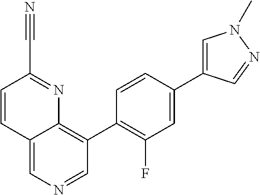 [SMILES: Cn1cc(-c2ccc(-c3cncc4ccc(C#N)nc34)c(F)c2)cn1]

A mixture of 2-chloro-8-(2-fluoro-4-(1-methyl-1H-pyrazol-4-yl)phenyl)-1,6-naphthyridine (38 mg, 0.11 mmol), Zn(CN)₂ (16 mg, 0.14 mmol), and Pd(PPh₃)₄ (13 mg, 0.011) in DMF (0.7 mL) was heated at 80° C. for 2 h under microwave irradiation. The crude reaction mixture was concentrated under reduced pressure and the residue purified by Biotage (SNAP 10 g column, CH₂Cl₂/EtOH 100/0->97/3) to give the title compound as an orange oil (18 mg, 49%). [M+H]⁺ 330. Rt 1.38 min (method M).

### Example 187

### 8-(2-fluoro-4-(1-methyl-1H-pyrazol-4-yl)phenyl)-1,6-naphthyridine-2-carboxamide (14)

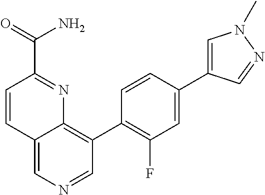 [SMILES: Cn1cc(-c2ccc(-c3cncc4ccc(C(N)=O)nc34)c(F)c2)cn1]

To 8-(2-fluoro-4-(1-methyl-1H-pyrazol-4-yl)phenyl)-1,6-naphthyridine-2-carbonitrile (16 mg, 0.049 mmol) was added an ice cold mixture of water (0.15 mL) and conc. H₂SO₄ (1.85 mL, 34.70 mmol) at 0° C. The reaction mixture was then heated at 50° C. for 1 h. The mixture was then dropped into ice cold 2 M aq. NaOH (20 mL), and the resulting mixture neutralised with sat. aq. NaHCO₃ (˜40 mL). The organic material was extracted with CH₂Cl₂ (3×15 mL), and the combined organic layers were washed with water (15 mL), dried (MgSO₄), and concentrated in vacuo. The crude product was purified by Biotage (SingleStep 12 g column, CH₂Cl₂/EtOH 100/0->50/50) to give the title compound as a yellow solid (12 mg, 71%). ¹H NMR (500 MHz, DMSO-d6) ppm=9.57 (s, 1H), 8.87 (d, J=8.5, 1H), 8.32 (d, J=8.5, 1H), 8.03 (s, 1H), 7.67-7.59 (m, 4H), 7.57-7.54 (m, 1H), 3.90 (s, 3H). [M+H]⁺ 348. Rt 1.34 min (method M).

### Example 188

### 8-(4-(1-methyl-1H-pyrazol-4-yl)phenyl)-1,6-naphthyridine-2-thiol (77)

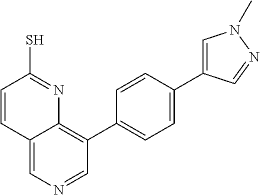 [SMILES: Cn1cc(-c2ccc(-c3cncc4ccc(S)nc34)cc2)cn1]

A mixture of 8-(4-(1-methyl-1H-pyrazol-4-yl)phenyl)-1,6-naphthyridin-2-ol (Example 114) (50 mg, 0.165 mmol) and diphoshorus pentasulfide (37.5 mg, 0.169 mmol) in pyridine (293 μl, 3.31 mmol) was stirred at 60° C. for 5 hr. The mixture was then stirred at 80° C. for 30 hr. To the mixture was added water and EtOAc and the layers were separated. The aqueous layer was extracted with EtOAc three times. The combined organic layers were dried over MgSO₄ and concentrated in vacuum. The resulting brown oil was purified by chromatography on silica gel (biotage, CH₂Cl₂/EtOH, 100:0 to 94/4) to give the product (30.9 mg, 59%) as a yellow solid. ¹H-NMR (500 MHz, CDCl₃/MeOD, 1:1) ppm=8.87 (s, 1H), 8.48 (s, 1H), 7.94 (s, 1H), 7.86 (s, 1H), 7.82 (d, J=9.2, 1H), 7.75 (d, J=8.2, 2H), 7.51 (d, J=8.2, 2H), 7.44 (d, J=9.2, 1H), 3.97 (s, 3H). [M+H]⁺ 319. Rt 1.29 min (method M).

### Example 189

### 8-(2,2-dioxido-1,3-dihydrobenzo[c]isothiazol-5-yl)-5-hydroxy-N-methyl-1,6-naphthyridine-2-carboxamide (63)

### 5-bromo-3-iodo-2-methoxypyridin-4-amine

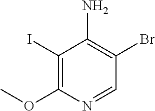

To 3-iodo-2-methoxypyridin-4-amine (1 g, 4.00 mmol) in solution in acetonitrile (40 mL) were added N-bromosuccinimide (0.783 g, 4.40 mmol) and acetic acid (1 mL) and the mixture was stirred at room temperature for 3 h. The reaction mixture was concentrated and then water and ethyl acetate were added to the residue. The layers were separated, the aqueous layer was extracted with ethyl acetate and the combined organic layers were dried over MgSO₄, filtered and concentrated. The crude was purified via biotage column chromatography (cyclohexane/ethyl acetate 100/0 to 95/5, Single step 40 g) to give the title compound (1.25 g, 86% purity, 95% yield).

### ethyl(E)-3-(4-amino-5-bromo-2-methoxypyridin-3-yl)acrylate

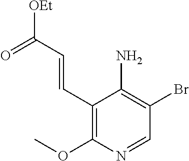 [SMILES: CCOC(=O)/C=C/c1c(OC)ncc(Br)c1N]

5-bromo-3-iodo-2-methoxypyridin-4-amine (1.210 g, 3.680 mmol), tri-o-tolylphosphine (0.090 g, 0.294 mmol) and palladium(II)acetate (0.033 g, 0.147 mmol) were introduced in a vial and then DMF (10.22 ml), triethylamine (0.718 ml, 5.15 mmol) and ethyl acrylate (0.598 ml, 5.52 mmol) were added. The reaction mixture was stirred at 100° C. for 15 h. Water and dichloromethane were added to the reaction mixture. The layers were separated. The organic layer was washed with water and then the aqueous layers were extracted twice with dichloromethane. The organic layer was dried over MgSO₄ and concentrated. The crude was purified via biotage column chromatography (dichloromethane, single step 40 g) to give the title compound as a colourless solid (684 mg, 62% yield).

### 8-bromo-5-methoxy-1,6-naphthyridin-2(1H)-one

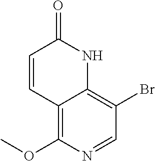

To a solution of (E)-ethyl 3-(4-amino-5-bromo-2-methoxypyridin-3-yl)acrylate (684 mg, 2.271 mmol) in ethanol (9.5 mL) was added sodium methanethiolate (164 mg, 2.340 mmol) and the mixture was stirred at rt for 1.5 h. Water and dichloromethane were added to the reaction mixture. The layers were separated and the aqueous layer was extracted three times with dichloromethane. The organic layers were combined, dried over MgSO₄ and concentrated. The crude was purified via biotage column chromatography (dichloromethane/EtOAc 100/0 to 80/20, single step 40 g) to give the title compound as a colourless solid (510 mg, 88% yield) 8-bromo-5-methoxy-1,6-naphthyridin-2-yl trifluoromethanesulfonate

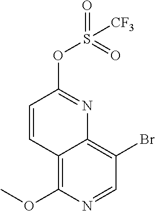 [SMILES: COc1ncc(Br)c2nc(OS(=O)(=O)C(F)(F)F)ccc12]

To a solution of 8-bromo-5-methoxy-1,6-naphthyridin-2(1H)-one (50 mg, 0.196 mmol) in dichloromethane (1.2 mL) were added triethylamine (55 μL, 0.392 mmol) and trifluoromethanesulfonic anhydride (40 μL, 0.235 mmol). After 30 min, saturated aq NaHCO₃ solution and dichloromethane were added, the layers were separated and the aqueous layer was extracted three times with dichloromethane. The organic layer were dried over MgSO₄, filtered and concentrated. The crude product was used without any purification in the next step. 8-bromo-5-methoxy-1,6-naphthyridine-2-carbonitrile

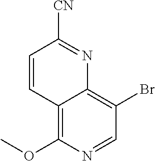

8-bromo-5-methoxy-1,6-naphthyridin-2-yl trifluoromethanesulfonate (215 mg, 0.556 mmol), Pd(PPh₃)₄ (64.2 mg, 0.056 mmol) and zinc cyanide (71.8 mg, 0.612 mmol) were loaded in a microwave vial and then DMF (3.7 mL) was added. The reaction mixture was heated at 60° C. for 2.5 h in an oil bath. The reaction mixture was concentrated and purified via biotage column chromatography (snap column 25 g, cyclohexane/dichloromethane 50/50 to 0/100) to give the title compound (99 mg, 67% yield).

### 8-(2,2-dioxido-1,3-dihydrobenzo[c]isothiazol-5-yl)-5-methoxy-1,6-naphthyridine-2-carbonitrile

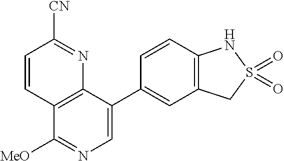 [SMILES: COc1ncc(-c2ccc3c(c2)CS(=O)(=O)N3)c2nc(C#N)ccc12]

8-bromo-5-methoxy-1,6-naphthyridine-2-carbonitrile (47 mg, 0.178 mmol), 5-(4,4,5,5-tetramethyl-1,3,2-dioxaborolan-2-yl)-1,3-dihydrobenzo[c]isothiazole 2,2-dioxide (58 mg, 0.196 mmol) and Pd(dppf)Cl₂.CH₂Cl₂ (7.3 mg, 8.90 μmol) were loaded in a microwave vial and then degassed acetonitrile (3.0 mL) and sodium carbonate in water (500 μL, 0.249 mmol) were added. The reaction was heated at 120° C. for 60 min in a focused microwave reactor. The crude was purified via biotage column chromatography (dichloromethane/EtOH 100/0 to 98/2) to give the title compound as a yellow solid (100 mg, 70% yield).

### 8-(2,2-dioxido-1,3-dihydrobenzo[c]isothiazol-5-yl)-5-methoxy-N-methyl-1,6-naphthyridine-2-carboxamide

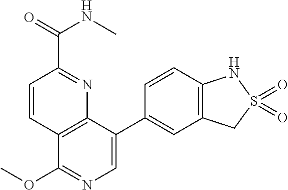 [SMILES: CNC(=O)c1ccc2c(OC)ncc(-c3ccc4c(c3)CS(=O)(=O)N4)c2n1]

To 8-(2,2-dioxido-1,3-dihydrobenzo[c]isothiazol-5-yl)-5-methoxy-1,6-naphthyridine-2-carbonitrile (95 mg, 0.270 mmol) in ethanol (2.1 mL) was added 2 M sodium hydroxide (2.1 mL, 4.18 mmol). The reaction mixture was stirred at rt for 1 h and at 40° C. for 2.5 h. 1 M hydrogen chloride in dioxane (4.2 mL, 4.18 mmol) was added to the reaction mixture and then the solution was concentrated. The crude was solubilized in DMF (2.25 mL) and HATU (123 mg, 0.324 mmol) was added. The reaction mixture was stirred for 15 min before the addition of methylamine in THF (2M) (410 μL, 0.821 mmol). The reaction mixture was stirred at rt for 15 min before the addition of DIPEA (206 μL, 1.177 mmol). The resulting solution was then stirred at rt for 1.5 h. The reaction mixture was concentrated and the crude material purified via biotage column chromatography (dichloromethane/EtOH 99/1 to 96/4). The product obtained was then solubilised in dichloromethane and washed with water to give the title compound (24 mg, contaminated by 3% of tetramethylurea, 23% yield over two steps).

### 8-(2,2-dioxido-1,3-dihydrobenzo[c]isothiazol-5-yl)-5-hydroxy-N-methyl-1,6-naphthyridine-2-carboxamide

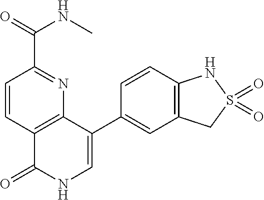 [SMILES: CNC(=O)c1ccc2c(=O)[nH]cc(-c3ccc4c(c3)CS(=O)(=O)N4)c2n1]

To 8-(2,2-dioxido-1,3-dihydrobenzo[c]isothiazol-5-yl)-5-methoxy-N-methyl-1,6-naphthyridine-2-carboxamide (14 mg, 0.036 mmol) in ethanol (0.6 mL) was added pyridine hydrochloride salt (68 mg, 0.588 mmol). The reaction mixture was heated at 150° C. for 40 min. Sat. NaHCO₃ solution and dichloromethane were added. The mixture was concentrated and the resulting residue was solubilised in water and acidified to pH 2. The solution was concentrated and the residue was washed with dichloromethane/EtOH, EtOH and acetone. The filtrates were combined (35 mg) and purified via biotage column chromatography (dichloromethane/EtOH 98/2 to 92/8) to give the title compound (8 mg, 59% yield). ¹H NMR (500 MHz, DMSO-d6) ppm=11.91 (d, J=6.1, 1H), 10.57 (s, 1H), 8.75 (d, J=8.2, 1H), 8.11 (d, J=8.2, 1H), 8.01 (q, J=4.9, 1H), 7.61 (d, J=1.9, 1H), 7.54 (dd, J=8.2, 1.9, 1H), 7.51 (d, J=6.0, 1H), 6.92 (d, J=8.2, 1H), 4.60 (s, 2H), 2.87 (d, J=4.9, 3H). [M+H]⁺ 371. Rt 1.93 min (method N).

### Example 190

### 5-Methyl-8-(4-(1-methyl-1H-pyrazol-4-yl)phenyl)-1,6-naphthyridine-2-carboxamide (61)

### 5-Bromo-3-iodo-2-methylpyridin-4-amine

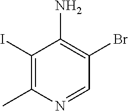

A solution of potassium iodide (1.065 g, 6.42 mmol) and iodine (1.357 g, 5.35 mmol) in water (9 mL) was added dropwise to a solution of 4-amino-5-bromo-2-methyl-pyridine (1.0 g, 5.35 mmol) and sodium carbonate (0.567 g, 5.35 mmol) in water (4 mL) at reflux. The reaction mixture was stirred at reflux for 20 h. The cooled mixture was diluted with water and EtOAc and the layers were separated. The aqueous layer was extracted with EtOAc twice. The combined organic layers were washed with sat. Na₂S₂O₃ solution, dried over MgSO₄ and concentrated in vacuum. The resulting brown oil was purified by chromatography on silica gel (biotage, CH₂Cl₂/EtOAc, 100:0 to 45:55) to give the starting material (544 mg) and the product (577 mg, 35%) as colourless solids.

### (E)-Ethyl 3-(4-amino-5-bromo-2-methylpyridin-3-yl)acrylate

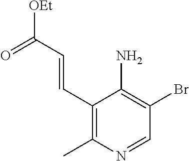 [SMILES: CCOC(=O)/C=C/c1c(C)ncc(Br)c1N]

To a solution of 5-Bromo-3-iodo-2-methylpyridin-4-amine (570 mg, 1.821 mmol) in DMF (5 mL) was added ethyl acrylate (0.296 mL, 2.73 mmol), triethylamine (0.353 mL, 2.55 mmol), tri-o-tolylphosphine (44.4 mg, 0.146 mmol) and palladium(II)acetate (16.4 mg, 0.073 mmol) under nitrogen and the mixture was stirred at 100° C. for 6 h. The cooled mixture was diluted with water and EtOAc and the layers were separated. The aqueous layer was extracted with EtOAc twice. The combined organic layers were washed with water, dried over MgSO₄ and concentrated in vacuum. The resulting brown oil was purified by column chromatography on silica gel (biotage, CH₂Cl₂/EtOH, 100:0 to 96:4) to give the product (489 mg, 94%) as beige solid.

### 8-Bromo-5-methyl-1,6-naphthyridin-2(1H)-one

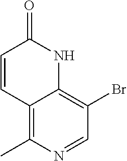

To a solution of (E)-Ethyl 3-(4-amino-5-bromo-2-methylpyridin-3-yl)acrylate (480 mg, 1.683 mmol) in ethanol (7 mL) was added sodium methanethiolate (122 mg, 1.734 mmol) and the mixture was heated at rt for 2 h, before the mixture was concentrated in vacuum. The resulting brown solid was purified by column chromatography on silica gel (biotage, CH₂Cl₂/EtOH, 100:0 to 90:10) to give the product (329 mg, 82%) as a colourless solid.

### 5-Methyl-8-(4-(1-methyl-1H-pyrazol-4-yl)phenyl)-1,6-naphthyridin-2(1H)-one

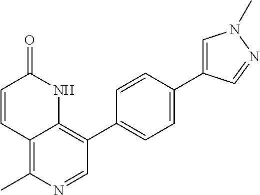 [SMILES: Cc1ncc(-c2ccc(-c3cnn(C)c3)cc2)c2[nH]c(=O)ccc12]

8-Bromo-5-methyl-1,6-naphthyridin-2(1H)-one (250 mg, 1.046 mmol), 1-methyl-4-(4-(4,4,5,5-tetramethyl-1,3,2-dioxaborolan-2-yl)phenyl)-1H-pyrazole (297 mg, 1.046 mmol) and Pd(dppf)Cl₂—CH₂Cl₂ (38 mg, 0.052 mmol) were loaded in a microwave vial. The capped vial was evacuated using high vacuum and purged with nitrogen (three times). Acetonitrile (6.5 mL) and aqueous sodium carbonate (0.5M, 2.93 mL, 1.464 mmol) were added and the mixture was degassed again by using the high vacuum and purged with nitrogen again (three times). The mixture was heated in a focused microwave reactor at 120° C. for 1 h. The cooled mixture was transferred into a flask with the microwave vial being washed with CHCl₃, and the water was evaporated by azeotropic removal with toluene. The resulting brown solid was purified by chromatography on silica gel (biotage, CH₂Cl₂/EtOH, 100:0 to 93:7) to give the product (310 mg, 94%) as a beige solid.

### 2-Chloro-5-methyl-8-(4-(1-methyl-1H-pyrazol-4-yl)phenyl)-1,6-naphthyridine

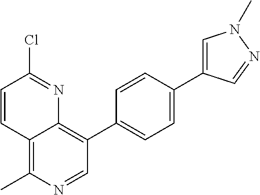 [SMILES: Cc1ncc(-c2ccc(-c3cnn(C)c3)cc2)c2nc(Cl)ccc12]

A mixture of 5-Methyl-8-(4-(1-methyl-1H-pyrazol-4-yl)phenyl)-1,6-naphthyridin-2(1H)-one (200 mg, 0.632 mmol) and 5 drops of DMF in phosphorous oxychloride (3.5 mL, 0.632 mmol) was heated at 80° C. for 6 hr. The mixture was transferred into a flask and the POCl₃ was evaporated by azeotropic removal with toluene. The brown oil was purified by chromatography on silica gel (biotage, CH₂Cl₂/10% NH₄OH (25% in water) in MeOH, 95:5 to 94:6) to give product (110 mg, 52%) and starting material (39 mg) as a beige solids.

### 133.6 5-Methyl-8-(4-(1-methyl-1H-pyrazol-4-yl)phenyl)-1,6-naphthyridine-2-carbonitrile

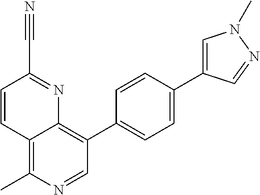 [SMILES: Cc1ncc(-c2ccc(-c3cnn(C)c3)cc2)c2nc(C#N)ccc12]

A solution of 2-chloro-5-methyl-8-(4-(1-methyl-1H-pyrazol-4-yl)phenyl)-1,6-naphthyridine (25 mg, 0.075 mmol), zinc cyanide (9.65 mg, 0.082 mmol) and Pd(PPh₃)₄ (8.63 mg, 7.47 μmol) was heated in a focused microwave reactor at 60° C. for 1 h. The mixture was diluted with a small amount of CHCl₃/MeOH (1/1), EtOAc and water and the layers were separated. The aqueous layer was extracted with EtOAc twice, the combined organic layers were dried over MgSO₄ and concentrated in vacuum. The resulting brown oil was purified by chromatography on silica gel (biotage, CH₂Cl₂/EtOH, 100:0 to 96:4) and further purified by using a scx2-cartridge (loading with CH₂Cl₂/MeOH 9/1, elution with CH₂Cl₂/1N NH₃ in MeOH 9/1) to give the product (19.7 mg, 81%) as a yellow solid. ¹H-NMR (500 MHz, DMSO-d6) ppm=9.01 (d, J=8.6, 1H), 8.78 (s, 1H), 8.25 (d, J=8.6, 1H), 8.23 (s, 1H), 7.96 (s, 1H), 7.73 (d, J=8.4, 2H), 7.70 (d, J=8.3, 2H), 3.90 (s, 3H), 3.00 (s, 3H). [M+H]⁺ 326. Rt 1.39 min (method M).

### 5-Methyl-8-(4-(1-methyl-1H-pyrazol-4-yl)phenyl)-1,6-naphthyridine-2-carboxamide

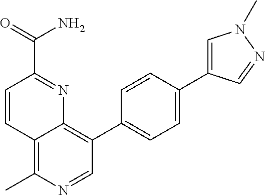 [SMILES: Cc1ncc(-c2ccc(-c3cnn(C)c3)cc2)c2nc(C(N)=O)ccc12]

To 5-methyl-8-(4-(1-methyl-1H-pyrazol-4-yl)phenyl)-1,6-naphthyridine-2-carbonitrile (5 mg, 0.015 mmol) was added a cold mixture of water (10 μl) and conc. H₂SO₄ (164 μl, 3.07 mmol) at 0° C. and the mixture was stirred at 50° C. for 1 h. The mixture was dropped into ice-cooled water and solid NaOH and NaHCO₃ were added. The mixture was diluted with CH₂Cl₂, the layers were separated and the aqueous layer was extracted with CH₂Cl₂ three times. The combined organic layers were washed with water, dried over MgSO₄ and concentrated in vacuum. The resulting residue was purified by chromatography on silica gel (biotage, CH₂Cl₂/EtOH, 100:0 to 94:6) and further purified by using a scx2-cartridge (loading with CH₂Cl₂/MeOH 9/1, elution with CH₂Cl₂/1N NH₃ in MeOH 9/1) to give the product (5.2 mg, 99%) as a yellow solid. ¹H-NMR (500 MHz, DMSO-d6) ppm=9.01 (d, J=8.6, 1H), 8.78 (s, 1H), 8.25 (d, J=8.6, 1H), 8.23 (s, 1H), 7.96 (s, 1H), 7.73 (d, J=8.4, 2H), 7.70 (d, J=8.3, 2H), 3.90 (s, 3H), 3.00 (s, 3H). [M+H]⁺ 344. Rt 1.33 min (method M).

### Example 191

### 4-Methyl-8-(4-(1-methyl-1H-pyrazol-4-yl)phenyl)-1,6-naphthyridine-2-carboxamide (64)

### 3-Bromo-5-iodopyridin-4-amine

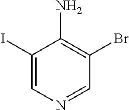

A solution of potassium iodide (2.88 g, 17.34 mmol) and iodine (2.75 g, 10.84 mmol) in water (21 mL) was added dropwise to a solution of 4-amino-3-bromopyridine (2.5 g, 14.45 mmol) and sodium carbonate (0.919 g, 8.67 mmol) in water (10 mL) and the mixture was stirred at reflux for 20 h. The mixture was diluted with water and EtOAc and the layers were separated. The organic layer was extracted with EtOAc three times. The combined organic layers were washed with sat. Na₂S₂O₃ three times, dried over MgSO₄, filtered off and the filtrate concentrated in vacuum. The resulting brown oil was purified by chromatography on silica gel (biotage, CyHex/EtOAc, 50:50 to 0:100) to give product (951 mg, 22%) and starting material (1.66 g) as light yellow solids.

### 8-Bromo-4-methyl-1,6-naphthyridin-2(1H)-one

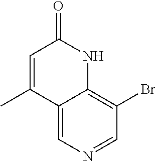

To a solution of 3-Bromo-5-iodopyridin-4-amine (1.5 g, 5.02 mmol) in DMF (12 mL) was added methyl crotonate (1.064 mL, 10.04 mmol), triethylamine (0.974 mL, 7.03 mmol), tri-o-tolylphosphine (0.12 g, 0.401 mmol) and palladium(II)acetate (0.045 g, 0.201 mmol) under nitrogen and the mixture was stirred at 120° C. for 48 h. The DMF was evaporated (V10 biotage system) and the resulting residue was purified by chromatography on silica gel (biotage, CH₂Cl₂/MeOH, 100:0 to 92:8) to give the product (0.82 g, 69%) as a red solid. This solid was further purified by recrystallization from hot EtOAc to give the pure product (503 mg, 42%) as a beige solid.

### 4-Methyl-8-(4-(1-methyl-1H-pyrazol-4-yl)phenyl)-1,6-naphthyridin-2(1H)-one

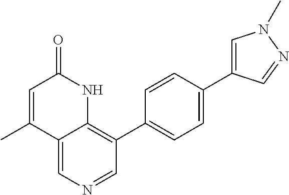 [SMILES: Cc1cc(=O)[nH]c2c(-c3ccc(-c4cnn(C)c4)cc3)cncc12]

8-Bromo-4-methyl-1,6-naphthyridin-2(1H)-one (300 mg, 1.255 mmol), 1-methyl-4-(4-(4,4,5,5-tetramethyl-1,3,2-dioxaborolan-2-yl)phenyl)-1H-pyrazole (357 mg, 1.255 mmol) and Pd(dppf)Cl₂—CH₂Cl₂ (45.9 mg, 0.063 mmol) were loaded in a microwave vial. The capped vial was evacuated using high vacuum and purged with nitrogen (three times). Acetonitrile (7.5 mL) and aqueous sodium carbonate (0.5M, 3.51 mL, 1.757 mmol) were added and the mixture was degassed again by using the high vacuum and purged with nitrogen again (three times). The mixture was heated in a focused microwave reactor at 120° C. for 1 hr. The cooled reaction mixture was transferred into a flask with the microwave vial being washed with CHCl₃, and the water was evaporated by azeotropic removal with toluene. The resulting brown solid was purified by chromatography on silica gel (biotage, CH₂Cl₂/EtOH, 100:0 to 96:4) to give the product (300 mg, 76%) as a beige solid.

### 2-Chloro-4-methyl-8-(4-(1-methyl-1H-pyrazol-4-yl)phenyl)-1,6-naphthyridine

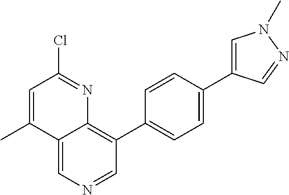 [SMILES: Cc1cc(Cl)nc2c(-c3ccc(-c4cnn(C)c4)cc3)cncc12]

To a mixture of 4-methyl-8-(4-(1-methyl-1H-pyrazol-4-yl)phenyl)-1,6-naphthyridin-2(1H)-one (200 mg, 0.632 mmol) in acetonitrile (4 mL) was added phosphorus oxychloride (0.28 mL, 3.00 mmol) followed by DMF (0.18 mL, 2.325 mmol) and the mixture was heated at reflux for 1.5 h. The mixture was carefully diluted with water (10 mL) and sat. NaHCO₃ (30 mL) before EtOAc was added and the layers were separated. The aqueous layer was extracted with EtOAc three times. The combined organic layers were washed with sat. NH₄Cl, dried over MgSO₄, filtered, and the filtrate concentrated in vacuum. The resulting brown oil was purified by chromatography on silica gel (biotage, CH₂Cl₂/EtOH, 100:0 to 94:6) to give the product (173 mg, 82%) as a colourless solid. ¹H-NMR (500 MHz, CDCl₃) ppm=9.43 (s, 1H), 8.88 (s, 1H), 7.85 (s, 1H), 7.77 (d, J=7.9, 2H), 7.69 (s, 1H), 7.63 (d, J=7.9, 2H), 7.37 (s, 1H), 3.99 (s, 3H), 2.85 (s, 3H). [M+H]⁺ 335. Rt 1.52 min (method M).

### 4-Methyl-8-(4-(1-methyl-1H-pyrazol-4-yl)phenyl)-1,6-naphthyridine-2-carbonitrile

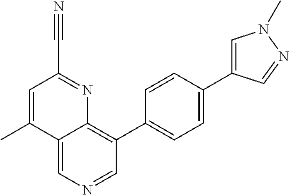 [SMILES: Cc1cc(C#N)nc2c(-c3ccc(-c4cnn(C)c4)cc3)cncc12]

A mixture of 2-chloro-4-methyl-8-(4-(1-methyl-1H-pyrazol-4-yl)phenyl)-1,6-naphthyridine (90 mg, 0.269 mmol), zinc cyanide (35 mg, 0.296 mmol) and Pd(PPh₃)₄ (31 mg, 0.027 mmol) was heated in a focused microwave reactor at 60° C. for 3 h. The mixture was diluted with EtOAc and water and the layers were separated. The aqueous layer was extracted with EtOAc twice. The combined organic layers were dried over MgSO₄, filtered, and the filtrate concentrated in vacuum. The residue was purified by chromatography on silica gel (biotage, CH₂Cl₂/EtOH, 100:0 to 97:3) and further purified by using a scx2-cartridge (loading with CH₂Cl₂/MeOH 9/1, elution with CH₂Cl₂/1N NH₃ in MeOH 9/1) to give the product (68 mg, 78%) as a yellow solid. 4-Methyl-8-(4-(1-methyl-1H-pyrazol-4-yl)phenyl)-1,6-naphthyridine-2-carboxamide

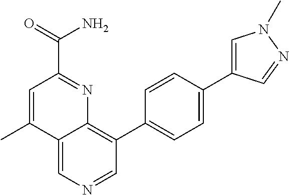 [SMILES: Cc1cc(C(N)=O)nc2c(-c3ccc(-c4cnn(C)c4)cc3)cncc12]

To 4-Methyl-8-(4-(1-methyl-1H-pyrazol-4-yl)phenyl)-1,6-naphthyridine-2-carbonitrile (52 mg, 0.160 mmol) was added a cold mixture of water (100 μl) and conc. H₂SO₄ (1.7 mL, 32.0 mmol) at 0° C. and the mixture was stirred at 50° C. for 1 h. The mixture was dropped into ice-cooled 2M NaOH. To neutralize the mixture, NaHCO₃ was added and the mixture was diluted with CH₂Cl₂. The layers were separated and the aqueous layer was extracted with CH₂Cl₂ three times. The combined organic layers were washed with water, dried over MgSO₄, filtered, and the filtrate concentrated in vacuum. The resulting residue was purified by chromatography on silica gel (biotage, CH₂Cl₂/EtOH, 100:0 to 94:6) and further purified by using a scx2-cartridge (loading with CH₂Cl₂/MeOH 9/1, elution with CH₂Cl₂/1N NH₃ in MeOH 9/1) to give the product (53 mg, 97%) as a yellow solid. ¹H-NMR (500 MHz, DMSO-d6) ppm=9.63 (s, 1H), 8.90 (s, 1H), 8.24 (d, J=0.9, 1H), 8.14 (s, 1H), 7.96 (bs, 2H), 7.81 (d, J=8.3, 2H), 7.74 (d, J=8.3, 2H), 7.57 (d, J=2.9, 1H), 3.90 (s, 3H), 2.95 (s, 3H). [M+H]⁺ 344. Rt 1.47 min (method M).

### Example 192

### 7-Chloro-8-(4-(1-methyl-1H-pyrazol-4-yl)phenyl)-1,6-naphthyridine-2-carboxamide (66)

### 3-Bromo-2-chloropyridin-4-amine

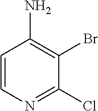

To a solution of 2-chloro-4-amino-pyridine (5.00 g, 38.9 mmol) in acetic acid (50 mL) was added N-bromosuccinimide (7.61 g, 42.8 mmol) maintaining the temperature at rt by using an ice-bath. The mixture was stirred at rt overnight. The mixture was concentrated under vacuum with the help of toluene for azeotropic evaporation of the acid. The residue was dried in high vacuum before it was purified by chromatography on silica gel (biotage, CyHex/EtOAc, 90:10 to 50:50) to give the product (3.80 g, 47%) as a colourless solid.

### 3-Bromo-2-chloro-5-iodopyridin-4-amine

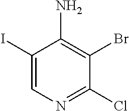

To a solution of 3-bromo-2-chloropyridin-4-amine (0.500 g, 2.410 mmol) in acetic acid (5 mL) was added N-iodosuccinimide (0.569 g, 2.53 mmol) and the mixture was stirred at 130° C. for 2 h. The mixture was concentrated in vacuum with the help of toluene for azeotropic evaporation of the acid. The residue was dried under high vacuum before it was purified by chromatography on silica gel (biotage, CyHex/EtOAc, 90:10 to 70:30) to give the product (0.722 g, 90%) as a colourless solid.

### (E)-Ethyl 3-(4-amino-5-bromo-6-chloropyridin-3-yl)acrylate

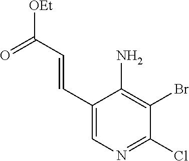 [SMILES: CCOC(=O)/C=C/c1cnc(Cl)c(Br)c1N]

To a solution of 3-bromo-2-chloro-5-iodopyridin-4-amine (500.0 mg, 1.500 mmol) in DMF (4 mL) and ethyl acrylate (0.244 mL, 2.250 mmol), triethylamine (0.291 mL, 2.100 mmol), tri-o-tolylphosphine (36.5 mg, 0.120 mmol) and palladium(II)acetate (13.5 mg, 0.060 mmol) were added under nitrogen. The mixture was stirred at 100° C. for 1.5 h before it was diluted with water and EtOAc and the layers were separated. The aqueous layer was extracted with EtOAc twice. The combined organic layers were washed with water, dried over MgSO₄ and concentrated in vacuum. The resulting brown oil was purified by chromatography on silica gel (biotage, CH₂Cl₂/EtOH, 100:0 to 97:3) to give the product as a light brown solid, which was used directly in the next step.

### 8-Bromo-7-chloro-1,6-naphthyridin-2(1H)-one

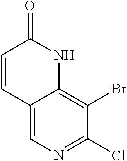 [SMILES: O=c1ccc2cnc(Cl)c(Br)c2[nH]1]

To a solution of (E)-Ethyl 3-(4-amino-5-bromo-6-chloropyridin-3-yl)acrylate (458 mg, 1.499 mmol) in ethanol (15 mL) was added sodium methanethiolate (110 mg, 1.574 mmol) and the mixture was heated at rt for 10 min, before the mixture was concentrated in vacuum. The resulting brown solid was purified by chromatography on silica gel (biotage, CH₂Cl₂/EtOH, 100:0 to 97:3) to give the product (264 mg, 68% over two steps) as a colourless solid. 7-Chloro-8-(4-(1-methyl-1H-pyrazol-4-yl)phenyl)-1,6-naphthyridin-2(1H)-one

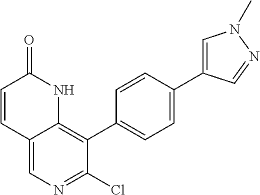 [SMILES: Cn1cc(-c2ccc(-c3c(Cl)ncc4ccc(=O)[nH]c34)cc2)cn1]

8-Bromo-7-chloro-1,6-naphthyridin-2(1H)-one (205 mg, 0.790 mmol), 1-methyl-4-(4-(4,4,5,5-tetramethyl-1,3,2-dioxaborolan-2-yl)phenyl)-1H-pyrazole (236 mg, 0.830 mmol) and Pd(dppf)Cl₂—CH₂Cl₂ (28.9 mg, 0.040 mmol) were loaded in a microwave vial. The capped vial was evacuated by vacuum and purged with nitrogen (three times). Acetonitrile (7 mL) and aqueous sodium carbonate (0.5M, 2.212 mL, 1.106 mmol) were added and the mixture was degassed again by using the high vacuum and purged with nitrogen again (each three times). The mixture was heated in the microwave at 120° C. for 1.5 h. The cooled reaction mixture was transferred into a flask with the microwave vial being washed with CHCl₃, and the water was evaporated by azeotropic removal with toluene. The resulting brown solid was purified by chromatography on silica gel (biotage, CH₂Cl₂/EtOH, 100:0 to 97:3) to give the product (155 mg, 58%) as a colourless solid.

### 2,7-Dichloro-8-(4-(1-methyl-1H-pyrazol-4-yl)phenyl)-1,6-naphthyridine

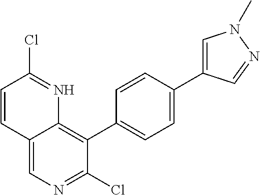 [SMILES: Cn1cc(-c2ccc(-c3c(Cl)ncc4ccc(Cl)nc34)cc2)cn1]

To a mixture of 7-chloro-8-(4-(1-methyl-1H-pyrazol-4-yl)phenyl)-1,6-naphthyridin-2(1H)-one (140 mg, 0.416 mmol) in acetonitrile (5 mL) was added phosphorus oxychloride (1.383 mL, 1.975 mmol) followed by DMF (0.191 mL, 1.530 mmol) and the mixture was heated at reflux for 2 h. The mixture was cooled to 0° C. and carefully diluted with water and sat. NaHCO₃ before EtOAc was added and the layers were separated. The aqueous layer was extracted with EtOAc three times. The combined organic layers were washed with sat. NH₄Cl, dried over MgSO₄, filtered, and the filtrate concentrated in vacuum. The resulting brown oil was purified by chromatography on silica gel (biotage, CH₂Cl₂/EtOH, 100:0 to 97:3) to give the product (107 mg, 73%) as a yellow solid.

### 7-Chloro-8-(4-(1-methyl-1H-pyrazol-4-yl)phenyl)-1,6-naphthyridine-2-carbonitrile

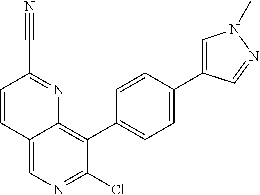 [SMILES: Cn1cc(-c2ccc(-c3c(Cl)ncc4ccc(C#N)nc34)cc2)cn1]

A mixture of 2,7-Dichloro-8-(4-(1-methyl-1H-pyrazol-4-yl)phenyl)-1,6-naphthyridine (107 mg, 0.301 mmol), zinc cyanide (38.9 mg, 0.331 mmol) and Pd(PPh₃)₄ (34.8 mg, 0.030 mmol) was heated in a focused microwave reactor at 60° C. for 1.5 h. The mixture was diluted with EtOAc and water and the layers were separated. The aqueous layer was extracted with EtOAc twice. The combined organic layers were dried over MgSO₄, filtered, and the filtrate concentrated in vacuum. The residue was purified by chromatography on silica gel (biotage, CH₂Cl₂/EtOH, 100:0 to 98:2) to give the product (91 mg, 87%) as a yellow solid.

### 7-Chloro-8-(4-(1-methyl-1H-pyrazol-4-yl)phenyl)-1,6-naphthyridine-2-carboxamide

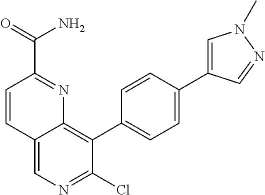 [SMILES: Cn1cc(-c2ccc(-c3c(Cl)ncc4ccc(C(N)=O)nc34)cc2)cn1]

To 2,7-Dichloro-8-(4-(1-methyl-1H-pyrazol-4-yl)phenyl)-1,6-naphthyridine (60 mg, 0.174 mmol) was added a cold mixture of water (120 μl) and conc. H₂SO₄ (1.85 ml, 34.7 mmol) at 0° C. and the mixture was stirred at 50° C. for 1 h. The mixture was dropped into ice-cooled 2M NaOH. To neutralize the mixture NaHCO₃ was added and the mixture was diluted with CH₂Cl₂. The layers were separated and the aqueous layer was extracted with CH₂Cl₂ three times. The combined organic layers were washed with water, dried over MgSO₄, filtered, and the filtrate concentrated in vacuum. The resulting brown oil was purified by chromatography on silica gel (biotage, CH₂Cl₂/EtOH, 100:0 to 94:6) to give the product (68 mg, quant.) as a yellow solid. ¹H-NMR (500 MHz, DMSO-d6) ppm=9.44 (s, 1H), 8.88 (d, J=8.4, 1H), 8.28 (d, J=8.4, 1H), 8.24 (s, 1H), 7.97 (d, J=0.8, 1H), 7.95 (bs, 1H), 7.74 (d, J=8.2, 2H), 7.51 (d, J=8.2, 2H), 7.28 (bs, 1H), 3.90 (s, 3H). [M+H]⁺ 364. Rt 1.31 min (method M).

### Example 193

### 7-chloro-8-(4-(1-methyl-1H-pyrazol-4-yl)phenyl)-1,6-naphthyridine-2-carboximidamide (68)

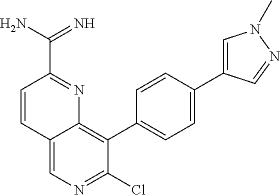 [SMILES: Cn1cc(-c2ccc(-c3c(Cl)ncc4ccc(C(=N)N)nc34)cc2)cn1]

To a solution of 7-chloro-8-(4-(1-methyl-1H-pyrazol-4-yl)phenyl)-1,6-naphthyridine-2-carbonitrile (10.0 mg, 0.029 mmol) in THF (0.25 mL) was added Pd₂(dba)₃ (2.7 mg, 2.89 μmol), CyJohnPhos (2.0 mg, 5.78 μmol) and LiHMDS (1M in THF) (0.032 mL, 0.032 mmol) and the mixture was heated at 65° C. for 1 h. The mixture was concentrated in vacuum and purified by chromatography on silica gel (biotage, CH₂Cl₂/EtOH, 100:0 to 20:80) to give the title compound (7.9 mg, 75%) as a yellow solid. ¹H-NMR (500 MHz, MeOD) ppm=9.26 (s, 1H,), 8.70 (d, J=8.6, 1H), 8.38 (d, J=8.6, 1H), 8.06 (d, J=0.9, 1H), 7.92 (d, J=0.9, 1H), 7.72 (d, J=8.2, 2H), 7.47 (d, J=8.2, 2H), 3.97 (s, 3H). [M+H]⁺ 363. Rt 1.12 min (method M).

### Example 194

### 1-Methyl-5-(2-methyl-1,6-naphthyridin-8-yl)-1,3-dihydrobenzo[c]isothiazole 2,2-dioxide (65)

### (E)-4-(4-Amino-5-bromopyridin-3-yl)but-3-en-2-one

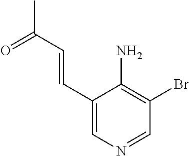

To a solution of 3-Bromo-5-iodopyridin-4-amine (150 mg, 0.502 mmol) in DMF (1.5 mL) were added 3-buten-2-one (0.061 mL, 0.753 mmol), triethylamine (0.097 mL, 0.703 mmol), tri-o-tolylphosphine (12 mg, 0.040 mmol) and palladium(II)acetate (4.51 mg, 0.020 mmol) under nitrogen. The mixture was stirred at 90° C. overnight before it was diluted with water and EtOAc and the layers were separated. The organic layer was washed with water, dried over MgSO₄, filtered and the filtrate concentrated in vacuum. The resulting brown oil was purified by chromatography on silica gel (biotage, CH₂Cl₂/EtOH, 100:0 to 94:6) to give the product (51 mg, purity around 60%) as a yellow solid, which was used in the next step without further purification.

### 8-Bromo-2-methyl-1,6-naphthyridine

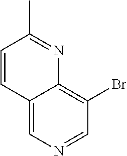

To a solution of (E)-4-(4-Amino-5-bromopyridin-3-yl)but-3-en-2-one (51.0 mg, 0.212 mmol) in ethanol (1.0 mL) was added sodium methanethiolate (15.3 mg, 0.218 mmol) and the mixture was stirred at rt for 30 min. The mixture was diluted with water and EtOAc and the layers were separated. The organic layer was washed with water, dried over MgSO₄ and concentrated in vacuum. The resulting brown oil was purified by chromatography on silica gel (biotage, CH₂Cl₂/EtOAc, 95:5 to 80:20) to give the product (13.0 mg, 28%) as a colourless solid.

### 1-Methyl-5-(2-methyl-1,6-naphthyridin-8-yl)-1,3-dihydrobenzo[c]isothiazole 2,2-dioxide

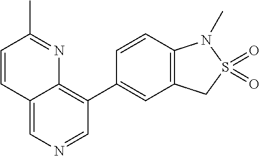 [SMILES: Cc1ccc2cncc(-c3ccc4c(c3)CS(=O)(=O)N4C)c2n1]

8-Bromo-2-methyl-1,6-naphthyridine (13 mg, 0.058 mmol), 1-methyl-5-(4,4,5,5-tetramethyl-1,3,2-dioxaborolan-2-yl)-1,3-dihydrobenzo[c]isothiazole 2,2-dioxide (18 mg, 0.058 mmol) and Pd(dppf)Cl₂—CH₂Cl₂ (2.1 mg, 2.91 μmol) were loaded in a microwave vial. The capped vial was evacuated using high vacuum and purged with nitrogen (each three times). Acetonitrile (0.35 mL) and aqueous sodium carbonate (0.5M, 0.163 mL, 0.082 mmol) were added and the mixture was degassed again by using the high vacuum and purged with nitrogen again (each three times). The mixture was heated in the microwave at 120° C. for 1 h. The reaction mixture was transferred into a flask and the microwave vial was washed with CHCl₃, and the water was evaporated by azeotropic removal with toluene. The resulting brown solid was purified by chromatography on silica gel (biotage, CH₂Cl₂/EtOH, 100:0 to 94:6) and further purified by using a scx2-cartridge (loading with CH₂Cl₂/MeOH 9/1, elution with CH₂Cl₂/1N NH₃ in MeOH 9/1) to give to give the product (11 mg, 58%) as a colourless solid. ¹H-NMR (500 MHz, CDCl₃) ppm=9.19 (s, 1H), 8.73 (s, 1H), 8.23 (d, J=8.4, 1H), 7.79 (dd, J=8.1, 1.8, 1H), 7.75 (s, 1H), 7.45 (d, J=8.4, 1H), 6.90 (d, J=8.1, 1H), 4.47 (s, 2H), 3.23 (s, 3H), 2.77 (s, 3H). [M+H]⁺ 326. Rt 1.09 min (method M).

### Example 195

FRET based Lanthascreen binding competition assay: A dye-labeled ATP competitive probe served as a FRET acceptor upon binding to CDK8 labeled with a strepavidin —Eu-chelate (via abiotinylated anti His antibody). The result was a fluorescence signal at 647 nm. In case this probe was competed by an inhibitor as such a signal cannot be generated any more. The CDK8 used for this assay was a protein co-expressed with CycC.

The assay procedure for an assay in a 1536 well plate was performed according to the following: 2 μL CDK8/biotin-anti-His Ab/SA-Eu mix in Assay buffer were pipetted into the wells of a micro plate.

1 μL compound in 20 mM Hepes buffer/5% DMSO was added. The plate was shaken for 30 sec and incubated for 20 min at RT.
2 μL Alexa647-probe in assay buffer were added. The plate was shaken for 30 sec again and incubated for 60 min at RT in the dark.
Then the plate was read out on a Perkin Elmer Envision (mode LANCE/TRF, excitation 340 nm emission 650 nm).
The assay buffer was 50 mM Hepes pH 7.5 (Merck #1.10110), 10 mM MgCl2 (Merck #1.05833), 1 mM EGTA (Merck #1.08435), 0.01% Brij-35 (Pierce #28316).
The final concentrations of the reaction components in 5 μl total assay volume were: 1% DMSO (Merck #1.02950), 5 nM CDK8 (CDK8/CycC Invitrogen #PV4402), 2 nM biotin-a-His Ab (Invitrogen #PV6089), 2 nM SA-Europium (Invitrogen #PV5899), 10 nM Alexa647-Tracer (Invitrogen #PV5592).

The data is interpreted according to the following:

| Compound | |
|---|---|
| number | IC50 CDK8 binding |
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | B |
| 39 | B |
| 40 | B |
| 41 | B |
| 42 | B |
| 43 | B |
| 44 | B |
| 45 | B |
| 46 | B |
| 47 | B |
| 48 | B |
| 49 | B |
| 50 | B |
| 51 | B |
| 52 | B |
| 53 | B |
| 54 | B |
| 55 | B |
| 56 | C |
| 57 | C |
| 58 | C |
| 59 | C |
| 60 | C |
| 61 | C |
| 62 | C |
| 63 | C |
| 64 | C |
| 65 | C |
| 66 | C |
| 67 | C |
| 68 | C |
| 69 | C |
| 70 | C |
| 71 | C |
| 72 | D |
| 73 | D |
| 74 | A |
| 75 | A |
| 76 | A |
| 77 | B |
| 78 | C |
| 79 | D |
| 80 | — |
| 81 | B |
| 82 | B |
| 83 | A |
| 84 | B |
| 85 | B |
| 86 | B |
| 87 | B |
| 88 | B |
| 89 | B |
| 90 | B |
| 91 | B |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | B |
| 108 | B |
| 109 | B |
| 110 | B |
| 111 | B |
| 112 | B |
| 113 | B |
| 114 | B |
| 115 | B |
| 116 | B |
| 117 | B |
| 118 | B |
| 119 | B |
| 120 | B |
| 121 | B |
| 122 | B |
| 123 | B |
| 124 | C |
| 125 | C |
| 126 | C |
| 127 | D |
| 128 | B |
| 129 | B |
| 130 | A |
| 131 | B |
| 132 | C |
| 133 | B |
| 134 | B |
| 135 | B |
| 136 | B |
| 137 | B |
| 138 | A |
| 139 | B |
| 140 | B |
| 141 | B |
| 142 | B |
| 143 | B |
| 144 | B |
| 145 | A |
| 146 | B |
| 147 | A |
| 148 | B |
| 149 | A |
| 150 | B |
| 151 | A |
| 152 | A |
| 153 | A |
| 154 | B |
| 155 | B |
| 156 | B |
| 157 | A |
| 158 | B |
| 159 | B |
| 160 | C |
| 161 | B |
| 162 | A |
| 163 | B |
| 164 | B |
| 165 | A |
| 166 | A |
| 167 | C |
| 168 | A |
| 169 | A |
| 170 | B |
| 171 | B |
| 172 | C |
| 173 | B |
| 174 | A |
| 175 | B |
| 176 | B |
| 177 | B |
| 178 | C |
| 179 | B |
| 180 | A |
| 181 | A |
| 182 | B |
| 184 | B |
| 185 | B |
| 186 | B |
| 187 | B |
| 188 | B |
| 189 | B |
| 190 | B |
| 191 | A |
| 192 | A |
| 193 | B |
| 194 | A |
| 195 | B |
| 196 | B |
| 197 | B |

D 501-1000 nM;
C 101-500 nM;
B 10-100 nM;
A <10 nM.

### Example 196

### Pharmaceutical Preparations

(A) Injection vials: A solution of 100 g of an active ingredient according to the invention and 5 g of disodium hydrogen phosphate in 3 l of bidistilled water is adjusted to pH 6.5 using 2 N hydrochloric acid, sterile filtered, transferred into injection vials, is lyophilized under sterile conditions and is sealed under sterile conditions. Each injection vial contains 5 mg of active ingredient.

(B) Suppositories: A mixture of 20 g of an active ingredient according to the invention is melted with 100 g of soy lecithin and 1400 g of cocoa butter, is poured into moulds and is allowed to cool. Each suppository contains 20 mg of active ingredient.

(C) Solution: A solution is prepared from 1 g of an active ingredient according to the invention, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilized by irradiation. This solution could be used in the form of eye drops.

(D) Ointment: 500 mg of an active ingredient according to the invention is mixed with 99.5 g of Vaseline under aseptic conditions.

(E) Tablets: A mixture of 1 kg of an active ingredient according to the invention, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

(F) Coated tablets: Tablets are pressed analogously to Example E and subsequently are coated in a conventional manner with a coating of sucrose, potato starch, talc, tragacanth and dye.

(G) Capsules: 2 kg of an active ingredient according to the invention are introduced into hard gelatin capsules in a conventional manner in such a way that each capsule contains 20 mg of the active ingredient.

(H) Ampoules: A solution of 1 kg of an active ingredient according to the invention in 60 l of bidistilled water is sterile filtered, transferred into ampoules, is lyophilized under sterile conditions and is sealed under sterile conditions. Each ampoule contains 10 mg of active ingredient.

(I) Inhalation spray: 14 g of an active ingredient according to the invention are dissolved in 10 l of isotonic NaCl solution, and the solution is transferred into commercially available spray containers with a pump mechanism. The solution could be sprayed into the mouth or nose. One spray shot (about 0.1 ml) corresponds to a dose of about 0.14 mg.

While a number of embodiments of this invention are described herein, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I, 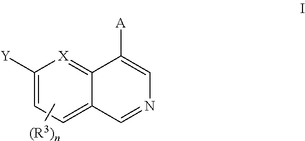
or a pharmaceutically acceptable salt thereof, wherein:
A is hydrogen, C₁₋₆ aliphatic, C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted by R¹ and/or R²; or A is halogen;
X is CR or N;
Y is hydrogen, OR, SR, SO₂R, SOR, C(O)R, CO₂R, C(O)N(R)₂, C(NR)N(R)₂, SO₂N(R)₂, NRC(O)R, NRC(O)N(R)₂, NRSO₂R, N(R)₂; —CN, halogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
each R³ is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R¹ is a 5-6 membered monocyclic heteroaryl ring having 1-4 heteroatoms independently selected from N, NR, O, S, SO, or SO₂, which is optionally substituted by 1-5 of R^{A};
R² is hydrogen, C₁₋₆ aliphatic, C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or R² is halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂; or
R¹ and R², together with the atoms to which each is attached, forms an optionally substituted 5-6 membered heterocyclic or heteroaryl ring having 1-4 heteroatoms independently selected from N, NR, O, S, SO, or SO₂, wherein the ring is not a pyrrole, dihydro-pyrrole, or thiazole;
each R^{A} is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; and
n is 0, 1, 2, 3, or 4.

2. The compound of claim 1, wherein A is hydrogen, halogen, C₅₋₁₀ aryl, optionally substituted by R¹ and/or R².

3. The compound of claim 2, wherein A is

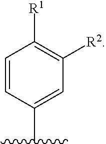

4. The compound of claim 3, wherein R¹ is

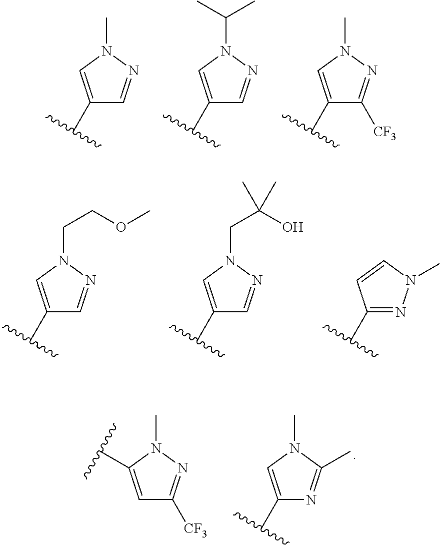

5. The compound of claim 3, wherein R¹ and R², together with the atoms to which each is attached, forms an optionally substituted 5-6 membered heterocyclic or heteroaryl ring having 1-4 heteroatoms independently selected from N, NR, O, S, SO, or SO₂, wherein the ring has at least one heteroatom selected from S, SO, and SO₂; or wherein the ring has at least one or two heteroatoms selected from N and NR.

6. The compound of claim 5, wherein A, R¹ and R², together with the atoms to which each is attached, is

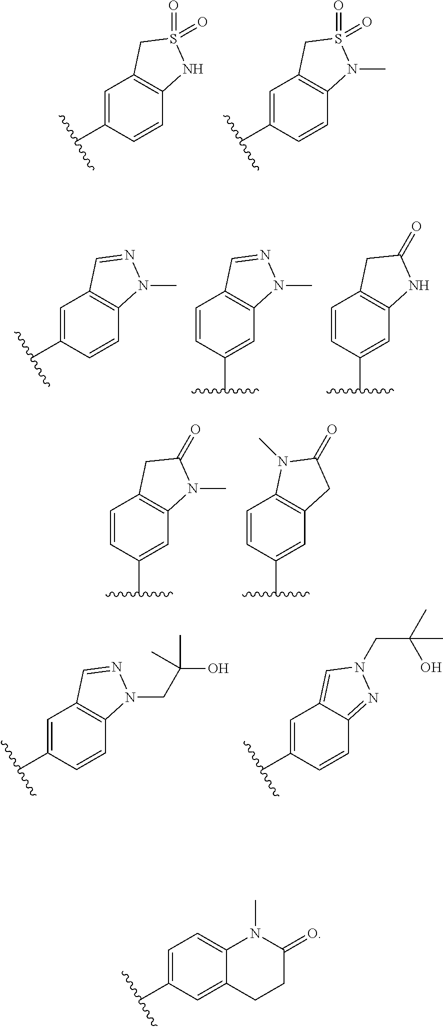

7. The compound of claim 1, wherein X is CH.

8. The compound of claim 1, wherein X is N.

9. The compound of claim 1, wherein Y is hydrogen.

10. The compound of claim 1, wherein each Y is

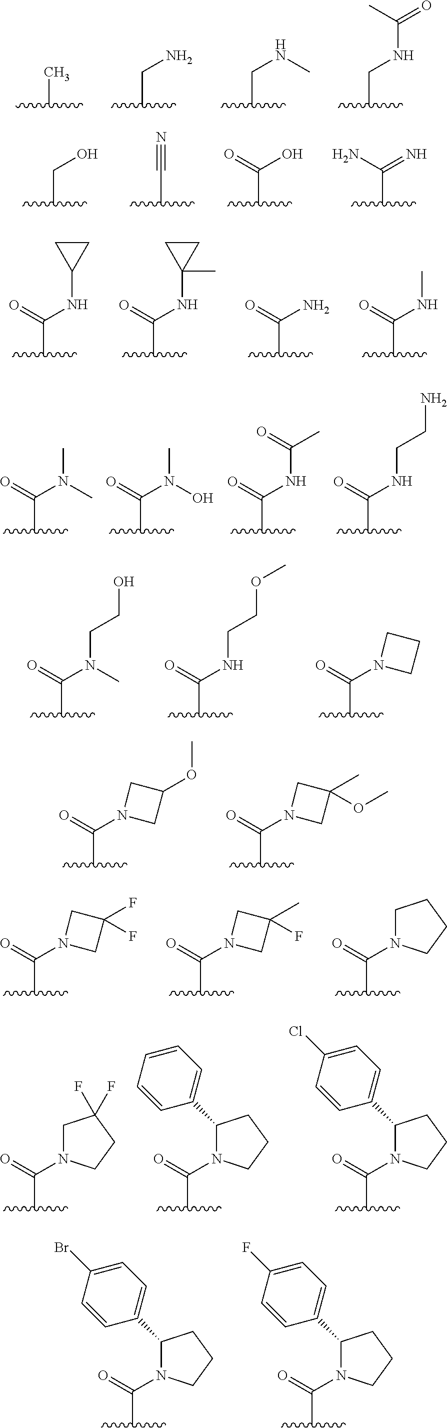

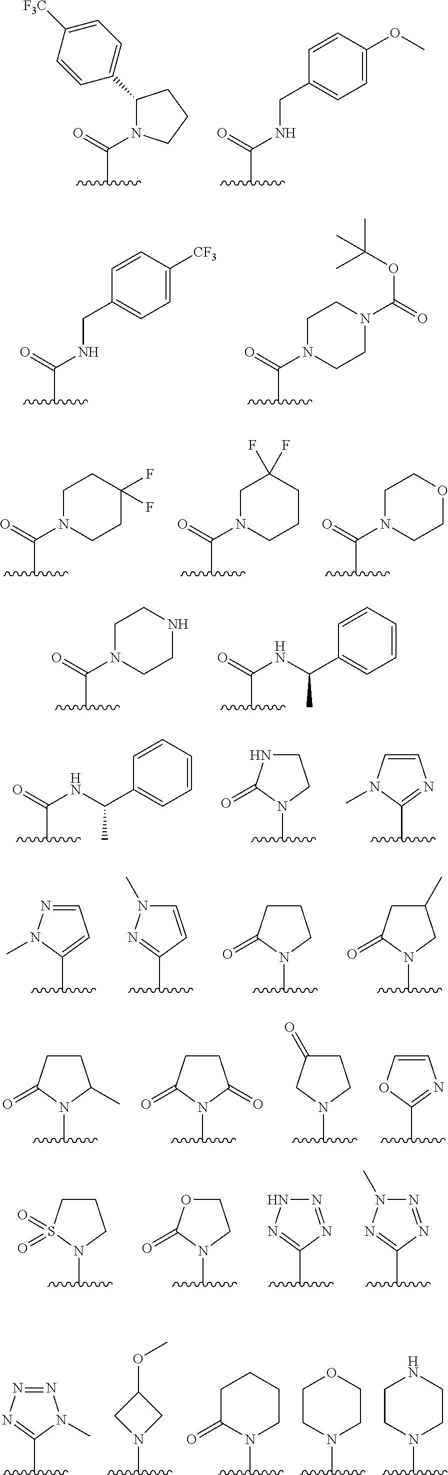

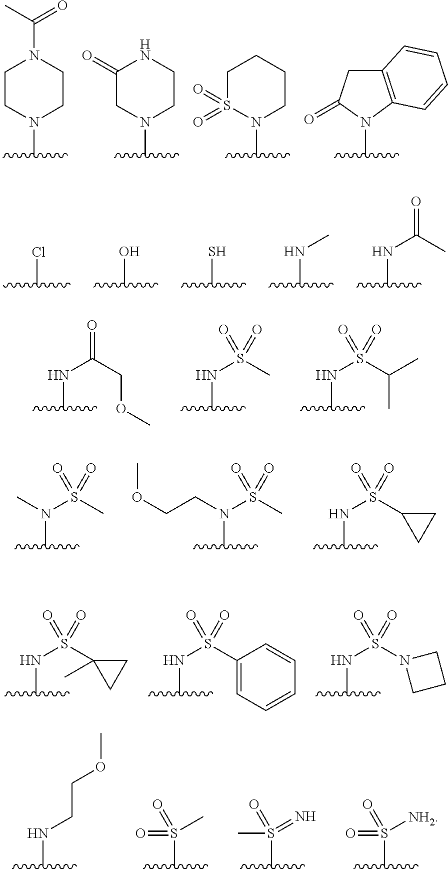

11. The compound of claim 1, wherein each R³ is independently —CH₃, —NH₂, —OH, or —Cl.

12. The compound of claim 1, of formula III, 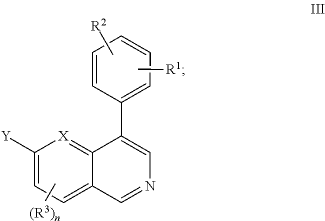
or a pharmaceutically acceptable salt thereof.

13. The compound of claim 1, of formula V: 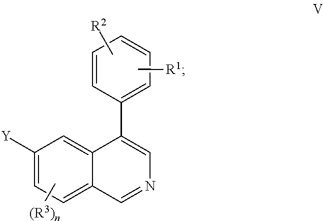
or a pharmaceutically acceptable salt thereof.

14. The compound of claim 1, of formula VI: 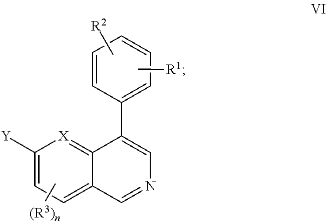
or a pharmaceutically acceptable salt thereof.

15. The compound of claim 1, selected from Table 1.

16. A pharmaceutical composition comprising a compound of claim 1, and a pharmaceutically acceptable adjuvant, carrier, or vehicle.

17. A method for treating a CDX8/19-mediated disease or disorder in a subject in need thereof, comprising the step of administering to said subject a compound of claim 1.

18. The method of claim 17, wherein the disease or disorder is Alzheimer's disease, other dementias, amyloidosis, atherosclerosis, renal disease, or viral diseases.

19. The method of claim 17, wherein the disease or disorder is tumor or cancer.

20. A process for manufacturing a compound of formula I, comprising the steps of:
reacting a compound of formula A: 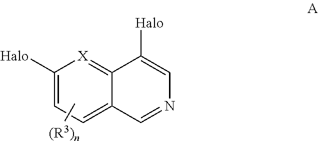
wherein X, R³, and n, are as defined in claim 1;
with a compound of formula H—Y to form a compound of formula B: 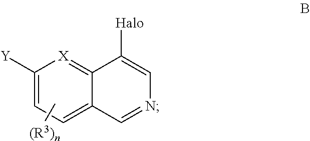
wherein Y is as defined in claim 1;
then reacting a compound of formula B with a compound of formula 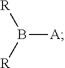
wherein A and R are as defined in claim 1;
to yield a compound of formula I.
